SEQUENCE LISTING (64 sequences)

SEQ ID NO: 1 - DNA, 92153 bp, Artificial Sequence
  Description of Artificial Sequence note = synthetic construct
ccactcgcaatacctcattagaggacaattctctccccaaaaaaggttattttaaaaacctacaaaacccacaatactatttgcataatgatatgaatctacttattaacttcctaagagatttgtgtttccaatctggaaaatggtcttaacatatgagttgttttgcctagtttgaagaacatcaatctattttagtggttttaaactctttctacattttttgcattaaaaatacttttgaaaatctgaggaaagttatgaatttcctcctcacacaaatatatatagacaaaattttgcataccattttaaaggttcatttttggaaatataattgacacaaagggagttacctagaaataacatataatagctttgtgttattacatgtaagattttttctcttaagtcgatgatttgttagatgcaaaagttaaaaatcctcacctacatggtatgaaataatatggataagatagtttactttaacaattttagtactttttcaatcttgtaaattcttcaagaaaatatgggtaaacaaaacagttaaacattctaattatactttataaaagaaaataattagaacctcacaatttttcttttaaatttacacactatgatgttataaatgtaaggctttcagaagaacattataaaaactcaagacctcataatcctgaccaaaacaaaaagacataccacaaaatttaattctttaaaagaaaaagatgacattcactttttatgaaagagaaggggtttcaccatccctgccaatatcttgggtaattgtgcagcaataacttcagacatcatttctggaaattcaacactcagtgcccgggactggataaatgtattcaagcagtacagatgaagttgtttgacaagctgttggtttaacaaatgagtagaaaacatgtaataaaaataataaattcatatgctattcaaacatggcaatttatttttctaactatttataatataaatacttctcaaggtgtttttttcttaccatagtgaaaataactcccaccattttctagaataatatgtaaaatgttatatactcatgatagaacattctgatattatggaaagatttatttaaagaaatgaaagttattcacaattctacaggataagctgtgtgaacattttggtgtattactcaagtttttttttctttttatacatgtatacagacatacccaagcatacacacaatattacacatgtttcataaattctatttgctaatctagctgttttatataacaacatattggttcttcctatgccaataatccaacatcgtttaatttttatcaaagtaattcatgtgtataatgttgaaagtcaagtagtaccacatgattataaaaacaaaaacagcagcagcagcagcatcaatagagcctagcagtttcctgatagacttcttcccatccctattccagctccctagacacaactaccttcaactttttgtagcctgtgtcttttgatatttctatatttccactaaagttagttttagtttctactataggaaatgaagaaaatgaagctctcttatactagacactctcttcctcaactacatacatgttctcttctccccagagagagcttttggctcaatttttagctaaagtcaatatccagcatttatatcataaagatttttttatggttttctgctggattgcatctccattcttacgacttatttttattttgcaaaagtgaatcacctcgtgttcttgtttgctttgttttctctatatttgtctcaaagccatagtcaaattctgcaacagaattgtaaaaatcacctcaattacagtcatacttagtggtaatctctcagttccatttatttttcctggagctggtatcctagaaccctttgtattactgctcccatctagactaactactgctctgcacagttattgttctgggacatcactttgcatctctgttggggtggatctttggcttaatcccatgttttgttataagacatcctgtagcagttcttgagaaagttgtatctgaggtaaactctaggagaccttgcaagttggaaaatactttcatgctgccattgtacttgtttgatggtttggctaggtatgaaattctttgaagattttcttttccatcaaattctgaaggcactgctccactgtcttctggcttccagtgttgctgcagagaagtccaacatcattctgaatcctgataacgtctatgtgacctgtttctttcatcactggtatactttaaaatattctcttcatttctggcctggtacaattttacaataatgtaccttagttcaattgtgcagtgcctctacaccggaccctttttgttagaaaacttgtatgtttcagttccagcaacttttgcgatttcttacacaaatttctctccttgattttctctattttttcttctggacattcctattaattggaggctagaatctcctgaatactaaatttactataactcttctttctaattttccatgttttgatcttttatttttcctcagattttcctttccatttcagccctttcattgaattttcaatttctgcctttcttcatttaattatccaaaagtattttcttgctaaatgtctgttttaaaaatgttttctgttctcatgtcatggataccgtatcattcttatttctctgaggatgttagaagttttcttctgttctctgcactggtgtttatttccttcaagtttctttttattttgctttctgcctttcattttagaggctttactcaaatagctcataatcttggctgtctgttcgtattcaggagtaagacctcaaaagttgacctggagagtggatagcttttttatgccttcactgtcattttaatgtgatctgaacatagtagcaataaaagcttgtatttcatatatcttgacaaaccacagatatcttgtcttagtaatccacctctggtgttgggcatttcttgtttgcttttcttttttccccctctgctatggtgaacatccttgcatgtatattacatttgtctaattatttctttaagataaatcctaaaaaatagaaacttgaaaagcatgcaaattttaacttttgctatgtattgcctgattgcccacccatcagcaatgttatttataattccactgaaaatggcatgagtcttatttttcacatacatacctttgtttttactgtatatataaaatcttttataattttaggcacaaatattttagaacatattcaggaaatgtaccataatttctatcagctattaaattaccctcacattatacaaatacttgtaaaagaatatttgagtggtctatattgaaaatgaaaaaaaaacacaataaaaagtctgagcagcatgatccaaacacttttaacatacaagtattaatctgagaaaatcatacaaagtaaaatttacatgtaacatttaaaaagttatagaaattcagagaataatcattgatattctggaatcaaccaaatacttacatcatgcaagttatcaagaagttttgtaagttgatagaaacgctgtgagctcgacacaactcctttttgcctcaaaccaattgccttgatgagctctctaatgtagcttgacctcatctcctcaaactgggtttgacttcgtagcccttccaaaggaactgttaagaagacaattaaaagtggcagttgtgtataaaaagatgacattaacaatttataacatatatttctgagctatataatgatttcttctgatacatgactctagaaaaaaaacaccttatataggaaagagatagtgttattgatatcactatcaatatcaggacatataaacttctagttcactggaagaacataagaattaataggtatattaaagaacacttcagctgacctattttagacctgtatcaatggattgtgcatttaacttagcatttgctcaaaagagaacctggccttattctacatgcttcttaaatacttggtttgacttagtttacagctgcatgtaatctggatcttttttttcccttctttttaatgaatacttggatctcttagtaaacttttgtcttctttatataaatctcaagtatttgccattaagattattcctagatgatttatctttttgttgtaattttaaaaattgcttcattaagttttctaatgccttcctgctgatatgggagaaaggtattaatatttaaatatttatcttatatccagccactttgttacacaggcatatcttggagatatagccggtttggttccagaccacggcaatgaaggaaatattgcaacaaagtgaatcacatgcatctttttgtttcccagtccacgtagaagttatatttacactatactagaatgtattaagtgtgcaacagcattatttaaaaagtgaacacaccttaactaaaaaacactttattgctagaaaatggtaacaatcatctgagtcttccctgagtcaaaatctttttgctgttggagggtgttgtctaaatgttgatggctgctgattggccagggtggtggttgctgaaggttggggtggctatgacagtttctcaaaataaggcaacaatgaagtttgctgcatcagttgactcttttgtgaataatttttctgtagcatgcaatgctagcagtttgatggcaatttacccacaatagaacttctttcataattggagtcaatcctcttaagtcctgctgctgcattatcaactaagtttatgtaatctccgaaatcctttgttgtcacttcaataatcttcatagcatcttcactagaagtagattccaactcaagaaaccactttctttgctcatccataagaaacaactcctcatctttcaagttttaccacgaaacctcttcaattcagtcatgtcttcaggctttacttctaattttagttctcttgctcttttcacatatctgtagttatttcctccactgaagttttgaacaccctcaaagtcatgcaagagggatgaaataaacttcttccaaactcttgttaatgttgctattttcaccttctcccatgaatcacgaatgttcttaatggcatctagaaaggtgaatcctttctagaaggttttcaatttactttacccagatccatcagaggaaacactatctatggcagccacagctttacaaaatatatttcttaaataataagacatgaaagtggaaaattactctttgatctatgaactacagaatgaatgttgtgttagcagaaatgaaaacattaatctccttgtatatatccataagagctcttggctgactaggtgcattgtcaatgagcaataatatttgaaagtagtattcttttctgagcagtaagtctcaatagtgggtcaaaatattcagtaaaccatgccataaatagatgtgctgtcatccagctttgttgttccattcataaaccacaggcagaacagattgagcgtaattcttaaaggccttaggatcttcagaatgataaatgagcattagcttcagcttcaagtcaccagcttcagcttcaagtcaccagcttcattagcccctaacaagagagtcaacctgtcatttgaagctttgaagacagacattgacttgttctctgtagctatgaaagtcctagatgacatcttcttccagtggaaagctgttttatctaccctgaaaacctgttttttagcacagctactttcataagtagcgataatcttacctagatcttctgggtaacttgctgcagcttctgcatcagcacttgctgcttcaccttatactttgttatggaaatggcttcttaaatctcatgaaccaacttctgctagcttcaaacttttttctgctccttccttacatctctttgccttcctagtattgaggagaatcagggccttcctctggatgaggcttaggcttaagggaatattgtggctggtttgatcttctattcagaccactaaaactttctccatatcagcaataaggcggttttgcttatcattcatgtgttcactggaatggcacttttaaagtccttcaagaactcttactttgtatttgaaacttggctagttgcttggctcaggagacctagttttcaacctatatcaacttttgacatgccttcctaattaagcttaatcatttctacctcagacttgcaactctttcttttactggaacactgagaggccattccaagattgttagttggcctaattgcttttttttttttttttttttttttgacacagggtcttgctctgtcgcccaggctggagttcagtggcacaatctcaggtcactgcaacctccgcctcccatgttcaagcaattctcccgcctcagcctcctgagtagctaggattacaagtgtgtgccatcatgtccgactaatttttgtctttttggtagagatggggtttcactatattggccaggctggtcttgaactcctcacctcaagtgattcgcccacctcagcttcccaaagtgctggaattacgggcttgagccaccatgcccagccttggcctaattccaatattgttgtatctcagggaacagggaggaaaattactatttcttaagcctgtatttatcccctgcaccccaatcaaaacttggtttccatttcccctagaaaagaaggccagagatacaaacttatgaaaaacaaataagttaattctttggttttgagttttcaagatcttgtatataaaggagactctgaagagtaattctggcaattcagtatatctatgcaaatttttgtcaattgaaaacttaactctagtatgtacatctatatgtatatctagaacctctttttattaaatttgtataaatttaatgaccatctgtgcagttttgttacatgaatatattgcatagtggtgaaatctgagcttttggtataaccatcacccaataatgcacattgtatccattaggtaatgtctcacccccctcctacctttccagccttctaaggcaccactgtcaattattctatactctatgcccatgtacatgcattatttagctcccacttataagcgagaacatgtggtatctgactttgtctctgaattgtttcacttaagataatgacttcctgttccatccatgttgctgcaaaagacacactttcattctttttgattgctgaatactattccattttgtgtgtgtgtgtgtgtgtgtgtgtgtgtgtatgtatgtatatgtgtgagtacatcacattttctttatcctattatctgttgatacacacttggattgatttcctatctttgctatagtgaatactgctgtaataaacaagagtgcaggtgtctttttgacataatgatttcttttcctttgggtagatatccagtggtgggattgctagatagaatggtagttatatttatagttttttgagaaatctccatatggtgttccatagaggctgtactaattttctggactacttcaaatagcagtcatttcaaactttcacagctctctttaaggttaccaaccaatccctatatttccctcagctgtaaacatgaatgtaaaatcattttttcttaatttttcatagtaaatccctttactaagattttagtgtaatttatttatttttatttatttatttatttatttttattttgagacatggtctcactcaatcacccaggcaggagtacagtggcacaatcatggctcactgcagcctcgacttcctaggttgaggtgatcctcccacctcagtttcccggctacctgggactacaggcacttgccactacgcctggctaattttttttttttttttttttttttttttttttttttttttttttagagacggggttttccaagttgcctaggctggtctcaaactcctgggttcaagccatctgcccagcttgccctcccaaagtgtgattagaaatgtgaccgactacatctggcctttagtgtaattttggagagaacaatgtaaaaatgtttgttcaacctactgtctattcctggagatttatattttcatatttaatggcaaatgagaggtttaagttcataactaccaaattatcaaagttttactttgccaatcttctccttgcatttattttataaaaggtatttgagattgaagattatttctatttcttctagcttgttttttaattatttgagtgtttacatttaactcaattatctgttattcaaggatataatttgagctacatatccaaacagtttttttccaaatataaaactatttttcttaacatttatttgtttcagactcattgatttgtggtttccactatactatttatattctaatatatacttgtctgatttgcccaaaagttcagtatatctcttctcccatgctgtattaattaacagcagctttctaattccctgcatatttttataatgcttatctatttattcttctagttttaaagatggattttcagtaggtttgtcaagttcaccctcttctccccaaatctcattagaatcttgattagaatttgtggaaagcgtaagtactattcattaaatagtttctttttcttcctttttgtgaagtggcaaatggctaaaaatcttactctctatggtatgtgagtggaagttgtatgaatctcacttccttttcttaccactgctctagtgatcgcctaagtttctaagtgacaacaatgagcaaagcctccctgttgattaattccattgttgtttaattccccagcataatgttgcctatccataaatagagaattatgttaaggatataagccagtttggaagatctaggactatctgtgacataagtgctataggattcattccttcatgcaaagaagggcaggagctaattactgtattattaaatgtattccagttgactctctgggccggttccacgtaaatatcctttgttcaattgatgctctgtcaccaaattagccttgaaattccttgtccaaatagggttcccaagatggttatgtattcatccctgtctttgcataaattctatgaggaagccttccatttcagggtttatctctcctgcccttccctcctgatctgcaccccctagccatttgagatttgggaccttagaaagaagtccctgtaaagatgttgccaagaaagaccttaggctcaccctcggcctgaggtcttagctccctgtggttgtcatagaaatcacaatgaatcacaaaagcatcagtttgtacatttgagggctgatcctgcctatgcaagggtcaaagcagctgctttttcttttttttaaaactttgcccacccagacccttggggatccactgaggcagaggtaagtatgttccttggatataagaaaaagtggaacggatatttgcctgtccttctggtgatcaatgcaatgctttataaaacataatttcctctttcttctgggcctatagctagactgtatttcccagcttttcctgcagcttaagtgccttggaatgtgggtgaaagtgatgtatgatagtttcagatctagccttcaaaatctctctcactcagtgctcatttctagtcatgtccaatagaggtttgcaagccactaggaaggggtgaagccactggctagaaggagcctgggtgtttcttgctaactcactggactgtgtcagtgataaagaaaccactattatattaagatgctaatctttatagttacatgttttaacaatttatcttgataaaaataattctaaacaaaattatgtattttatatagtaaatactttggggatggggtgagagtggtggtagaggagagtatggtttgtgcttatccaatattaattgcagtcgtgtatcacttaatgatggggatatattctgagaaatgtgtccttatgtgatttcatcactgtgtgattatcacagagtgtactcacacaaacctagatggtatagactactatacacctaggccttattcctaggctatgaacctgtacagcatgtgattactgaatactgtaggcagctgtaacacaatggtaagtgttttgtatctaaacacagaagaggcatagtaaaaatccaatataaaagatacgaatgggacacttgcataggggacttaacatgaatggaacttgcaggactggaagttgctctgcgtgagtcagcgagtgagtggtgacagaatgtgaaggcctaggacattactatacactacgatagcctttataaacactgtacacttaggctacactacatttataaaaatatttttatttcttttataataaattagctttaccttaccgtaacttttttactttataaactctagttttttaactttttgactcttttgtaataacatttagttaaaaacaaaaacacaaggtacagttctctttctttctttttttagagacaagttctcacatgttgcttaggctggacttaaaactcctgggctcaagtgatcctctcacctcagcctcccaagtagtggggactacaggcacatgccaccgtgcctggctttctttctctatatctttattctataaactttatctatttttaaatattttcttttttgcttaaactttttttaaaaaaactaatacccaaaacacacattagcctagacctacacagagtcagaatcatcaatatcactgtcatccaccccaacatcttatctcactggaaggtcttcagggggcagtaacacacacggagttgtcatctcccagggtgacgtatgcccttttctggaatccctctgacagacctgcctgaggctgttttgcggttaacttaaaaaaagtagaagtagtgactctcaatagaaaagtatactacagtaaatacataaaccagtaagagttttttattatctttaccaagtactatgtactgtacataattgtatgtgctatactttatatgactggcagcacagtaggtttgtttacattagtatcaccacgaacatgtgattagtgcattgtgctatgatgttatggtggctataacaggactaggcgatgataatttttcagctccactatcatcttagggaaccattgtatatgtggtctgttgttgaccaaaatattattatgtggcacatgactgtagtttatattactgaattaggtgaatatgaaagagtaattaaatcattttatctaaaataaaaagaaattgatgaggctattttagtattatatggtaggtaaaagatgaaaaaatccagtaattttattaatgtctataacaatatattgggtatttcttctttatacttataatccaagacgtctacctcatacataactatataatcatattgtttgcaacttttctaatgaattaagaaactagatatcttgcattaagttacttgaactcactcacttgtattaagaagtaacaatactttcatacagaggaactcttcttggctaacttgaagcttgacaaactcctgtgggatctgccacatggtaaggcataatgaatagaatgatgattctttcatccgctgtcttgcatcacacacaatacacaaaagaaaaagcaacaggaaaaaaaatagtgtctcagccagtttaggtaatttatcagggaatatggttttggtaattaccttacatattattgaaaatgactactacttttaataaacaaacccaatatttggaattttaaacatcttctaaacttttcacaatactgaatttttgaggttaagaaatcttaaaactagaaaattatattaaatacacagtgaccaagattaacattgtttctcacttttattatcacactttaaatacctaaaccaagaaaacacagcagaggtgtaggagttgagaatgtgaagctgaagaatgctcagaaatgttttatttacttacaatttattttctatgaaaaattatataattttgtgtgcaattaaataggagaagtcagttcacaaagcttaccatattttaaacttttctagggaaatatacaatgatacagaaaagagtaaaagaggacattggcaagcattaattaattgacaaagttaaacacaagtgctgaaggctgagaaaatttaattaaggcattttcccaaccttgtgttgttttataatgtccacattagtcaaatgaagctaacaagaataatgaaaactagattttggctaaaaagagataaggaatctcactgtcattcaaattatttggcagattatataattctcctgagactcagttttctaaactgtacaatgaagaatgctgattccttacttatctcaaggaattgttggataaaatgtaatgaaacatgaaaattatctaaaaactgtaaagacttatataagtgtgagttatatacttctaagtcagcatttaccactagaacaatataaatattaccatgaaaagattcttattcagttaaaggttgtaggtagaaaattaattaaaattatttaaatataaagcaaattgaaatatttatctttcaattttttaaataaattagcagtctaatttgtcatgtatgaagaaagatgtttttactttttgaatgtaagtcagtaacaaaaatatttctcatttattattgtcattagtaccaagctatgaaattttaaattgattaatgaaacatttaagtttatctttaaatatatttcttgatacattcttgatgtgtaagaaaattggatttgatataactatattttgaaacaagaggaaaaagctccaaatataaaattaccgtaaaatgacagtaatgtcatcatatcaagaacactaaatatgtgatcatctattgaaatataactttcaaaattatcctacatgtacacttaaaataacaaaaacaacaaaagttactacttactcatttagtattagatcaggtgcaaaatacagcatctgcccactgacgtgtttgtaggatctccatcctagaccaaacaccattaagctcatccaagaatactgaatgagagttatctggtcatcaatatgtaagtttcgaaaacctacaaaacaaatttaaaaatacagtgaccataaaaatgactgaattatctcaaaaatgttgaaaatacatataaatctgaaaataggtatgcgtagggtaataaaatgttttcaatcccaacatattttacactgaaaatcagaaaacattaataaataatacagcctaaaatatcagcaactttggaagtcacttgaaattagtcactgtgatttagcatttctgggattttgctaagtgaatccatcatccatcactcattcatgcaacacccatttaataagaaccatttgggtgccaggcactgtataaaaatgaagatgagtaagagtcaatcctagcctcaaaagcagctcacaactcaacaggagaaacaagcacatgagtgctggggtagagaaggttaaaagggagtgtatcagtcaatcgggagggaggtcaggaaaaactttctagaaaaaaagagagataatgcctgaatggaatataatcagttcgttaaatctcagttctttgggatttaaacataaactggtcttactttgtaaaaccactgctttcagttgtgttatatctaattttcttttaacaacccttaactatgtcgggtagattccacagtcatcaggtcactttcgttctctgaaacttaatccagtcttgttttaaacttccgtgtggcttaagagatttagaatctgtgtgtgtgtgtgtgtgtgtgtgtgtgtgtgtgtgtgtgtgtgtgtgtgtattggggggtatggtgtgaatgctttggtctttcatcctcttttaggctgttccatggtgttggtggcagaaggatggcagggaaagggcaaatgaatcttgacttaatcatcacaagtctctccatgtcttcccaattctcaagggaagatcaccagttcaacaatgagtaacttaaaggtgggagaagcaaagatgtgtttagagatgtaggctgggtcagatcatccaaggtcttgcaagtctattaaggagcaatgggagtgacatagccttgcattgtagatggatcattcttagctatgtggtaggtggattcaagggaataagtctggagttagggaggacagttgggagatactgtcaaagcccaggtgagagatgataaaagcctgaagtaagtcaagtcaacacattggaattaagaggagagagtgcatcagaaaaatatttaaagggaaaaataaaagacatttgtcaactgattgccggtagatgggaagaagaaggaatcaaatacatctttcagtatttgtcttgaagggtagatgatgataacagcattgctgagataaaataaaggagaagaaacttgctagggagaaaatagttttggataggattcaggggtcaacatcctggtatcagctgcacatatgaattagaagtatggggagatatatgggctaaaaatattaatataaatttgaaagttatttgtatctgagtataactgaaatgatgaaaatggagaatttttaaaattaaaggtatggagacaatgttttgctgagaacatcaaccactttctataaagctgttttaaaaacatctattttctctaaaaatccactgatgttcttacaaataatctgtttcaaaagtaaaaccaaagcaaaacttaattcattagctatcaagagcaaaagaaagttatttcctgacaatgttaatcatgtcaaggtaggtagcaaaaaagaaggctcttctcttcatacacttcagatcgtcccatcttcatgtgaatagtagaaaaattgctactctcttcttagaactccttagcctcaataagaccaaaaactcaaatgcatattcataattagagatgccatagtgagcatttaccttttactgagaagggatgctcttcaatgtctaaccttttctccttcccacctttcctccctccccttctccctcctttcttccctccccttctccctttcttccctccccttctccctttcttccctccccttctccctttcttccctccccttctccctcctttcttccctccccttcttcctcctttcttccctccccttttccctcctttctttcttccccttctccctcctttcttctctcactcccttcctccctccctcctttcttccttccctctctttcttcctttcctctttttcctcctcctccctcactgctccctaataccttcctgctttccttctttccttccctccttccttcctcccctccttcctttctctgtgtgggcagagttccccattggttgtagtttttgcgagagcaaatgccttaccttcctactttcccacggagaaagctgggtaaacctcctccttctttgggcttggggcataggtttggcccatggaacatatactaacaggaatttgaatcagaaaatactaaagcaaagagaaagatatgactatttattcttgatgttggtggtgtccagataatgagaaccaatgggaggatttgtgatgcttgctttttagcccttcagatgtgccttggcttctgcccatattctaagcttgtcctctagcttctattcagtgaacccctgttgtcctctcagtaaattattttttttctttttcttttgtttttaaaagtcagggttttttgttgttgcttgtgtgttttataattaacatagtttatttttaatactggcatccaagaatcctggtttactcaggtgcagaaagactctctaactaagcagccaaaaaaatttttggtatgcaagttttatcattttttaatttgcatatgacttgaacgtgtcttcaagtataggtctacataataactttttaagaaaattataaagctcaatacaataaatctaatacataaatgctgcttgtaagtcaaatatttaagagactataaaaatgggtaattttgtgataaaatttagaatcatttgacaagagatcaatgaattgtcttaatttttgaaaaggaagcctttctaaagggaggcacatataaacctccctattgctgatctgtctcaacaaattggaaggcattcaggacagaagacccaagttccagtcctggctctaccacttactaacagccactttggaaacatcacttattatcattgaatctaaatttcttcataagtaaaaatcacaattcacaatgataactaactcagtgagttgctatgagacaaatgggataattatgcacactgtagaacactatggagatatttattattggtctatcaatgcaacagaattgctcattggagtaatactagtatgaaatccaccagtcagtttaatgggaagtctctaatactaaaacacaatgccataaaaaaaagtgttaggaggcattttatatattatagaacatataagatagaactgaagtataaaaaataatggtgaaaaaattttaaagataaattatgtgtttgttctggaatatatattttccagaatatatattttatgtgtgttgttatatataacaaattatatatttcatataatacaaaaaaatgggctgggcgcagtggctcacgcctgtaatcccagcactttgggaggccgaggtgggtggatcacaaggtcaagagattgagaccatcttggccagcatggtaaaaccctgtctctactaaaaatacaaaaattagctaggtgtggtggtgtatgcctgtagtctcatcagctacttgggaggatgaggcaggagaatggtttgaacccaggaggctgaggttgcagtgagccaagaacgtgccactgcactccagcctggtgacacagcgagactccatctcaaaaaaaaaaaaaaaaaaaaaaaaaaaaaggctcatagaaagttcgttttcattaatcagatgtattaaaacattaaccaagtgttcacaataatctccacaaatacatttattattctctatatctttcttggtgaactttataactttctaaaaaaagtattttataatatttaaatatttgacttaactatgctgtaatttttcacttcaggcatttgttttatgttaacctttgagtagttttaaacatttaaacattttacaaaattatgtaagaattaaattgttctttatccaaaaacagatcctgtctacatttacatgtttacattcactttatcctactcttagtttttgttttcgtctttttgcttttttctttgtttttgcattctgggactagaaatccaggcttacttttattttatgacaacattaaaggcaaatcttattacaataactttaaatttataaaattaccaatatttatagcagaaactatatttctgtttgtttgacagaatggaatagaactcagaaaataacctttatttactccaccttttacagtgcttatctgtgataaatgtacaatgttaaattgacttgaacatgcctagtatagaaatgctatttttttccttattaactgtaatatcattcattactttatgctgaaacaaagatgggaacacagagattaaattcccagtgatgataatgatcaggtaaacttagtacatttgagatgaccaaaatgaaaaaccttcacatggagaagtgatggattatcactaattgtgtttaacaaatgttaaaaaaaaaaacacttttccttttaagtgacttgacctgataatttctgacaaaagccctgttacagctgtccaaatttagcttaaagaattaacagaaagtatatagtgaagagatggcctggggctgggtgtggtggctcatgcccgtagtcccagtgctttgggaggcgaggcaggagagtcacttgaggccaggaatttgagaccagccaagacccagtctctacaaaccaatttaaaaagcttgtccaggtgtggtggtgcacacctgtagtgctggggaggctgaagttactggggaggctgaagcaggacgatggcttgagcccaggagttctaagctgccgtgagctacaatcatgccactgcactccagcctgggtgacagagtgagagcctatctcaagaaaaaaaaaaaaaaaaaagatggcctgtatgacagtactactaagttccccaatcttactcccagagttttgataaaatatgtaggttttggttctgtaaaaaatgtcatgtaattgaaacagataatctacaacaatggcagagcattaaatatctcagcaccttctgacacaaagcaacaactttcatacacaagtatcaatgttgagaagcacaaagaagcttcaatatgattaagtataatcaccaacgtatgtttgatgatttatgctgaataagaagttaatgtctaataatctaaggtagtacagttctttgcaaatcaattctgtaatttaaatttctataaatattctttaaccatctatttgggaggaagcattatacttgcaattaggatcaaggttagaaagatgctgtttctgcctttaacactataggcttgtgagacagatagaaaagtagaacacaattgtaactcagagtcatagtgttatggttgacaagcacaaaggagacaagggagggtacagggtttcctcacccagtctaggaaggctcagagaaatcttcccagagcaagtgacaagtaatgcgaatcctgaaagatagttacaggtcagtcaaaggaagacgtgggaaaagagatgggctgccaggtaggaaaaataatatgggcaagagaagaaggctagaatttgagcaaacagcatttagggaactagatcttagagtcgtgtgaagaggaatgatgagagattaacactgcaaattcaggagccagatcacagaaagcccttggatattgaggccatggagtttggatgctatttggaaaaagaaatggaaaatatgattgattaaaattgttttagaaaaatgattctgggggtagaatagaggatcatttgggcaccacgtgaaattgatggtaatagaatcagtcaggaggctgcaacatgaatccaagtaaaaaaagaccagcaccaaactaagtcagggaagtagcaacagaagtagcatgtataatgaatcatatatttaataagtggagttgatagaatttgacaatttattgggtaaggggtgaaggagaaactgagccaaggatgataccaaaaattcttgctgaggtgccatttcaagatactggaaaaggccttaggtttagggggtcatgctgacttagaagtcatctgggtggagctgtgtaataagcagttgaaagttcagtagttttgtagagggtctggctaatattatgtatttaggagtcatcgaaatatagtggtagttgaagacaagactatgaatgagcttatgcagactgtgtagagtgagaaatgactggaaaagatagaacccagaacacaaacaaatttatagtgtgggtggaagaacaaatccacacaggagtcgggaaatgtacatagggagagaaacatatctacctggaagggaagtgacaaatacctaagcaagagtcttagcaagaaatagttactttgccaatagtcccagaggtaggtgaaacacattaagggctggaaatattcatctaatttgacaagtaaaaatttttgttgccactggcaatcataatttcagtggaatagggtgtgagaaaaaccatggtttagtctttatcaacacatttattaaggcctttactctcagtatgctactctcatttctctctcatcatttttttctggagagcaataatttaatttagtcttctgattcgggaaggaagagaaaaaatcctctgtcaagatgcaatgactcactgactcaaaagttgttttggcacgcttcaaaagcagaaaaatgaggcaaaatactgttctaatattctgatactgataattgcaagtagtctggtgaaatgacacaaagaataaagtatgtcctaaaaaatgcaaggtttaaaaattcaaagcattgacctaaatggagttttcactaagttaactgaaatatcaagacaaaatttatgcaacggaagtgtttccctcataccccctctgtcacatcaatttcactgcaacgtattgatgtacaggatgaggacaatttaaaggtgacaccgaattgtgaattagtacttttaatttaaactctaacatctacagtaatttttaagaccctccttgatcagcctataacttacacctctaagtttactttccattattcagttcaaaataaatttcttttcaatggtttcttaccatttgggtgattaaatctcccgaaggccagtacttactgtagcagtgtccgagggactgtatgtacaaatgtgttttctcaatccatagctactaaaagctttaattactaccctatggggagactataaaatttttgatgttactaagggaccccccacacttgaaaaggttgagaaccattaatatgctggtttaatgcattattcagcaaacctatctcatgcttttcttatttcagccatactgttccttctatctagaatgctttctcttatcatgatttatcaaagtcttgtttctcctgtaagatgcagtttaactgacatttttgtctctgaaggcttctttagtccttgaagccaaaaatgttcttctgtccccactttcattatatctgtatcttatatcactttcacataacactatagttgtttgggtatcttctctctgctactggattgagagttccttaatagagggaatatgtcttgttcattaaaaaaattcaaatatcgtgttgctctacatttcaaataagaaaatattgcccactaaggctacagataaaagcccatgcaaaatcactcatttgcaatgtatcctaagagatggtttataaacgtgctgacataaggctagagcatcttgcaagtctctattaaagtaaaaggggtggggtgcggtgagtcatgtttgtaattccagcactttgggaggctgaggcaggcagatcacttgagtccaggagttcaagaccagcctgggtaaaacagttaaacccaatttcaactaaaaataaaaaaaatttagccaagcaaggtggcgcatgcctgtagtcccagctaccagctcatcaggaggctgaggtggtaggataacttgagaccaagaggtccaggctgcaatgagccaagatcatgccactgtggtccagcctgggcaatagagtgagaccctctctcaaaaaaaaaaaaaaaaaaagaaaaaaaagaaaagaaacaaaaggatatatctgaaatatttttgaagaaatataaactaccattattataacagatccaagataatggaagagtgtagacaaagaagaaatgagaaagccctaccaggcaataagaattcttagggcacctgcaaatatatgcctaggtgtcttcataaacgctgtcacctttgttatgatggtcattctgcatcagatcttaatgtgactttccaaatacagtttcctattgtgaaagaatgatctgatctgttaatatattctcctagcaacataagtattatgtatatttatattgtgtgtgtgtgcgtgtgtgtgtgtgtgtctcactgtaaataagcaagttagttgacattctggctaattagtgaggatcacttaacaaaagtctgtcttccacagacttattagttttattacacatgagtgaatgaacgaaataaatttcttttacatgcaaaactgatgaccagatgctagcatgtgctgtaccaaaatgcatccacaccatgagcccagccttgtggcttttctttctaaaccactgtataattattagatattgaacacagcaggtacataatagacatttattgagtgaataagtgaataaaaactgtaagcttatttcaagtcaattgaaatcagttttaagtagctaataatactaaagtactttctatacatgtgtccatccaagtcctttctcagaggtcaaatagtcactctagctcactgaccattttcataattcattcaatagtgctattcccaaaatgcagaccatgaaaatatggctttaaaagtccacaggccacaagagactttgataatctcaatgtttccctgtttgcaattaatggaaacagtatgggtaatactagtgttgccattattctgatttactacacaaaaattacattttgcagcattaaaaaagttgccaccaaattcaatgtaagagttttctctcattcctttttgtggttgtgtgggggtcaaaaagtcctgatagataacggtttccggttaaaaaggtcacttagcttttatccaaagtgaataagtcatcttttcaagtataactggttattttctaacgttaccagctgatgctagaatctttttaaatagtaaagaacctccaaaattgcaaacaaaaggaatataaactatatcaacaaaaaccaaagaaatcatattttccaaataatagaacaatggagaaaaattaaacacatgtttaaacactcatttttaaacagaaaaagttcatcaatgctattgggttatttttatttattaaagctctgtcaagtttgataaccactatgtgataaatcttaaacatttagaatgttcaaaaaattattgtctgtttaaggccttttggtgagactgaagccagtaagaatctgggcattttgatttttctataaaattacttcatctccactagaatactgaattctattaacttttcttaatatgtattttttgaaagattcaacaaacaggtatctatttttctttctgtttcatgtaattgaattttcagtaacaattcatctattgtatttcaaaccttttaggtagaataataactgagaattatattaaggagaattgggaagaaattctttttctaaaagtgccattgaatttacttggggactaaaaggacataatatgtttaattcttgatcagatattagaaaaataatttgtctatagcagtataagggtattactgatgtgaagagaatgtagttttttaaaaagttctatttctaattataggagtaatgccttcacaaacaattgctatacatgtattctcctggctctgcagtctaagatttaaccaagtgagaatttcatccttgttattctttcaagtacttggctcctacagagctgtgctgagggagcaaaaatagctgataagtaaagtgactgaaattcttctgtgatgatataatccaagttctctctgttgctgggcgatatactctctttgggtataaattagaggaatagtgactagagattcttttctctgtgtgctattattttttaaagtaatttacgttagggaattttgttttcttaatttacttttgacaccttactgtactctgaatactactttgatttgttatatgactttatacattcagttttattttcaaccaaatgaggttggttgaattaagggttagaataaatagggcatttataaaaaatggtccatttatcatttctgagctactctaatatctgtggttgaacacataaacctaagctatttcttattagcataagtttaatgtatgcaatttctgactcatcagtaagactcagtgtgttctctaattttttatatttctgtaacacatattagggtcctagacaaatattttgaactgctcaaacacatactatcacatactgttttatattgtagttaatttactgcatagagtgttttatttaacaatgtactaatacaacaaacatagtatattttttattacatgctgtatataaaaattattattacctggcaatgatttagaccacttgactactgaaagaagttgcctctcgcctagttgattaagacttgtcagcaaagaactggaggtgtcaggttttgtgttgtcatgtcctgcatagatcacatctggttcaatgctcattaacaggttgatcagtggtggaatcaactgtatgtcttgacctggtgaaaaagtgaatctctggcttagggcttggctttcatttggaacgcccactggctgtgggagagcaacagcatccagtgctctcacaactctgactttattgaactttttaaattttcgacctacagagaagaaaaaaaagaaattcatgtaaatatatataaactccatatcccagtatagtattatttttcctaagtcttagaatcatagcattatgcattttagcatcaatcgaatgaaatagaataaaagtgttagatattaaaattacaaaaaataaattaaaacatgtaactaactcttgattaaaaaaaatttaggcctcagacttggattaaataattagttacatcaacaatcactgggtgggtgaggtgtgggtaattataccagtattctgcaggggtcatggagctttttaggagaagatatgaataaaaagacaaagagttttagtagcgactacgctcagtattggtccatattctgatagaacagccttccaaaatctaatgctacagactacattaattgaaacttagtctatagtggttgttaaatcttctttgaggttttgttcttttaagaatatgttaaaagaaaagcatttgtttgccagccccacaaaatctttttatacaatttcagtaggttcattgatcccttaaaatcaatccagctctacttttatgagatctaagactcaaaattaataatcctttttctaaattaaggaaagagaaagtttatacccgtgctggtcagatcacatttgtatttctggatgatttctggagtttctactttgatgaattctgatatgttaggggaaaaagatggatggtgagatgagtagaatctgctttatgagaaatgattgaaggagaaaaggatgcttatttgggctgctggtagttcacagacgccttgaggagaattagagaaagttgtggtgatgtgatatcaggctggattcagccccacctgctttgtgccaggcaagggctacttatcagtatgaagtagctgtttagccaagggaaggctgatacctgcaattatctaagcagaggttgcaatctgctggctctagtatctgatcatgcccatagatacattttgtttggctcagacaatgctgaccagaattatgagaccatccgtgaattacttaggtggcaacaatctgttaaagtttagttgcaatggttgcttttggatggggcatgtgtattccagtttttccagttccctccacaaactggtggtacgccaatggtctcttttcttaagtcttgtcactctagctacttatttacctggtctctgtaaacatttgtctctgagatttctgaactcagggtattgtcataaagaaaaaggattatacttgttctataaagccccaaggaatggccaggcatggtgagctcatgcctgtagttctagcactttgagaggccgaggtgggcagattacctgaggtcagaagttcgagaccagcctggccaacatggtgaaaccccgtctctactaaaaatacaaaaattagctgggtgtggtggtgggcacctgtaatcccagttactccagaggctgaggcaggaaaatcacttgaacccaggaggcagaggttgcagtgagtcaagatcatgccattgcactccagcccaggtgacaagggagaaactccacctcaaaaaaaaaaaaaaaaaaaaaaaaaaaaaaaaaaaaaaaacagccccaacgagttgagctaaaacttgtaggagatgggaaaacaaaacaattgccatggttaaaaaacaaaggggattcaaaattatttgcagagatcaaatgagctttctgggggaaggaatgagtcacaaagggaagggaaggaacagttatggagtatatagcatatgccaggaattgtgtgtgtgtgtgtttgtgtctaagggaagataatgttaagaagagggctaaagtttccctgtccatgactataatcttcaacaaaggcaccacctgtttgtcacctttatttattttttccagtgtcttttggcaatacatgtctgctaaatgaatgaaggaatacattattgtcttttccatctcccactctgaatatcttgtctttattatctgttgctgtatggtttgcactacataatattgtttcagtcaatgacagaccacatatacaacagtgttttcataagatagtattttttactgtactgtctctatctttagatatgtttaggtacacaaatacttaccattgcattacagttgcctacaaaattcaacagagtaacatgctgtatcggtttgtagcccaggagcaatgggctacaccaaacagcctaggtgtgtagtaggctataccatctaagtctgcgtaaatgcattctatgatgttcaatgatgaaattgactacttcatgacacatttctcaaaatgtatccctactcttaaatgacgcatgactgtttttgttgccttcagagaacatagcttcctccaccaggcttgctgccctgtaaggataggggctaggtccatttcactcactaatgtagatctgaagctttataaactgctaactacacagtggactcctgatagatatttgctaagttattaaagtagttgttttgacctaacatagcttaaaaaagaatatgagaagagagtactaaaaagggaatgtgaacagcaatgtgatttttaaaaaccactttgttgtttttttatataggatatttgttcaaagaagttattcctaacctttatgcataagaaccattactaagtaatgcaagtaacaaataaatctgtagaacttcatataattgtactcttagcatttgttgattgagaaaataagacaatggcaaaaagtactctgaattcagtgactagacaatggcaaaaagtactctgaattcagtgacagtttagaatgactttgtattatatttttgcacctctgaaaaataacgacacctggtattttgggtaatctaagcagagaataggcatgtggggtcagtggtcccccactcccctgcccgctgcattgacatcaggagatgacaggcatgggagctttgttggctggaaggagggaagatttgggctgagacgctgggggtgagcaaggggggataagaaagcggatggacatggtggctgaatagttgtctctcattcaagctgatggatgaggttttgagggattgaatcagaagcagaggtaatgatttgggggaggcttagggggaaagaagctccatttggagaatattttctttgtggccaatctacagtatggctacccctgtactctgccttccctcctgttacaattcatactcctgacatttcaataccaaatcttagtccttctcacccactcagggacttttctctgaaatcatttgccctcttgtctgcatcctctaatagtccctctcacacataatgtattccgtggagaaatatcactctcttcatttcctctcctccaattctcccttgaaccttctccagtcaggtcattcctctccttatcccaaccaaattattcttggcaaggccttcaaagatttctaaatgtcaaaaaacaatggccaattctcagtcctggtcttaacccgggtaagtacttaacacattgatcgtttccttcttcttttaacgctcttattcatttggcttccacatatttcattcttgtagtcactctttcagtctctctgtctagctctttttcatcttctcaacttctaaatctttgagtacaccaagctcagtcctcagacttctcttagatatctatgctctttctaggtgtctgcatctagttctatgtctctaatatcatctagacattaatgattaccaaatttacagtcctagacccagcttttccaagaaactctagactcaactatgcaactgtattctcagcatctccacttgtatttctaacagacccctcaaattaatatgtttagagcagaactttgatgcattggtccaaacatgcatcttctaggtttgtaacttcatttacacacctgctgctctagccccaaactctagagtcattactgatacctcacttattttctcacctcatatctaatacatcagcaaatcctagtggataaccatgaaaatataacccaaatccaaatacctgtttccactgcttgcaccataatttatgtccccacaatcacagttggactattgtaacatatgctggctggtttcccagctttcactttttgtagactattttctatacaactttaaagacttaatcagactatgtcattcttgcttgatattttcagtgactcctgatcctacttagaatgaagttaaaattcatatcaagctctacaaggcccaacttacccttgcatagatggtctctcttctattctccctcatgctccacccatattgatgcccatcttgcccttgaagtgtgtttctgccttggggtcttacactgacaagttcctctgcatgaaatgattttttccttggatatttgcatggcttgcttcctcacctaatttatatttctgcatatcgccttatcagatagcttgtttttatctatcctatagatatttataacatatatacccaaaatacacaaagaattcctataaatcagaaagaaaaggcatgaacagatatttcacagaggaagaaatatatagatgacaaaatacaacaatatgactgacctcattattaaccagagaaatgcaaagtaagaccacatgagacatcatctacctaaaacggcaaggacacagaatacaggaacacacaaaacaatgaagagctgccatggagtgtaaactactactactttaaaaacaatctagcattatctagtaaagcccagcatgagcatgtattacagtcagaaatttcatgcccaggtgtgcaggttacctacagaaactctcacacatgaaccaccagaatacatgtaaaataatgttcacaattgaactgtaggtaaagcacagcaaatatcaagtggaagtacatgtcaatacatgagaataaataaatgggagatactcatacaatgaaatcatacagtgtaggaaaaaacaaaccagaataatacaaatcaaaataaaaaacacaaatataatattaggcagtaagatcaaattgcataaatatacatagcatttctatggagtagaaaaaaacatctcaaaactaaaaaatctattagtgagaaatacatagatattaaaaaatcctacagaataatataagaaaatgaccaactccaaattaaagatatagaggtttctggagaaagataaagaggaatgtgataaggaagaaatatgcaatcaatttcaagtgtagtgataatattcatattttcttaagccaactgagtccagaggtggatgttaatcatattgtttctatatgtcatttgctttatgtgcatttttttatgaataaaatatattaaaaagtatggcttattcttaggatacaaaatcaatgtgcaaaaatcacagtcattcctatacaccaataatagacaaacagagagccaaatcatgagtgaactcccattcacaattgctacaaagagaattaaatgcccaggaatacaacttacaagggatgtgaagggcctcttcaaggagaactacaaaacactgctcaaggaaatcagggcacaaacaaatggaaaaacattccatattcatagataggaagaatgtatattttgaaaatggccatagtgaccaaagtaatttatagatccaataatagctccatcaagctaacattgactttcttcacaaaattagaaaaaaattactttaaatttcatatggaaccaaaaaagagcctgtatagccaagacatcctaagtcaaaagaacaaagctggaggtatcatgctacctgacttcaaactatacttcaactctacagtaactaaaacagcatggtactggtacccaaacagatatatagaacaatagaacagaacagaggcctcagaagtaacgtcacaaatctacaaccatctgatctttgacaaacctgaccaaaaaaaacaatggggaaaggattccctatttaataaatggtgttggggaaactggctagccatatgcagaaaactgaaagtggaccccttccctacacctaatacaaaaattaactcaagatggactaaagacttaaacataagacctaaaaccataaaaaccttggaagaaaacctaggcaataccattcaggacataggcatgggcaaagacttcatgactaaaacaccaaaagcaatggcaacaaaagacaaaattgacaaatgggatctaattaaactaaagaccttctgcacagcaaaagaaactatcatcggagtgaacaggcaacctatagaatgggagaaaatctctgcaatttatccatctgacaaagggctaatatccagagtctacagggaacttaaacaaatttacaagaaaaaaaaaacatcaaaaagtgggtgaagggtatgaatagacacttctcaaaagaagacatttatgtggccaaaaaacatggaaaaaagctcatcttcactggtcatgagagaaatgcaaatcaaaaccacaatgagataccatctcacaccagttagaatggcgatcattaaaaagtcaggaaacaacagatgctggagaggatgtggagaaataggaaagcttttacactgttggtgggagtgtaaattagttcaaccactgtagaagacagtgtgatgattcctcaaagatctagaaccagaaatgccatttgacccagtaatcccattactgggtatatacccaaaggattataaatcattgtaccataagacacatgcacatgtatgtttactgcagcagtattcacaatagcaaagacttggagccaacccaaatcctcatcagtgagagactggataaagaaaatgtggcacatatacaccatggaatactatgcagccatcaaaagggatgagttcatgtcttttgcagggacatggatgaagctggaaaccattcttagtaaattaacacaagaacagaaaaccaaacactgcatattctctcataagtgggaggtgaacaatgagaacacatggacacagggagaggaacatcacacaccagggcctgttgaggggtggggagctaggggagggatagcactagtagaaacatctaatgtagatgatggattgttgggtgcagcaaaccaccatggcacatgtatatctatgtaacaaacctgcatattctgtacatgtatcccagaacttaaattaaaaaaaaaaaaagtagagtggttgctggctggcaagatggctgaataggaatagctccggtctgtagctcccaacgcgatcaacacagaaggtgggtgatttctgcattttcaactgaggtacccacctcatctcattgggactggctagacagtagctacagcccatggagagcgagctgaagcagggtggggcattgccttacccgggaagtacaaagagtggggaaactccctcccttagccaagggaagccgtgagtgactgtgctgtaaggaacaatgtgttccggcccagatactatgctttccccatggtctttgcaacccacagaccaggagattcccttgggtgcctaaatcaccagggccctgggtttcaagcacaaaactgggcagccatttgggcagacaatgaactagctgcaggagttttttttcataccccagtggcacctggaatgccagtgagacagaaccattcactcccctggaaggggggctgaagccagggagccaagtgatctagctcagcagataccatccccacagagcccagtaagttgagattcactggcttgaaattctcgctgccagcacagcagtgtgaagtcgacctgggacgctggagcatggtggtgggaggggcgtccaccattactgaggcttgagtaggcagttttcccctcacagtgtaagcaaaaccacctggaacttcaaactgggcagagcccactgcagcatggcaaagctgctgtagccagactgcctctctagactcctctctgggcagggcatctctgaaagaaaggcagcagccccagtcaggggcttatacataaaactcccatctccctgggacagagaacctgggggaaggggtggctgtgggcgcagcttcagcagacttaaacgttcctgcctgctggctctgaagagaccagcggatctcccagcgcagtgctcaagctctgctaaaggacaaactgcctcctcaagtgggtccctgacccctgtgcctcctgactgggagatacctcccagcaggggtcgacagacacctcacacaggagagctctggctggcaatctggtgggtgccctgctgggatgaagattccagaggaaggaacaggcagcaatctttgctgtactgcagagtccgctagtgatacccaggcaaacagggtctggagtggacctccagcaaactccagcagacctgcagcagaggggcctgactgttagaaggaaaactaacaaacagaaaggaatagcatcaacatcaacaaaaaggatgtccacacaaaaaccccatctgaaggtcaccaacatcaaagaccaaaggtagataaatccatgaagatgaggaaaaaccagtgcaaaaaggctgaaaattccaaaaatcagaatgcctcttctcctccaaaagatcacaactcctcaccagcaagggaataaaactggacggagaatgagtttgacaaattgacagaagtaggcttcaggaggcgggtaatgacaaactcctctgagttaatggagcatgttctaacccaatgcaaggaagctaagaaccttgaaaaaaggttagaggaattgctacctagaataaccattttagagaagaacataaatgacctgatggagctgaaaaacacagcacaagaactgtgtgaagcatacacaaatatcagcagccaaatttatcaagcagaagaaaggatatcagagattgaagatcaactgaatgaaataaagcatgaatacaagattagagaataaagaatgaaaaggaacaaacaaagcctccaagaaatatgggactatgtgaaaagaccaaacttacgtttgattggtgtacctgaaagtgacggggagaatggaaccaagttggaaaacactcttcgggatagtatccaggagaacttccccaacctagcaagacaggccaacattcaaattcaggaaatacagagaacaccacaaagatactcctcgagaagagcaaccccaagacacataactgtcagattcaccaagattgaaatgaaggaaaaaacgttaagggcagccaaagagaaagtcgggttacccacaaagggaagcccatcagactaacagcagatctctctgctgaaaccctacatgccagaagagagtggaggccaatattcaacatttctaaagaaaagaattttcaacccagaatttcatatccagccaaactaagcttcataagcaaaggagaaataaactcctttacagacaagcaaaagctgagagattttgtcaccacgaggcctgccttacaagagctcctgaaggaagcactaaacatggaaaggaacaaccggtaccagccactgcaaaaacatgccaaattgtaaagaccatcgatgctaggaagaaactgcatcaactaatgggcaaaataaccagctagcatcataatgacaggatcaaattcacacataatactattacccttaaatgtaaatgggctaaatgccccaattaaaagacacagaccagcaaactggataaagagtcaagacccattggtgtgctatattcaggagacccatctcacaggcagagacacaatatgctcaaaataaagggatggaggaatatttaccaagcaaatggaaagctaaaaaaggcaggggttgcaatcctagtctctgataaaacagactttaaaccaacaaagatcaaaagacaaagaaggccattacataatggtaaagggatcaatgcaacaagaagagctgactatcctaaaaatacgtactcaatacaggagcattcagattcataaagcaagttcttagaggcctatgaagagacttagacacccacacattaatgggggagacttttaacaccctactgtcaatattagacagatcaacaagacagaaaattaataaggatatccaggacttgaactcaggctctggaccaagtggacctaagagacatctacagaactctctaccccaaatcaacagaatataccttcttctcagcaccacattgcacttattctaaaattgaccacataattggaagtaaaacactcctcagcaaatgtgaaagaatggaaataataacaaacagtttctcagaccacagtgcaatcaaattagaactcaggattaagaaactcactcaaaactgcacaactacatggaaactgaacaacctgctcctgaatgattactgcataaataatgaaattaaggcagaaataaataagttatttgaaaccaatgagaacaaagacacaacataccagaatctctgggacacagctaaagcagtgtttgtttagagggaaatttatagcactaaatgtccacaggagaaagtgggaatgatctaaaattgacacgctaatatcacaattaaaagaactagcgaagcaagagcaaacaaattcaaaagctagcagaggacaagaaataactaagaccagagcagaaccgaaggagatagagacatgaaaaacccttaaaaaaaaaaaatcaatgaatccaggagctggttttttgaaaagattaacaaagtagatagaccactagccagactaataaagaagggagagaagaatcaagtagacacaataaaaaatgataaaggggatatcaccagtgatcccacagaaatacaaactaccatcagagaatactataaacacctctatgaaaataaactagaaaatctagaagaaatgcatacattcctggacacatacaccctcccaagatgaaaccaggaagaagttgaattcctgaatagaccaataacaagttctgaaattgaggcagtaattaatagcctaccaaccaaaaaaaccccaggaccagacagattcatagctgaattctaccagagatacaaggaggagctggcaccattctttctaaaactattccaaacaatagaaaaagagggaatcctccctaactcattttatttggtatcatcctgacaccaaaacccggcagagaaacaacaaaaaagaaaatttcaggccaatatccctgatgaatattgatgcaaaaatcctcaataaaatactggcaatccaaatccagcagcacatcaaaaagcttatccaccatgatcaggtcagtttcatccctgagatgaaagcctggatcaacatacgcacattaataaacgtaatccatcacataaacagaaccaatgacaaaaaccacatgattatctcaatagatgcagaaaaggcctttaacaaaattcaacaccccttcatgctaaaaactctcaataagctaggtattgatggaccgtatctcaaaataataagagctatttatggcaaacccacagcgaatatcataatgaataggcaaaagctgaaagcattccctttgaaaaccagcacaagacagggatgtcctttcccaccactgctattaaacatagtagtggaagttctggccagggcaatcaggcaagagaaagaaataaagcgtattcaaataggaagagaggaagtcaaactgtgtctttttgtagaggacatgattgtatatttagaaaactccatcatctcagcccaaaatctccttaagctgataagcaacttcagtgaagtctcaggatacaaaataaatgtgcaaaaatcacaggcattcctatacaccaataatagacaaacagagagcccaaacatgactgaactcccattcacaattgctacaaaaggagtaaaatagctaggaatacaagtcacgagaaatgtgaaggaccccttcaaggagaactacaaatcaacgctcaaggaaatcagagaggacacaaatggaaaaacactctatgctcatggatcagaagaatcaatatcatgaaaatggccatactgaccaaagtaatttatagattcaatgctatccccaacaagctaacattgactttcttcatagaattagaaaaaactactttaaatttcctatggaaccaaaaaagagcccatataaccaagacaatcctaagcaaaaagaacaaagctggaggcatcaggctagctgacttcaaactatactacaaggctacagtagccaaaacagcatggtactggtaccaaaacagatatatagaccaatgaagcagaacagaggcctcagaaataatgccacacatctaccatctgatctttgacaaatctgaccaaaaaagcaatggggaaaggattccctatttaataaatggtgttggggaaactggctagccatatacagaaaattgaaagtggaccccttccttacaccttatacaaaaattaactcaagatagatcaaagacttaaatgtaagactgaaaaccataaaaaccctagaggaaaacctgggcaatatcattcaggacataggcatgggcaaagacttcatgactaaaacaccaaaagcaatggcaacaaaagacaaaattgacaaatgggatctaattaaactaaagaccttctgcacagcaaaagaaactatcatcagagtgaacaggcaacctacagaatgggagaaaatttctgcaatttatccatctaacaaaggactactatcaagaagctacaaagaatttaaacaaatttacaaaaaaaaaaacaaaaaacaaccccatcaaaaagtgggcaaaggatatgaacagacacttctcaaaagaagacatttatgtgggcaataaacatgaaaaaaaagctcatcatcactggtcgaaatgcaaatcaataccacaatgagatgccatttcatgccagttagaatgatgaccattaaaaactcaggaaacaatagatgccggaaaagatgtggagaaataggaaagcttttacgctgttggtggcagtgtaaattagttcaaccattgtagaagacagtgtggtgattcctcaaggatctagaaccagaaataccatttgacccagcaatcccgttactgggtatgtacccaaaggattataaatcattgtaccataaagacacatgcacacgtatgtttactgcagcactattcacaatagtaaagatttggagccaacccaaatgcccatcaatgatagactggataaagaaaatgtggcacatatacaccatgcaataacattgagctatcaaaagggatgagttcatgtcctttgcagggacatggatgaagctggaaaccatcattcagagcaaactaacacaagaacagaaaaccaaacactgcatgttctctcataagtgggaggtgaacaatgagaacacatggacacagggaggggaatgtcacacacagggatctgtcggggggtggggggctaagggagggataacattagaagaaatacctaatgtagatgatgggttgatgggtgcagcaaaacatcatggcacgtgtatacctatgtaacaaaccttcacgttctgcacgtgtatcccagaacttaaaatataataaaaaatttgaaataaataaatataaattaaaaaattgaaaacattaaaaagtatggcttatttaaatatcatgcattgaaaagataagatgaagagtctaggaaacagtattgcacaattattcttgatgtatatgtgactgaatctggatgatgatactatgagaagaaatttttatgacaagtgatatgaaaggtttttgagagagattagaagtgaaagcttaaatataacctaaaaataaaaattttgaaatatctcagcaaaaatcctatagagaatgggagcagggagaagaaaagcttataaacttctttagcatgttttaataaaataatttggtaaaatagggcaggcataattatttaattctgataaaagaagaatacagatttttgtattttctactaatttttctatatctttactatttttttaaaactacaaatggaattttttttttttttttttttttttttgcgacagagtttgttttatactgcctcataattacctcttttccattttctttctttgctaccttggtcaagtttccctttattttctctttcctatttaactttttctattaattcagaagttttaatttgcttttccagtttcctagtgtttgtattcccatcctgaacaatctctgtatttttcttttattatatcagaagtctctctgtagttcaggtgaatttatagacagtcatccttacactacctatataaaattatggaaggatgtctagaggcatgcttctacttcagattaccaaaatgataatgaccactgcgcagtgctgggatcagtccactggcactacactttaacaatcctttatagcactgatagtgcttgccttccatttttcaggattcacctccaatttccagatcaaaacacagtgtgcctttgtatatacttcaaagaaaacaaagatgctagacaacaggaaatataaagtgaaatcaggtattttccaaaaaccaaaactatactagaattgatttttttaaatgtatatgtcttaatcattcaaataaagtcaactacaatggaggagatatttgaacccctcaattctaactttttactgagtctagaaaataataaatgcttgtagatactactgaaaagttctagttggcaaaccataaaagacatcccatacaaacacagaaaaattttaagcaaagaaatgagaatgaatagtctgaggaccatccccagctagcatgtgtgagatggtgtttagggtaaacaaccagactgatgagaaggacatcagttggctgcaggctccaatgcagtgaacttggggagcaatgtgagaggtgctgtctctgagagatgctgattcaaaggccaggatttgggtggaaaaagagatgcccattaagaagaagattctgtgaggtctagagaggtaggtggtagccatcacccacagtagatcttgccttaaagaggaaaaatgatttttgttcttagggtattaaaggcacagagcaggactctgtacttctagaaaagataccactgtgtcctaaattcaagaattttgcagttcttccaaagaagacttgtagccttgttggctgtcaagtgtaggaggagaaaacaaaaaaacaacaaaaatctttactacacaacccatactctctagcccagaaagttattctctttgccataagaagtggttgaatctaggtggaggaggaggtagggggagaaggagagaaactggctttctaacacaaccttttaaaatgtgaagataaggcttgctaccaaaaaagaaaataatagaaaaaaagaaaaacccataaaacctgtaactgtggatttaaaaaatttcaaagaagataactattcatttaatcattcttttatgtagaaagcgacttatttttgaaagagctaattaccttatactagtttagcatttttacaactgcaaaatataaagctgtattcatttcagatatttttttgtgagtcattaaacaaaaatgttttctagaagaaagaagatggaagcagaacacctaaagtagaagatatattatcatccttaagggtgtaactatcttcccactcagtgctgtacagcagtgaactaaagcaaaagttcactgtgacctgttcaaggtagggaagtgattcattaaagacaagaatcatatgcaacgaagaaatcaaataactcaaaacataatttttataatcaaaacatagccacggaagcagataagaacaaactgctcctaacaaaatctatgtcatgacatgtcaaaacatatttattacctctattattctactgtaagattttattaaatcttttaaaagctttctaatattctctcatgttttatcttttgttatttaaatggtctctatgtcagaaatgagaattcttaaccataacagcatgatattggtataaaaacagacacaaagaccaatggaacagaatagagaacccagaaataaagccatgcattaacagtcaactcatttttgacaaaggtgccatggatatacattggggaaagggcaatctcttcaataaatgctgctgggaaaactggctacctatatgcagaaaacagaactagatcactatcactcaccacatacgaaaataaaatcaaaatcgattaaagacttaagtgtaagatctgaaactacaaaactactacaagaaaactttggggaactgctttaagacttttgggtaaatatttttgtagtaagacctcaaggcataggcaatcaaggcaaaaattgacaaatggaatttcatcaagctaaaaagcttccgcacagcaaaggaaatagttaacaatgtgaagagacaaagaatgagagaaaatatttgaaaactattcatctgacaaggagttaattatcagaatatataaggaactcagctcaatagcaaaaaaaaaaagtaatcccatttaaaaataggcaaaatgaacagaaatttcttgaaagaagacatacaaatgccaacagatatatgaaaaaatgctcaacatcactaatcatcagggaaatgcaaataaaaccacaataagatattatctcactccagttaaaatggctgttactaaaaagacaaaaaataaatgctggtgaggatatggagaaagagaaattctcgtacactgttggtagtaatctaaattagtactgtaactatggaaaacagtatggaggttcctaaaactaaaaacagaactaccacgtgatgcagcaatcctactgttgggcatatagccaaaagaaagaaaactggtatattgaagatattatctgcattcacatgtttcctgcagcactatatataattgtcatttacccctcctgtgtagagatcttggtgcagggggcccctctctattccatgccaaagcagatctccaggcatctggagcacccactctcctgaatcaggagcttaggatgcctcccatgcagagaatttgcagctgaggaggtttccaagcttcaggtctgtgtacaactctgggtgcttggtggccacccactggattcttccttggcactggtgcttgtgcttgccatcaggggacgcaggtggacctgctcggtccaaacttgccctttgtggttcccttctccttggggctgagcagggagttccaactactgtgcattctatgaatcagaatcagcctattgcctaagacaatggagagcttttgctcggaaacaaggatcaagtatatatccagccacattggtcacagccagctcttacctatacgtgccgcctatgggcctgtgggatgacctgcagagcccagtataaaacctgctgaaagaagcacatagggctgtagaatcaaagtgaaaagatcctacccagcattctttagaggcacaagccctagaaaaggggtaaaagggaaacaaaaaaaattattaaaataataataataataccattatagggaaagaaaaaaaaaagaaaaactactacccacatgaaaataagtacaaaaatttgaagtgccaatgtctccagatgagaagaaaccagtgcaagaattctggcataatgaaaaatctgattgcagtgacatcactaagggattgcactagctttccagcaatggtccctaaccaagatggaaactcagaaatgacacataaataattcaaagcatggattgcaaggaagttcattgtaatcaaagacaaggttgaaaaccaacataaacttctaaagcaatctaggaaattaagaaagacatgaacatcttaaaaaaaatcaatagtagcttctggaattgaaaaactcacttaaagccttcaattatattttgaaattcgttatcaatagactggatcaagcagaagaaaaaattttaaagcttgaaggctggtcttgtgaactaacccaatctgataaaaataaagaaaaaaattattttaaatgagctgtttttgagaaatataagattatataaagcaaaaaaaccctatgaattattggcattcctcagaaagtaggagaaaaagaaaacaaactggaaaatatagttgagggaataattcaagaaaactttcctaatcttgctagagagctagaaatccagagaacatctgtgagacaccacaaacatgaacatcactggctgggcgtggtgactcatgtctgtaatcccagcactttgggaggctgaggcaggtggatcatgaggtgaggagttcaagaccagcctggccaagacggtgaaaccctgactctactaaaaatacaaaaaattagccagacacggtggcaggcacctgtaatcccagctactcgggaggttgaggcaggagaatccatggaacttggagggcggaggttgcagtgagccaagatctcaccactgcactccagcctgtgtgacacagtaaggctccatctcaaaaaaaaaaaaatcaccaaggtatatagtcaccagaatgttcaaaggcaatattacagaaaaaatcttaaaggcagccagagaaaagtgcagatcacataaaaagggaactctatcaggataataacagatttctcagcagaggtttgcaagctaggagagattgggggcctgttttcagcgttcttaaaagaaatttcaaccaagaatttcacatcctgccaaactatgcttcataagtgcaggacaaataaaatcttttccaggcaatcaagtgctaaaggaatttgttaccactagaccatccttacaagagatccttacatgggagttctaaacatggaagcaaaagaaccatacctgcttccacataaacacacttaagcacatagcctgcacaccctacaaagcaaaaacacaatagaaacaacaaagcaaacagctaacaactaaacaagggatcaaaacctcacatataaaaattaaccttgaacgtaaatggtctaaatgcctgatatggtttggttctgtgtcctcacccaaatctcacgttgaattttaattcttaatgtttggggagggacctgatgggaggtgattggatcatgggggcacatttcccccatactgttgttgtgataatgagtgagctctcaggagatatgatggtttaaaagtggtggcacttcccctctcactctctctctctctctctgctgctctgccctggtaagacgtgcttgcttcccatttgccttccaccatgattgtaagtttcctgagacctcctagctatgcttcctgttaagcctgcagaactgtgagtcagttaaagttcttttcttcataaattacccagtctcaggtagttctttataacagtatgagaatgagctaatacagaaaattggtactgggagagtggagcacagttaagaagatacctgaaaatgtgggagtgactttggaactgagtaatgggcagaggttggaacagtttgcagggctcagaagacagaaagatatgggaatgtttggaacttcctggagacttgttgaatggttgtgaccaaaatgctgatagtgacatggacaatgagtctaggcttaggtggtctcagatggagtgaggaacttattgggaactggagtaaaggtcactcttgctatgctttagcaaaaagactggtggcattttgcccctgtcctagagatctgtgaagctttgaacttgagagagatgatttggggtatctggtggaaaaaatttctaagcaacaaagcattccagatgtatgtgacctggctgtttctaaacatgtatgctcatatgcatgaaaaaagagattatctgaacctggaacttacatttaaaagtgaagcaaagcaaaaaatatattggaatatttgtagcctggccatgtggtagaaaagagagagacaaaaaaacatttcctggggagcaattcaagcctgctgcagaaatttgcattagtaaagaggagtcaaatgttaacagtcaagacaatggggaaaatgtctccagggcatttcagagaccttcacagcagcccctcccatcacaggcacagaagcctaagagggattaatggttttgagggccaggcccagggcctagctactctgtgcagccttgggacatggtgccctgcattccagctgctccagctccagctgttgctaaaaggggccaaggtacagcttgggccagggcttcagaggatgcaaacccaaagccctggtggcttccacatggtattgggcctgcgggtatgaagaaggcaagagttgaggtttggaaacctccacctagatttcagaggatgtatggaaatgcctggatgtccaggcagaagtctgcttcaagcgcagagacctcatggagaacctctactaggacagtgtgaaagggaaatgtggggttggagcccctgcacagagttcccactggggcactgcctagtggagctgtgagaagaggaccaccatcaaccagacctcagaatcgtagatccatcaacagcttgcattgtgtgcctggaatacctgcaggcattcaatgccagcctgtaaaagcagctgtgggggctgtatcctgtagagccacagggtggagctgctgaaggccttgggagcccatcccttgcatcagtatgctctggatgtgaggcatggagacaaaagagattattttggagctttaatatttaatgactgccctgctgggttttgaacttgcttggggcctgtatctcctttgttttggccagttactcccatttaaaatgggagtatttacccaatgcccatacccctattgtatcttggaagtaactaacttgtttttgatattacaagatcataggtggaagggacttgccttgtctcagacgagactttggtcttggacttttgggttaatgctggaatgagttaagactttgggggactgttgggaaggcatgattgtgttttgaaaactgagaaggacataagctttgggaggggccaggggcagaatgatatggtttggctctgtatccccatccaaatctcacgttgaattgtaattcccaatgttgagggaggaacctggtaggaggttattggatcatggaggcaaatttcccccatgctgttgtcatgatagtgagtgggttctcccaagatataatgatttaaaagtgtgtggcactcccctgacccacctctcctactctgccatggtaagatattcttgcttccccttcaccttctgccttgattgtaagtttcctgacgttgcctagctatgcttcctgttaagcttgtagaactatgagtcatttaaatgtcatttcttcataaattacccagtctcaggtagtactttatagcagcacaagaatggactaatacaatgtcccatttaaaagtcacagagtgggtccaggcacagtggctcacacctgtaatcccagaactttgggaggctgaggcgggtggatcacctgagaccaggagttcaagaccagcctgaccaacaaggtaaaaccccatctctactaaaaatacaaaaattagccagccgtggtggcaggtgcatataatcccagctactccggaggctgagacaggagaattgcttgaacctgggaggtggaggttgcagtgagctgagattgcgccactgcactccagcctgggcatcagagtaagactccatctaaaaaaaaaaaaaagtcacagagtggcaaaccagctaggaaaaccagactcatccatcttctgtattcaagaaacccatctcacatgtaatgacaaccataggctcaaagtaaagggttggagaaagatgtagcatgcaaacagaaaacaaaaaagagcaggggttgctattcttagattagataaaacagactttaacaacagtaaaaaaggaaaaataagggcataacgataaagagttcaataagaagatttaactatcctaaatacatacgccctcaacactggcacacccagattcataaaacaaggacttctaaggacctagaaagccacacaacaatagtgggggactgatagcattagatcatcaaggcagaagactaacaaagaaattctggacttaaactcaatgcttgtccaattggatctaataaacatctacagaataatccacccatcaaccacagaacgtaaattcttctcatctgtacacagaacatatactccaagattcaccacatccttggccataaaccaagactcagtacattttaaaaagtcaaaatcatgccagtcatactgtcaggtcacagtggaatacaaagagaaaccaacatcaagaagatctgtacaaaccacacaattacatggaaattaaaaagtttgctctggataactttgagtaaacaataaaatcaaggcagaaataaaaaaaaattaaaataaatgaaaacagacacaacataccaaaatctctggaatgtagcaaaaacaatattaagaggagagtttacagctctaaatgcctacctcaaaaagttagaaagatctcaaattaatgacctaacatcacacctagaggaactaaaaaaaaaaaaaaaaaaaaaaaaacaagaacaaactatccctaaagctagcagaagaaaaggacgaactaaagagcagaaccaaatgaaattgagacccaaaaatctatacaaagaatcaacaaaaccaaaagttatttttttaaaggataaataagattgatagactgctagctacattaacaaagaaaaaaagagagaagatccaaataaacacaaacagaaatgacaaaggtgacatcacaactgatcccacagaaatatgaaagatccttagataactattgtgaacacctctacacacacaaactacaatatctagaggaaatgaataaattcctgaaaacacacaatgtcccaagtttaaatcaggaaaaaactaaagcactgaacagaccaatattgggttccaaatggaattagtaataaaaaacttaccaaccaaaaagaaagccctggccagagagattcacagctaaattctaccagatgcacaaagtagagctggtaccaattctactgaaactatttcaaaaaatcgagaaggaaagactcctccctaactcgttttctgaagccagtatcaccctgataccaaaacctgtaagagataccatgagaaaggaaaactataggacaatatctctgataaacacagacacaaaaattctcaacaaaacactagcaaaccaaatcaaacatcaaagttaatttaccacgatcaagttggcttcactcctggtatggaaggttagctcaatatatgcaaatcaataaatatgatttaccacatgagaagaattaaaaataaaaccatatgatcatctcaatagacacagaaacagctttcgataaaatgcaataccacttcatgataaaaaccctcaagaaactaggcaacaaagaaatatacctaaaaacaataagagaccaaaatgacaaactcacaaccaatatcagactgaatgggcaaaagctggaagaattttccttgagatctggaaaaagttaaggatgctcactctcaccactcctgttaacactggaagtgctagccagagcagtcaggcaagagaaggaaataaaaggcattcaaacatgaaaaaaagaagtcaaactatctctcttgcatattacatgattttataactagaaaaccctaaagactccaccaaaaggctcctaaaactgataaatgacttcagtaaagtttcaggacacaaaatgaacatgtaaaaattagtaccatttctatacaactatagcattcaaggtgagagccaaatcaagaaagtaatcccatttaaaatagccacacacacacacaaaatacctaggtatacatataactaagcagatgaaagatttctacatggataactacataacattgctaaaagaaatcacagatgacactaacacatgtaaaaacattccatgttcatggattagaagaatcagtattgttaaaatggccatactgtccaaagcaatttacagattcaatgttattcctatcaaactaccaacgacattttttccagaactagaaaaagctattctaaaatttatataaaatcaaaagagcccaaataactacagcagtcctaaacaaaaagaaaagtcagaggcatcacattacccaattccaaactatactgtaaggctacagtaaccaaaacagcatgatactggcacgagagcagacacatagaccaatggaacagaatatagaacccagaaataaaggtgcacacctacagccatctgatctttgacaatgttgacaaaaataagcaatgcagaaaggattccttattcaataaatggtgctgggatacctacctagtcacatgcagaataatgaaattggacccctacatttcaccatatacaaaaattacctcaaggtagattaaagatttaaatataacacctcaaaccataggaatcctaaaagaaaatctaggaaatatgattctggatatcagccttgggaaataatttctaactaagttctcaaaagcaattgaaacaaaaacaaaaattaacaagtgagacctaattcaactaaagagtttctgcacagcaaaataaactaccaatggagttaacagacaaccaacagaataggagaacatgttcacaaactatgcctccaacaaatgtctaatatccagaatctataaggaacttaaacagttgaacaagcaaaaaaaaaaaatctcaccaaaaaaatgggcaaaagacttgaacagacatttctcaaaaggagacatacaagcagccaacaaacatttaaaatgctccatatcactaatcattaagatatgcaaatcaaagcgacaatgacataccatcttacaccaatcagaatgactattattaaaaactcatgccaggcatggtggctcacacctgtaattccagcactttgggaggatcacttgagcccagaagtttgagacctgactgggtaacaaagtgagaccttatcgttacaaaaaaaaaataaataaaaagtttgccaggtgtggtggtgcgtgcctgtagtctcagctactcaggatgctgaggtaggaagatcacttgagcccaggagattgaggctgcagtgagccataattgtgccacagcactccagtctgggtgacagagtgagactctgtctcaaaaaagaaaactcaaaaaactcaaaacctaatagatgctggtgaggctgtggaaaaaagggaatacttatatactgttggtgggaatgtaaattagttctgccacggtagaaattggtttgaacacttctcaaaagacatttttttttcttttgagacagtctcgctctgtcgcccaggctggagtgcagtggcgcgaactcagctcactgcaagctccgcctcccaggttcatgccattctcctgcctcagcctcccaagtaggtgggactacaggcgcctgccaccatgcctggctaatttttttgtgtttttagtagagacggggtttcactgtgttagccaggatggtctccatctcctgaactcatgatccacctgcgttggcctcccaaagtgctgggattacaggcatgagccaccgctcccggccctcaaagaacttaaaacagaactaccattagaccagcaatcccattactggatatatagccaaaagaaaataaattgttctactagaaagacacatgcactcacatgttcatcacagcactactcacaaaagcaaagacatggaattaactgaggcacccattaatagtggattggacaaagaaaatgtggcatatatacaccatggaatattatgcagccatgaaaagaacgaagtcacatcctttgcagcagcatggatgtagctgtaggccaagaccctaagcaaattaacacaggaacaaaaaaccaaatactgcatgttctcacttataagtgggagctagacattgggtattcatgagcataacaatggaaactgaggactactagaggaggaaggaagaagaagaaagggttgaaaaactattgagtactatgctcagtacctcagtacctaggtgatgggatcatttgtacctcaaacattagtgtcacacaatatacccaggtaacaaacctgcacatgtacccactgaagctaaaataaaagctgaaaaaataaatagagctgcgatagctggaaaaaaatggtctaatacactctttcccagagtttaacacataatcatttatggtatacaagcagtttttaggtggtacatcgaatatatatttaaatagttaagaatgtatattaatgtcttttagaaaaattataactggcatatcaaatccattatttcagagatattattgcttaggaagaggctaagcaaaacaaaaatattagactaatgagtttagtagtgagtctgcttcctgataatccccactaactctgcttaatatctttccacagcacttatcattatttgacatgtacatttgttgctcgtcctcccacagtgtaagatctatgagggcaggaattttgtccattttctttgctgctatatgcgcagaacttagaatagtatctggtaactagaaagggctcgatatacagttactgaaagaaagggatggggtgcaaattagaaagcgattgagggagaagagggaggaaggaaagaagaaggaagggagggagggagggacggaaaggagggagagatggagggagtagagttaaacaaataagtaatataaaaggtatgaaaatgcagcaatagctgtgaagcccgtagaatatggcttgggaagcataggaataacaggctaaagtaacaagcagtttggttagagaagataaaggattacagatgcttgaataatatgctaaaggccgggtgcagtggctcacgcctgtaatcccagcactttagaaggccgaggtgggcagatcacctgaggtcaggagttcaaaaccagcctggccaacatggtgaaaccccatctctactaaaatacaaaaattagctgggcgtaatggcgggtgcctatgataccagctacttgggaggttgtggcaggagaatcgctggaacctgggaggcagaggttgcagtgagccaagattgcaccattgcactccagcctgggcgacagagtgagactctgtctcaaaaataaataaataaagccctagaatattaaaagataacataaatttctctaggttatattgctgatccccaggagtattctgaaggttctatccagaactccgccattttcattctttgataaaattgaagagtcttttaaagtcattgggaaaacactcaagattaaagatcttaatggaatctaagtgtcagcattagatgctctgctactttctaaggcaagcatgccaaggagaaaacaatggctgcgccagggtcagaataccttcaggtctttcttctggctccgacattgactagatgtgtgattttgtgaaaaagttgtattttctaaaatttaggaggttccttaagacttctgagttgaaacagaaatacaaagaggtggtaattgaggtgtgggactcaaaaacaattttaatctgctgaggttgtggttttattcatttaatagaaagttactgagcacctccttaagccaggcactactgtcaacctaatcactgcccttaacttaggaaaacaaatctttaatcaagaaattataaaattctataagtgatctaattacatgaattactgtaaagtgcccagtatgttacctgggtgattaattttggtccagcacatttgcaaaaacaaatgaggtgacagttgtatacgctttaaacttgacaagcattggtctaaatatagcatttcccaaaggaaaatacacacaacgtccatggaacacaagctgtttatagttagtgcactgaaaaaacatttttagctttaacaattatgtaatatttttattatgtttttaaaaagttatatctagatgtaaaagctacgattcccagacagtgttgtgcaggatgtggctaagtgaaaaaattcaagggagtacgggtgtcttgcataactgtggctcgcttgatctccctcactctgctatgttttcattcaaactatctgtcactatctgacattaattttttaaatgtgtgctatattactgaaagttttagagattctaataaaaatatgggaaaagggaattattaatataactcttcatcaaatggattcctaccccagttacagtaatcttgctttaatcaatcacttggagaagctagtaagcaatgtacctgaccaatttgcagagtcaattttttgagctgaaaggaatgttaatgctctcgttttacagataagtagactgatgcccaggaagagctgtggtcacacaggagatgggcagcaaagcctagactcccacacatgttacttaatgacaggtcagtacattgcccattacctctacttataggcaatatagatgaattgagttttcttctcttaacagacctcaatgtgctagaaaactgtaaagttaatggaatggtattggttgcttcattcagacactcacatgtgcagagtcactgccaataatcaagacagaaaaaacaaaaacaaagatgaataagaaattgcaattttctgacacacagttttatagtataaagattacacagtatattattgatgaaaacatcagaattacctccaaggaccatgccagcctgacagcactttctaaggcgacatgctgggcagttttttctgcggattttatcaacgatgcagtcatttcttccagcacataagtagttgtgctgccctaaaaaaacaaaatgagtcaaaattatttacaatatctaagattcactggaaattctatgcagtgacaactgaaatagagactagatacacatcagcaagtgcatgactcagccagcttcctgcctggcacagctgaatgttggtgaagctataaaactgctcagtactttacctgtgcctttaggagcctaaattaggtgagcatagctgaagaatcaatcctagggctcctttaaaaatcttttgcaagagatttctgtttatcttcttaatggtgtcagtatcagattgcctttagatgtcatttcaagattagctacacgtttctgataagcattagttgtaccaggtggtcaaactctagtaaaacctttctttctgacattgatgccttcaagaaaacagatacagactaaagaattgaagaggcctctggaaaagaccttttaaaaaaaaatctaaatgcttagttctattggaaaaatcccagagctggttctctctttaaggaatcaatggaagagcatgtaaaaaagatcacttattgtactgaatttaatacctgccttattttgactacacattagaaaataccttgaacagaaataatgataaaggaagaacttaaaacagatagaagtactcaataaagaggagaaactttatagaagtatagacaatacacatcgcacatcaccctggggtcactttgtgtagaggaaactatggatgcctttaaaaaggtctggcacatgaatttaatattcaataatgatttttaaaccacaaatatgttaataaggcacggaacaatctaatttatttagatttaacaaaaataaaaaaattcagcttaaacatgtaaacgttaatttactatttttgacactctctttttttcccaataaatttctttaaaacagactgtgagtcttttaatatcatcagcctacttcagcctacttatactgctgaagcatgacacataattttaaaagatcatgggattttcctgtagtattttacattagttaagattaaaatcagagtcttctggccaaagttggtagtaaaggaactcagaggtacctaaaacatgtacttaatcaagcaggggaaaggattagcctaaacggcaaagggcaaaaatgccaaaaggcctatatatgtgacagctgatatcaagaaggttcctgacctcatgcatttctggaaaagcacaaatattttcttaaagatgcatgcctctataaacttatgttcatgtacactcacatagtgttcatctcttgtttagagtcccaggaattaagccttcacctaataaagggtaactgtgaagcctcatatctttgctgacttttgtgcacatagtgccagtcctacaggaggttagcagagtgtactgcatactactttctgatttgactctaattttttttttttttaccacaagttatatatttaggctataggagtgactgtgagagcaaggtgtgtgtaagtcagtctatcagttgatcatctatttctaccctatactgatatctcacaagaagagagaacagaaaatagcaagattttctattctttttctttcagacggagtttcgctcttgttgcccagcctggaaagcaaaggcacagtctcggctcaccacaacgtccacctcctgggttcaaggaactttcctgcctcagcctctgagtagctggaattacaggcatgcaccaccacgcctgggtaattttgtatttttagtagagacagggtttctgcatgttggtcaggctggtctcgaactcccaacctcaggtgatctgcccgccttggcctcccaaagtgctgggattacaggcatgagccaccacgcccaccaaggttttctctatacagaaaacagacttcgagaaatagagcacgcagatgtaattgtttgaagagaggctgtcctactgtctgctccttggtcataaatacgcaggaaaaaggtctgatctaaacaccagaagctggcatgctgctgatctgtctcacttacatttctaaaatggcctacgatatcatctccttattcagtgtctcaaagagtcctccttaccaatggtttttgcttttgtccttcaaacaattccttcctatctgctttcaagtcttgctccatcaatttcctcctttctctcctaaagtctttagtctctccagtccagtgatgtcctgctctctcattatttttccatccaaaaaagttccagcaccaaaaaaaggaaccctagctcactctagattctccctgaatacattcccctttttttggtcctcaaagttgtcgaaaacatcatctacatttgctgtttctacctaacttcctttattcctatgtggtaatttgacttgtatattcactattcttataaaatggcttaacatcatcaaatattttttcattcctaaatgctgtgaatgctttcctgacctgaatcatttgcttcaggtaatttctttccccacctgtcccattgattcatattttggggcctctcttttctgactcttgacctgtctttccagcacttccttttaaattccctttgtcaactcctcttactttgcttattccttatatgtggtgtgccactgcacacactttgtgggtagtctgtctcattatcttctgatggtcccaaaatctcaatatgagctctgaccttccagtcatatttctaggtacctcaaactcaatatatacaaaatacaactaagtggttatctgtacaaattatatcatgaattgtattattagagaaattatataagaacgtaaggaaattcaaaacagaaaataacctacaatttgataatcatgcatctattctcatttttgcttccttcttgctatggtttgaatgtttgtgtccccttcaaaattcatattgaaacttaatccccaatgtgacaatattaagaggtggggtctctgcctccatggatggaatcagtacccttttaaaagggcttgagggagtgaatttggtcctttttatcctttctgccatgtgaggatgcagcaaggggcgccatcttggaagaagacagcaagcttttaccagacatggaatctgtgccaccttgatcttggacttctcagcctttagaaccatcagaaaatatttctgtatttataaactacccagtctaaggtatttttttattgcagtggaacagactaaagtatttccaaactgtctatacaaatctaaatggtatttaatatctatttgttaatatttaagttaatgtatttatacttttataaaactctcattcattcaacaagtattgagtatctactatatcccaacactatttactgactaatccatgttttcccccactaaattaaaatgccatcttaattgctgacactacattgtagtaccgaagagcaaagttacagaggccagactgcttgttcaaagctcagatctgcaacttactatccacaaataaaaataataaaagaacaacagacaattattaagcacttactatgtgccaggcattgttatgagctattgacatatattaactcatttcatcatcaaagcaacctttggaggtattaatattctcattttatacagagttaactgaggcatctagaagttaaataacatgctcaaggtcacacagttaaaacattttaggttaattatcttgagaggtagtatagaaagttactgtttaagtgtgaatgtcaatctcatcacttagtagctagatgacatttggcaattaaaatgaagataacattagtaccttggattggatctaaggaatacacaagctcatgtaagagcttagaatagtctggcacatagcaagtacttaacaaatgtctattataattatgattatcacacatctaaatctcaaatatagttagattttttctagatgtggtccaagaagtctatatctagtcaagttattcagttttaaaggcaacttaaagatattctcaaacttgcatgaaattgggaaataagaccccagtatgctcttccaaaagatgtaatccagtcaacaacagatacgttatagcagacaatgtatgaggtaggaagtctggtatgaagagactgatgataagtaccaaataaatttaaacataagtataaatttaaacatgagtataaataaggaaattatagttaccaaacagaatgcaattcttataagtttggaagaaaaaatgacttagatgtccaatatttaaagtttgagttggaaggataatttttaataattaagggcactggcatgaagtcacgaccttgaggggttggcacgactcatatcattgtagactgcctgtgacaggctggaatggcttaaagaaatctggaagctcaattcactctaacttttcatcatctttaacattaagtgatgagccatcaaaactgcaaagtagatgcattatcatgcaaacaaaatatggggagtacagttaaaggtttatcttctcaagcaatgttttggcttaaagaaatgttgtagccagccagttccaactagagccgactaagggaatttggcatggggcccaggcattgcctctgaaccataatgcatggccaggactcagagccaccaagaatgcaatgaatgcagtgggagccactcgctacttcaggtacctggctgtatatgcaggtgaagaaaggtcaggcacaagtgcattagccctgtcacaacattttcctggaaatctagaaaaagatgaaaatctaacataacatcagttgctaccattttcttcagacaacagcccacagtttatattctggttttgagaaacacatccctgtctagatccactcactcactcataaaccgtaggttgatggctcaaattgcgtattatttcttctcctcagtccaacataacattgtgattctacaacaagtgtaaaacacgtcactatattaaaccactcagagcattccatttgctgccatctttgctgtttacctacattatcaccaccaggagttataagacatttaggaacacttataagctataagccaccacaaacaggttacatggcataatgataaataggggagttcaccccactaggagccactatctagctctctcccttaatatatgcatcatctgcccctgttgaaacctggggagcttaatagagcactatgcttctttctccttgcaaaactgatctattttagggtggggaaagcagggggtgagttcaaactgttatgtcttttctttctaatcacatactctttcccatgttctttatgctccagagtactaacatccttggattacccccaaactctctcatgcctctatgactttcctcccttccttccaatttatcaacttaacaaaatctcactcgtctttcaagccttggcttaactgctcccctcatctggtcttacctgactgctctcataaaggctttagacatttcttctgtgtaccaactgcacattcaatgtattcctattgtatactttgtgtctatagctactaggttctataagagtaggaactactcagtttgatacttttaactcttgtgtaaaatacagcttgataaatattttttagataggtgtatatacaattatacctattgtttcgcttatatttgtaaatatagttatactcaacagtataagacatatgtcacttattgtcatatatagtaaatagtcacttgcttctgattttttaccaccttttaactttttttcttacagaatgctttttattcagtaatttctcacctttttatagtaatagaccataattaaatgtgaagtctaacaagattatttccaagtgttactctttttttttttttttttgagcggagtctcgctccgtcgctcaggctggagtgcggtggagccatctcagctcactgcaagctctgcctcttggattcatgccattcccctgcctcagcctcccgagtagctggaactacaggcgcccaccaccacgtctggctaattttttgtatttttagtagagatggggtttcaccgtgttagccaggatggtctcgatctcctgaccttgtgatcctcctgcctcggcctcctgaagtgctgagattacaggcgtgagccaccgtgcccagccccaagtgttactcttaataaaaaatactctgtgaaaataaaagcactatcaaaatgtgattagatcattttatttgcctagtgacttacaaaaattttagatgacaaaattccactttaaaggaaataaaatatctttattaggaaataaaatatctttattagaaacaagaaagggacttacctgtaaaataagagacctatattgtttgtgttgcttctcaatatgagcacaagtgaacatagttcacttaggtctgagagagttcacaatttttcatggcatttttgaaaagaaggggaaaatacatagtcaaatggcaggaaataatatatgttcagtagtttaaggaatgaaatagtgaaagtcagtatggtatgacccacaagtcacgggcttgaagcaacatgagctggtccatatgagggcaatctttctaccacaaacacagaaatactggataaaatataatagaagcatgttgcttaatttcagtctggaaaaaaaggtaggtaaatctctataagccagaaaagaaaaaagaaatgaaagctggaggaacaaacttaactggactctgcagtgatccaaaaaagtttcagatacagatataagaaatgcctaaaagataagaaatggggtttaaagcccatgagaggatgggatatattacttcagtcccaatggaagagagtggcagggctgaactatatgcataaaaatgtagaaccttaaaagactgccttagcaatgaaaacagggctagaaaacactacacagaaaatgccaggcagccaggaatctgtccaaaattcagggtgaggacaaaaatcatttatgccaaaaacaacataaacaaaataaaatattggcctttaatgggaggtgaattttcactatcacatggtataggaacctgggccaaggaacaaagactggtacaggaccagtgaaattgccagggtccttggagaggcaaatgaaaagcttatctgtagggacaggtgtgtccacatgcaaaaaataaaagatttgtacaagaatatttatagcagcagtgttcataatagcctcaatctgaaaacaacccaaatgatcacctacagcataatgtgtaaacaaatggttttatttattcaatgcattattatacagcaatgagaataaatgtctagctccatgcaaaatgtggatgaactacataagtataatgttgtgtaaaataagcagacatagaaaaatactatctgattccattaatataaagttctaaaatagttcaaaccaatctatcatgttagaaattgggatggcggttacttttggggaatgattggaaggggcaaaatggagcttctgggaacctaatgatctttaaattttttggtctgggtgataataacatcgtttcatttacctactgataagctctacatctttgagtgcttttctgaaaatatttatcaataaaagtttctttcttttaaaaactggctccagctaagaggacaacctagagagtattgtgatccaacagaatctgtgtagggtcagtcagcggaagtccagaagcagagggtcacggtcatgtccagagagaagagaggtgtctactgcattacagcattacaggattacagttagaggctctcagtaaagtatctccatgaatctcaccaaagtatcccactagaggaagagcagcctgtaatcacccaccaaacacagataaccccagtgacactaaaattcctacttgttgtgtaaaactgtgctctctaacttctcttcccacacgttggccctggaagggtcaaaatcagctgtcattgggtgagtgtaggcagcataaacagagaagaagcagatcttatttgttcttccctttaggcttcaagctcaaagcagtgagtgctgaattggggcaaagaagaaatctaaacttagaagaggatttgtaaatttggcataatgcttccctgggcattttgattactgatatggtttggctttgtgtcaccacccaaacctcatcttgaattgtaatcccataatccccccatgttgtgggaggaactcagtgggaggtaattgaatcatgggggtggtttccccccatgctcttctcgtgatcatcatgagcagtgtgatcaatcagtgaattctcatgagatctgatggttttataagagtctagcatttcccctactggcactcattctctctcctgctgccttgtaaagaaggacatgtttacttccccttgggccatgattgtaagtttcctgaggccttcccagccatgtggaactgtgagtcaattaaacctctttcttttataaattacccagtcttaggtatttcttcacagcagcatgagaacagactaatacggttagtggaatgagattgttcttctgattaaaatctttgggactggcctaagattttatgcaggagatggaagaactagtctacaaagcaggttttgttgttagaggtaataatgctgatactagtattttgaatccacacacacacacacacacacacacacacaatacatacagataaaacaactagtaattattttgattttgctagaaatacaaacataaaccagtaattctcaactagaggcaattttgctcccttcctcaccaggacatttcacaatatctggagacaattttggtttatgcaactagagggtaggtattattgcagagaggccaaaaaggctaataacatcccataatgcacaggacatccccccctgcaacaattatgtggcccaataggtcaatagtgtgaggttgagaaatcatggtataaactcatgatccccttactttttcaaaaagaggtattttctaggtctcttcactgaaaatgcccaaaagtaaagacaactccagcaggaattcagattttcacatgtaatataattttcatctaaaacgaaccagagctccttggggaaatgactgatccaggactagggcaggaaatgtgcaagttgagcctgggatgctttgctgtgacagatgatttgcttttactgagggctaataaaattgtgtttcattgaccactgataattcacaccaaccaaaattaaaaccacttgaacatcaaaaagaactggttcaagtaattaaaacacattgaatatatattttaaaaaacatgagtacataatgatatgtaagaagagaaagaatgaaagaaacaagaggaactcactggaaaccactggagattactagggcacaaacttaacaactttgaaaactgaaaattaaaagtaaataataagtatctgttaggctttccattattaagtgtattttaagattaccagattgcctcttggaagagagagagaactattaaaaatatttatagctgataaaatcagaagggataatagaaatagagaatcatccttttgcaattcctaatgaaatggccaccacttgtcaagtctgaaaagacaattaaaatactcctccattttccaactacatatctgtgtgaggccacatttccttcccatgcttccaccaaaacaacatattggaacagattgactgtagatccagatatgagaatccagctgtcttctattaagatagacattaaaatgacttataaaaatgtaaaacaatgtcattcttgtcattaaattttttggtttgaaaaattagtatctaatgggtttgctgttttttaaatgagctaataaaaagttttaaaacattatgagttttaatttctaacacagtaaatactgagagagagaattcacgtatacagaagctctttgggcttgtagcaatttttaagcacataagggggtcatgagatcagtgtctgagaaccgttggcctgtgtaggattcttgtcaaaacgtttaactggcatataattatgtgggggaaaaatcagacaaagccagattatggaactttctacaaaacggctggcctagactcccaaaaaaatgccaatgtcctgaaagacaaaaaaaggtgggagatctttttagattaaagaaaactaaacagagttgctaaccaagtaaaatacacaattcctgactggatcctgaataaaaaaacattctatacaaaacattttgggaaaaactgaagaaattttaatactagctgaatattagaattgttttatcaatgttaaaattttggggtgataatattgtggttgtaatggaagatgttcttgtccttaggacatgaattttggagccttaagaggtgaaatgtcatgatgtctgcaacttaccttcaaattgttgattacagatattcgctgtactattccttaaagttttttgtatgttttctggaatccagcatattctgaagatgcatgcagaatgagacaaaggttcagaccagtttttcacgttagtaagtcaggggatggtcatgtaaagtggaatgaagagtgactatctagcagagtgtgaaagacagctggacaataacaaatcagtaggttttaaaatatcttcatgaagatccaaggatttttgataaaagtgaaggaaaaaggtgtggggtgaaggagagaaggaaaatcttattgagtatattagagaccaatatactaagcatttaactggtttatttcatgtaaccttgtaacaaagtagacataaaaacatttaatttctctagaacagcagtccctaatcttttttgcaccagggacaggtttcatgaaagacgaattttccacggactgggggcagtgaggggtggggggggttgggggaagcggtgatttcaggatgaaattgttccacttcagattatcaggcattagttagattctcataaggagcacaacctagatccctggcacgcgcagtttgcaatagggtttgcgctgctatgagaatctaatgccaccactggtctgatgggaggtgcagctcaggcagtaatgctcactcacctgctgctcacctcctgctgtgtggcctgattcacagcaggaggtgagcagcaggtgagtaaaatggcaggagaaatgataatgtatgttcaatagttcaaggagtgaaatggtgaaagtcagtatggtatgacccacaaatcgtaggcttgttctaggttggggacccctgccctagaagaaataataaagtgcgtggaaattcccaaggatggagcagatctaaatagtctttggactggaatctaagacgacacagaatatactggctagagaacagaccactgggcactgaacaccaggtgtgagcatctgagaccttaagggtgatcctgaggagggatacagcacactggtgagcagcagggcagagaatagcacctcctgtctcttacccccgcatgttactaaacttcaccctgaactacaggacactacaggaatcccaaattagtccaaccatcagacaaacaagtacttcggtaaagactgggcatattcctgacctctcacatatataagaatatatgttatcttcacccaagaatagcaactcaacctgatggaatttttgccaatctcagcatttagtactcaaagaggaaatctaatgtcaacccaatttttatgtcttagtagttaaccagttttattaatcctacttaaatgcatatagggtgtttttccttctcatgaaaaattttaaaaaattaccaatattgcaaagttttagctttttaaaattacatttacctgggatgcagtgagtccttttgatctaaaaagtaaaatttttatttaactgctaaacttacttctgttatacgtgttctaccacctattattagctatataaccttgagcagatcatttaacttcttggcactacattatcctcatctataaaataacagtttaaaagttgctttgagggtaaaataatgtatataaaatctatagcaccataacagacacagaataaatgcttaacagtaattatgacagttcaataaacaaatatttcttgaatatctactatgtgccaacattgttctaggtgctggtgacacaatgtagaaatgatctcgtaaaaaagatacataaggaagagtcattttctataatcaattaatacttttgctgccttgttttttagtttcttcttcagaatcactggatatttatgggttgcatatgtgctctctattcatcgtatctgttgtccaacagcatagtgataaggccatatgctttagagtcaaatagactttcattcaaattcagttctgtcactttctgcagcatgataaaattgtttaatcaaaatcttagtttttcccacatataatatagagataaacattttataaggctgttatatggattaagtgagctaaattagatgaaatgttaccacaatgactggtacataataaaaacaataaatcatagtgtgtaattttcaccagtttaccaatttctcttcattccgaagtaactccttaaatgtgaactctacataattggtgttatttcctgctctttatgacgaccaatatggattttaactctgttactgcattttggatcacactgcagtattttcttatctcatccagctatgcttttatttaatctggttgtttccttatgacactcagtcttgcttctcagatactgctgtgtttgtaataactcaaagcagattattatccaaatttttcttttctttttttttcagcaaataaattttaggcatatttttcctaagatagaatctggctattactgatattaaatgttactggtgttaaatgaaacaacttgacaacactgtagtgggggaatgtgttaagtgagacactggcagtaaccaaagtttgttatgtttcaggatactcatggaagctggaagctggaagctggaatgggttggagaagtggggagtgttggggaaaggaattagagaggaaagaagaaattgctaagatgctaagagatgacagggattgtttgaggatgtgagtaaggctagagctggctaagccagggagaagagtagtagatagtacaatcagagtgcagcagagtgcagatgctgtagggccttgaaggccactgtaagaaatttgatttttctctaaaagatatggtaccactggagacactggagcagacggctaacatgatctgacttatattttaacagtatcacaatggcttctaagtcgagggcagactctcaacctctggaatctaggaaggtaaggatagaaatgagactgtcaggagaccactgcaatacatccgaaggggaataagagtggcttaaaccaaagtggtcagtggaggtggtgataagaggtcaggttctggatgttttctgaaggaggagacaattgatttaaagaatcctatttggtagcatattccttttttttttttttttttagaaatgaagttttagggaccttttcaaggagaactacaaaccactgctcaagaaaatcagagaggacacaaacaaatggaaaaacattccatgctcatgggtaggaagaatcaatgtagtgaaaatggccatactgcccaaagtaatttatacattcaatgttatccccatcaagctaccactgactttcttcacagaattggaaaaagctactttaaacttcatatggaaccaaaaaagagcccacatagccaagacaatcctaagcaaaaagaacaaagctggaggcatcacactacctgacttcaaactatactacaaggctgcagtaaccaaaacagcatggcactggtaccaaaacggatatatagaccaatggacagaaaagagacctcagaaataacaccacacatatacaaccatctgatctttgacaaacttgactcaaacaagcaatggggaaaggattccctgtttaataaatggtgttgggaaaactggctagccataagcagaaaactgaaactggaccccttcctaacaccttatacaaaaattaactcaagatggattaaagacttaaatgtaagactgcaaaccataaaaatcctagaagaaaacctaggcaacactattcaggagataggtgtaggcagagacttcatgtctaaaacaccaaaagcaatggcaacaaaagccaaaattgacaaatgggatctaattaaactaaagaacttctgcacagcaaaagaaactatcatcagagggaacaggcaacctacagaatggaagaaaatttttgcaatctatccatctgacaaaggactaatatccagaatctagaaggaacttaaatttacaaggaaaaaacaacctcataaattgggcaaaggatatgagcagacacttctcaaaaaaagacatttatgcagccgacagacatgaaaaaatgctcatcatcactagtcattagagaaatgcaaatcaaaatcacaataagataccatctcatggcagttggaatggcaaccattaaaaagtcaggaaacaacagatgctggagaggatgtggagaaataggaaagcttttacactgttggtgggagtgtaaattagttcaaccattgtggaagacagtgcagcaattcctcaaggatctagaactagaattaccatttgacccagcaatcccattactgggtataaacccagaggattataaatcattctactataaagacacaggcacacatatgtttactgaagcactattcacaatagcaaagacttggaaccaagccaaatgtccatcaatgacagactggattaagcaaatgtggcacgtatacaccatggaatactatgcagccataaaaaagaatgagttcatgttcttcgcagggacatggatgaagctggaaaccatcatcagcaagctaacacaagaacagaaaaccaaacaccgcattgtctcactgataagtgggagttgagcaatgagaacacatggacttagggaggggaacattacatacctgggcctgtcaggggatggggggctaggggagagatagcattaggagaaatacctaatgtaggtgacgggttgacgggtgcagcaaaccaccatggcacgtgtatatctatgtaacaaaactgcacgttctgcacatgtaccccagaacttaaagtataagtaaaaaaaggaaattaagttcttcatgtcatcattctaaataatgtgttcttttttcttcccttcaactatcttaaattgaatgaatcagcttttcatctatctttcctgaagctgttctgctcctctctttctaaataaaaaaatctctctggaactcttccaaatgccctaattattcacatatcttctaacttataaaacgtgaaattgtatgaagtacttagttataattgacatccataataacatagttagctaaacagtaagcatttagagaatgtaaggaactacacattacatgcaaaattttaaaaagaaaccacaatcatttgacctcacatactattgagaaaggagtttaagacagctacataaaaaacattaaaaaattacaaaactatttgtcaacaaagttgttcatgtccttgtgctactctgatgtaggattataacagttacatacatgcaaatggttaattccttccctcatatctatgaagtttagttttttcttcctgattacagaaatcctaagtttatcaatgtgttcaacttcattatttcttcaaattctgtttaggcatatcaagtgacttaagattctgttataactttctagaatctccccagtcttacttgaaactggctttgtaaaaaaaaaaaaaaagaaagaaaaaggtagaactatatggataattaattaacattttcctaaatgttatttaagtctaggcaccaaaatatataagatttgaactggccaggtgcagtggctcacgcctgtaatcccagcactttgggaggctgaggcaggcagatcacttaaggtcaggagtttgagaccagcctagccaacacagtgaaaccctgtctctactaaaaataaaaaaattaggtgggcttggtggtgggcacctgtaatcccagctactctggaggctgaggcaggagagtcacttgaacctgggaggcagaggttgcagtgagcagagattgtgccattacactcctgggagacagaatgagactccgtctaaaaaaaaaaaaaaaaaaatgagcctaggcaaggtggtttatgcctgaaactccagcactttgggaggattgcttgaggccaggagttccagaccagcctgcaacaaagcaaagccttgtctattaaaaaaatatctggaggttgaggtgggagtatcacttcagcccaggagttcaaggctgcagtgagctatgatcatgccactgcactccagcctgtctctaaataataataaattttaaaagtaaaaaatatttttaaacgacttgactacaataagagttttaagtcttttcaataaatcttcaatcagttgctaatatcatctcgtgcttcaaacacagtgcccagtggtgtgctttagtgaaagcggtttaatttttggagtctgccagaactgggtttggttaccagctccattactaactggctaggtatctttaggaaagttaacttccttccttgttttaattcttatatctgaaaaaaggagatggcagtaacttctcaaacagctcttagctgaaaattaagggagatacaagcatatcgtatcttattgcgctttgcttaattgtacttcacaaatattgtatgttttacaaattcagggtttgtggcagccctgcatccagcaagactatgggcatcatttttccaacagcacatgttcacctcctgtctctgtcacgttttagtaattctcaaaatatttcaaactttttcattatcatctctgctatggtgatctatgatcagagatctttgatgttattactgtaattgtttttgggtgccacaaaccttgcccatgtaagacaataaacttaactgataaatgtgtgtgttcagactgctccaccaattggcattctgtccttgtctctttcttcatgatttcctattccctgtgtcacaacaatattgaaatcaggccaattaacaaccctacaatggcctctaaatgttcaagtgaaaggaagggttgcatatgtgtcaccttaaattaaaagctagaaagattaagcttcgtgagggaggaatgtcaaaagccaagacaggccaaaagctaggcctcttgcaccagttagccaagttgtaaatgcaaaggaaatgttcttgaaggaagttaaaaagtgctactccagtgaacacatgaatgataagaaagcaaaaaaacaaaacaaaaaaagccttatagctgatatggagaaacgcttaatggtctggatagaggatcacgccagccacaacattcccttcagccaaacctaatccagagcaaggccataattctcttcaattctatcaagggtgagagagggatgaaagctgtaaaagaaaagtttgaaaacagaagctggattatgaggttaaaggaaagaagccattcccataacacaaaagtacaaggtgaagcagcaagtgctgatgtagaagctgcagcaagttatccaaaagatctagctaagcccatggatgaaggtagctacactaaacaacagattttcaatgtagataaaacagtcttctcttggaaaaagatgccatttagaactttcagagctagataggagaagtcaatgcctggcttcaaagcttcaaaggaaaggctgactctcttgttaggggctaatgaagctgggcatttgaagttaaagccaatcctcatttatcattctgaagttcctaaggcccttaagaattatgctaaatctactctgcttgtgttctagaaatggaacaacaaagcctgagtgacagcacatctatttacagcatggtccactaactactttaagcccactgttgagacctactgcttagaaaaaaaaattcccatgagctctgatggaaatgtacaagaaggttaatgtattcatgcctgctaacacaacatctattctgcagcccatggatcactgagtaattctgactttcaagccttatgacttaagaaactcattttattaggctacagctgccatagattgtaattcctctgatgaatctgggcaaagtacattgaaaatcttttagaaagcgttcatcattttagatgtcattaagaacatttgtggttaatcggagaaagtcaaagcatcagcattaacagcagtttggaagaagttgattcaaacccttatagatgactttgagggattcaagacttcatggaggaagtaactgcatatgtggtaggaatagcaaaagaactggaattagaagtggagttcaaagatgtgactgaattgctaaaatctcataaaatttgaacacatgaggggtcttgtctcatggatgagcaaagaaagcggtttcttgagattcaatctgctcctggtgaagacgctgtgagcattgttgaaatgacaacaaaagatttaggatttaggatattatataaacttaggtgatagtgcagcagcaggttttgagaaaagtgactccaattttcaaagaagctctactgtgggtaaaacacagcattgcatactacagagtaatcttttgtgaaaggaacagtctatggatatggtaaacttcactgttgtcttatttaaagaaattgccacagacaccccaacctccagcaatcactacctcgataagtcaatgaccattcacatcaaggcaagaccctctaccagcaaaaacattacaactcccttaaggctcatgtgatcattagcaattttagcaataaagtattttcaatcagggtatgtacattgtttatttagacataaagctactgcacacttaacagactgcagtatagtgtaaacataacttttttagataccagaaagcaaaaaatttgtgtgacttgctttattgcaatatttgctttattgtagttgtctagaacagaacctgcgatatctctaaggtatgcctataacaaatacatataatgtgtctactatagtgcctggtatatagtaaggacacaaaaagtggaagtctccaacctccctccctagcctctcacattttttagaattcttcctagagaaaggagcattcttggtaggggccacagcctgcctgccttctatatttcaggttcactgctttagggaatgaaaagtagtgttttctcagcataagactgaaggcccccagatctcgatcaaagagtattttaaataatcttaggtctgatctccctggcatgaccttctgcctctattccccattgcttctagagttaatgttctcaaaaacaaagccaaccatttcacctctcagctttgtatctggctcccctctgagccaagcagaactttaagaaacaaccaaacagctagctgaaatgccatttctgggtccctttcttagatattttgacttggcttacctggggcgaggtgcagaaatcatctcaaaccagtacccaggtgattctgaggcagatagcttgaggaccaaaagaacactggcttaccagagaaaaccctaagtagatcaggagccaactttgggatgtgttggtttgagatattcagtctggagttcaggggaaaggtctgggctggagatagaaatttggaagtgattggattttgggtggtttatccacagtttggagactggattagataaccaaggaagtaagtatataaacagaagggaagaggaccaaatactcagaatactcagcactggaccctctcatattaaaagctaaaggagaagaacagtttgaaggggagctagtgaggaaaatcaagagagggtcgtgtcttgaaaaccaagcgaataaagtatttgcaggaaggtggagtgatcaattgtcaaatgctaccaatgagtcaagtaacatgggactgacaattgcccataagatttagtaacatggtaaccttgacaagagaagttttggtggagtgacagaggtcaacaactgattgggttaggtttaagagtgaatgggagaagaaaatgtgttagaagagttttgctgtaaaagaagaaaaatgattggtggctgaagagtaagtgaaatcaacaaaagtagtttgtttatattttaagatgagaaaaggaagagcatgttgtaggctgacaggaattatccagtagagaaggggaaaaatgatttaaatgttgcgggaggattggtagagcctgtccttgagtcagcaggagtaatctggatctagtggtcaaggagagagggctggacttgagaactgcagagctctggtaaccccagaaaaatgggattatagttcagaggccacttctgtccatacagatggtggtagcaatttcagaaagtttttttccgaaaattaggaagcaaggctgttagctgaaagtgaggacaggggagtgctggcaggctgagaagagaaggtggaagacagccatcccagaagccatggagaatgaggggactgtctgcagggcagattatctagtcataatattcactttcaattccacataccagttatgaaaagcttcttttaccccagaaattcagctagcaagtatctactctccctgatgtcaatcaattttcattacaaatacttaggacaaagagacactagataatgaataagaggtaatattaacaaataagagagaaggttctgtgggttccaatctcagctctaccattttatttactatttatctgtgcaagtgatttaaaatctttgtgtttaatataacacatttagccaagttcctgacatatgtgttcaataaatgccactattatgaaatgtgttcagaacctaactgaatttttttggaagattttttagcaagttagaaaatcttatttcaaaatgtttatgtatgaaactataaaattttgatctcttataataccttcacaacaaaatcaaataacagtggaatcatccccaaagacttgttttcaaaatgctatctacaaaacaacagtgtctttcaaacagaagttacctccttttttctatcatgaacattatctttaacttgaaggggcagattaactgtaccttctgctaaatatcttctttttttttttcccagagtcttgctctgtcacccaggctggagtgcagtggtgtgatctccactcactgtaagctccacctcctaggttcatgccattctcctgcctcagcctcctgagtagctgggactacaggcacccaccaccatgcctggctattttttttttttgtatttttagtagagacggggtttctttcaccgtgttagccaggatggtcacgatctcctgacctcgtgatctgcccacctcggccttccagagtgctgggattacaggcgtgagccattgcacccggcctatatatcttctaagtatcatactactgagacacttgtcatcataggaaaggtcagcaatctgaaatgcatatccttttataaaggaaaatgtaaacagcaaaaatttacttaagggttgggaggtagctggaggtaattaaaacatgagatagtaaacaattccaagtttgctatcaggcactaccagcctagggatttatggattgttgcctgcaatagggaaattctcacaggcatctactctccttgagcccttgcttcctactcagttgttaggccctttctatttcctactccttgccccagctgctagcctacaattccctaatttctattaaaatagtacatagagaggttcagttcttcatactttcatccgcccccccatccaaagtggcctctggatgggcctttgcacaaagaaacactcactgaagtgctctgcttaaagaagataaaccattgtggtctccactgttaattacaaaaaagttgccacaaactttgaaattacaataatttttaagatttaatatttttagtctaatacttagtcaggattcagactttctccttccataccccgcatttaaacaccagggttagatcttcctgtcctatacttccacaattctcagctcacacctctcatagatattaccagactgcatttctgcaacgtgtttatgtgcctttctcctaagttatgaacttggctcatccatttttgagtctcaatatttaacacaaatccaggcacaaaacggcattatttttggtataaattaataaataagcagacgactaaatgaagtgacaaaccagaacatctcaattctcataagttattttgtgcacaaaaattatattatttcaatatcctgctgaggacattatgtagaatataatacaaacaatttgaaattaaactacttctcagaatctgtgttaccagtttcctagctgttctctctgacttcaagctatctaatctctgtacaaatatattctgcatggtgcttttaggataatccttcctaaatactactttcaacatcactcctttactgagtaaaatgatcatgtacttcctatttctgaaggtgacgtcccccacccccacaattctctgtgtcgctttcatcacccttctcaattggttccaatctacccattgaacccattcctctctccacaaccctggctcaggtccttagtgcaccatccccagactgttttccttgtcccttacctttctgacattaatctttccaatatactgctgatatactaattttcttaaatattaaaatatgccactacactgcccaaaaaatatttaggggcttctcactctttactcaattaattgtaaacaccccattgtgcattccacattttcttgaccttctgttttataataatcctcaccattcctttaaagcagtggtaagctaacttttttgggggtgggaataaggggtcactgatctctttcaaaacctcatgaaaagaattatcctagaaaaaacacaaataatttatatataatcgtaagggattctgaccatgttccatgctttagccaaattgtccttctgctcagtctgtagtttttgtttacaaaagccattacaattcttgctatcaaaaatttcatgcattctcagcagcccatctagcatgctatcctttccataaaaacacattttggtctccctagctaaatatgatctttttccccttgaaatcaaaaacacatttgatttatatattacctatattatacttgaatatgggataaactccatgattgaacgaaaactatatgaagttaggtaatatggcttatttatcttttatagctccctgtagaacccagcatggtactaagaatttattaaaaatgataagaataggctgggcgctgttgctcacgcctgtaatcccagcactttgggatgccaaggtgggaggatcacttcaggtcaggagttcaagagcaacctggccaacatggtgaaaccccatctctactaaaactacaaaaattagtgtgtgtggtggtacatgcctgtaatcccagctactcgggaggctgaggcaggagaatcgcttgaacccaggaggcagagtgcagtgagctgagattgtgccactgcacttcagcctgggctacagagcggggactccctctcaaaaaaaaaagataagaataaatgataagaattgtttataaaaattttcagggctgctacattgcaaaaagccagcaggcaccactctcaacatagtctataaatcatttctgagtacatgtccaaaatctcttagctgaaatgtattgggccaattgtgtttcaaaattcaaaattgtttgtattttagaaagataaaatagagcaacacttaataaaacattatatttatgcagcaaaatgtacgaatatttacatagactacaagatttcctcatgtgatttcagatcagattttgctgccaaacaaattcacgtcaggttaggtattgttgacaagtgactacaacaaaatttttgtttgcagtgctttttgaatattgagattgtgtgtgaggaattgtgggcctgtgtccattcttggctccatctagtcaggaaggattccttaattcatgatacagagtaacctcaagcttttgcttcaatattcttcaagtttcattctttgttagtctctttcaacattctagggtacctgaaggtgaaaagtggcacatatggtcactggacaccattagggcagcagaggctagctgagactaggcggggccagtgtggaggaaagtcaggcaccaagttggtgtgtaattgcagccccttcagtaaatacttgcttaaagaatgaagctgggtgcagtggctcatgcctgtaatcccaacactttgggaggccaaggcgggaggatcacctgaggtcaggagttcgagaccaatctggtcaacacggtgaaaccctgtctctactaaaaataaaaaaattagctgggcatggtggtgggcacctgtaatcccagctattcgggaggctgaggcaggagaatcacttgaaccagggaggcacaggttgcagtgagctgaggtcaaaccactgcactccaggctgggtgacagagctagattccacctcaaaaaaaaaaaaaaaaaaaaaaagaccaaaacatgtcatcagaaattggaagtctctcatcctgacagtaagcacttagttattttcttttagatagctctctatacctagcaagatcaaatgggtacaattttttacccagaatgatattgttccttgctttttcataaaatcaaatattttagagacttcttgacatctgtagtatagacagctaactcactcttttcattgcaagggtataccacaatttgctaatctctttttaacagacatttgggctgtattcagtttctcagtattataaataatgtaatactgcaataaacatccacctacaggtattctagagttctgtaaggatgagtttctagaagtagaattgttgggccaaagtaacgcatattttaattgacaataccaaactatcttcagattctccaatggtcattatttgttattcattattattccagtactcaaattgctgaaattcccattattattttaatcagtaatgcttattagtgagttgagccttcttcatacttctgtgggctgtactttgtgaactgtcttaatattttttgttcatttttcttcctgtttgtctttttcttgatttgtaagaatattttgtatatgggtaataatttccttttgatttacatatatgttacagtttcctaatttgttgttgtctttgactttatgaattttttgccttatataagaaatttgagattacatagcaaatggagcaaatttgtttttgaattctggattttatatcaaatttagaaagttttccatcccaagactataaatacatacctccatcatctttccatacttttaggtttcttttcttcaaataaaaaaaaaattaagttgttgacccacctgaaatttttcttgtgtataatacgccaggagtcacaatattattatattaaaaaaactatcaaaaataaacgtttcttaattactgtctctgtttttaaaggctttgaataaatcttactagctcaataaactaccttttctaactactgcagacacatctctgccagtactactattaattggggtaagttaactaacttctgagtctttgcttacatatgaagtggaaatggtaaattctactttttaggattgttgtgaagaatagaaatattaaatataaagcatttagcatggtttctattatatataggcaatcagtaatgacaactcttggtactttcctctctagttcacttatagcaccctaagtctgcaacacacaatttaatcaaacactaaatttgcagtgttgtctgttaatcactcacttccacacacacactgcaggctccttgaaatcaaagactgtatcttttcctgcaaaggaatatccagtggaagttaagggcattgactccaggagtggagttctgctctactgttgactaactgaacaaatcttatctgtaaaatgagaaaaatagaacctgtctcataggtttgttgcaagagttaaatgagttagtaaacaaagagacattagaagagtacctgaatttagtaaacactcaataaatgataaccattatttttaccctttaatttttatatttcgtacaaatgctacagcactgttttgagctcacggtccatactcactacatgtgtattctatctcataaataaatcacttggttatagaagagtaaatagcatgaaaagtatttgcatttttttcctaaagaaataatgattactagtaaatactatgctttaagaaaaaagttatgattccttaaaatgtcatataaagtacacaacacaagacgctgtagtagcagttaaaaataaagctcaaaacctgttaattttttaaaattgagcattaaaatgtacttgttttgcaggagttaaaataaaaaggaaaacccaataggctatttacatatcacttattaaaaagcataatatctcatcagaggagaaaaaaatgtcttgatgatagattatatgaagtatgcattatatagttaattatatgagggccatctagccacaagaaggatattgtgttcatcggcctctcctcaaattaggtagatagggaagatatacagagtatgtgactttctgatgttcttaaatgacaatggagtttaaaaaccaagatgtaaaccagcagtcctcaacctttttggtatcagggaccagttttgtggaatacaatttttccatggatggtgtgggatggtttctggatgaaattgttctaccacagatcatcaggtattagattctcataagaagtgcgcaacctagatcccttgcatgcagaattcacaatagggttctcactcttatgagaatctaaggctgctgctgatgtgacagggaggcggagctcagtctgtaattctcgcttggccactgctcacctcctgctttgtggcccacttcctaacaggccatgggcccgcaccggaccatggactggctgtggtaccagtccacatcctaggggttggaaatcccttgtgtaaatgattagtgtgcctatttttgagtgtctgtcttaagtgtcaattatttcaatcaatcctttaaagaagtgactgataaaagattccctggagttactttgttttttttctgacaaaagcaaaagctctataactcagatgtgattttcggctaataatgtggccatgctgttagaagagtgagcagttggactttaaatcttgatcacaaatggtcatctcgtctttgcacaaatgccaaaaagataccagcaaatagagccaaaaaacttcttaggagtcactcgaataaatacttgattgagatctaagggtttcctgttataaaggagtgctttttaaaaaaaggttggctgagaatcctttgtcccaataaaaatctcaagtggaaatataaaccaaataaagaaatacagagctgttctgaatagaagaaggaacttgactgagttaaggagcacaatttgaaaaccagtctcctataagagaccatgtatttatctgaaaaagtataaagtaattaatatttgctcaaatacaaaacagtctaggaaaacagcacagaagcaggaatttaagatacagaagttagagtcagacctcctgaatagtagaaacctctctttacacccatctgtaaaatatagatactaacagaacctccacctcaaaggactcttacaaggattaattgagctaatccagtgaagcagttaaaacagtgcctgggttatagtgaaggcgacttaagtgactattaaatgaataagcctcttgcacaacatcaagatatgttagcagaataaactgccattcttctccatgtgtctaactgaatactacggtgtgttggccacggctctggcataacttgtatcaaggtattctcatgttcactagttgtaccacaggtttgttctgttttgttttcttcttcttggttttgtgcatgtcaaagacagggtctgcatacattttcatcttcagtcccagcacaagcaacttttaaatgtaggatcaatacatgtatctgaagtgaataaattagtgaatcaaatcatcttagcagcaggtagaaagctaatcatctagttcatttagaactgtatcaaaatacttcaatatattctggaaagagaagcaagacatgtaccaaaggagaaatgtacaaaaagggtttactcaagtaatagtgtaattagtaattcctaaatataatgatttttcatattatgaaatattaagcagtggttaaaaaaacaagttggaactacatatatgaacaatagagcttcaagtcatttttgtatcaaaataacatgatgcataaattatgatatataataccatttatatattaacataccccagagaacatattccacacacacatacacatgggaaaaactgaggtagaggtatagaaagatggtaggcaagggaacagattagaggtattgatcaagaggacttaagccttattgataacattttcattttttaaaggagatggatttttgtgttacttgagtaattaaagtacaatgattcagtggccagaacaggagcagttattagcactagcacgcatggctaataccactgcttctcctctggctcttgtagtggtattaaagtacatgttctgaaagagtattaaaaatctgggagctctaaataaccaaatatgaacagatgacaccaaatgctatctttttaaccatttctttttcagtacagtagagatttcaatgaaccctaatgtaaagtggtgcttaaaaccagtctatcattatccatgctaaatagtaaactgagagacctcagcttgaaaagaaattggatatagagttttgaaataaaatctagtgaattactacatatttatttttagggtaatgtgaaagctgctatagagcagactctattggagattaaatgaatataaataagattattggcaaaatatcttctcagagcccaaatattattcaaattcaaatagattatagtagaagagattgctggatgatctgtgcactacaaaagatggagaaagcattacagagctctaaatatagaacaaataaatcacagaagttgctgatgaaaaattggatgttcaatctttctctttctgatgctgtatcaaggagaactatgctaccaaaccagttttcattactagcctatgacaccaagtatacccaacttttgaaagaaacactttattcataaaagtatttcatttctgtctagtttcctctgcagtctatttattgtgatgtctaggtctgaaaaactttccccattatctgacagaggggtcaattctctagttgtccaaacattgcataaacgaatcacttaccacatgtgcaattagtcatgtgtacacaaataggtaaaatgtctctctccaccaccacactataaactttagtcaacagccagcaaggtctctcgttttactgtggtttcaccagtacctaacatagtgcctggcgcatggcaggtactcaataaatacatgaactaatgtaattaaggtaattacttcatgaacaattaatatatgaataaaagaatgaggacaggccaggtgtggtggctcacgcctgtaatcccagcactttgggaggccgaggcaggcggatcatgaggttaggagattgagaccatggtgaaaccccatctctactaaaaaatacaaaaaattagccaggcgtggtggggggcacccgtagtcccagctactcgggaggctaaggcaggagaatggcatgaacccgggaggcggagcttgcagtgagccgagatcacaccactgcactccagcatgggcaacagcgagactccgtctcaaaaaaaaaaaaaaaaaagtgagcataatatgcaatacaattgaaaatccttcaatacctacttagcttagtgtcttctaagtaatacatttcaattgtattataacatgtgtttaatgatattacaccaaatgtattcacaacaaatgctgtcactgagtaaagcatccaaaaaataaaagtgtggcgttatcagttatgatgactggatcatgtagcagttatctactggatgcctagtatattcctagcactgggctggttactgtgtactggcaaagataagccattgagaaatagtcctttccttcatcataccaggttagtagtctgcatttgtaggactggcaggtcatgattacttcctctctttggaaggcattaagtcaattcctggtcctttcatggttcactccaaacctacaagtcttggtaaaggaatgcaatgggctatccaaccaactggtttttggccaatactatatcctaaacagttcaattagcttaaaagaaatcatttttcccaactcctttctcccatttcctgagtggtatcttatttctttatctcttctctcttctgtctcatatccagggaaacccagtcatccagaatgactttataggctttatcacctccctctcctaacatgggagggaggtgaagaaactggctgttggctcctgcttatgtcttcttattgctgccctaaatgtgaagttaatatgatccatccttttatatcacacacttctcattacccctgctcttgcattctcctttaggattcacttttaccttcctagatgtttcatcacttggctcacatcttttcttctaaccctctattgtttttctttgactgcatttcctattatgagaaaggaagcacagtggtcaagggccgggattctggattcataccacctggatttgaatcctgactttaccatttcctaagcgtattaagacagcttaacatctacaggtcttagtttcctcatttgtaaagtgggagtaataataataataccaacattataacaactaaataagttaagacctgcaaagcactgaaatagtcactggcacagagaaagcattttataatgccatcattttcattctcatccttggcaacattagtatccatattgttgcctgtttactttaaacttctggccatattggcatccacacacagcgccaccttgaaccagctcatcacgcagcattgctttattgaagattttgaacctgatataagtccttctacttatctttctcattcctgaaaactcctagaacattctctatattctcttgccttaaattaccgattctccaaattcacccaaacactagcaatctagtttctcaaactttaactctctcaatttcctaatgtgaacattcaatgcttagtcagtgtaatcttaatttcttcactcttctttgactgtatacacttaaagctctcctcaatgcctagaaagcttatttgtcctttactaatcaaatgttcatatcttccatgtagacttcttcatcattcaagaatatacatatttcaacccataaccacctcgctctgtccaaatacaaaaactcacaaaccacacagatctcatatacattacagaattgcttacttgggcatttactgagaatctttatttagatgttttatgtgcatatgttacctccctaactatattacaagttccttgaaagtagaaatgtcaatggaaactgataatatgtcaggccttcttttacatactttacaagtatcagctcatttaatctttatagcaaccctaagaaatagccactgttatcctcattttatctcaaggcagagaaaagtgaagtcagggaaagtgtatctttggcataaattgctgaatgaaggaacttactaatgtagttcataatcttgttctagtttcatgaatgatttatctgcatcaatcttaagtaactctttgtaattactgctaaataaaggtgcttcacttctagtactcaagactataggaccatgccaacagccggagtccaacaaatgctttctaagagtacatttggaaagacttcgtctacagttttggatgctattctaacaaaatgacgagaaagcatttgtattttagttagggtgctctttgttctatatatgcactttatgaaataacttctgtgaatcaaacagtttatatatctacttttgcatatcatctctgatttacaagcgctgcaatgtaaaaatatttaacctatgtttactaacaatcagctactaactagttttgtgacctttggaaactcatatttatgtctttgagatttgcttatctttcaaaagaaggagctggggtaggacaaattgggcttaagaggattctaagggagtcattcacttataaaattatactatccttaggagactttaagaagagaggaaatcatattaatgcagaaggcaatgtttaagaaaccagaaccaaagtcagataaaaatggtaactccaattacccattggaaaactgacttttccacacaaagaacttccaactgtaactgtaaataaataatgtcaatgggtaagagccactatcagttgccctattttctcaagtaccacttttaacactgcctcaattagtttaaaatgcaatcaaaccccctaaagctgaatatgttcaacatcagtaaaatacattcatcttttgtgagaaaaaatcatgttctaaaaatatacaaagcaaatatttgttatgaagaactcctttgaaagctcccttaaagtgaaatttcctactacctgcttttctgcttgtaaggagagggctgttgaaacaaacaaacaaacaaaaatcctcagaacaggaaacaaaaagccaacattcagatccttggttccttgtctaggtgtgatctcagcactccagagggaatcctaagcttcattttacatagttatagaatacatttattgctggtattaaaatatgaaatctttcaatgtatttttatcttcaaatgtaaagatgtttggctgcttagactttaggtagaaagaaattcaaaaatattttctttgaaagttaaaatctgaaacactatgcaagtaaatctgaaatcattaagcatgtaagttcaatgagtttaaaaacatgaaagttttacttaaagcaaaatacttctttagaaattatgttactgtgatgtccaggttttttattttaaactgtttaatcctgtcctcttatacacttacaaaaatatactgaacaaaaagagaattcaaaaatttgtttagaattacttctttgtaaatattacataaatattcatgtagtcaaaagacagtatcatgtagaaaactgcatcatatacatgttgtgatatatgttcgtgtatgtacaaatatgtatacgcatacatgtacatatgtgtcaactaccatttttcttttaatcaaaattttaaagtactgcatacacatatacctacataatagatatataaaagtactattagaacaaagctgtttcctcaaagctaagcagttaaggaacaatagtagcactgtcctcaagatttttatgacttatgtctaatactgaaagtgttagtcaaagagtatgcaattatagtacccaaccacattccattttgagtatcataaataaaaacatattgttaaaatgtcatcatatattttaaattatatggaaatatcaccatttcttataagaaacaaggaataattgaaatctatacaatattagatggtctccatttacaattaaaccaataataaaggatcaggggaaaggagcctaccttccattgccctcttaaagaagaccttacagctcccacaggtaaggacaccataatgacagcctgatgcttcatccccacagattaaacaaatcttctgaggtaatgactcgaagctgtattgtgggctctggctggcttctgaatccggccttgaaatgcaaaacaaagtactccatttatttttaagtgcaccactatctaatactaaaggtttcttaaaccagtattaacagggtgaaaggagattttaaaaacagaactgtatatatacacttgaaagacatgcaaactaaacttggtaatagactatccgtaactggattcaccatacttgcaaatgattagccattcatcagtagcaccaatataaaagaacaataattagtgtataatattatgtcctagaaagttaaagtcggttaaaaggaaatctttctacaaaactctttatatagaaacaatttagtctttgacaaatgaaatgttatgtccttcctgtctgtatgctgggtcaaaacaaatatggtttggctttaaaagttcgatggtactttgcaatgttttggttttgagtttttaaaacacaaccctgtaatcattgcttacttaagcaagtatatgtttgtcattttaaagtattgtccaatacactgacactgttttgcctattcacaacataagattccaaacagagcattctttttttacattccattacttcagtatgcaaagtagtgttttttaaacctgtatataaaaaaaattgtagtgaatagcaagcaataataagaaaacggtgcaatgcagtgacaggcgtttaaatccacagtttaaacatttcccacgtgaacttaattttaatcttgagattcttaagaaaactgagccactgaagtgttcaaatccctaagatggtgctgctttcggttctggccggccacctctcaagtcccacccctcccgggggaacggtgccttaactcctctccaagacaagcactcctggtaaacgtccttcttctaagaaagaagcatttcataaagcccccaactcgccgcgcactttctgccccactgggctacagcggacctagtgtgacgctgcacgttttaaatcggggttggttctgcttcggaatctggaagattcggaaagttaaaaaacaaacaaaaaaacagctttcagggaggcacgccccaaaaatagtatattctgcgcccaccttcgtgtccccaagagtgaggagaggagggaagaagaagggaggcaactgcccctgtgctgtgtcccgctgcccaccctctccggccgccccggggagcgcagcggtgcgctggggctggggctgagggttggcggccgccgccgccaacggaaccgctactcccggacgcgctgggcgtgccccgtcccgggccctcacctcaggtagttgagatagggcgggtagacctgcggcaggccctccttgagcacggcggcctggtagccgagctgcgggagcccgttgaggccgagtgcagggtagagcgcgggggccgccccggcggcggcggcagaggcggaggtggagggcaggccgtcccgcgggagcaggcagccgctcgcgcccggcgccttgcagggcggcggcgcgaacgggccctgctggggcggcgcgccctccgctttgtacaggatgcactccagggtcgaccccgaggaggacgcagacgagactgaggcactggcgggtgcggccgtcaccgccgcttccccgggtctggatggggtcgctcgcggcggcagcgggggcggtggccccaacgggaaatccgggaaggctgcggggttggcaccggccacaaggtaggaacgcggggagcgcgcggaggcctccgcgccttcctcctcctcctttatctttagagcgggcggctggaagtcgctatagagagggtacgcgtcgtccttgggctcggcgtcgggcgggtacgcgcagtcggggaagtcgcctacagcgaccggggtggacgaggcacagggtgaactccgcggcggggcaaaggcgctggcagccccggccccgccgtcgtaactttcgtcttccagcagctgccgagtgcgggctgccaataaggcgtgattgagaggcaggataggcacgtggatgaaatccatcaccgtggtggccagcggggagcgcccgggcgccatcggcgcgtcctgctccaccagggcgaccctgggcgctgagaagcgggaatcttccttggggaccagggcgacgcctcctgctgccgcccccggcgggacagccgcggctcctcctccagccgccgcgccacccagagcccgaggtttgcccttcagaagcggacccgcagactcctcggactcagagccatcctcctcctcaacctccaccgcagcggcctgcggagacggcttcactggggccccggaccagtgagggctctcagaggccgggagcagcagctgccgggctggtgacaggccccggggcagcactttatgggcagctgccgtcccggagctgtctccaaccttgcacccggaccggctcatgagcggggacaacacccgctgggtggcgggggcagccggtggatcttcgggaagttcggggccaaacaggcaccaagagctggtgacctcgcaggcgggagggctgggttggctctgcccgggacctgagggcgccaacagagtgtccaagacactgtccagcagtccgctgtccttttctgggggactagaactgctgcctccagcaccccttgtagcttcagctctggaatatgcgccctccacgtccgacagcgactgctggtcctgcgtcttttcgtcggaggggtcctgtccctggcagggccgagggaagagtagcccgtccagggagataggtatggccgaaacttcaggcaaggtgtccgaggtctggctccccgggaacggacctgcggctgggcgacacagcagtggggatccgacctcgggggagggcgggccgcccgccacgtggggagcccggggaccctttgccttcagctcagtcatgacgactggactccccttttctcctcccccgtctccaggaggagggaaaagggaaggaggagggggtttcgggaatataggggcagagggaggagaaagtgggtgttgaatgtggctggaccggagggatctccacctcctgggtcgggggcgggggagggcggcgctggtcagctcctgcccttggcctccatcctgtcgtcaggggaactgtggctgtcgtttgtcccagcgagcggcaagtggggagcgcaagaaaaagtagtaattgttaggagatctcgtctcctaactcggggagttctccaagagagttctccaacttctgtccgaggactggagacgcagagtactcacaagtccggcacttgagtggctgcggctgcgacggcaatttagtgacacgcggctcctttatctcccgactttttctctggcatcaaactcgtgcatgctgtgaagctctcagtccctcgctgagttccactgccccctcactaaaaccctggggctagtcggacctctcggtacagcccattcccaggaagggtcggacttctgctggctccgtactgcgggcgacagtcatctccgaagatctcagatcccagtagtgcgggagcactagccgcctcgggttgtagatttcactcaaatgacaagtgaagctagttctcattgagaatgccacccacacgcacaaatacaacaaggcttaccccgattagtgacagctgtggactggccagacagctttctaacaacgcctcctcctctagggaggccccgcccaaagcccctccctaccccaattaccggtaggatctgaaactctggagttggcatttccacccgttattctgaatgctactctcaatagcaggttctttgggatggaacctcataagcatattacgttttgttttgcaaattaagaattatgccctatctaattggaaaaatgaatagattctatcagaagtagaatttttgtcaccattttaagatttcagttttgtaaagatttaacacagaggaagacatttggctacattatcttttaaaataataaatgtatcaaggacgatactaaataagagattctctcccttatgagttccataaaagtgaatgctttcaagtttctcctgctgcgaccaacatgtcctgctcctcttgagtatcctcagcgtgacaaagaaatttgggagtgggagatggtaacacctctgtatctatgatggaagtttggatgttgtgtgccacacttcgatttgtcttaaggaatgtgttccaatttttagtaaatagcactttaaggaaagtttcctttagctccagcttatacagtgtgctacatcataacatcctcagtagagatgttgaacagtactttttaccctaaatattcttagtctgaaaagtaattatctttgagcttataaaatggatcc

SEQ ID NO: 2 - protein, 933 aa, Artificial Sequence
  Description of Artificial Sequence note = synthetic construct
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 3 - DNA, 391 bp, Artificial Sequence
  Description of Artificial Sequence note = synthetic construct
tcacttcagtatgcttatctcatggtaacacaaaatatagattcaggaaaatcaactactcttattcattaaaacattaagttcagttgtgtaatatttacagttatgcctcaaaattatgggaaaacaaggggaaacgttgttccttaatgtaccaaatagcagacaacataaacccctcagttcactgtgacacagtttccyaattaccctacttgatatatttttgcactccattttaacttacaaatagtatgatacatattcttctcaagatataaatcaattgaaatatgtgataactctatacagtttaattttttttaaagcaaatggtagcagtattaaactgtatttatttaaataatttaagaattgagagctttaatgt

SEQ ID NO: 4 - DNA, 391 bp, Artificial Sequence
  Description of Artificial Sequence note = synthetic construct
tcacttcagtatgcttatctcatggtaacacaaaatatagattcaggaaaatcaactactcttattcattaaaacattaagttcagttgtgtaatatttacagttatgcctcaaaattatgggaaaacaaggggaaacgttgttccttaatgtaccaaatagcagacaacataaacccctcagttcactgtgacacagtttcccaattaccctacttgatatatttttgcactccattttaacttacaaatagtatgatacatattcttctcaagatataaatcaattgaaatatgtgataactctatacagtttaattttttttaaagcaaatggtagcagtattaaactgtatttatttaaataatttaagaattgagagctttaatgt

SEQ ID NO: 5 - DNA, 391 bp, Artificial Sequence
  Description of Artificial Sequence note = synthetic construct
tcacttcagtatgcttatctcatggtaacacaaaatatagattcaggaaaatcaactactcttattcattaaaacattaagttcagttgtgtaatatttacagttatgcctcaaaattatgggaaaacaaggggaaacgttgttccttaatgtaccaaatagcagacaacataaacccctcagttcactgtgacacagtttcctaattaccctacttgatatatttttgcactccattttaacttacaaatagtatgatacatattcttctcaagatataaatcaattgaaatatgtgataactctatacagtttaattttttttaaagcaaatggtagcagtattaaactgtatttatttaaataatttaagaattgagagctttaatgt

SEQ ID NO: 6 - DNA, 401 bp, Artificial Sequence
  Description of Artificial Sequence note = synthetic construct
aaccaagaaaatggaaggcatcaagaatccagtcacaactaggattgcatggcataaaaatcaattaaaaagtatttcacctatttggagagtaatagaactataggtcagaaaataagagtcctgatatcacaggaaaatcaatattataccattagcaccactaccactcctatgaccattactttaacctttatgacratgtatgtttactgcaatatacccaatgaaatgcctagattttgataaaggtgcttagtgtgcctgtttacatgtcaaaaatactcatcaggcctaggtacctgccactataaactagtaaagctatctgagtattttctttgcattcagaatcctttaaagtggtatttttcttattcttttataatagttaggatgag

SEQ ID NO: 7 - DNA, 401 bp, Artificial Sequence
  Description of Artificial Sequence note = synthetic construct
aaccaagaaaatggaaggcatcaagaatccagtcacaactaggattgcatggcataaaaatcaattaaaaagtatttcacctatttggagagtaatagaactataggtcagaaaataagagtcctgatatcacaggaaaatcaatattataccattagcaccactaccactcctatgaccattactttaacctttatgacaatgtatgtttactgcaatatacccaatgaaatgcctagattttgataaaggtgcttagtgtgcctgtttacatgtcaaaaatactcatcaggcctaggtacctgccactataaactagtaaagctatctgagtattttctttgcattcagaatcctttaaagtggtatttttcttattcttttataatagttaggatgag

SEQ ID NO: 8 - DNA, 401 bp, Artificial Sequence
  Description of Artificial Sequence note = synthetic construct
aaccaagaaaatggaaggcatcaagaatccagtcacaactaggattgcatggcataaaaatcaattaaaaagtatttcacctatttggagagtaatagaactataggtcagaaaataagagtcctgatatcacaggaaaatcaatattataccattagcaccactaccactcctatgaccattactttaacctttatgacgatgtatgtttactgcaatatacccaatgaaatgcctagattttgataaaggtgcttagtgtgcctgtttacatgtcaaaaatactcatcaggcctaggtacctgccactataaactagtaaagctatctgagtattttctttgcattcagaatcctttaaagtggtatttttcttattcttttataatagttaggatgag

SEQ ID NO: 9 - DNA, 431 bp, Artificial Sequence
  Description of Artificial Sequence note = synthetic construct
atgcgtaatttttaaaaattcaaaatattttccagatgaatgagttaaaagcataaaactagttaaataaaagcacaatttaggaaatactgaattgaaaataaaacactaaaagattttagtaaagaattctttttgaatgttgcaaatagagaattgaaacaataagttaaattggtcatattagataytaaaaacatacttacagaagagtagcaaatatacaatttggatatgttcttggcattcatacatcagctagtaagattaaaaactatgttgagtaattagccaaagaggagacctctgtgttacttacacggatgacacctaagttatccaaaaccctgataaataacaccagggatttatttgcagtgctagatttgtgtttcatgctaccttaaagaacctttctgcactgaaaccac

SEQ ID NO: 10 - DNA, 431 bp, Artificial Sequence
  Description of Artificial Sequence note = synthetic construct
atgcgtaatttttaaaaattcaaaatattttccagatgaatgagttaaaagcataaaactagttaaataaaagcacaatttaggaaatactgaattgaaaataaaacactaaaagattttagtaaagaattctttttgaatgttgcaaatagagaattgaaacaataagttaaattggtcatattagatactaaaaacatacttacagaagagtagcaaatatacaatttggatatgttcttggcattcatacatcagctagtaagattaaaaactatgttgagtaattagccaaagaggagacctctgtgttacttacacggatgacacctaagttatccaaaaccctgataaataacaccagggatttatttgcagtgctagatttgtgtttcatgctaccttaaagaacctttctgcactgaaaccac

SEQ ID NO: 11 - DNA, 431 bp, Artificial Sequence
  Description of Artificial Sequence note = synthetic construct
atgcgtaatttttaaaaattcaaaatattttccagatgaatgagttaaaagcataaaactagttaaataaaagcacaatttaggaaatactgaattgaaaataaaacactaaaagattttagtaaagaattctttttgaatgttgcaaatagagaattgaaacaataagttaaattggtcatattagatattaaaaacatacttacagaagagtagcaaatatacaatttggatatgttcttggcattcatacatcagctagtaagattaaaaactatgttgagtaattagccaaagaggagacctctgtgttacttacacggatgacacctaagttatccaaaaccctgataaataacaccagggatttatttgcagtgctagatttgtgtttcatgctaccttaaagaacctttctgcactgaaaccac

SEQ ID NO: 12 - DNA, 394 bp, Artificial Sequence
  Description of Artificial Sequence note = synthetic construct
ctcagtacatttttaccattgaaagtccccacttcagttcctggcaaactagtttggttggtttgtgtgactctgggcctgtatacccacaattaagaataaatccaatcttgtgtggttgacttaaggattatatataaaatacataaaacacttgtcacattggaggcagtaaatatatggtatttgttatytgattgatggggtcaagggaagacttcatggaagtggtagggtttgacttagattttgaaagaataatgaaatcttatttgatatagggcaagataatgcttaactggcaaagatggatagagatagaaaatagggcaaagggaacaggatgacccaaaacacaggaaaagaaaaattgtggggcatcttctagggattg

SEQ ID NO: 13 - DNA, 394 bp, Artificial Sequence
  Description of Artificial Sequence note = synthetic construct
ctcagtacatttttaccattgaaagtccccacttcagttcctggcaaactagtttggttggtttgtgtgactctgggcctgtatacccacaattaagaataaatccaatcttgtgtggttgacttaaggattatatataaaatacataaaacacttgtcacattggaggcagtaaatatatggtatttgttatctgattgatggggtcaagggaagacttcatggaagtggtagggtttgacttagattttgaaagaataatgaaatcttatttgatatagggcaagataatgcttaactggcaaagatggatagagatagaaaatagggcaaagggaacaggatgacccaaaacacaggaaaagaaaaattgtggggcatcttctagggattg

SEQ ID NO: 14 - DNA, 394 bp, Artificial Sequence
  Description of Artificial Sequence note = synthetic construct
ctcagtacatttttaccattgaaagtccccacttcagttcctggcaaactagtttggttggtttgtgtgactctgggcctgtatacccacaattaagaataaatccaatcttgtgtggttgacttaaggattatatataaaatacataaaacacttgtcacattggaggcagtaaatatatggtatttgttatttgattgatggggtcaagggaagacttcatggaagtggtagggtttgacttagattttgaaagaataatgaaatcttatttgatatagggcaagataatgcttaactggcaaagatggatagagatagaaaatagggcaaagggaacaggatgacccaaaacacaggaaaagaaaaattgtggggcatcttctagggattg

SEQ ID NO: 15 - DNA, 531 bp, Artificial Sequence
  Description of Artificial Sequence note = synthetic construct
tcacagaatcatactctttgatccagaaggcacaactggtctaccccctccattttcttctatagagttgtgcatgtaaaatgcttaaacattatctggtacataggagcatataagaaacactacctataatctttactgctgctattcatcaaggtctagagaattttacctaccaaaatcttctaataaatacatctctttcctcatatatggacaaaataagctttatttatattaatgagtcattaatggtaactgtaaagtttatcaatatgtctgtattgtacrtaatttacttataaaatatattattttctgggtaggcatgtcaagtagaagctattaagcacattttaaattaaaattacataatgttagacataccttttccacaagcatactgtaaccttacttttcatattcaactactcccaagctgtgtgtcgtttttagaacataaaaggaagacagaatatatttaaccaaatgcattaaagaaccaggagcagtttctggtcaaggatgtac

SEQ ID NO: 16 - DNA, 531 bp, Artificial Sequence
  Description of Artificial Sequence note = synthetic construct
tcacagaatcatactctttgatccagaaggcacaactggtctaccccctccattttcttctatagagttgtgcatgtaaaatgcttaaacattatctggtacataggagcatataagaaacactacctataatctttactgctgctattcatcaaggtctagagaattttacctaccaaaatcttctaataaatacatctctttcctcatatatggacaaaataagctttatttatattaatgagtcattaatggtaactgtaaagtttatcaatatgtctgtattgtacataatttacttataaaatatattattttctgggtaggcatgtcaagtagaagctattaagcacattttaaattaaaattacataatgttagacataccttttccacaagcatactgtaaccttacttttcatattcaactactcccaagctgtgtgtcgtttttagaacataaaaggaagacagaatatatttaaccaaatgcattaaagaaccaggagcagtttctggtcaaggatgtac

SEQ ID NO: 17 - DNA, 531 bp, Artificial Sequence
  Description of Artificial Sequence note = synthetic construct
tcacagaatcatactctttgatccagaaggcacaactggtctaccccctccattttcttctatagagttgtgcatgtaaaatgcttaaacattatctggtacataggagcatataagaaacactacctataatctttactgctgctattcatcaaggtctagagaattttacctaccaaaatcttctaataaatacatctctttcctcatatatggacaaaataagctttatttatattaatgagtcattaatggtaactgtaaagtttatcaatatgtctgtattgtacgtaatttacttataaaatatattattttctgggtaggcatgtcaagtagaagctattaagcacattttaaattaaaattacataatgttagacataccttttccacaagcatactgtaaccttacttttcatattcaactactcccaagctgtgtgtcgtttttagaacataaaaggaagacagaatatatttaaccaaatgcattaaagaaccaggagcagtttctggtcaaggatgtac

SEQ ID NO: 18 - DNA, 481 bp, Artificial Sequence
  Description of Artificial Sequence note = synthetic construct
atttttaaaatacaaaattgttgtgtagatgcagtgattattctttttgtttctcacacttgatttaattttatatttatgaaccagctgtttacaagaatctcacgttggggaaaggggaaagaccagggtaacctggcacctctataactcaaaggctgtcacctctgttacagtgacagagctaggttttatttcattttctctactactttttgaatctatcttggctcagaggagrttctactgccatacctaaacttcttcaggttctaatctacctgtatcaccaaggaagagatcatgccttctggaaaatctcccttacatatagacgatttttaaaattatgttattatctactagccctgggtcacttctgagctctctttccctgcctccaagtgtgctccccagccttgctcatagcagttcccattcagagtatagtcctctcctgggtatgtttatgtggtgatag

SEQ ID NO: 19 - DNA, 481 bp, Artificial Sequence
  Description of Artificial Sequence note = synthetic construct
atttttaaaatacaaaattgttgtgtagatgcagtgattattctttttgtttctcacacttgatttaattttatatttatgaaccagctgtttacaagaatctcacgttggggaaaggggaaagaccagggtaacctggcacctctataactcaaaggctgtcacctctgttacagtgacagagctaggttttatttcattttctctactactttttgaatctatcttggctcagaggagattctactgccatacctaaacttcttcaggttctaatctacctgtatcaccaaggaagagatcatgccttctggaaaatctcccttacatatagacgatttttaaaattatgttattatctactagccctgggtcacttctgagctctctttccctgcctccaagtgtgctccccagccttgctcatagcagttcccattcagagtatagtcctctcctgggtatgtttatgtggtgatag

SEQ ID NO: 20 - DNA, 481 bp, Artificial Sequence
  Description of Artificial Sequence note = synthetic construct
atttttaaaatacaaaattgttgtgtagatgcagtgattattctttttgtttctcacacttgatttaattttatatttatgaaccagctgtttacaagaatctcacgttggggaaaggggaaagaccagggtaacctggcacctctataactcaaaggctgtcacctctgttacagtgacagagctaggttttatttcattttctctactactttttgaatctatcttggctcagaggaggttctactgccatacctaaacttcttcaggttctaatctacctgtatcaccaaggaagagatcatgccttctggaaaatctcccttacatatagacgatttttaaaattatgttattatctactagccctgggtcacttctgagctctctttccctgcctccaagtgtgctccccagccttgctcatagcagttcccattcagagtatagtcctctcctgggtatgtttatgtggtgatag

SEQ ID NO: 21 - DNA, 392 bp, Artificial Sequence
  Description of Artificial Sequence note = synthetic construct
gtgctgggattacaggaatgagccaccatgcctggccaagtcttcttaataactagttttcatcaccatttcttcatgtacactaaggttaaggcctaaagaaaacatctgcggttatttctgtgattacaggagttttaattcttaaaatttttttattatcaaaaacttatttcaaagcctataccatgwcaaactttgtttttgtaattacagtttttaccaataaactgtttctctcattctttcacaaataatgatcaagactttaggaagagagacatatggtgtagaatgaaaattggcagtcaactgaagataaccatctaggaaactagagttgaagaccaccttaatgtggaatagcaacaaaaatgcatgaaccagattga

SEQ ID NO: 22 - DNA, 392 bp, Artificial Sequence
  Description of Artificial Sequence note = synthetic construct
gtgctgggattacaggaatgagccaccatgcctggccaagtcttcttaataactagttttcatcaccatttcttcatgtacactaaggttaaggcctaaagaaaacatctgcggttatttctgtgattacaggagttttaattcttaaaatttttttattatcaaaaacttatttcaaagcctataccatgacaaactttgtttttgtaattacagtttttaccaataaactgtttctctcattctttcacaaataatgatcaagactttaggaagagagacatatggtgtagaatgaaaattggcagtcaactgaagataaccatctaggaaactagagttgaagaccaccttaatgtggaatagcaacaaaaatgcatgaaccagattga

SEQ ID NO: 23 - DNA, 392 bp, Artificial Sequence
  Description of Artificial Sequence note = synthetic construct
gtgctgggattacaggaatgagccaccatgcctggccaagtcttcttaataactagttttcatcaccatttcttcatgtacactaaggttaaggcctaaagaaaacatctgcggttatttctgtgattacaggagttttaattcttaaaatttttttattatcaaaaacttatttcaaagcctataccatgtcaaactttgtttttgtaattacagtttttaccaataaactgtttctctcattctttcacaaataatgatcaagactttaggaagagagacatatggtgtagaatgaaaattggcagtcaactgaagataaccatctaggaaactagagttgaagaccaccttaatgtggaatagcaacaaaaatgcatgaaccagattga

SEQ ID NO: 24 - DNA, 401 bp, Artificial Sequence
  Description of Artificial Sequence note = synthetic construct
agacggggtttcaccatgttggccaggctggtcttgaactcctgacgtcaggctcaggtgatccgcccaccttgacctctcaaagtgctgggattacaggtatgagccactgtgcccagcctacatggtatattttcatattaatagaataaaataaaagaatacaatgataggtatagttttcacagataaatgtatttyggcacaagctgctttttggtgttggctctccactatgacagtgagcatagcatgcaaagaaagtcctgaagcttccttaaacttgaaaagatgattcagttttgtcacgtttacttttcagtttttatagagaactaaggaatcattaattcatttttacaaacacctgcctatactatgtcattaaacagcttgctttg

SEQ ID NO: 25 - DNA, 401 bp, Artificial Sequence
  Description of Artificial Sequence note = synthetic construct
agacggggtttcaccatgttggccaggctggtcttgaactcctgacgtcaggctcaggtgatccgcccaccttgacctctcaaagtgctgggattacaggtatgagccactgtgcccagcctacatggtatattttcatattaatagaataaaataaaagaatacaatgataggtatagttttcacagataaatgtatttcggcacaagctgctttttggtgttggctctccactatgacagtgagcatagcatgcaaagaaagtcctgaagcttccttaaacttgaaaagatgattcagttttgtcacgtttacttttcagtttttatagagaactaaggaatcattaattcatttttacaaacacctgcctatactatgtcattaaacagcttgctttg

SEQ ID NO: 26 - DNA, 401 bp, Artificial Sequence
  Description of Artificial Sequence note = synthetic construct
agacggggtttcaccatgttggccaggctggtcttgaactcctgacgtcaggctcaggtgatccgcccaccttgacctctcaaagtgctgggattacaggtatgagccactgtgcccagcctacatggtatattttcatattaatagaataaaataaaagaatacaatgataggtatagttttcacagataaatgtattttggcacaagctgctttttggtgttggctctccactatgacagtgagcatagcatgcaaagaaagtcctgaagcttccttaaacttgaaaagatgattcagttttgtcacgtttacttttcagtttttatagagaactaaggaatcattaattcatttttacaaacacctgcctatactatgtcattaaacagcttgctttg

SEQ ID NO: 27 - DNA, 481 bp, Artificial Sequence
  Description of Artificial Sequence note = synthetic construct
gggggcggtggccccaacgggaaatccgggaaggctgcggggttggcaccggccacaaggtaggaacgcggggagcgcgcggaggcctccgcgccttcctcctcctcctttatctttagagcgggcggctggaagtcgctatagagagggtacgcgtcgtccttgggctcggcgtcgggcgggtacgcgcagtcggggaagtcgcctacagcgaccggggtggacgaggcacagggtgaastccgcggcggggcaaaggcgctggcagccccggccccgccgtcgtaactttcgtcttccagcagctgccgagtgcgggctgccaataaggcgtgattgagaggcaggataggcacgtggatgaaatccatcaccgtggtggccagcggggagcgcccgggcgccatcggcgcgtcctgctccaccagggcgaccctgggcgctgagaagcgggaatcttccttggggaccagggcgacgc

SEQ ID NO: 28 - DNA, 481 bp, Artificial Sequence
  Description of Artificial Sequence note = synthetic construct
gggggcggtggccccaacgggaaatccgggaaggctgcggggttggcaccggccacaaggtaggaacgcggggagcgcgcggaggcctccgcgccttcctcctcctcctttatctttagagcgggcggctggaagtcgctatagagagggtacgcgtcgtccttgggctcggcgtcgggcgggtacgcgcagtcggggaagtcgcctacagcgaccggggtggacgaggcacagggtgaactccgcggcggggcaaaggcgctggcagccccggccccgccgtcgtaactttcgtcttccagcagctgccgagtgcgggctgccaataaggcgtgattgagaggcaggataggcacgtggatgaaatccatcaccgtggtggccagcggggagcgcccgggcgccatcggcgcgtcctgctccaccagggcgaccctgggcgctgagaagcgggaatcttccttggggaccagggcgacgc

SEQ ID NO: 29 - DNA, 481 bp, Artificial Sequence
  Description of Artificial Sequence note = synthetic construct
gggggcggtggccccaacgggaaatccgggaaggctgcggggttggcaccggccacaaggtaggaacgcggggagcgcgcggaggcctccgcgccttcctcctcctcctttatctttagagcgggcggctggaagtcgctatagagagggtacgcgtcgtccttgggctcggcgtcgggcgggtacgcgcagtcggggaagtcgcctacagcgaccggggtggacgaggcacagggtgaagtccgcggcggggcaaaggcgctggcagccccggccccgccgtcgtaactttcgtcttccagcagctgccgagtgcgggctgccaataaggcgtgattgagaggcaggataggcacgtggatgaaatccatcaccgtggtggccagcggggagcgcccgggcgccatcggcgcgtcctgctccaccagggcgaccctgggcgctgagaagcgggaatcttccttggggaccagggcgacgc

SEQ ID NO: 30 - DNA, 481 bp, Artificial Sequence
  Description of Artificial Sequence note = synthetic construct
atataaactgtttgattcacagaagttatttcataaagtgcatatatagaacaaagagcaccctaactaaaatacaaatgctttctcgtcattttgttagaatagcatccaaaactgtagacgaagtctttccaaatgtactcttagaaagcatttgttggactccggctgttggcatggtcctatagtcttgagtactagaagtgaagcacctttatttagcaataattacaaagagttrcttaagattgatgcagataaatcattcatgaaactagaacaagattatgaactacattagtaagttccttcattcagcaatttatgccaaagatacactttccctgacttcacttttctctgccttgagataaaatgaggataacagtggctatttcttagggttgctataaagattaaatgagctgatacttgtaaagtatgtaaaagaaggcctgacatattatcagtttccattgac

SEQ ID NO: 31 - DNA, 481 bp, Artificial Sequence
  Description of Artificial Sequence note = synthetic construct
atataaactgtttgattcacagaagttatttcataaagtgcatatatagaacaaagagcaccctaactaaaatacaaatgctttctcgtcattttgttagaatagcatccaaaactgtagacgaagtctttccaaatgtactcttagaaagcatttgttggactccggctgttggcatggtcctatagtcttgagtactagaagtgaagcacctttatttagcaataattacaaagagttacttaagattgatgcagataaatcattcatgaaactagaacaagattatgaactacattagtaagttccttcattcagcaatttatgccaaagatacactttccctgacttcacttttctctgccttgagataaaatgaggataacagtggctatttcttagggttgctataaagattaaatgagctgatacttgtaaagtatgtaaaagaaggcctgacatattatcagtttccattgac

SEQ ID NO: 32 - DNA, 481 bp, Artificial Sequence
  Description of Artificial Sequence note = synthetic construct
atataaactgtttgattcacagaagttatttcataaagtgcatatatagaacaaagagcaccctaactaaaatacaaatgctttctcgtcattttgttagaatagcatccaaaactgtagacgaagtctttccaaatgtactcttagaaagcatttgttggactccggctgttggcatggtcctatagtcttgagtactagaagtgaagcacctttatttagcaataattacaaagagttgcttaagattgatgcagataaatcattcatgaaactagaacaagattatgaactacattagtaagttccttcattcagcaatttatgccaaagatacactttccctgacttcacttttctctgccttgagataaaatgaggataacagtggctatttcttagggttgctataaagattaaatgagctgatacttgtaaagtatgtaaaagaaggcctgacatattatcagtttccattgac

SEQ ID NO: 33 - DNA, 401 bp, Artificial Sequence
  Description of Artificial Sequence note = synthetic construct
tcttggtttttaaactccattgtcatttaagaacatcagaaagtcacatactctgtatatcttccctatctacctaatttgaggagaggccgatgaacacaatatccttcttgtggctagatggccctcatataattaactatataatgcatacttcatataatctatcatcaagacatttttttctcctctgatgagatrttatgctttttaataagtgatatgtaaatagcctattgggttttcctttttattttaactcctgcaaaacaagtacattttaatgctcaattttaaaaaattaacaggttttgagctttatttttaactgctactacagcgtcttgtgttgtgtactttatatgacattttaaggaatcataacttttttcttaaagcat

SEQ ID NO: 34 - DNA, 401 bp, Artificial Sequence
  Description of Artificial Sequence note = synthetic construct
tcttggtttttaaactccattgtcatttaagaacatcagaaagtcacatactctgtatatcttccctatctacctaatttgaggagaggccgatgaacacaatatccttcttgtggctagatggccctcatataattaactatataatgcatacttcatataatctatcatcaagacatttttttctcctctgatgagatattatgctttttaataagtgatatgtaaatagcctattgggttttcctttttattttaactcctgcaaaacaagtacattttaatgctcaattttaaaaaattaacaggttttgagctttatttttaactgctactacagcgtcttgtgttgtgtactttatatgacattttaaggaatcataacttttttcttaaagcat

SEQ ID NO: 35 - DNA, 401 bp, Artificial Sequence
  Description of Artificial Sequence note = synthetic construct
tcttggtttttaaactccattgtcatttaagaacatcagaaagtcacatactctgtatatcttccctatctacctaatttgaggagaggccgatgaacacaatatccttcttgtggctagatggccctcatataattaactatataatgcatacttcatataatctatcatcaagacatttttttctcctctgatgagatgttatgctttttaataagtgatatgtaaatagcctattgggttttcctttttattttaactcctgcaaaacaagtacattttaatgctcaattttaaaaaattaacaggttttgagctttatttttaactgctactacagcgtcttgtgttgtgtactttatatgacattttaaggaatcataacttttttcttaaagcat

SEQ ID NO: 36 - DNA, 465 bp, Artificial Sequence
  Description of Artificial Sequence note = synthetic construct
cttgacaagcattggtctaaatatagcatttcccaaaggaaaatacacacaacgtccatggaacacaagctgtttatagttagtgcactgaaaaaacatttttagctttaacaattatgtaatatttttattatgtttttaaaaagttatatctagatgtaaaagctacgattcccagacagtgttgtgcaggatgtggctaagtgaaaaaattcaagggagtasgggtgtcttgcataactgtggctcgcttgatctccctcactctgctatgttttcattcaaactatctgtcactatctgacattaattttttaaatgtgtgctatattactgaaagttttagagattctaataaaaatatgggaaaagggaattattaatataactcttcatcaaatggattcctaccccagttacagtaatcttgctttaatcaatcacttggagaagctagtaagcaat

SEQ ID NO: 37 - DNA, 465 bp, Artificial Sequence
  Description of Artificial Sequence note = synthetic construct
cttgacaagcattggtctaaatatagcatttcccaaaggaaaatacacacaacgtccatggaacacaagctgtttatagttagtgcactgaaaaaacatttttagctttaacaattatgtaatatttttattatgtttttaaaaagttatatctagatgtaaaagctacgattcccagacagtgttgtgcaggatgtggctaagtgaaaaaattcaagggagtacgggtgtcttgcataactgtggctcgcttgatctccctcactctgctatgttttcattcaaactatctgtcactatctgacattaattttttaaatgtgtgctatattactgaaagttttagagattctaataaaaatatgggaaaagggaattattaatataactcttcatcaaatggattcctaccccagttacagtaatcttgctttaatcaatcacttggagaagctagtaagcaat

SEQ ID NO: 38 - DNA, 465 bp, Artificial Sequence
  Description of Artificial Sequence note = synthetic construct
cttgacaagcattggtctaaatatagcatttcccaaaggaaaatacacacaacgtccatggaacacaagctgtttatagttagtgcactgaaaaaacatttttagctttaacaattatgtaatatttttattatgtttttaaaaagttatatctagatgtaaaagctacgattcccagacagtgttgtgcaggatgtggctaagtgaaaaaattcaagggagtaggggtgtcttgcataactgtggctcgcttgatctccctcactctgctatgttttcattcaaactatctgtcactatctgacattaattttttaaatgtgtgctatattactgaaagttttagagattctaataaaaatatgggaaaagggaattattaatataactcttcatcaaatggattcctaccccagttacagtaatcttgctttaatcaatcacttggagaagctagtaagcaat

SEQ ID NO: 39 - DNA, 456 bp, Artificial Sequence
  Description of Artificial Sequence note = synthetic construct
aggtgaatttatagacagtcatccttacactacctatataaaattatggaaggatgtctagaggcatgcttctacttcagattaccaaaatgataatgaccactgcgcagtgctgggatcagtccactggcactacactttaacaatcctttatagcactgatagtgcttgccttccatttttcaggattcacctccaatttccagatcaaaacasagtgtgcctttgtatatacttcaaagaaaacaaagatgctagacaacaggaaatataaagtgaaatcaggtattttccaaaaaccaaaactatactagaattgatttttttaaatgtatatgtcttaatcattcaaataaagtcaactacaatggaggagatatttgaacccctcaattctaactttttactgagtctagaaaataataaatgcttgtagatactactgaaaagttctag

SEQ ID NO: 40 - DNA, 456 bp, Artificial Sequence
  Description of Artificial Sequence note = synthetic construct
aggtgaatttatagacagtcatccttacactacctatataaaattatggaaggatgtctagaggcatgcttctacttcagattaccaaaatgataatgaccactgcgcagtgctgggatcagtccactggcactacactttaacaatcctttatagcactgatagtgcttgccttccatttttcaggattcacctccaatttccagatcaaaacacagtgtgcctttgtatatacttcaaagaaaacaaagatgctagacaacaggaaatataaagtgaaatcaggtattttccaaaaaccaaaactatactagaattgatttttttaaatgtatatgtcttaatcattcaaataaagtcaactacaatggaggagatatttgaacccctcaattctaactttttactgagtctagaaaataataaatgcttgtagatactactgaaaagttctag

SEQ ID NO: 41 - DNA, 456 bp, Artificial Sequence
  Description of Artificial Sequence note = synthetic construct
aggtgaatttatagacagtcatccttacactacctatataaaattatggaaggatgtctagaggcatgcttctacttcagattaccaaaatgataatgaccactgcgcagtgctgggatcagtccactggcactacactttaacaatcctttatagcactgatagtgcttgccttccatttttcaggattcacctccaatttccagatcaaaacagagtgtgcctttgtatatacttcaaagaaaacaaagatgctagacaacaggaaatataaagtgaaatcaggtattttccaaaaaccaaaactatactagaattgatttttttaaatgtatatgtcttaatcattcaaataaagtcaactacaatggaggagatatttgaacccctcaattctaactttttactgagtctagaaaataataaatgcttgtagatactactgaaaagttctag

SEQ ID NO: 42 - DNA, 454 bp, Artificial Sequence
  Description of Artificial Sequence note = synthetic construct
aataaataaatgggagatactcatacaatgaaatcatacagtgtaggaaaaaacaaaccagaataatacaaatcaaaataaaaaacacaaatataatattaggcagtaagatcaaattgcataaatatacatagcatttctatggagtagaaaaaaacatctcaaaactaaaaaatctattagtgagaaatacatagatattaaaaaatcctayagaataatataagaaaatgaccaactccaaattaaagatatagaggtttctggagaaagataaagaggaatgtgataaggaagaaatatgcaatcaatttcaagtgtagtgataatattcatattttcttaagccaactgagtccagaggtggatgttaatcatattgtttctatatgtcatttgctttatatgcatttttttatgaataaaatatattaaaaagtatggcttattctta

SEQ ID NO: 43 - DNA, 454 bp, Artificial Sequence
  Description of Artificial Sequence note = synthetic construct
aataaataaatgggagatactcatacaatgaaatcatacagtgtaggaaaaaacaaaccagaataatacaaatcaaaataaaaaacacaaatataatattaggcagtaagatcaaattgcataaatatacatagcatttctatggagtagaaaaaaacatctcaaaactaaaaaatctattagtgagaaatacatagatattaaaaaatcctacagaataatataagaaaatgaccaactccaaattaaagatatagaggtttctggagaaagataaagaggaatgtgataaggaagaaatatgcaatcaatttcaagtgtagtgataatattcatattttcttaagccaactgagtccagaggtggatgttaatcatattgtttctatatgtcatttgctttatatgcatttttttatgaataaaatatattaaaaagtatggcttattctta

SEQ ID NO: 44 - DNA, 454 bp, Artificial Sequence
  Description of Artificial Sequence note = synthetic construct
aataaataaatgggagatactcatacaatgaaatcatacagtgtaggaaaaaacaaaccagaataatacaaatcaaaataaaaaacacaaatataatattaggcagtaagatcaaattgcataaatatacatagcatttctatggagtagaaaaaaacatctcaaaactaaaaaatctattagtgagaaatacatagatattaaaaaatcctatagaataatataagaaaatgaccaactccaaattaaagatatagaggtttctggagaaagataaagaggaatgtgataaggaagaaatatgcaatcaatttcaagtgtagtgataatattcatattttcttaagccaactgagtccagaggtggatgttaatcatattgtttctatatgtcatttgctttatatgcatttttttatgaataaaatatattaaaaagtatggcttattctta

SEQ ID NO: 45 - DNA, 441 bp, Artificial Sequence
  Description of Artificial Sequence note = synthetic construct
gtttgtaggatctccatcctagaccaaacaccattaagctcatccaagaatactgaatgagagttatctggtcatcaatatgtaagtttcgaaaacctacaaaacaaatttaaaaatacagtgaccataaaaatgactgaattatctcaaaaatgttgaaaatacatataaatctgaaaataggtatgcgtagggtaatawaatgttttcaatcccaacatattttacactgaaaatcagaaaacattaataaataatacagcctaaaatatcagcaactttggaagtcacttgaaattagtcactgtgatttagcatttctgggattttgctaagtgaatccatcatccatcactcattcatgcaacacccatttaataagaaccatttgggtgccaggcactgtataaaaatgaagatgagtaagagtcaatcctagcc

SEQ ID NO: 46 - DNA, 441 bp, Artificial Sequence
  Description of Artificial Sequence note = synthetic construct
gtttgtaggatctccatcctagaccaaacaccattaagctcatccaagaatactgaatgagagttatctggtcatcaatatgtaagtttcgaaaacctacaaaacaaatttaaaaatacagtgaccataaaaatgactgaattatctcaaaaatgttgaaaatacatataaatctgaaaataggtatgcgtagggtaataaaatgttttcaatcccaacatattttacactgaaaatcagaaaacattaataaataatacagcctaaaatatcagcaactttggaagtcacttgaaattagtcactgtgatttagcatttctgggattttgctaagtgaatccatcatccatcactcattcatgcaacacccatttaataagaaccatttgggtgccaggcactgtataaaaatgaagatgagtaagagtcaatcctagcc

SEQ ID NO: 47 - DNA, 441 bp, Artificial Sequence
  Description of Artificial Sequence note = synthetic construct
gtttgtaggatctccatcctagaccaaacaccattaagctcatccaagaatactgaatgagagttatctggtcatcaatatgtaagtttcgaaaacctacaaaacaaatttaaaaatacagtgaccataaaaatgactgaattatctcaaaaatgttgaaaatacatataaatctgaaaataggtatgcgtagggtaatataatgttttcaatcccaacatattttacactgaaaatcagaaaacattaataaataatacagcctaaaatatcagcaactttggaagtcacttgaaattagtcactgtgatttagcatttctgggattttgctaagtgaatccatcatccatcactcattcatgcaacacccatttaataagaaccatttgggtgccaggcactgtataaaaatgaagatgagtaagagtcaatcctagcc

SEQ ID NO: 48 - DNA, 436 bp, Artificial Sequence
  Description of Artificial Sequence note = synthetic construct
cctacgcatacctattttcagatttatatgtattttcaacatttttgagataattcagtcatttttatggtcactgtatttttaaatttgttttgtaggttttcgaaacttacatattgatgaccagataactctcattcagtattcttggatgagcttaatggtgtttggtctaggatggagatcctacaaacaygtcagtgggcagatgctgtattttgcacctgatctaatactaaatgagtaagtagtaacttttgttgtttttgttatttttaagtgtacatgtaggataattttgaaagttatatttcaatagatgatcacatatttagtgttcttgatatgatgacattactgtcattttacggtaattttatatttggagctttttcctcttgtttcaaaatatagttatatcaaatccaattttctt

SEQ ID NO: 49 - DNA, 436 bp, Artificial Sequence
  Description of Artificial Sequence note = synthetic construct
cctacgcatacctattttcagatttatatgtattttcaacatttttgagataattcagtcatttttatggtcactgtatttttaaatttgttttgtaggttttcgaaacttacatattgatgaccagataactctcattcagtattcttggatgagcttaatggtgtttggtctaggatggagatcctacaaacacgtcagtgggcagatgctgtattttgcacctgatctaatactaaatgagtaagtagtaacttttgttgtttttgttatttttaagtgtacatgtaggataattttgaaagttatatttcaatagatgatcacatatttagtgttcttgatatgatgacattactgtcattttacggtaattttatatttggagctttttcctcttgtttcaaaatatagttatatcaaatccaattttctt

SEQ ID NO: 50 - DNA, 436 bp, Artificial Sequence
  Description of Artificial Sequence note = synthetic construct
cctacgcatacctattttcagatttatatgtattttcaacatttttgagataattcagtcatttttatggtcactgtatttttaaatttgttttgtaggttttcgaaacttacatattgatgaccagataactctcattcagtattcttggatgagcttaatggtgtttggtctaggatggagatcctacaaacatgtcagtgggcagatgctgtattttgcacctgatctaatactaaatgagtaagtagtaacttttgttgtttttgttatttttaagtgtacatgtaggataattttgaaagttatatttcaatagatgatcacatatttagtgttcttgatatgatgacattactgtcattttacggtaattttatatttggagctttttcctcttgtttcaaaatatagttatatcaaatccaattttctt

SEQ ID NO: 51 - DNA, 461 bp, Artificial Sequence
  Description of Artificial Sequence note = synthetic construct
attttaatatctaacacttttattctatttcattcgattgatgctaaaatgcataatgctatgattctaagacttaggaaaaataatactatactgggatatggagtttatatatatttacatgaatttcttttttttcttctctgtaggtcgaaaatttaaaaagttcaataaagtcagagttgtgagagcactggatgctgttgctctcccacagccaktgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggtaata

SEQ ID NO: 52 - DNA, 461 bp, Artificial Sequence
  Description of Artificial Sequence note = synthetic construct
attttaatatctaacacttttattctatttcattcgattgatgctaaaatgcataatgctatgattctaagacttaggaaaaataatactatactgggatatggagtttatatatatttacatgaatttcttttttttcttctctgtaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggtaata

SEQ ID NO: 53 - DNA, 461 bp, Artificial Sequence
  Description of Artificial Sequence note = synthetic construct
attttaatatctaacacttttattctatttcattcgattgatgctaaaatgcataatgctatgattctaagacttaggaaaaataatactatactgggatatggagtttatatatatttacatgaatttcttttttttcttctctgtaggtcgaaaatttaaaaagttcaataaagtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggtaata

SEQ ID NO: 54 - DNA, 461 bp, Artificial Sequence
  Description of Artificial Sequence note = synthetic construct
ccataatatcagaatgttctatcatgagtatataacattttacatattattctagaaaatggtgggagttattttcactatggtaagaaaaaaacaccttgagaagtatttatattataaatagttagaaaaataaattgccatgtttgaatagcatatgaatttattatttttattacatgttttctactcatttgttaaaccaacagcttgtcaaacarcttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctga

SEQ ID NO: 55 - DNA, 461 bp, Artificial Sequence
  Description of Artificial Sequence note = synthetic construct
ccataatatcagaatgttctatcatgagtatataacattttacatattattctagaaaatggtgggagttattttcactatggtaagaaaaaaacaccttgagaagtatttatattataaatagttagaaaaataaattgccatgtttgaatagcatatgaatttattatttttattacatgttttctactcatttgttaaaccaacagcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctga

SEQ ID NO: 56 - DNA, 461 bp, Artificial Sequence
  Description of Artificial Sequence note = synthetic construct
ccataatatcagaatgttctatcatgagtatataacattttacatattattctagaaaatggtgggagttattttcactatggtaagaaaaaaacaccttgagaagtatttatattataaatagttagaaaaataaattgccatgtttgaatagcatatgaatttattatttttattacatgttttctactcatttgttaaaccaacagcttgtcaaacagcttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctga

SEQ ID NO: 57 - DNA, 481 bp, Artificial Sequence
  Description of Artificial Sequence note = synthetic construct
ggacaagcttgggcgcagcccctgtgcctgtgacctgagctctgccttggaatgaggtcaactccaaggaggagaacaaaccccttggtgtttttctttgctttggttataggatattcagagaaggtatgtattgaataatttctgccatgaacagctgctgtgtttaacttactaccaacacccccaaattcttaaattttctccagtgacagggtggacaaattattgaagaaaactrttctttcctgcttcttttcagcttttaaggccacattgattctaggccgaactttagaagtgtaggttctattagctccagctaactgatggtctgtaaggccttctttcctaagtttattaataaatagggatgtgatccaacctgaactcaataatctaggcccagttcagcttggattcatcttatggatgttgactaatctggcttggattatattttataatatgggtgaagagt

SEQ ID NO: 58 - DNA, 481 bp, Artificial Sequence
  Description of Artificial Sequence note = synthetic construct
ggacaagcttgggcgcagcccctgtgcctgtgacctgagctctgccttggaatgaggtcaactccaaggaggagaacaaaccccttggtgtttttctttgctttggttataggatattcagagaaggtatgtattgaataatttctgccatgaacagctgctgtgtttaacttactaccaacacccccaaattcttaaattttctccagtgacagggtggacaaattattgaagaaaactattctttcctgcttcttttcagcttttaaggccacattgattctaggccgaactttagaagtgtaggttctattagctccagctaactgatggtctgtaaggccttctttcctaagtttattaataaatagggatgtgatccaacctgaactcaataatctaggcccagttcagcttggattcatcttatggatgttgactaatctggcttggattatattttataatatgggtgaagagt

SEQ ID NO: 59 - DNA, 481 bp, Artificial Sequence
  Description of Artificial Sequence note = synthetic construct
ggacaagcttgggcgcagcccctgtgcctgtgacctgagctctgccttggaatgaggtcaactccaaggaggagaacaaaccccttggtgtttttctttgctttggttataggatattcagagaaggtatgtattgaataatttctgccatgaacagctgctgtgtttaacttactaccaacacccccaaattcttaaattttctccagtgacagggtggacaaattattgaagaaaactgttctttcctgcttcttttcagcttttaaggccacattgattctaggccgaactttagaagtgtaggttctattagctccagctaactgatggtctgtaaggccttctttcctaagtttattaataaatagggatgtgatccaacctgaactcaataatctaggcccagttcagcttggattcatcttatggatgttgactaatctggcttggattatattttataatatgggtgaagagt

SEQ ID NO: 60 - DNA, 436 bp, Artificial Sequence
  Description of Artificial Sequence note = synthetic construct
ggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaargagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagct

SEQ ID NO: 61 - DNA, 436 bp, Artificial Sequence
  Description of Artificial Sequence note = synthetic construct
ggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaagagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagct

SEQ ID NO: 62 - DNA, 436 bp, Artificial Sequence
  Description of Artificial Sequence note = synthetic construct
ggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagct

SEQ ID NO: 63 - DNA, 4409 bp, Artificial Sequence
  Description of Artificial Sequence note = synthetic construct
tacaataccatttaaatattttattcatatctatccgaatattgaccaggacactaatgccacactgcagagttaataatctgtgcattttctttaccgtaatggacagagtatgctttcttagctgcctgattcacatttctctaaaaatgctttatcggttaaagctttcaaccagcttaaaaataatgcctctcccatgtctccatgagtggaaaaaaagcaaacaaacctgtgtttaacaataaggtcagcatgacatacagcaacaagagccagtaaatcgaaaatgaggctgacattctgggactaggccagcagtcctgcaacagtcttccagactccacagctgcataaggctgcggacaagcttgggcgcagcccctgtgcctgtgacctgagctctgccttggaatgaggtcaactccaaggaggagaacaaaccccttggtgtttttctttgctttggttataggatattcagagaaggtatgtattgaataatttctgccatgaacagctgctgtgtttaacttactaccaacacccccaaattcttaaattttctccagtgacagggtggacaaattattgaagaaaactgttctttcctgcttcttttcagcttttaaggccacattgattctaggccgaactttagaagtgtaggttctattagctccagctaactgatggtctgtaaggccttctttcctaagtttattaataaatagggatgtgatccaacctgaactcaataatctaggcccagttcagcttggattcatcttatggatgttgactaatctggcttggattatattttataatatgggtgaagagtcagttgttttactgctttttgttcaattttgtatttagtaaggaccatagagtgtggaataatggtacattaaatagtccacagatgctagggagaaacagagattgttgttttcactcagccatgaataagaaaaaaagaaaccacaaaacctaggtgatgttcttttcattaaaatgatttcatgacttagtgaagtaaggataagcaatttaggacttggtgaacttttgctagactatctggctatgtattttctgtaaaattttaagagatgtagaaatttcccacatccaatggctattgaacaggcatactttgggaaaaacaaacttatgccattacttgtctccttgtccacttcactttatcagtggggaccacagttgttgagctattgaatacaaaaatagagtatctttaaatttgaaaaaaaatgtatgttttggcaagttttgaatgcttcttatgcacatggctctggagttgtaaaagtttttaaaatgattcatattctatcctgacttctgatgtgtgaaggataagtatggatgagagaaactgctcaggtattcacagcaaaaactaaaaatgtaatagccatccttccaaagcaataaaagacagattaaagcagacatagatttcttgtgggcattttatatcataaccttcttagggatagggatgttttttgggttgatgacttcattgtaatttctaaacccattgttcccttttgtggaattgctgccatagtaaaaataatttgagcatgggtttttggaggggctgtgttctagtcaggctctctgttctaaaaagacacttaaaaatgttaaaatcagtgtcattattttaaaatgtctacaatggaagagtgataggctctttagaactttgataaaacaggccctaaactcaaaacactgtattttttcagcagaagaaattacttgaatacttccagccctaatttcaaatgaattcaaccaaatatatcatagcacatggtgacataaataaaacagtgagactttctagaatcctgctttcactgtagaatatagctggtgagtttgataaaattatagccagaactatggcttaatgaaaacaaaatagtagcaattactatttctgaaggcctgtatacgttttttttggtttccatttctattcattacaaagcacttgggaaaggcaggtttaatcagagcagttaaatgacttgcttaatctcatccagctagtaagtggcagagaaggggggcacagtcctatgtcagttatctccacagtgcccatcatcaaacacatgactatctgtggataatttaagttacaggttgacattccatggctacttacaaggcatagttttggcaaaaaaaatattgttcatcatatttccatatcatctgtatagcaataaattatcttaatgttaaatgggaaaaaatagcatagcattgccttattaaaaagtgccacatctttaaacattaaacacacacacaattgtcacataactactgataatggaggtataatgctttagactcaaatggcttttcaaacctttttacagaaaatattttcattaactcaagaatctatttcagtaaccttcaagtatttcctgatatgtaacctgtcttcttatgatttagtgtcattttagagctaccagaaagaacacgacatgtaaaatgaagataatggttatgatgactaaatagtaatggagacattagtagataggactttgtagagaaaaagatttagaaattaggtattttccatttggtgaagccatattctttttaaaaaggtactttatttttaaagcttagccttaaatattaaatcctagtgtttagacatctacattgcagacagagatcaacatcgaagatatcaaacattttaacaaaagccaaacctgtggccaccacattctattccctcatagttgagtgaaattagaaaattttaaagttattatggaaattaaatatatgctggtggaaatgatttatatcatcaaaatattggtttaattcaaagtaacaagaatcagataataaaatgatcacaacatcaactgttccctgagtccatcacttaagcataaagggctaataatattgtttttgtttcttggggttttttttggatagacctacattcattttcttgtgtcacacagttcattctgagaggtgtcactctcacagaggtagatttcctgtaactattatatttgggagatgttaaaaatacttttttcttgggatgacatttgtggggaaaatatattgaaagccttttcaataagatttaatgcaaatgaattactgacacataaaggtgttatttaatatttaaagttagtacctttgtgctgcatgggtatcagtaaaattatatgtaaatgttttgcacctacctagtatacgttgagtgtacaaatattaaaatgcctatttatcaggcactgtaatttatacctactactggactgtaactttcctgagttatatgtctttcttcagctccaaattctcataaggcttattagcacatttttcttgtgctaataccaggtattcaatcaatatttgttgaatgaaattagaaatgaagaaagtttttatgctgaggtttacagtacaaaacaatttaaggctacataaacataagaaataattgttttgtggaattttgcttagggagagaaataatattccattcattcagtttaaaaacttagaacaaggaatctaaagtattgtccttaagacacataaaatacacttctattcttctatgttatagtcagtaaagggagagatctcacaaagtaggaagcaaaggtggaaaatttaaaagtaatgaagataattgatggagataaagcatgggagaagaaagtgataaagtcatgggtacagaaatatgcagaaatgtaaccaaagaatgatcgaaatctacagtaataccaagacagagagaaaatgtcattcaactctcagtcagcaaacactattgaccactttatatggctcccagactcccaggagagtcacaattgtaaaataacatgctactgtaactcaaatgtgcacaaaatggcaggtaaactctaggagaaggcaaattactgcttggaagactcagggaagattttacagaaatgacatttggctgagtctcgaaggttgaaacatgcatggaatttgtgtcaaagggggaaaattctggaagaaatttcatggtaaaaagatttgcatggctgaaacataatatgaattcatgatagtggaggataaagaggagaaaggtatgtgaattgatacctccctcctcctcctctccctttcttctttcctttgatttgaaaagatgttaataagctctgacccaaaggagcaattggcaggaaagatattttactactttcaaaggtgagagaattgagaatcagtaagagtgactaactttcagcatgccacagttggtaaggagtacagttaggccataatcttctgatttcttatgt

SEQ ID NO: 64 - DNA, 66635 bp, Artificial Sequence
  Description of Artificial Sequence note = synthetic construct
ccgctaaattcatgaagtcagtagaaaaagggaagaatattgcaatatatattttagatacacagtcataaactgtatttgacccaagaccattgggtattttaacatcatcttaaatataccttgttcaaaacagagtgaatgatttgcaaattttcttgtaggacctcaaggtctagctgtgctaatgactcatagtttatctcaaccatgtataaagaatgcagaagactccagaaggtgggggagccactagaggtaaggagcctggacccctcgatcatctggggaggggctatatgagtgagaaacaaacctttatcatgttaggttactaggatttggagttggttgttctagtaaatagtttactctgattaattttagatatgtggaagttaggcagatagaggaaaggtaaagagaattctgggaagcaggtatagaatgttcaccgggcatcaaatggcaggatataatatggcaactgcaaggggaccattatgaatagaacaggagcaagtgtgggcacagaagtatatgagactagagaagtaaactaatttattaatatcattattgttgttttcaggctttctctttccagcctatcttaaaacagttaccatgctttattgaggtgtaattccagtatcatacagctcactcaaagtatcagttcattgatttttcatgtactcagagttgtgcaaccatcagcactattaattttgtaacatgagtatgactctaaaatctcaatacccactagcagttattccacatttccgcctaaatctcccagcagccactaatatgctttttgtacctgtgatttggctattttggatcaaggttcatccatgttccacatgaatcagtacttcactccttcactcctttttattgacaaataatatttcattgtatggataaaccacattttatttatccatctctatcagttgttggacatttgggttgtttacaccttttggctaccatgaaaaatcctgctaatgaacattcatgtacaagtttttgtgcagcgtatattttcctttctcctggatatatgcataggagtggaattgctggttcatatggcaactctaggtgtggtctttggagtaactgccagactatggttcaaatgattgtacttcttacatttccaccagcagtgaataagggtcctagtttcttcacatctttgacaacattttttatctgtcttttttgttatagccccccaagtgtgtgaagtggtatctcactgatttatttatattttcctgatggctaatgccacacagtgtatagaatttgtaaaatcaggacagcattgccaggtagtcttgtaagccttttacctgtgttaactgtaatccttcaacagtctcacaaaataggtactgttatcccaatttcatagttgggaaaactgaggcacaaggttacccagtattataaagttaagtactgtaacccaaacccaagacatcaaatgacagccttcttaggagtgtgattttaaatgtcagatgccatataatttgattttccggtcgctgctgtaaaattggcacttggcctcattttaaatctgacaaactatatttggcttgattttggttggttataataattcccttgtcagaatgtctggggaggttgtttccaaaagattgaaaatcttgaagtgggaaaacaacaataaagatgtccagcatgtcctaaatcaccattattccataaattttgatgctgggtgattagcatttttggcacaggaacttcccaaatgtatgaatctttgaatgaacaaaattcactgattaaattcaggtgtgatggggctaacttcaaatattcctgaagactgtttttggagaagaggatttaaatgtggaagtattttttctatagtttggggaaaaaaaccactttatcaagatatagttgtcatataataagctgcatatatttaaattgtataatttgttgttagacctgtgaaactaaaatgataaacataccctttgcctccaaaagtttcctcatgccattttttaaatcccttcatactgttctgtcccatccccttcccctagtactaatcaccactttctggcaagccactaatctgctttctgtcaccaaatattaggtttttaaaaatatttatatctgatttattaaacattaaattatacatttgcatatgccttttaggtctgtttgctttagatgttttattaatagattttacttttgggaaacgattttggatttacagaaaaattgagcagataatatagagaatgctcatatatcatttgtacaattttctctgttagcattttaaattaatagggtacatttgttacaattaatgaactaatactgataaattgttattaactaatattcatagtttatttagattttcttcattttcacttaatgtcctttttcctttccaggattccatccaggacaccacatttaattgttatgatgtcttgtgctcctcttggctgtgagagtttctcagattttccttgtatttgatgaccttgacagtccttaggagtactggtgaggtatattgcaagatgcccttctattgggatttgatgtatcctcatgagtagactagggttttgggttttggcggggtggaaatcaaagaggtaatgtactgtatttattacatcatatgaagggtacatattgtcaacatgacttatgactgttaatgttgttctccatcacctggctgaagtagtgtttgtcaggtttctccattgcatagttactcttttttccctctttgcatactctgtactctttggaaggaaatcattatgagtcacttaaggaagtgtgggggagtagggagttatgctagccctcctttggagtggagtatctacataatttatttggaattctgtgaggaagatttgcttcttctcccccatttacttctttattcagtcttttctttgtatggattcatgaatactttgttgctcaaatttttccagtttttggcactgggggctttttcagttgactcttgtgctccttgacccactctctgtattgtgtgtatgtgtgtgtatgtgtggactttcctactttctggtattacaagatgctacagactcatcagactcatcttgtatatttcctgccccagtcgtagaatcagccatttatccaaggaagtttgagactttttatttgaaactggtatagaaatgaagatctgggtgccaaataaattaggttttatttcttagaattgtatataatatagtgtatctttttttcatttggtttctctcactcagcataattattttgagattcagctaaattatagtatgtatcaatagttaattctttttattctttttatttatatactatagtttgtttatccatttaactgtggataaatatctgagttgtttccaatttttttggtattataaatagagctactatgaaaatgttgtacaagtctttgtatgaacatgtgttttcattttttctgggtaaatacctagcagtgaaatggctggataaattagtaggtgctgtgatatgagtgtttgtgttctccccaaatctgtgtgtcaaaacccaatcaccaatgtgatagtcttagtaggaggaggtgttggggagtgattaggtcatggagcactgccctgccccttctaccatgtgagtacacaataagaagttgccatctaggagctacaaagcaggtcttcaccagacattgaatctgtcacctttattgtggactcccagcctcctgaactgtgagaaataagtttctgctgtttataagctacctagttcatggtattttgttatagcagcccaaatggaccaagacaatacatgtacatttaaccgtcaaacaattttccaaagtgattgtatcattttacatttccacccagtatattagagttctaattcttccaaaagctggacaacaactgatatagtcagcatttttaattttaccaattctaacaagtagatagtggtatctcattatggtttcaatgtgcatttcattaatgactaatgatgttgatcatgttttcatgtgcttatttgtgatatttatatattcctcttctgtgtgtctgtgtctgttcaaatcttttgtttgcttttttttaaaaaattgaattgttttgttattgttgaattttcagagttctttatgaattctgttagaagtccattgtcatatatgggatttgaaagtagttttttctagcctgtggcttgtctttaagttctcttaacaggatcttttgaagagcagatgtttttaatattgataaagcctctttatttacatttttatgaattatgcttttggtgtcataactgaatattccttgctaaacccaaggtcataaatgttttctgtcatgttttcttttggctttatagttttaggttttacatttatatctgtgatacattttaaagttactttttaatatggtataaggtctagatcaaaattcacttttttgcatgtggatatccagttgtttcagcgccatttgctgaaaagaataccccttcactaattgcctttgcacctttgttgagaattagttattcaaatatgtggagatctgtttctgtaccctccattccatttaactgtattattggacagtttttatgtcaatacattttcttgattgatgtagctgtaattttttaagctgttattttttaaatccctctgtataaatgatagcattcttcctctaaccttccagccctgggggtgaggaggggcagcccacagctgtttgagggaagagacaggatcgaaggtgtgctctgaagctacagattgcatgagaggcaacccataaagtttctaagcagagtggtaacagtggccccccacttctgtttctgaactacagctctcagcgtgttggtgactttgaaccccctccccagaaatggaaatgccaaaatggaagagccctcagccacctagctcccttcccagtaggacagagttaaaggctagaccatcttccctcctgggtggagccccccttccccccacggatacttaatgtactagtgctccaactttattcttctttttcaaaattgttttgtctaggtcatttgcattttcatgtaaatgttagaaacagtttgttaatatctgcaaaaaaaacccttctgcaatgttgattaggattgagctgaaattatagatgaatttggagagaactggcttctcactaaaattaagtcttccaacccatacaggtaatatatctctccttctctcagcaatgttttcaagctttcggtgtacaggtctttcacatcttttgtcagatttctctccaattatttcatatttttgctggtattttggatgatgtatatattagctttccattgctgagtcaggaagtaccacaaacttactagcttgaaagagcactcagttattatcttacagtttccatgggtcaggagtctgggcatcggttggatggatccattgcttagggctcacatggctgcaatcagtggattagccaacactgcgttgtcatctatggcttagggtgcccatctagacttattcaggtgactggcaggcagtgttcagttcctggcagctgtagaactcatgtgactttcatcttctttgaggtcagcaggaaacaattttactcatgctttttaagggtttctgcctgattaagtcagaccctcccagatgatgtccatacccatattatcaaaactgatctgggatttttttttttttttttttttttttttgcagagtctcattctgttgcccaggatggagtgcagtggcatgatctcagctcattgcaacctcaaactcctgggctcaagcaatcttcctgcttcagtctcccaagtagctaggactataggcatgtaccactgtgcttggcaaatttttatttcttatttttttttatggagatagggtcagtcttgctgtgttgccctggcagctcccagactcctgggttcaagtgatcctccccactcagcttcccaaagtgcggggatatgcaggcatgagccacctcacctgacctgatttgggatcttaattacatcttcacatttccttttgctgtaaatgtaacattatccacagaagtgacaccgatcatattgatagtcaagtccacattcaaggagaggcaattatacaaatactgtacaacggggggcaggaattttggagaccattttataattatactgaatttctatattgtgcattcttagtgtttacacaattgatctttagctggagtaatgaggttgggaatccttgccagttttttgattttaggaagaaaccattcagtcttttaacattaaacatgatgtttgctgcaggtttttcatagataccccttctcaggttgaagaaattcccttctattctgagtttgctgaaaattttattaagggtaaatgtaggtttttgccgaatgttatttcagcatccattgagataatcatacagtttttcttttttaattggttaatatggtgaatgacagtacttgagctttgaatgttaaatgaaccttgtattctttggctaaatcccacttcatcataatgtattactttcatatattgcattttgatttgttaaaagcttgtttagaagtttaaaatttctactcattaggaacactggcctgttggtttcgtttcttgtaatgactttatctgattttggtatcatgaattccactgtatacataatatattttctttctttttttgagatggaatctcactctgttgcccagactggagtgcagtggtgcaatctcagcctactgcaacctccgcctcctaggttcaaatgattcttgtgcctcagccttccgagtagctgggactacaggcgcatgccaccatacccagctaatttttttttttcttagtagagacggggtttcaccatgttggccaggctggtcttgaactcctgacgtcaggctcaggtgatccgcccaccttgacctctcaaagtgctgggattacaggtatgagccactgtgcccagcctacatggtatattttcatattaatagaataaaataaaagaatacaatgataggtatagttttcacagataaatgtattttggcacaagctgctttttggtgttggctctccactatgacagtgagcatagcatgcaaagaaagtcctgaagcttccttaaacttgaaaagatgattcagttttgtcacgtttacttttcagtttttatagagaactaaggaatcattaattcatttttacaaacacctgcctatactatgtcattaaacagcttgctttgtatgttattttccaagttgtactgcagtttctgcactgtcatatataatgttcataaaacttcacgacatatgtattattatctccattttgcagatgaggaaattgaagcttgctgtcttagagatagttgttaggagagccagaactcactactagggcttctgatatcaaatctagtgtgatttttattttaaatattgtgtttctttattttttacagtaccaagtgttggatagttgacaaggttcttgaaataatgtagaaacatttagttgctctgaggacccagaaatacaaacacattcagtagggcatattgggccacccagacctatgacttggttagttggaaatttttattgtctatacaagtttgagaaataggatttgatatctgtggttctagcttgaggcatttcttctatatttacatagggctaattgcaggtgtaacttgggacttgatggatactacaagagaaggagtatacaaaggtcacttctctgaccttcctggtgctccctcggtttctgaattcactgccccagtacaaccactgggttactaaggcccttccacagatgtactgaataacctgcaaagtatacctgatttttatacccatgtcagatagtatatattttgcctgaagtaacaggatactcaacaaaaagtgagttatataatagatgtttattatccctcataacaagaagtctggaggaaagtagttgtagggttggttaatcattggctcagtaatagtctcacagtcccagactccctgccttttcagtgagctaacctcagcatccaaagatggctactaaagctgcaggcatcagagcctcacataacctcatacagaattaggaataaggaaaatttctcttggcattattttttcaaatcagggggaaaaattcttacccagaagcccactccatagacatcctatcaaaacatattggctagaactgagtcatctgtccaccactaaacaagttactcaaacaagaatttgattagactaaccatggttcatctcctgcagctgtgtccactgctctgcgtcctttgctatcaatatgtgaacacaactggggtttagttagaaagaagtaagagtagggagatggctgttgggaattcaacaaatagtgtctgacattataccaagatcttatttccctcagagtcctaagtgatacttaatctaaactgagaaagaaattcacactaatgtatgaatgaatttaatagcccacttatttttataaatgtaatcttttcatatatttatcctgattattttttatacatgcatatgtacatatatattggcaattctattttacatgatatttcacaataactatctactccaaatatccctttagatgccttgaattagtcttcaaccttatgcactaattgctgatggctatggctctcatttctttggttctacagtttagcatatagttgttcagagtttacgcatagtttcatttgttacacttttgtgtccatgggactatatacacttaactagacctggcttgtattgcagtttggaaatacttttaatttttaattttgttgtctagttttttctctttaatgtaatatagcttttagggattttttttatattaccattaaaagtttataggctgggcacggtggctcacacctgcaatcccagcactttgggaggccaaggcgggcggatcacgaggtcaggagatccagactatcctggctaacacggtgaaaccccgtctctactaaatgtacaaaaaattagccgggtgtggtggcgggcacctgtagtcccagctactcgggaggctgaggcaggagaacggcgcgaacccaggaaacggagcttgcagtgggccgagatcgtgccactgcactccagcctgggcgacagagcaagactccatctcaaaaaaaaaaaaaaaaaaagtttacatagaatattttaggataaggaaagattggcaaatgtaagtactatagataaaaatctgccataatctgtagtttatatggttaatatgaatgatacattttgtgcaatttagaggaaaggtatcatgatactgggcaggagtatgaaggtcaagaactggaaggtaattcctctactacattccacaatcaaatcttttgaagcaggagtcataaataccagctgtttatcttaacatatatatattttttaaggtgaagcaagtttgttaagagagtaaaggaataaaagaatagctattccataggcagatcagcccccaagggctgctggttgcctgtttttatggttatttcttgattatatgctaaacaaggggtggattattcatgagttttctagaaaaggggtgggtaattcctggagctgagggttcctcccctttttagaccatatagggtaacttctggatgttgccatggcatctgtaaactgtcatggcactggtgggagtgtcttttaacatgctaatgcattataattagtgaataatgagcagagaggatgaccacaggtcactcttgtcaccatcttggtttaggtgggttttggccagtttctttactgcaaactgttttatcagcaaggtctttgtgaactatatctgtgctgacctcctgtctctttctgtgacttacaatgactaactgcctggaaatgtagcccagtagatctcagccttattttacccagtccctattcaagatagagtcactctggttcaaatgcctctgacatatcccccctctcttttataagagaacccttaatcctaagggttgcagaagtataaagatccactttctgtaacttcttcatgctgaataggggcgatgatattcctgcctaagtattagggtcttttgaatttcagtgtagagaggagaggccattcataactctgagttctgacaaaaggtgacttttggaagatgaataagtgtttaatttaagaaaacattcagtaagcttatcctgcattcctacacaaagagtacaacagcaatatatttcacaacagtaaaacaaaataagtgaaattatcccaagtaaactaaataagaaggctttccatgaactgggcaactggtggaaccaagctgatatggggttgatagctgattctaatgtgtgcccagaattaggatattgattcatatttatacattacccatccttcttgtttttcctgagctgcagtcagagatcactggctggttcatggtaataagcaaggtcagcctaaattgcaggaaaaaaaaaaaaacaactgataagactagaatataataataagtataccatagttcttgaaacataatttttttctctctctcgtcttccatttttactaaagacaaatgatggtagtcgaatttgctttattatgctttatttgtataaagtgcagcaagaataattatttttcacaaggcttgtaaaattggctttgatggaactctcttccataaggaatttcagataagaccttttcttaaagctgagctcagccgtgggtttgtaccctccactacctatgagttgggtaaattcctctcctcttgaggtccaaagataacttggggctcctgggtctgtcagaaagtgacattctttacttaccacagttcaggaaccttgcacagggacagggtagacaaggtatgaagccagttttcccaaggggcttttattggctctgtaagtcaagttttatttctcaaagaaaagcataacattcaagtcaaagccttggtaaaataaccattgtctccaactgtgtcctgttacaaatgaaaacagatttattgtacttatggaaataactatattgccataagtcaataatacttacaaatagtttctaaattatggagaaatcaggtagagagaaacaagtatggtccaaattttgttcacaggaatatactttactcaagtgttaaaagttgtaaatagcttttaaaaaaagcttccttgactctgaaaaacaaagcaaaggatcaacaacattttaagcaaaatgtcacataaattaattcagtcttctgttacttcattccatacagttaactcctgtcctgcttggtagtcagggacatttcagctctccctgactcctgaaagttttttttctctatcctaatgtcacacagtctccaaagttattagaaacctgcattcaagaacacctgttagagtcctgtagttgattataaaccaccttttaaagaggattaaaacaagacaacaattatctgtgtatgataaaaagttttagtacagccactattaaagccacaattgactgggaattttgattacttctgtggcatacaacagttttacataacaattgtaattattaataacatactaagtcatatgagaattataggagtttctcataattttggaacacataccactaacacatttttacaaatacaacccaaagaaagccaaacaccgtttcatatttgacaatgctttctgtatgatttttataccaaataagccaaatgtcacttttggagtttaggtgacctaatacctaaaaaattaattaggtcagaaaatcacatactttataatttgattttggaaagtttgtaaaatatcaaagatttaagacacttgatattataaaatagaattccaggttaccgtaagtcattcatttagtcaaaatcataacttaaaatttttgaaaagagaaaaacctttactcattaatagagggaagacagcttttccaacaatcttttttcttttctgtctttttcctacagtttattcaaaaaggcagacaaaaatctttcattccctaatttgtctaggaggaaggaaaggaatataatactgttatgataattttgataaaaaaggaacaaatgctatgtaaaatagtggtatctttacaagcttaaacaccagaaataatactttctcatgaggttaacaagatcaatagtaattttatagtcgagtttaaaataaaatgataattttaagtttaaaactataacgtgagacaaaaaaggctttctcttacttttacagttatactacatataagaactattactattgagaaggacattggattcccaataaaaagaaaaggagtgtatacatacaaatggaaaatcatacgttaaggaaaatataagatgcacgattaagtgcaaaaaaaaaaaaatccccctatcaccagaccatttctggagctgcaggcctttttcagttgtcagccaaactcattccttacaaaaagccttcattttttcattattctgacttaaactaaaaactactcaattcattgaaaattagaaaattttggcttagcaaaagccattatctttttacaataccagttattgtgaaagcacaatataacagcatagttaaatttagaaaatataaaacatggtagtagttctcatcttaaatagctacctgtatagaacaattccagtaaacaggaataaaagagaaggaagggtctgaagctcgcatagtgatttatattttattgccacaggagaatctacttccctttcttttctctgacattctctttctatgttgctaattctcaatctggttcatgcatttttgttgctattccacattaaggtggtcttcaactctagtttcctagatggttatcttcagttgactgccaattttcattctacaccatatgtctctcttcctaaagtcttgatcattatttgtgaaagaatgagagaaacagtttattggtaaaaactgtaattacaaaaacaaagtttgacatggtataggctttgaaataagtttttgataataaaaaaattttaagaattaaaactcctgtaatcacagaaataaccgcagatgttttctttaggccttaaccttagtgtacatgaagaaatggtgatgaaaactagttattaagaagacttggccaggcatggtggctcattcctgtaatcccagcactttgggaggccgaggcgggcggatcacttgaggtcaggagttcgagaccagcctggccaacatggtgaaaccctgtttctactaaaaatacaaaaattagccaggcatggtggcacatgcctgtagtcccagctacttgggaagctgaggcaggagaatgccttgaacccaggaggtggaggttgcaatgagccaaggtcgcgccattacactctagcctgggtgacagagcgagactccatttaaaaaaaaaaaaaaaagatgacttaattttaattttagtattcaaataatgtatcacttaaagatggttgtatgtttttcttcctcctctttcttccttaaatcaatttaatcatgttacctttactagtgcttcccattgtattttttaatatgcttctacatttatttgttctcagctttatcttaatattcttcctttcctacttatacaaacaggtcaatcatagagcattttctatttttcacatttcttcccattctctttccacttataatattctatgcagtgattcttattccaatgttggttttaattttaggttttctttggttaaaatatctaatgccaactgctgtagatttcatcaccaaatgtggtttctttagtatttttcaaaaggtgaaaaatcctcactaagttcttacatgttcaaagttcttggtcttctgtctttatacttgcagaagtgcttgactgtgtatgaaatctggggactttcttttgttgtgtatcttgtaggtgttgctcctctgtttcctgaatgtggagaagactcatgccattgtgtttttctttccattacaagtttcttaaggagcctttcttcatcactgaagtttaatagactttctaagataaatattattgttgactcttctgaatcagattgtgatatgattttgatgtgctgtttcaatatgtagattaaattgttttattataggaaagtatttcctttttaaatgatagcttaaaatattcatcgttttccattgttattttcttttttggagactacatttatgaaattattaggtctcttccgcctgtgttatatgtctattttctttctatttttaaagttttctttatttctgtttcttttttcttctttttgctgtttatttcctataattacttatgtgtcaatttcccacaatttacgtaacaaattaccacaagtttgatggcttaaaacaacagaaatatattctccgatagtgctagagactagaagttcaaaatcaaagtgttggcaaggtcatgctctatctgaaggttctagggacgaatccatttttgcttcttcgtagcttctagtgattctcagaaatccttggcattctttgacttttatctgcatatatcccatttctaccttcatttttacatggcctttttctctgtgtgtctctctgtgtcctcttctcttatatgggtaccagtcattagatatagggctcagcctaatccagtatgatttcatctcaagatccttgactaattacatttgcaaagaccttataaggtcaaattcacaggtatgaaagcttaggatttgaacatatcttttagggagacataattcaacccactacacagggtgtctattttatttgttctcatttaattttttaaaactttttttttttagattcagggaggcacatgtgtttattcattacataggtatattgcatagcggtaaggattgggcttctagtgtacccatcacccacatattgaacattgtacccaatagatagtttttcaatcctcttgcactacattttggagtccccagagtcttttttctccaactttatgtccttgtgtactcattgttaatgtttttatttggaaataatttcaaacttactgaaaaatttcaagaagggtacaaaagctcccatatatccttcatccagattttccaacatcttatccaatttactttactgtttgctctatcaaatctaatctgtctaatctattatataatatctattcatcatttacagatacagattgaaatacatttttttctggaccatttgggagtaagttgcacaaatcatactgctttactcgtaactacttcagtgtgaacaaggacattttcttatattaacatagtatggttatcaccctcaggaaacttgacactgaaacaatacattaatctctcatttttctaattttttcaattgatcaaataaaattccttatagcattttttttccctccagttcaggatcatgtgttacatttagttgtcatgtctttttaatctcctttaatctaaaatagctccttagcttttcttagtttttcatgactttgacatttcgaaatatgccggttcttttaaaacaatagactaatcctcatttttgtttagtctgttgtttctttatgattacattcaagttaagctctctggttggagtactgtattagtgattttgtgtccttctgaagacattacatacatacctctgcctgggaggcagaggtttcagtgagccgagacctcgccactgcactccagctgggtgacagagtgagagactctattttcccctcacccccccgcgaaaaaaaaaagaaagaaatcatgagttcttactgatactctcaaattcagttgattgccatagaggtgttcttgcctttccccattctgtttgtatctcacttcttccatagggaaagataacattgattcccagcaacatcaaagtatttactctcttatacataaaataatctcctaattattttatgcataccactataaaaatacctacacaaaattccaatttagtttgctcttctccccttcatccctacccacagactgaagcgctatagttaaatattgtgttttaaaaaaatttctctccaggatgattatgttattcatttctaatagagttgtgtacatttgtttcaatttgctctaagtattagatattttaatagtttgtttctcaactccttggtgatttatttttatagaagatgtagaacattaacatacttccaaaagtcataactacacaaaaagttatcctcagtaaattatgatgccctctcctgtccccttcacttactcccaatttcatttttttctagttaatgccccttttctttttgtttacacattcagatgtgtttgcttttttatttctttttatttctcacacaaaaggtagctctttttatttactatattccagaaataattttatatcaggccagagagatcttcttcattaatatttacagccacatagtactctgttgtctgcatataaacaatagtttattcaaacaatatcttatgtttgaacatttaagtagtttctaatattttacaagaaagaactatctcgtgcatatgtattttcatattcttggaaatgtacgttcaaggtaaattcaagaggtaggattgctaggtatgaaagataaatgaatgtgtggttttgttaagtaccaaattctcctccatagaagttgtactgttttacactcctaccagcaatgcaagagagtgcttgtttccctgcagtttcatcaacaaagtgaattttagcttttgacattttgccagtctgatagatgagatatgatatccttttatgtgctcatattttcatattttaagtggtctttatatcattatgaatttcctattcttttgcccattttatgattttttggagtttttctttcaattattaaaatctttttatatttagaatatttatattttatatatttatttgtcatatatgttgtaaatttttttctggcaaattatttatattcccaatttcttgtcatttcttgccatgaaaaatactttttaaaaattttgtgtagttcagtttgttaatttaaaaaattacatctggaatttttttttaagagacagggcctcactctgtgcaatggcacaatcgtagctcactgtaacctcaaactcctaggctcaagggatccccctgctcagcctcctgagtagctaggactatacataggtgccactatgcctggttaattgttactaactttttttttttttttttttttgcagagatgggggtttcacgatattgcccaggctggtcccaaactcctggcctcaagctatcctcctgccttgacgttcgaaagcactgagattgtaggtgtaaaccactatgcccagcctggaatttaagatataattgcaaagtcttttgctaccctaactttataaaggaatttacccctgtttttttatctagtattcacaaaatttaaaaccatttagacctttaattcgtttggagcttattcttatttatggcatgaggaatggatttaagtttatcttttccaaatgacttttcagttgtcccaacacccttaaatataaagtccatttctgcccccagtgatttgagatactgtctttgtcatatattagattttccataagtcatatggtttacttttgtactctctattctgttccattggtctttctattcgtatagtagtccgcattgttttaattacaaaggttttataatatgttttaatatcttatggggctagaacatcttcaaaactattctattttaggttttgctcactatctcttcttgtctattttttatgtgagctttagtatgaacttacctcgctctataaaaaaaattgttggtatttttattggaattgaatacttcataaataaaattatatattaatttatatgaaaataaatacatggcaaattaagttagcaagaaatgaaatcttgatgatattgataagttctaaccaagaacaaaatatgtctttccatttgttttgttttttttcttatattacgaaatttaatgagaaacaaagtctttttgtaggcccgatttcatgttttttcctacattagtccagtacctagactagtttattactccatatgccccattcttttccttttttttttttttaattatactttaagttttagggtacatgtgcacaacgtgcaggttacatatgtatacatgtgccatgttggtgtgctgcacccattaactcgtcatttaacattaggtatatctcctaatgctatccctccccccaccccacaacaggccctggtgtgtgatgttccccttcccgtgtccatgtgttctcattgttcaattcccacctatgagtgagaacatgcagtgtttggttttttgtccttgcgatagttttctgagaataatggtttccagcttcatccatgtccctacaaaggacatgaactcatcattttttatggctgcatagtattccatggtatatatatgccacattttcttaatccagtctatcattgttggacatttggcttggttccaagtctttgctattgtgaatagtgccacaataaacatacatgtgcatgtgtctttatagcggcatgatttataatcctttgggtatatacccagtaatgggatggctgggtcaaatggtatttctagttctagatcctgaggaatcgccacactgacttccacaatggttgaactagttgacagtcccaccaacagtgtaaaagtgttcctatttctccacatcctctctagcacctgttgtttcctgactttttaatgattgccattctaactggtgtgagatggtatctcattgtggttttgatttgtatttctctgatggccagtgatgatgagcattttttcatgtgtttgttggctgcataaatgtcttcttttgagaagtgtctgttcatatcttttgcccactttttgatggggttgtttgtttttttcttgtaaatttgtttgagttcattgtagattctggatattagccctttgtcagatgagtagattgcagaaattttctcccattctgtaggttgcctgttcactctgatggtagtttcttttgctgtgcagaagctctttagtttaattagatcccatttgtcaattttggcttttgttgccattgcttttggtgtttgggcaaagacatgaagtccttgcccatgcctgtttcctgaatggtattgcctaggttttcttctagggtttttatggttttcagtctaacatgtaagtctttagtacatcttgaattaacttttgtgtaagatgtaaggaagggatccagtttcagcattctacatatggctagccagttttcccagcaccatttattaaatagggaatcctttccccatttcttgtttttgtcaggtttgtcaaagatcagatggttgtagatatgcagcattatttctgagggctctgttctgttccattgatctatatctctgttttggtaccagtaccatgctgttttggttactgtagccttgtagtatagtttgaagtcaggtagcgtgatgcctccagctttgttcttttggcttaggattgacttggcgatgcaggctcttttttggttccatatgaacttgaaagtagtttttttccaattctgtgaagaaagtcattggtaacttgatggggatggcattgaatctataaattaccttgggcagtatggccattttcacgatattaattcttcctacccatgagcatggaatgttcttccatttgtttgtatcctcttttatttcgttgagcagtggtttgtagttctccttgaagagatccttcacatcccttgtaagttggattcctaggtattttattctctttgaagcaattatgaatgggagttcactcacgatttggctgtctgtttgtctattattggtgtataagagtgcttgtgatttttgcatattgattttgtatcctgagactttgctgaagttgcttatcagcttaaggatattttggtctggaagtcaaattgtccctgtttgcagatgacatgattgtatatctagaaaaccccgtcatctttccatttgttaacatgttgttccataagttaagactacatatgtgtttttaaccagtgttttatagttttctttgtatagctttgaacatttgaagtttattctaagtgttttatgttttctgttgctatgtaagtgtaattttctttttatttttatctcctcatggctatttgtgtatactaattctactgatatttgagtgttaatttcataacctactactttgatgaattctattgagttagtaggattttccaagtataccaccatattatctgcatataaagatgattttatttcatcttctgtaatttttatgcctcttctatcttttctaaatgcagtgacttatgtgtctagtattatgctaagtagtagagataatcatccttgccttatttctgaacttaattggagttcctgtggtgtttcctcattgggtccaatgctgctttaggattgtgagagatatatatatgtatatgtatatatacacatatgtaacaatgttatatgctgatgataagttatatgtaatatgtatatatgtgtgtgtgcatgtgtgtgtatgtgtgtgtgtgtgtgtgtgtgtatatatatatatatatatatatgcccacacactaagtattcatagcttcttattgtctctgaatgttctctgaatgggaacatacacagacacagtataaaatatatacacacagtaatatatgtacatatatgtaatatatgtacatatgtacatatatatacagatacacatgtattactaagtgtttatagctttttactgtcttgaatttttcattgggaatgagtttgaattttgtcaaaggctttttcatatttatttttcatcaaatttagtcttgatttctgaaattttttttttggccttttttctatttttaatccttgagtttttcaagttcttgattcatctacacttattgttctatcataagccactgttttctggccatctcttccctgagtttttgtatttctgctttgtggttttctttcttcaatatttgtccaaagcttagtggtttacaattttcatatgatcagtgtcaacattgctgatttttaaaataaaactattttgttatttccaatatttatttatttatttattgacatgcaattgtatatatttatgggtataattttatgttttactatatatatattgtataatgattgaatcagggtatttcgcgtatccattgcttcacgcatttatcatttctttgcggtgaggccattcaaaagcctctcttctagctattttgtaatatacaataccttaatattaccataattaccctactgtgcaatagaataccagaatttttttaaaattcttattttaagttctgtgatactatgtgcagaacatgcaggcttgttacataggtaatcttgtaccatggtggtttgctgcacctatcaacgcatcacctaggttttaagcccagcatacattagctatttatccttatgctctccctacccccacctgactcccacccgctgacaggccctggtgtgcattgtttccctccctgtgtccatgagttttcattgttcaactcccactcaggagtgagaacatgaagtagaacaccaggatttatttctcttatctaattgtaactttgtacccattgagtatcctcttcccatcctcccctttcccctcctctctccagtttctggtgaacattctactatctgcttctgtgatatcaatgaatttttaaaattctgcatatgagtgagattatgtggtatttatctttctgtgtctaacttatttctcttaacataatgtcctccaggtccatccgtgttgtcacaaattacaggatttcattctttattatggctgaatagtattccattctgtatatatgccacattttctttatcgattcatgtgttattggacacttgggttgattacaaatcttggttattacagatagtgctgcaataaacacaagagtgtggatatctcttcaatatgctggtttcatttcctttggatatataccaataagtgggtttgctgaatcatatgggagttctgtttttaattttttcaggagcccctatactgtttttcatagcagctctactaatttataatcccatcaatggtgtctatgtgttccttcttctctattcttccacaacacttgttttctgttgtctttttgataatcaccattctaactatagtgagttggtattttattatggttttgatttgctttcccctgataaactcttttcccatttttaaaatacaaaattgttgtgtagatgcagtgattattctttttgtttctcacacttgatttaattttatatttatgaaccagctgtttacaagaatctcacgttggggaaaggggaaagaccagggtaacctggcacctctataactcaaaggctgtcacctctgttacagtgacagagctaggttttatttcattttctctactactttttgaatctatcttggctcagaggagattctactgccatacctaaacttcttcaggttctaatctacctgtatcaccaaggaagagatcatgccttctggaaaatctcccttacatatagacgatttttaaaattatgttattatctactagccctgggtcacttctgagctctctttccctgcctccaagtgtgctccccagccttgctcatagcagttcccattcagagtatagtcctctcctgggtatgtttatgtggtgatagttgtcacactattctcccattgtcccctccttcaccttgagttttatcatatctcatttcagttttctgcaagatgtcggtaacaattttgctacaggcagcatttacacaaaatttgtagcttggaggtatttattctgtgttctgcctttgtcgaccattgaatgtgcattttttttctgattattcttatttccctatggctttttggagaagtggaataattcataacttagttgccaccaagtttttcctggaagttgaaacatattttacaatattttctctattttacttgtcatatgactagttatttaaattctttgtatggctgtttactgattttcaaagtggattacttagtatctagtaaggttgatgcaggagcacattgagatactaaagttggtataatatgtggcatgtggtaggtgtttattatggactgaattgtgtgctaccccacccaattcatatgttgaagcattaacccccaatttgactatatatggaggtagggcctttaaggaggcagtaagattgattgaggttataaggatggagcctaatctgatatgactgctgtcgttataaaaagagaaaagatagatttctctctctctctctctctctctctctctctctctctctctcttttgctctcttcccccctctacatacatgtgaacccagaggaaggactgggagggcacactaagaaggcagccatctgcaagccaggaagagaggcctcaccagaaaccaactctggcaccttgacctcagacttccagcctgcagaacagtgacaaaataaatcttgtcatttaagctacccagtctttggtattttattatgtcatcccatgcagactgtattagtcagggttctctagagggacagaactaataggatgggtatatatataaaggggagtttaccaaatagtattaactcacatgatacaaggtcccacaatagtctgcctgcaagctaaggagcaaggaagccagtctgagtcccaaagctgaagaacttggagtccgaagggcaggaagcttccagcatgggagaaagatgtaggtaggctgaggtcttttcctgtttttttttgcctgctttatatcctggctgcactggcaactgattagatggtgcccacccagattaagggtggatctgccttttccagcccactgactcaaatgttaatctcctttggcaacaccctcatagacacacccaggatcaatactttgcacccctcaatcccatcaagttgacactcagtgttaaccatcacacaggctaatagagtgttcattagattgtaactgctaataataacagttggtggtgatggtgatgatgatcctagaagtccaggcctatggatacttactgataaggaattgaaagacctgctttgaaaacggagtttttaggagggatataaataaactttctgctctgttactctcccctttccttttattaactggatttagaaatacttttatttggtaaaagaaatgttataaatcttcatacatatggctaatatttctggctaattaccatgaagcagaataaccattaatcatattattagagatacccaattaatttcattttgtgcttgattttagtactaaatttaagcagtttatgccacctcacttaggtcaactccaattatgatacatgtctttctagtattaaatgcaaaaacaatgagatgtctgtagctccctttattattacttgcatttcttcatctttcttttttttttggaatacacaatttattattttgatactgaatgaataaaattgttctttctttctcactgggaggcctgccttccaagctgcttttttgccaaaaatatttaccaggactttcaaagtcatctcttgttttcagttattattctcttttatgacaactatctttgaagagatttggaagttagtctatatttgaaattactgtgtgtgttttctgtgtttatatgtaatttcaacttaaaaatcaaaacatcttcacaattcctttggtatctcattttaaataaaagtatctttttttttttttttttttttttttttactgaaagtctagattagatattacttcctctgtgaaacctacctggacactaccagtgcaatcagagaaatttattgttgtatcagagcactttccatgttgtgttgtattttgtgtacagcatatggcatgtagtacagaatttaatggttaaatgtgtctccagttagactccacgtttcttgggaatatagactgtatcttttcacatcctcagaacctagcatttctctatgtcgatttcattattgtcagatcatactttgcatgctttcaattcttataaatttaagttttctgattcagaatatggtctgtgctggtgaatgttccatctgtggtttaaaagaatgtgtattgtgctgtttgggggtgtgaggctctatagcatttcagttggttgatggtgatgttcacttcatctatatccttgctatttttctgtctacttctatttattactgagggagaagttctgaaggctccaactgtattgtgaatttgtctatttttcctcagttctattagttttggcttcatgtatttgcaaacactgtttgatgcatatacattcagaattattgtattttcttgatcaaatgccccttatattattatgtaattttcttcttattctggtaattttctttgctgtgaagcctactttgaacaatatcaatatatccatttcaaggttcttttcattagtgagtgcgtagtatatctttttttgtccttttgctatttttaactgaatagatgtacctagttttggagtacatgtaataatttaatacattcatataatttacagtgatcaaatcagtatacttgatatatgtatcaccttaaatatttttcttttctttatgctaaaaccattttatagtttacaatactgttagatgacaaataactagaagacaagaatttgtgcaataccttattgtaaactatagttaccctgctgacccatcaaacactaggtcttatttattctatcaaaccatatatttgtactcatttgatatgatttggctgtgtccccaccaaaatctcatcttgaatgtatttcccataatcttcacatgttatgtaactgaagtaattgaatcagagaggcagataccttcatgctgttctcatgatagtgagttctcatgagatctggtggctttataaggggcttttcccccttttacttggtacctctcacctgctgccatgtgcagaaggacgtgtttgctttcccttctgccatgattttaagtttcctgcggcctccccagacatgtggaactgtgagtcaattcaacctctttccccttatagattacccagttttggatatgtctttattagcagcatgagaatggactaatatagtaaattggtaccaggtagtggggtgctgctgtaaagatacccaaaattgtggaagcaactttggaacttggtaacaggcataggttgaaacagtttggagggctcagaggaagataggaaaatgtgggaaagattggaatttcctagagaattggcgggctcagaagacaggaagatgtgaaaagcttggaacttcctagagacttgttaaatggctttgaccaaaatgctgatagtgatatggacaatgaagtccaggctgaggtggtctcagatggagattaggaacttgttgggtactggagtaaaggtcactcttgctatgcaaagaaactggcagtatattaccctgccctagagatctgtggaactttgaacttgagagagttgatttagggtatctagcagaagaaatttctaagcaacaaagtgttcaagaagtgacttgtttgctgttaaaagcattcatttttatgtagtcacaaagatatggtttggaattataacttatgtttaaaagggaagcagagcattaacatttggaaaatttgcagcctgaggatgcaatacaaaagaaaaacccattttctaaggagaaattcaagctggctgcagaaatttgcatgagtaatgaggaaccaaattttaatcaccaggacaatggggaaaatgtctccagggcatgtcagagaccctcacagcagcccctcccatcacaggcctggaagcctaggagggaacaatggtttcttgagctgggcccagggcccccctgctctatgccacctcaggacatggtgccctgaatcccaactgcttcagctccagctgtggctaaagtgggccagtgtacatctcaggcctttgcttcagagggtacaagcccctagccttggcggcttacatgtggtgttgggcatgcaagtgcacagaagtcaagaattgaggtttgggaacctctgcctagatttgagaggatgtatggaaatgcctggatgtccaggcacaagtttgctgtaggggtggagccctcatggagaacctctgctagggtagtgtggaaggcaaatatgggaagctaaaagggtgaacggtccagtggggctccccacacagagcccccactggagcacagcctggtggagctgtgagaagacagccaccatcctccagaccccagaatggtagctccaccaacagtgcacctggaaaagctatagacactcaatgccagccatgaaagcagccaggagaggggctgtactctggaaagccactgggacagagctgccaaatctgtgggagtccacctcttgcatcagcatgaactggatgtgagacatagagtcaaaggagatcattttggaactttaaggtttaatgactgccctattggacttcagacttgcatggggcctgtagctcctttcttttggtcagtttctccaatttggaatgggtgtgtttacttaatggtcccattgtatctaggaagtaactaacttgcttttgattttacaggctcataggtggaagggacttgtctcagatgagactttggacttggacttttgagttaatgctggaatgagttaagactttggggactgttggaagggcacgattgtgttttcaaatgtgaaatcatgacatttaggaggggccaggggcagaatgatatagtttggctgtgtccccacccaaatctcatcttgaattgtagttctcataatccccatgggtcatgggagggacccagtgggaggtaattgaatcatggagacaattaccctcatgctcttcttatgatagtgagttctcatgatatctgatggttttataatgaacttttattccttttgctcagttcctctctgtcctgccgccatgtgaagaaagatgtgtttgcttccccttccaccgtgattgtaattttcctgcagcctccccagccaagctgaactgtgagtcaattaaacctctttcttttataaattacccagtctcaggtatgtctttgttagcagcatgagaatggactaatacaccattcatcaacttctcttcattcctccctcccccttacctttccttgcctctataaccaccaatttattctctattttcatgagattcactcttagcttccacatatgactgagatcatgcaatgtttcttttttatggctgaataatacttcttggtgtatatgtacaacattttctttatccttttgtccattgatggcacttaggttgcttctgtattttagctatttagagtagtgctgcagtaaccatgggagtgcagatgtctttcccatatattggtttcttttcttttggctatatatacagtagtggaattactggatcatatgacaattctatttttcattttttgaggaaccttcatactgttctccataatgactgtactaatttacatttccactaacaatgtacaagagttcctctttccccacttccttgccagcattcattattgcctttttgataaatgccattttaactgggataagttgatatttcattgtagttttgatatgcatttctctgatgattagtgatgttgagcatctttttatatacttgtttgccatttgtatgtcttcttttgggaaatgtctattcatatcttttgcccgttttaaaatcactttattagatttttttcctattgagttgtttgagctccttacataatctgattgttaatcctttgttggatagatagtctgcaaatattttctcccattctgtgggttgtctcttcacttgttgattgtttcctttgctgtgcaggagtttttggcttgatgtaatcttatttgtctattcttactttggttatctgtgcttttgaggtcttacaaaatctttgcctagaccaatgtcctggagtatttttccaatgttttcttctaatagtttcatagtctgaggttttagatttaaatctttaatccattttgatcagatttttgtatatggtgagaggtaggggtctagctttcttcttcttcatatagctatccagtttttctagtatcatttatgttagagactgtcctttccccattgtatgttcttggtagctttgtccaagatgaattgactgtaagtgcatagatttatatctgggttgtctattctgttccatttgtctatgtgtctgtttttatgccagtaccatgctgatttggttactacagctttgcaataaattttgaagtcaggtaatgtggtgcatccagctttgttcttttttgggattgcttcaagtattctgggccttttgtggttccatataaattttaggattattttttccatttctgtgaataatgtcattggtgttttgataggggttgcattgaatctgtaaattgccttgggtagtagtgacattttaaccatattaattattttaatccatgagcatggaatatttttccatgttttcatgtgtttacaatttattccatgaatgttttatagcttttcttgtatttatcgttcacttttttggttaaattgataccaaggtattttaattatttgtagctattgtaaataagattgcttactttattgtttttcagattgtttgcttttggaatatataagtgctactgattttttaatgttgattttatgtcctataactttactgaattcatttatcagttctagcagtttcagtgtgggttctttaggtatttgtaaacataggattatatcttctgtgaacaaggctaatttgactttttccttttcaacttacatgcccttcattcctttctctttccaattactctggccaggacttccagtgctatattgaataaaagttgtgaaagtgagtatccttgtcttctttctgatcttaggggaaaagccttttatttttccctattcagtatgatgttagctgtgggtttgacatatatggtctttattattttgagttatgttccttctatatcaagtttgatgagaatttttataaaggatgttgaattttaccaaatacattttttgcatctattgaaataattatttagtttttgttcttggttctattaatatgatgtatttttttaatttatttattattattatactttaagttttagggtacatgtgcacaatgagcaggttagttacatatgtatacatgtgccatgctggtgcgctgcacccactaactggtcatctagcattaggtatatctcccaatgctatccctccccactccccccaccccgcaacagtctaatatgatgtgttacatttattgatttctatatgttgaaccatccttgaatccctgggataaatttcacttgatcatggtatatgatttttttattgtgatattgaattctgttcgctagtattttgttgaggatttttgcctatgttaatcagtgatattggtcagtatttttcttttttcattgtccttgttttggtatcagggtgatgctggactcacaggttgactttagatgtataccctcttcaatttgtttgaagagtcagaatagtattggtattagttctttaaatgtttggtagaatgtagcagtgaagccatcaggtcctgggcttttctttggtgggagactttttaccacagcttcaatctcattgctcattattggtttagtaaagttttttattcctggttcaatcttgataggttatatgtatctgagaattcattcatttcttctagcttttccaacttgttggtatatgattattcataatagtttctaatgattcattatatttatgagatctcagttgttttgtctcctttttcatttctgatgttacttatttgggtcttttgtttttcttagtctagcttttagtttgttttttagtttgttgatttagtttgttgattttagtttgttgatttgggtctcttgcttttcttagtctagcttttagtttgttgattttgtttatcttttaaaaaacaatttttcattttattgatcttctgtacttttttagtctcaatttatttttgctttgatcattcttatttctttcctttactcattttggatttggtttattcttacttttcgatttccttgaggtgcaccattaggttatctgaaatctttctacgtttttgatataggtatgtattgctataaacgttcctcttagtactgcttatgctgtattccatagattttactatgttgtatttccatattaatttattttaagaaattttaaaatttccttcataatttttttattgacttaatatgttcaagagcatgttgttgaatttccatgtgtttatgaagcttctgagattcgcttggttattgatttctagttttatttcattgtggtcacaagtgatacttgaggtgatttctacttctctgaactctttgagacttgttttgtagtctaagatatggtctaatctggagaatgttcatgttctaatgaaaggaatttgtattctgcaacagttgggtgaaatgttctataaatgtcagttagacctactaggtctagtgtgtagtttaactctgattttttttttttgtttggatgatctgtgcatactgagagtggagtgttaatgttccttacaagtactgtactgcagtcaatctctcccttctaatctatcaatgtttggtttatatgcttgggatctctggtgttgggtacatagatatttataatttttatttccatttgttgacttggcccctttatcattatataattactttctttgtctctttttacagtctttgatttgtagtctgttttttttttcaaggtggaatctcgctctgtcgcccaggctggagtgcagtggtgcagtctcagctcactgaaacccccaccttctgggttcaagcaattctctgcctcagcctctcgagtagctgggattacaggtgcccgccaccacacccagctaatttttttttgtatttttagtagagacgaggtttcaccatcttggccatgctggttttcaactcctgaccttgtgatccacctgtctcagcctcccaaagtgctaggattacaggcgtgagccatggggcctggccgatttgtagtctattttatctgaaataagtacagctactcctgttcttttttggtttctagctgcatggaatatctttttcaacccctttactttcagtctataagtgaaatgggtttcttattggcagtatatagttgcttcttgattcttttttttttttttttttttttttttttttttttttttgagacagagtcccgctgtttagcccaggccggattgcagtggcgcaatctcggctcactgtaagctccgcctcccaggttcacgccattctcctgcctcagcctccccagtagctgggactacaggcgcccaccaccgcgcccggctaattttttgtatttttagtagagacggggtttcaccgtgttagccaagatggtctcgatctcctgaccttgtgatccgcccgcctcggcctcccaaagtgctgggattacaggcgtgagccaccgcgcccagccgattcttgattctttatctattcagccactgtatgccttttaattgagtcaatttacattcagtgttcttattgataaataaggacttactactgccaatttgttgtttgttttctggttgttttgtaactcctttctttctttcttattgcctttctttgtgattaagggatttttctctggtagtatgttttaatttgttgctttttattttatttatttatttttattttttttttgagacggagtcttgctctgtcactcaggctggagtgcagtggcacaatcttggctcactgcaacttcctcctcctgggttcaagtgattctcctgcctcagcctcccaagtagctgggattacaggcacatgccatcatgcccagccaatttttgtatttttagtagagatgaggtttcaccatgttgatgagcctggtctcaaactcctgacctcgcgatccgcctaccttggcctcccaaagtgctgagattacaggcatgagccaccgtgcctggctgctttttatttttagtgaatctattatcagtttttgtattgtggttaccatgaggcttatgaaaaacatcttatagatataacaagttattttaaagagatggcagcttatctttgatcataaataaatgaatagaaacaaaggaaaaacaacaaaaagctccttacattttaactttatctctgctacattttaacttttagttgtcttaatttacatattattatattacctatctcttaacaagttgctatagctattactgtttttgatagatttgtcttttgggtttcatactagagttatgggtatactctataccataattacagtattattttgggtttttctgtgtagttttaccagtggattttatatcttcaaatgtttgttgttttcattgatggtggcttttttttattgcatattactgtttctttttttctttcagatttgaataactccctttatcatttcttgtaagacaggtctggtagtggtaaatttgctcagcctttgtctaggaaagactttatttctccttcatattagaaagataactttgctggctgctatattcttggatggcagtagatttttgttttctttcagcactttgaaaatatccttctgcttcctctgggcctgtatggtttccattgaatgaagtctgttaacagataaattggagtgtctttgttcattattagcttcttttctctctctttgttctttgtccttgacctttgcgagatagtttttttgtgtttgttttttttttttttttgaccaggttttgctctgtcacgcaggctgtagtgcagtggcatgatcgtggctcactgtgacttcaacctcctgggctcaagcaatcctcctgcctcagccttctgagtaactgggactacaggtgtataccaccatggccagctatttttatttttttaattttttgtagagcttgggtctcactatgttgcccaggctggtcttgatttcctggcctcaagaaatcctcccatcttggccttccaaggtgctgggattacaggcgtcagccactacagctgactctggggtagtcttacttgggtcaaatctgcttgggattctctgacctttctctacctggataaatttatatctttctcatgttgtagggcgttttctattgtttcttctttgaataaactttcttccccttgttggtcatctctctctctctcgagcattaataattcttagatttgatcttttgaggtaattttctatcttataggtgatcttcaatgccttttttcttcatttttctcctctgtgtattttgaaatagcctgtcttcaagctcactgatgtctttcctctgtttcatccattctattgtcgatagcctctaatgaatttttcagttaagaaaatgtatttctcagttccaagatttgtgtttgatttcttaaaattattatttcaacctctttgttaagtttcttcaataaatttgtgaattgcttttctgtggtagcttggagatcactgagtttctttaaaactactatctcgaattcttggccggagagctcaaatattgctgtctcattagggtcaattcctggtttcctgatttgtctatttggggagatcatagtttggttccctgtttgctgttatttcttgtagatgtctatgtctttgcattgaaggattatttattccagtcttctctgtctggtttgttttggtttgtattggatatatttgtatagagtttctttactgctaggttgctgcctcctttttggctctaggtggtgccttaagccaacaggggtgatcctaaaagggatgtgtggtatgggaaggttggttaggggttcatgcccaagggacctattagaagaaccccgtacagcatggtgatactgaacagtcactctgatttggtgtcttctttggccaagttacagagcagtttccaggactggggatggtagttccacctccctgctttgtttctgcctgtcttcagggttatacctcccttcatacattcgcaatgcttcacatgggttaaggcagggacaggtctcctgccagggaacccacaatggtggggaaactaggtttcaccatgagctcactttttccagtgtagaaatagtgaatttgggggaaattttccacatgcttagtgctgggcagaattggggagaggtatcatggatgtggatatctaattctcttacagtcttctgagagttttttcacctctcttttgctctgtgaagtgtctcattttcatatttaaattctgggatatttctgatggaaagtttggtgtggtgtatttgtttttttgttttcagagattgcagagagtgaagctagtttgcttctatgcttccattttggaaccaccgtctttttgcttttaacatacatatgtcattatatttgaactgagtttcttgtaaaaaaacatatattttggtcttttattttttggttttggttttattctgaaattttctgtattggtgtttttagagaatttatacttatatttgagtttccatcaatcattttgttatttgatttgattgttaactattttttattccatttctcttttctgccttctttttactcattcgaaccttctcagcattccagttttgttaacctagcatgtttttgattatgtcccttaatctaacattgtaaatggatgctctaggaattaaaatgaaatcacttaactttgacagtttacttagaatcaatatcttaccacttcaagtgagatgtagaaaccttaccagtatataggtcctttaccatctctccttaatttgtagttgccttgtgtactatatatccatacattaaaaatcccatcagaaaaatgtaacaattttgttttcaaccatcaaatgtattttaaagagcttgaggataaagaacttgaggagatttaaccagatatttaccatttctgttcttctttcattccctacattcaaggtttcctgctcacatcatttcccttctctctgaagaacttccttcagcaattcttttagaccacatctgcttgtgtgaattctcttattttatttttttcctgcagagaatgtctttatttcatttgtctttatttctgaagatgttttcactaaatataaaattctaggttgttggttcttttctttcaacactttaaacctgttctatttccttcaggcctccatggcttctgatgagaaattcacaattatttgaatcatagctagtgcattgtttatctcttcttgctttcaatttttttttcttgtctttagtcttcagcagtttgattatgtctcttgattttgatttctatgggtgggtttattctatttagtgttctctgatacttttaaatatgtaagtttatatttttcactaaatttcgagggtttcaggcattatttactctaattttttttctgcttcatactctttattctcttctttagatattccattgacataaatattagaccttttgttattttctcacaagtctccaggctctattaatttatttaaattttttgttgttgttcaatttggataatttattttgatctattttcaaattcaatgatatcttgtgtcatctttattttacttttgagttcattcaaatttatatatacattcaaaatgagttattgtatgctgtagttctaaaattttaatctggatcttatatttctttgctgatagtttataccatctgttcatttcaaaagtgtgtggctttacttcttttaacatttttataatacctgctttgaagtctttgataattccaacatctgtactatcttggttttggcatatgttaattatattttcctatggaaattgatattttcctatatttttgaacatggagtaaatatgaattatatcctaaacattttgaatattatgatataaatcatatatatcatattacgtataaatcttgatatttttagtttagtagataatccacctggttcattttagacacaagtcatgactgacctattttctgttgtcatgtctggttcttgtcagttccacatataaagtttttgcagtgttgtttagatctgtcctataggaggccagtctgagattagggaggtggccaatccataggtctcttcttgtagcctttgatatgttgtttagtgtctgattcataaatataagcttaggggtaagctccagagttcataaacaactctacggggtctttttcatgagttattctctctctgtgatctccttggtatagtagttttcaggtcccgaccttccttgttctggtcctctggccaaagacttgagactttaattacctggtctgctgcacatgcacactttagctgaccatgcccatgtctggagcttagcaccaggaagacaaataattaaactaacaaacaaaacctccaaactgaaaagtagatgtttattctaccatttttggaacacagctatctgatcagagaggatgtttcccctttgctaatggtctgaatgtttgtatacccccaaaattcatgtgttgaaaccgaattaccaaaaaaagagaaaaagaaacctaattaccaatgtgatgatattagaaagtgaggcctttgggaagtaattaggtcatggaggcagagccctcaagaatgggattagtgcctttatgaaagaggccccagagatctgcattgtcctttcaccatttgagaaggtaccatcaggatcaggaaatgggctcttaccaggcactgagtcggccggtgccttgatcttggactttctagcttccataactgtcagaaaaatatttctgttgtttataagctcccagtctaaggtattttgttgtagcaacctgaacagactaagacagaaaattggtctgaagaaatggattgctgctatattaattacctagaaatatggatgtggctttggaactgggtaacagaagccagaagagttttggggtacatgttgtacacatctactcctgaagaaaggaaggataaacagatgagtttttgaagaattttaattgagaaggagatatggcagtaactaactacaaggtcaaggaaggattttatttttcttagaggagagaaacttgaaatgtgtatagggtgaggagaaggagcttgtagaaatggaagagaatgagaatgtatgaggaagaaggactaagatcctggaggatatgaggagaagatggagttgaaaccattggtggagaaatgggctttgatcaagaatgaaatctcttgctaaggtcgaagaaaggaggcaaggaagtcatctcttaaagctcaattcatgcatttactggacatgatatgtgttcaggactttgttggatgctggggtgggatggagaagggaatagtaatttaagacaagtacaaaacactgttctcaataattatgcagtatgttataaggtagaattaagttccagcttcttagtgcctcaggagaaaaagaaagacaatcttgaaagtataaaaggctaacaaatataagcttaatagcactgagtttaaaaattgtaatcagagaatataaatggatatccactctgtatccttgttttgttagactctcacattgtgctaaagcacgttctgtttgttttgaacacatttgaagagggaccttcgctctctgctatggtacacacactccacttcagttttctgcctggcctcttaaagcattaaacaattggactagaaaatctgcatgatcttttttagctttagatatcatgattctcaggcagcttccctggagatcatatttgttagcagaaaactgtatgctagggatcactagaaacaaaggataaatgtaggaggggaaccaaaggagagctattcctttccttttgaacttgacttctggtaccttcctaagcatcatatattaaattattttttcccttataaagtacaatctcactagacatctgttttcctctcctatcaaattttgaagtaaaaatgaattactgccaaaacaaagcaaaaagaattattttatactagtctaaaataacaccatctaaattatgtgtgttgcttaaccttatatggaaaactttccatttgtgggtagatgtttttcacctgtgggtattataaatcatgtatgaaaataatggaggatatttgtatgactcggaaaatttttaaaaaaatatttagcacctaggaaaaaatcaactatttaaacatttttaaaggtattctaacattatgtactctgcttccctttctctctgaatggactatgtagactactgaaattaatatttaggaaaggaggacaatgattagtataactgaggctgaagagaaatcctttattatccaggaagatcatctctgtgatggcagagagaggaaggacaatcttcctgggcaatcagggaagctagttcaagattattctcacaaatagttgttcctttgtcactccctcaacataccttgttttgcaccattataagacaaataatgcaaatatgaaagattatactaaatcttctcctcacttttacttttgtataacatgccaggatttgggttaatttaccatgttttagaaatagtatattctgtctttatagcatatgtcccaactaattaattggcctggacttgataagcttttatgcattttcattttaaatttgaatttcaaaattaaacataatctttatgaacagttgtaatgatcaccatctcatatacacagaaaaaagacagtcaaagttgagctatattcagagctatttcagaattcattaaagactttaacctcagaaattatttttccttggtaccctgatgtcagacttttttatttttattttttaatggtttgtttatttttaaacgttgtggttatacagtaggtatatatagacctctttaataactgcagtacctgttaatcaagtttttccccatataccttcattccttccatttcttccttaaactagttttcgtctttaccaatgaagtgcaagactttggaatgaacaacaagtatttgggtagaatgataactcaaagtgaagactcaaagtgaaatagtcaattggaggagcctgcctgagagactagagtataaaacctgttttaatatggaaaaacaaaaatgcagagaccacattgaaaagcagtgaatgggaaagggaaaggaagagaacaagagaaggaagtaggctttcaaggactccttcatagtaataaatctaaatcacttggacctctttctttttcagtgacttttatttctgcctgtagaaattgttatatgtaggtagcagtttagaatgaggaatactattatgtttcctatttttaaagatttgtattgattaaattgaactttcataataattgataaggtgagaagataatgacttcttctaaactcagatattgccatatttaaaaatgagctagtattacaaaaaataaacaaacagcgtctttgtaaataattggcttggcaatgaagttggaagattttgcaattaattttaattgatcaatctgatattgtattttgtattcatcttataatttgctgctaatatgggtacattctgtttctcaacattaataatatgtgcatatttatatacatgaaaaattagtatagacaagcattatgagaatcctgcacaatacagaaaagttcaataagtatgttgactatgtaatacattttaaagtacattagcctcaattaaataaaatgtctatagctattcactggtacaatttatttagggattcattcattagactccccattttcagtccttttccttggaaaatatacctttgaaaacaagttttgccaaattgaccatgattagctccgcttaatgtattttattataaacctttaatcaccttaaatcaatttcaatggattcattttacttaaagaatctctccttgcctattttaaacaaactgtcttagacatgattcattgttatcttcaggttaccgtgtcttttgtttttcttataggatcttattcttctctgttgtatatttatatataaggttccaccaaagaagaatctccttggtgtatgtgaccttcagactgtcaggaaaatgttacctgttggcataaacaaaccaccacatctcagtggctttccacagtagaagttcatttctcactcactcaaagtgcagtagtggtgtttctgatgtggacagcattcatcatggtctttcaggtgcagaggctccttataacttgttactcatcaccatcctctaggtctctgaggcctctgtatttagccttcagatgagaaaacaaaatggagaatttttgcttggggttttaatgcctaggcttgtaaatagtgcttatcatttgtgctcacattccattgtttagaacttagaaggccacacctaactgcagggcagcctgggaaatgtggtctagttaggtgcccaggaagaagaggttgtgttttccaaatttcagtcacgtcatgcttttttcttatgcttcttttttttcccttaaaataattctcattttctttttgttgaatttaatcattcattcattcaacagatgcttaatgaatgtatactacatgatgggtacataccgttctaggcacttgggatgtatcagtgaacatttctcatttggggtttgtctgattttttttcatgattagattcaggttgtacatccttgaccagaaactgctcctggttctttaatgcatttggttaaatatattctgtcttccttttatgttctaaaaacgacacacagcttgggagtagttgaatatgaaaagtaaggttacagtatgcttgtggaaaaggtatgtctaacattatgtaattttaatttaaaatgtgcttaatagcttctacttgacatgcctacccagaaaataatatattttataagtaaattatgtacaatacagacatattgataaactttacagttaccattaatgactcattaatataaataaagcttattttgtccatatatgaggaaagagatgtatttattagaagattttggtaggtaaaattctctagaccttgatgaatagcagcagtaaagattataggtagtgtttcttatatgctcctatgtaccagataatgtttaagcattttacatgcacaactctataatgtagatactatcatatctaatctatggtgggacactgaggcacacagactgagttacacacctaacaagtggcagtcaatatttaaactcaggcagtttgacttcagtgcgcattctcctaatttctatactactgtagactctgaaaatgtttgctgagtgggttaatgaatgtatgaagaaaatggagggggtagaccagttgtgccttctggatcaaagagtatgattctgtgatatgatacttatgactctttgggttttattccaattggattttaatgggttttattccacttgataaaattattaattttatgatgctaaacatgttttataatactttttagctacaggtaaaaaaggaaaaccctataatgacccatcttattatttatgtaattatttattcattatttattcattcatcagatagatatcaagcctctactgagtgccaggccttgtggttaaaacacttaacaacgaacaacaacaaaacctaaacccatatagggttcatagtcttaggggtggagttgaaattgatataaacaataaatgattacccaaatagaaattaattcctatagtggtaaatgtagttagaaaagcacaagacttttattagaatatataaaagaaggactcaacctgatttgaatagtcaggaaatgcctccctgaagaagatgtttaagttgaaagcttgagtggaaatttaaagttaaaaatcaaaagttgaaagctacgttaaaagaatgagcagcagataaccagggagggaagagggaaaaagacttattctaggcagagaatatgtgaactgaggcctgatagaatgagatgtatggatttttctcaacacgtatctatcagtctatcatgtgccaggtaagatgagaactgaaaaagggccttgaattaaaaaacctggtcattggtgcattgatgtccttaatgaaggcaggcattggattggtgggggtggaggggctaaatgggagataaggaagtagagatagtattgttttatattggttaaagttgtgatttatagctattgttttttccccaggttgggcttacaattctgctcctcagtttacatcctccatggtcaggtcttgttctaacttcccagcaagtaatggatttgtctcattgtttattgtattccatgatactcctataattctgctttatagcacagtaggaaagagtttagactcttgagtcaaagtagattgagtctgaatcctggcctcagaacttgctggctgatttgaccatgggtattatataaacatgatgaacctcagcttcctcagaggtaaaatggagctattaatggtatctcccttgtagcatttttgaagggactaaataaaattttctgtgaatctggcatagggtagtgcctcagttaaaaatagtagttttttcccccatatcttgactattgttaataatcctacaatgaacacgggagtgtggatatctcttcaacatactgatttcagttcttggatattgagtgctggatgacatggtgattctatttttagactttctttttttttttttgaggaactgatatgggagtggggcagggaaatgctaggtggtgaagggtgtggggtccctggcgagggctccacccttgggcctgtacccacagacctaagtgaggacaggcactcctgttttcacgcccaagtgttgcactttccaagaccactctggcctgccatgcccccccatcctgtgcctgtaaaaaccccgagaccctagcaggcacataccacaagcagctggatgttgagaggaacacactggcagaaaaacacactgacagaggtcagcaggccatcgatggcagaatgacgtggacgccgaaggaaattcagccaatggtggttggaggagagcccggttactacgcggccagactccaggggaagaccatcttcccactccatcccctttctggctccccacccatctcctgagagctacttccaccactcaatgaaacattgcactcattctccaagctcacgtgtaatccaatttttccggtatactagggcaagtaccccaggatacagaaggccctctgtccttgcaataaggcagagggtctaattgagctaattaacacaagctgcctgcagacagctcagcagaaagagcacagtttaacacatgcccactgggacttcgggatctgtaaacaatcctagacactgccgtggtgttggagccccatgacttgcccatctgcatgcttcccctagaggactgagcagtggggcactgaagaaatgagccacccctatcacatgccctgtgagggggataagggaaaactcctcccattacagagcctttatacttttttccaaaatggctatactaatttacaataacatttattaaaataacactttgaaaaagtgtcagttttgttattgttctatttctagtagctaagccccctttaacagtctttatagaatgaatatagtgaaaaactattttcaaatttactagaaaatagtttgtcatttgaataaagtaggtctgaggacatttgtgccttctcagagctcagttcatggcagtgactgaggaggaggttattaggttcataattttgagggtgtgggagaaccaggattgcctgaagtcttactactagggttttcttcatctagttagttgacaggagccaatgtatctgttgggcattctgccccacaactcctcgatcattgccattggctcactcatgactttgggaccaatattcttcatagggatagggaaatatgaaagttgccccatcagcagctcagtgactcagctgtcaggactttgcacactgtacatataccgcagtggtaataactgagcacttactgtgttctttactaatgtcctgtatgactaaggtcttgccctccactcttacctagaactcggttcattctcctcaattccagagctgaagtcttcctaagattgggtcttttctgttgccctgagtgacttttgattctgattgcttcctttcctggatctctactgattctatactatcaggctccaccctgaaagaattcatctgcctgctgggatctgtgtctattgccacttcagacctctctgatattgagtacttacctgcctatgcatcacatcattgtctagggttatagctttgcaaattacctggtttcccctcatgagactttgtgttgattggatcctagcctaaccaagtctggtctgttctgctgccagccaatcccaggttctgctatccttgtccttctcatggcctttgcatcttgagttatggaactctttaaagcaattctgcctttggacccataaaattttaaagtttgtattagccacattttaaaacatacaatgaagcagctgacattaattgtaatttaatccaatattttgttcattttatattatttatctaactgaatatatacaaaaataccatttaaatatgtaacaaatgtaaaaatactaataagatattttgtcatgccaagactttgagatctggtgtgtattttatactttgtcacatctcaattcagagtagaactgctggaatgtaattcacatttagagtgcctcataggtggtggctactgtactgaacagtgttgttccagaaatggatgggagcatctgtccttcacaggcagagaagaaaagctcatctctgatacactgaatttcaactaagaggaaatccacatcctgacatatctgtatatgtctcttgcattatttacaactctgacaactacattcttggccactagcattcttgttacagtatgcatgtatgcatttatgcatataataaaataacctcatttattcattcattgatttatttgccaaatatttattgggaatgttccagttgttactggcactgttctagcttcttggaatacatcagtaacgaaagagagaaatattttgcccttgtggagtttacatcataccaggaagaagcaggtaataaatcagtaataataaataattatataacatgaatgttagaatgtgatagtggtatgtaaaaaaggaaaattagggaagcctaagtgggttgggagtctggggaggggaagtgagttgtagctttaaatagaacataaaatatacaagataataatatagaagataatagaagaaatatgagaagaaatatagaagataatagaacataaatggaagataaagaagtaggtgatgagatcacagaggttactggggtcaagtcaggatcctaggctaggtgctgtctgagattcaaaggtgagtcagacaaagccccattcttaagggagtctatcaagagcaacataacaaatatattgatagttatgatgcaatggtaaatataacaattgtcatgaaagaatttgcaaaagataccacaggaattaaaaagaagattcctaaaggaggcaatgtggtagaagaaagaacatgggctttgaagttaaatagacctggatttgcatcccagctctcatcttaccagctgtcaataggatgaacaatgatcctggattgcccaggtcagtctggttttaacactgaaagttctacatcctgagaaacgactcagtctcagttttagcactgaaagcccaacatgagaagcatctcagtacatttttaccattgaaagtccccacttcagttcctggcaaactagtttggttggtttgtgtgactctgggcctgtatacccacaattaagaataaatccaatcttgtgtggttgacttaaggattatatataaaatacataaaacacttgtcacattggaggcagtaaatatatggtatttgttatttgattgatggggtcaagggaagacttcatggaagtggtagggtttgacttagattttgaaagaataatgaaatcttatttgatatagggcaagataatgcttaactggcaaagatggatagagatagaaaatagggcaaagggaacaggatgacccaaaacacaggaaaagaaaaattgtggggcatcttctagggattggctaactgtggggcatcttctagggattgggttaagtgtgggatatgataagtacaagaaaggattaaggtttaaaaaggtgatttaagacttcttcctatatatgttaaatgcaaagaagaggagtttttaaattcaaatcgcaaatattgtttgatttcccaagttttgtcttggtaaatatgtttaattaatgcttacttgtgttgctattaagccagataagccaagctcagagacataatttctggtttttaatagagtgttgcagagtcatattgttcataaattacatgtaaattatggtgagctgtacttatttgtagtttgtaaagagtgttatagcatgaaagttatttcatagccatttttatttttattggtattatctaacatttggcattagacctaggtgattgattattttaaaatacagattattcactgaaaattacagcaataatcccatgaacacatgttaaactttcacagtaggataaattatatttcaattctgtggaagcaatcaaatgataaataagtttttggtttcaggaaaattggggtttgtcttagtccttttgtgccactataagagaatatctgaaactggataatttataattaacagaaatgtattggctcacagttctggaagttggaaaatcactggcattggtctgatgagggccttcttgctgtgtccttctatggcacaaggtacaaggacaagagagcaaggagggggccaaactcacactttataatggcacaaatcctgccccgagggtggagccctcacaccccatcacctcgcaaaggtctcatctccaaacactgccacaactgcaaccaaatttcaacattagtttagagggaagagacatttgaactatagcagggttcaacttcagaacaccacattttattaagttagtatattaactctgctgtgctttaattaatttttcttgtatttctaggtgttaaaaaggcctagcaccttgtaaggagagagtgtgtggtatagtggttaagagggaaaactctttaatcagactgcctagttttgaataatggctctccctggtggtgaccccttgctgcagtgcctatatctccattcaaggaaggatttgttagcccctctggattgacaaagctgtagagagcccccttgcccaagttcatactgcccctctaccctgcccaccactacccagaggtagcccacatgcatttactgattgagccagtggtataaagacctggccatttttgcctaatgaaagacaacaccattgggccatttttgctctagccctgtggggtttgttgttgaaattatatcacagatgaacatctccgcctgtgcactctgtatatcttctcctctctcacacacagttattgatcccaagggtaatccttaatgaacagttctgcatagcgaacctcatatcagagtctgattatcagatgactcagtctgccataattgatgcctagaatggtctgagaaagcaggtaataagatggggtttggtggtgagattgcttgctgcctgactggcaatgttctgaggtttgtggagcacagacaaccctggacatgtggtggtcctatagttaaaacttttactggtggtaaatttggctggtaaatgggtagaaggggatgcagtagctgattctatatatcaagcatccacaagtatgaggagaaagtaatgttaggggaagtgaaattggatggctattgctaagctcatttgacactctagaaaaagagaacaaaagacagactgcttaatagtcaatcgaaagcctgtgtgcaagccaatgggcttccttcctatcatacaaacagcccattgtgtcctgcagagggaggtcagagaaagctggatcaggcatagtacttaatcatcagagtggctgagcctcaaagaagattaaactcccagtcaaggtaagtctgccatgaaagaataggacactggcacatgcaatagcaacatctgggttgttactccagaacttttaaacccctagacttctctgaaagtcctgatccttcagaactggccagctctctctctctcttaatagcttccaccaaatcccttgtatttgcagacagtagagaggtctcttcttagcaagtcaacatgtgacccctcagaatgtctcctcatatccctgccaccacaatgaataaaactaatgaggattaaataacagcataatcaatctgggaagcactgagcttggaaatggaggaaaggaactataccccaaaggagctgcacgacccagctagactgtgtggacaggagccaggggagacatgagggactggattctgaagggtgcttgatcagaggggcaggaacataagaatgaagaaggaagaaggagagtttgataattctgggagtacactcccaaaatatagaatttataccgtgacagagaccccagaaaatggtgaggaattgttattacagtgggctagaagcatgggaaaagcaatggcccatgctcagtgaggttgaaatgctgggtttgctgtggaagatggtgaaagaagggatttaaaaattcagggaactgggcatgtagagtaaatattctgtgtaaggctggaaaacccaccagatgattaggttttagggaaagggacagagaatacaccatttacaaaggctataaaaattgcaccagcatcatgtagactctcctctgtaggccagagctggtgataggagagaccaatctagaactggtctcattgatagcaatggggatgataggagcaaaaacaattgaaaccaggtggtagcactgaacttttaaaatccaggtgaatgcagttatcataaagaccattaaggtcagagaagcagtgaagggggtgtgatcctcagatcattatgaaggtgctaaataaagaacatgttgctagggcaagataagaggcaatccagaaagatagtgctcagctagcaccatcagaagaaatcaagtatggatggttaaggagactgagtgtagttttgccaacaaaaagccataatcactagccaaatgtttgaactagagccagcatttggacctagaacccaatgatggaagaagatcctgggtcctaagggagctggcaatatctcagtaagcagtggcagtaatgattccccatccttccccaatgaaagcaacaaccatgttccagggcaactgaagatagggaaagaggaatatacaaacgtttggaagattgctagacacaaggcctgagttgacattgataccttgaaacctgaagcatcacatgtcctcctacactccctcttttcctcttctaatcagtgtacatgggatccactcaataaatggagttctagccaaagtatacctcaaagagggacttttgggatccttaagctcacctgttggtcagttctttggtcactgaaggcacaatttgaatgggcatatttgatattcagtacaactatcacattggacctttaacctgtgggataagagctatcgtagtgagggaagaccaggtggagaactttcacatgctctcccctgcctcatccaaattaatgaatcaaaacaatattaaatcctggggactgggagtggtggaaattaatactgtcctaaagattctaaatgacacaggggtgatgtttcacatcatatctccatttaatccccagtttggaccctgaagaaactagatggatcttggaagatgacagcatgctatcataaactgaaatcaagtagtagtgctaattgtagccaccacaccacatgtgacatctgtgctcaagcagatgacatgccctcaggcacgtggtatgcagacattgatctggcaaatttattctctttttcctgtcagaaaagaaaaacagaaacagttttagttgtgaaaaggacaagagtatacatttaacagttttgcctcagtggtataactgccattctgtcatgatatggtttgaagagatatgaactgttatatcaataatatcaaccatttaatatgtacatcaaatattcactgggtagtgagcattctaagatgttgtcatagactgctgaagtcccagctgaagcagcggtttggagatctatattatgaggatgggatgctgtcctccaggatttagtgtatatcttgagtcatatctttatacagcactgtgttctcaataggaagaatacttgagtccaggaactaaggggtggaaagaggagtggctctgcttgtaatctctcccacaaatccccttgggaaacttgtgcttcttgtccctacaacttttgtatgtgcagtatgatcttcacatcaactttgggatttgcagttctcatggcccaaagaggggacactttcaccaggggtacataaaaatgatcattgaactatatgctgtggttaatatctgggtaattcaagcttattattcatagggaacattagacaagaagtgaagtcagatcttgggtaattaactctgatgatcaagaataggaaatgcagttacaaaatggggaaaggcagaaatatgtttggtacccagttgaggtactgctcaactgggtgcctcttggtgctttctcaaccagttgtgacagtaaatgtacaatgcagccaccaccccactgtgaggctcaggggttcagacccttcaggaacgattgttcaggccaatagttaagtaactgaaactggcagaagtgccagccaagggtgagaggaatctggaatagataatagaggagggagagtatcagtattagttgagaccagctgtagaccaatctttttgtaaatttcccccaggaagataggttactagattttggacatgctgccctcttcatataaatgaaagagaggattcaagcaacatgaggagtagaatgggttggacactgtgatgtgtcaccaagatctctctttaatgaagggcctgttgcccgctctctgcaaatgctgttgacaaacagcttttaggtctcaagaattgcttcaggtctaatgagccactttgcttatggtcccacccttctgagtgggcccctgattgagacgaggttaccaagttctggccctttttgtcccaatgtagaaccattcttgtgagctgttttctctcttttttcaaatgattgataaagctatgatcaggtctgcatcagagctcagcttcagcatttgcattcagtaaacactagctattgttgattaacaaacaggattattttcaggatagagtaagaaaaaagaggtgagactattgtgaaaatattttaatgatcagtgttcatataaataacaagtttcctatattaacatattttttgcctgacacaaaaggtagaagtaattttatgccattagaaatattttattttcctctcttgcctctactgttgtgtttcccatggttacagttacctggagaaactgatttctttcacacattaaaccctgcgaaatgaatgggatgatgtaaccaatcttctaatcacaactgtgtgggtgtgtggaggggtgtgtgtgtgtgtatattcaaaccatagacacttaacagaggttctgtgacagagttttcactcttgaagtgtcagcgctgcaaacagtataaatattaaacaacaatacagtgttagcttttttttaactctaaaagaaaaataactttgttccaccaattcagctaataattttaacacccattacgttccttttgacacccattacgttcctggtcccaatctagaaatgagggatatgaataagtgatccccaaaatcttactctttagaaatttttagatacctatttttatgttttctaatttcctggtaattcctactaatgtggtcattgaggagtcaactaaaaaaggagtcttttaatttaataataatataataactgccatctgtgaagcacttactgtgtggcagacatcatgctaatagttttattaatatttaagcaaacttaataaatatttatgaaacagcctctgtgtaccaggcacatagtgaacactagcacgatggtctatgcatcaggcacattgcctgtccttgcagcactgattgccttgttgggaagacaaacctacaatataaaaaatgctacattatggaaagaaaaggatagtattgaacaaagaataacatagaagaagtctgaaactacttttgcatggtcaggaaaagtttttcagaaaaagtgatatctaagctgagacctggaacataagcaggcattggctaagagattgaaggcttcagaggagaaatagcatgcacaaagagcagaggaaccagacaattttatcaagctggaaagcaatgtagttagtgggaaacagtggtaaaggcatctagagaagggagcagaggaccagccaatcataacagaccttgcatacccataaagcagtacccatgaatgtaccctctactttttcctagggcaatgggaagccattaccaggattttggtaaggagacagatgactttttgtatttgcagttgaaaagtttcattctggctgcatcataggttacagaggtaagcaagaccagaggcagcaaggtggctattcagatcgtctctgtgttagaggttagtagtttggataaggagaactttgggattagcaagacatttagacgttcaaagcatgtttagaaaacagtagtaatagttattagggaatgttttgggatgtggtactgaggccaaggaagaacaggaatgctgcacttagtcctgcagatgttgccttgaacaaaggatggctgctcaggaaatgagtaggtgctgatttttctgtgagcccagcttacctgattgtgtgctctggcatggcctatgtccatcaggagaaaagaagatcttttctaagttgcacaaagatgccatatgggctactagtgaccgtgcaggatgttaactgtaatcccaaggtttctagtttggacaattggattactgctagcaccatttgctgagactggggacagaagtggtcgtggggaggaaagtcatgaacgcagtttgcactacaggctgggaagggtgtgtggtgtgtgtgtgtgtgtgtgtgtgtgtgtgtgtgtgaagttggttaatcgatacaaatacatagttagatagaagaaataaattctaatgttcaataaatagggtaaggtgactatagttaataacagtatattgtatctttcaaagtagctagaagagaggatttgaaatgttcccaacacagaaatgatacatacttgaggtgatggataccctaaataccatgacttgcacattctgtgcatgtagcaaagtgtcatatgtacactataaaaatgtacaaatattgtgtatcagtaaaaacttttgaagtaatagtaagcccattttataagagaaattgagtcgtagagagattggtgtttttcttgaggacatatagttagtaaatggtaaaactaggattcagatcttgatgctatattttttcattcctataattcctacgaaataagaaagacaaaagtcaattaccatattgtggtgtcaattaccatattatgggaactacattatgaaaatagatgtgattttatttgggaaatgtacctgaaaaattgttaagtcatttgtgtattgtatatttcacttctgaatatccagaaacttaatttcaagaaatgtttttgcctgtaagctttcttttcacttttcctgtcttccctcctgctgttgtattgatttttttttttttttttttttttttttttttgagatggagtctcactctgtcacccaggctggagtgcagtggcacaatctcagctcactgcaacctctgcctatggggttcaagcaattctctgccccagcctcctgagtagctgggattacaggcatctgccaccatgcctggctaatttttatatttttagtagagatggggtttcaccacgttggccaggctggtcttgaactcctgactttgtgatccacttgcctcggcctcccaaagtgctgggattacagacatggattttctctctctctttttttaaagccctttctcttctctgttattcaagctccaactgtagccttacttcctccaagaaaagattccaaacatcctacatgatgtaaatattcctctcatgagcttcctgggcatcttgtgtatactttcacttgaattatgacattaccctaatgttggtttacttttagttttatattcctagtacctggcacatagttactgttcagtaaagagtgtttgatagaatactgtgtgagtgaaccacttctcaaggcacacctcttctaataagccttcattgatttacccattctatcacctagtcctgtgactcccagacataagtggacataaaaatgaactaggaatttttttaaaaacatagatttacaggcttcactccagacctactgaatctgagccagggccaagttatctggacttagaaaatgtttcccacatgactatacagtccgaccacccatttaggaaccactgctttagcccccagcacagtctgataaaactcttctaccaatgtgtgcttacagttgttatattttatatggtggtttaggtaatcaatttgttatatcatcattaaggaattttgagataattatatatgtctaaactttttaatataacaccagaaactcagtagggtgataatggtgataatacatgcttgtccacagcttgtccacagagtattctcctatgtctcacatcttaatttacttagttacttacagtcctttcattttaagagagttgcatgtcacactttgtatattaagtaaaatttatataaacatacacatgaaacaggtattttttctacttcttggtaaaacacattttgtatgatcatattattgttttagaaaatatacaaaacattttttatcttgctgtctgttatgcactttataaattttgccaacacagaatccatctgggctaaggccagcagaactgtttcacttttgccttgtttgcgtattaacaacacagctaatgtcaacatggctttatttgctcttgctccaaaaaagctgaccagaatccatgtagataccttataaatcatttagattttcttctgcaccattaagaaagtgttgacattttttaaaggaaggcttttttttttcagactacagtaattctatcattgagcaccaaaatcattttcaggatggcattattcatttttctaaaatgccatgtccagattcccatttttttcaagaagcttggctgaattaaatgagtgacttggcttgacttaacctcataaacttcaatgtcttaacattttgccgtggaagtacattatcattgacagggcatgttttatgttttttgttcccatttcctctgttagtaatcaagaaaacctggaatttcttgatgcagtaaatatctctgcgtcaataatgtagtcatactttctggattatttctcaacacttcagttaatgtgacatcattctagattatcttactaagtcttttatactctcaccatactttcggatctaagatttatcacttgtccttccttctgcaagtatgctgcttcctagggctgagatctctgaaggagttgatgataatttcaacatcccaagattttgctataccttctttggggaagagcatctctcaagagtggcattttccaaataatctcataggagccctgtcaaatgtaagcacacttctggaacaagttaattcatttttacccattttttgagatgatatttactggatgggttgagttactgaacctgttcctgctgcaatctccagtcatatcacaaatctgtcatccaattgcctgtgtcacaacattagtgtccctacctccattcttcctaattctacctcatcctctatataagggccatcataaggtcttcaaacatcatatttaggtcattcttttgcaaagtctgcagaaatgccttctttcatattaaatcaactttgaatttctcagtctacactgagtcctcttgtcactaagctgcctccaggccgtcccacgtcatcatttgttgttgtccaacatgatgagctcttggttcctagccattctatttttctttaagtttctgaacttgttctcacatttcattccttctgtcatgtttaatgcattccattttaatcacctccttgttctgtactccttcaacaattatatttagccatagctaagggagagctgaatttctgaaaatatttgtggggctctaaaattagcaaggcaagttgcattttcaattcactagagggagtgaatttaaatacttaaaataaccttcctagaaattactctaagaaattaaacgagtccttttaaagagtaaaaaattcctctttaaaagagcaatttagaatcacagaataatagaagcttagtctaaggagatcacttaatcctaaacactattttttttttttttgagtcggagtttcacccttcttgcccaggctggggtgcaatggcatgatattagctcactgcaacctctgcctcccaagttcaagccattcccctgcctcagcctcctgagtaactgggattacaggcatgcaccaccatgcccagctaattttgtaattttagtagagacaggatttcaccatgttggccaggctggttttgaactcctgacctcaggtgacccacctgcttcagcctcccaaagtgccggattacaggcatgagccactgtacctggccctaaaccctaattttaaagcaactaaattatatcattgtagtttgtgttatattacatgttgcttatatgttgccttgtgctgtggtctcaatgtttgcatgcccccaaaatacatatattgaaacctaatcactaatgcgatgacattaggaggtagtccctttgggaagtgattagatcatgaggacagagccttatgaatgagattagttctcttagaaaagaagccccagagagtggccttgcctcttccaccatgtgaggacacagtaaaaaggtgttgtgagaaagtaggccctcaccagacaccaaatccctgctggtgccttgatcttggacttctcagcctccagaactgtgagaaataagttcctgttgtttataaactactttgtttacaatattttgttataccaggccaaatgaactaagacactttaaagctgatttcttaatgtgtcatatcttgttttcctacttgctcctcatgtattttctttcctagttattggcactttcatcaattcagtggcttgagctaggaaactagaagtcatcaagacctcgctcctttgctccactctacaccataatctcttcaactagactgtgatctcttgagaacaggatagtgtgtatttgacatcctatcttgcctacctagcacagtaattagaatacaggaggtagtcaataaattcttgaattagtaaatgaaacttatccttgtctctaccctatatctaaaccattatcaagttcttttgattttatcccctaaatagttatttaatctgatctctcttcccacctctgtttattccttctgattttaccccctaaatggttatttaatctgatttctctctcacctctattctcattatctcttatctagactgtcaaaataatctgtttattcaatcttgcccccctaccctctattaccgtttctcaaaatgattcattttgaattaaaatatgatattgccactactagcagaatcttttcaatggctttatatcatccacaggatgttttttaagtcccgtagcattagacaaggccctatgtgagtccttcaacttttccagtctcaggttgtgttactgtctcactcttcttttcaaatagtaccaaactgatttggtattcttgtatacgccatgaggtatatagcagatggactctttttttttttctttttttctttttttttaattattattattctttaagttttagggtacatgtgcacaatgtgcaggttagttacatatgtatacatgtgccatgctggtgtgctgcacccattaactcgtcatttagcattaagtatatctcctaatgctatccctcccccttccccccaccacaacagtccccagagtgtgatgttggactcttagcctgggttggtgaaaaggtcagagaaggattcctaaagaggaagacacattagagagacctgaaaaattagtaagagttagggggaaagaaggcagagactttgaaaaaaaaataaccttacctctttttgcccctatttggttctaagttataattgtttaaaccttgaaaatttattgcctgaagtataagcaagatgctgcatcaagccttttttcagttaagtcatgtggttggctgcaggatctttcattcattttctcattcaacaattattcattgagttctgtttatgcgccaagcattgtgttacacactgatggtacagaggtgggcaaatgaggcaagcaccctgacctcatggaatacagacacagtcattactcagttacccttcaaagcctaagacctaaagacttgattggaccacaagaggaaaccagaaatgccaaagatgtatctttagccctttatctatgtaaatgttgtgctaatccaataaggaatgaataacaattctccttagtatatggtggtgagaataactgatacttttaactctttgacaatgatactattgtacagatgaatcacaagagatcagtttatttaggtattattctaataatggttagtggacagttttgccttcaagcatggaatcagtctaagaaaatcttaaaagctttttgaattcacatcaatgtcatcacaggagtagttttttttaggtggaattgatgtttactatgcccttaaagcaataatattgcactgtaagaggcttaacacatttttaacaacctctcttaccatcctagaagcactgctggatacatgttttcaagttgatgtggtttcagtgcagaaaggttctttaaggtagcatgaaacacaaatctagcactgcaaataaatccctggtgttatttatcagggttttggataacttaggtgtcatccgtgtaagtaacacagaggtctcctctttggctaattactcaacatagtttttaatcttactagctgatgtatgaatgccaagaacatatccaaattgtatatttgctactcttctgtaagtatgtttttagtatctaatatgaccaatttaacttattgtttcaattctctatttgcaacattcaaaaagaattctttactaaaatcttttagtgttttattttcaattcagtatttcctaaattgtgcttttatttaactagttttatgcttttaactcattcatctggaaaatattttgaatttttaaaaattacgcatatctggtcatatcactccataacaagttaaatgaatgaagaaaccagcagtaagaatgaaatgtgcagatgggagggaaaggcaaaagagaacccatacatattctcaggtaggcatcagaggccaaacataggcaaagagtcagggtaattgtttaccgaatgtcagagaaagaaaagagatccagaaactgtagaatcaaatctagagccaggtgtgtaactatggttaccacggttactgtctgttactttaaccaagaaaatggaaggcatcaagaatccagtcacaactaggattgcatggcataaaaatcaattaaaaagtatttcacctatttggagagtaatagaactataggtcagaaaataagagtcctgatatcacaggaaaatcaatattataccattagcaccactaccactcctatgaccattactttaacctttatgacgatgtatgtttactgcaatatacccaatgaaatgcctagattttgataaaggtgcttagtgtgcctgtttacatgtcaaaaatactcatcaggcctaggtacctgccactataaactagtaaagctatctgagtattttctttgcattcagaatcctttaaagtggtatttttcttattcttttataatagttaggatgaggtagaattcacatgacaaagaaaagtttcaacatacactgagatggaagggagaatggcagaaacagagagaatatctatagttaggacacagggaagggagaaagcacagtgagggagataaagaaggactggtcaaagagtaggagagaacgtaaagaatacagtcttaccaaagccaagataatagctattgaaaagaggagacatgtgtgaaggtgagaaatgcttaatttcatcaagtgctatagaggaaacaagaatggtgaatttggtaattaaaaaggcattgaaagcttggggaaagtggaaataataataattaatgtccattgagtacccactgcatagtagctattattttaagaactttttatataacatcttgtttaatcttcataacaacactaagaagtttaggaggtaggtactagtactaatctcattttgtacatgaaataactaaaacacaaagatgttgggttacttactcaagtatatgacactaatggagaagccatgatttaatcctcacagtctagttctagaaccaggacttttaatggaggctgatttgaattaggctgaatttgaattaggtgctaatgttcttgagaaaatgtgggaatgagccagagatgtcagtagaaggtgacatggtgatgagaaaggattcttacaacagaagggagtggactgaaaagaagagagtagttggaccagctatctatggaatgagtctagaagtactagtggagaagaagggcatgtagacttttcacttctttaggagtgaatggccctgacatgattcggttgggaaggaagtaggaatcttaaggaaaacaaatgattcaattaagatgaagagaataaaatggtcaaatattaaaaaagaatttgttttgggggccatagtgattaggcttgcagaaaatgccaacaggttggcagaaggttagagctgtgctgaaagggatgagatcacgagacaacagccatgaagaaagggtagtaagtgattatgtacctcaggactgaagggacaagaggtcaggaggcactctgtaggggcctagatgtctacagggatatgtttaaagggggtgttgtatagatagaaaagtactttgtttccaggttccaaatataatgttattatgttggtagaagcatctccaaaactctggccctagaaattaggtcctaagtaagctttaaaagtggtactcccagtcttgggagattcacgttaaaattaaactataaaaaaactaagagaattcagaaaattatttgaatgaagaagtgctttataagtatgacaaaaagttagaattcttaaaagatttttaaattaaattgaaaacaatttcaaaatagaaaaaaaaaacccactatgagcaaagtcaaaagataaactcagtaaatattggcattttttaaaatcataaatacattttctgatatgttaagaatttccataactcagttaaaggtaattagattaatagaacaatggccaaaagacgtatttatgttcacagaaaacaaaatagaaatggtttttatacataaggaagatgttcaatctcaaccacaagaaacgttttgagtaaaccataaccagctatcattttttcttatcagcttaaccaagataaaatagttgataagttactatgttggcaaaggtataagaaagcacatactttcatacattattggtgaaagtataaattatatatttggcatatgtttaaaattagtaatgcaccatactctttgacccagcaacttgacttcttgtaatttatcccataggtatagttgcacatatgcaaaatgacatatgacaagttattcaatgaagcattatttgtagttataaaatattgtaaacagtctaaatggaaccagtgcaaatttggaattagagaaacaaattgtggtaaaatggcatgccttctaattattacagaaaagaagtaaagtataaggaagctgtgtaagtacccataaggaacaatctccaaggcaaggggtatatagtgcattatcctttgtgtaaatataggagatgcaacagaaatatatgtgtttattatttttacatagtataaatctggaagaatgtccatacatagaaaataaatccaacagacttccctggtttcctcaagggatctaggtggcataggagatgtttacccgttgtctattcttctatgtcttttgtatttttaaatgagttgccctgtcactgggattccttgtttcaggataaagataaggccaagagaattgtaattttcaaaaacaaaacttgatcagcaacaggtccccagcacagagcttaaataaattggaatggtggcttaagggtaggtttatgagagtcataaattccaaatcaagtcttgtgcgttctattttgcaggcaatgaggaagcaataaaggactctgagcaagagaacgatgtatataggccattgttttggaagattcatcacaaattcatgtgcaagtagattggaggaaggaagtatcaacagaaagaaaggccaattatgagaccattgcaatactatatggtacatatgttgcatatgtttaatattatgtataacatatataaacacttcaaaaagtcagttatcgctaacttccttcctcctttctttcattcctactatacagtccagaaagaattgaaattgataatatcttgtcacctaacttccatattccacttttctccccagtgtttggcatctttgacattttaataaaaaacaaaacagttagaaagatcaattaatattgtctttggaaaaattgttaatttgtgtttatttgcttgactcttttttccaattgagctaaatgtgattttgaaggaccattcatcatatacttcagtttaaaagtttgagattaagatttttagaccaagtgagaatttgaacaggaataggatctaatttttaaaagtaagatttggaatttcccatggaacttaacatttatgcttacttgggagttagtgaagaccttgcttatgaatgtggcaatggaaatggagaggaaatgatacatttgagaggaagctttgactacgcttatggactaagtggattttagaaaatacagaaaagcagtggttacttttgaacctggtattaggagaattactttgactctaaaatgaaaaagaaagtgggaggaaatggaggaaaggcaagcatttcatagaattactattttatgtttctaataaatcaaatttggtttggcagtagacatcaaagtggcaatagccaagcaacagaccttgagctcagggagattaacatgactatgcagattagggaatcatttgtacagaggcaggtgagttttctgagagtatagagagtttaaaaagggatagcttgaaggaaatgctccttaccacttcaataatgtgtagcaaaattattcagctggtgtttgaattggcatacaaagtcattctgataatgatagaatttgtgtaatagttttcatgtgaaagctgagactaagataagcgcttgcatgcaccatgcatcatagcagttctcctaacacatatctgtccagaaaaaccaggatttagaaacaataacaaatttgttcaatggatgttgtatgttttcatggctcttttttacaaatgaggtcagtgtatgtgtaattagattaactcacttcagtatgcttatctcatggtaacacaaaatatagattcaggaaaatcaactactcttattcattaaaacattaagttcagttgtgtaatatttacagttatgcctcaaaattatgggaaaacaaggggaaacgttgttccttaatgtaccaaatagcagacaacataaacccctcagttcactgtgacacagtttcctaattaccctacttgatatatttttgcactccattttaacttacaaatagtatgatacatattcttctcaagatataaatcaattgaaatatgtgataactctatacagtttaattttttttaaagcaaatggtagcagtattaaactgtatttatttaaataatttaagaattgagagcttt

(19) United States
(12) Patent Application Publication
(10) Pub. No.: US 2012/0046261 A1
(43) Pub. Date: Feb. 23, 2012

(54) COMPOSITIONS AND METHODS FOR DIAGNOSING AND PREVENTING SPONTANEOUS PRETERM BIRTH

(75) Inventors: Tracy Ann Manuck, Salt Lake City, UT (US); Michael W. Varner, Salt Lake City, UT (US)

(73) Assignee: UNIVERSITY OF UTAH RESEARCH FOUNDATION, Salt Lake City, UT (US)

(21) Appl. No.: 13/203,684

(22) PCT Filed: Feb. 24, 2010
(86) PCT No.: PCT/US10/25249

§ 371 Date: Nov. 3, 2011

Related U.S. Application Data
(60) Provisional application No. 61/156,080, filed on Feb. 27, 2009.

(51) Int. Cl. A61K 31/57; C40B 20/08; A61P 15/06; C40B 40/06

(52) U.S. Cl. 514/178; 506/16; 506/6; 514/177

(57) ABSTRACT

Disclosed herein are compositions and methods of identifying a subject at risk for preterm birth and selecting effective therapies for preventing preterm birth in the subject. The disclosed methods generally involve determining the identity of one or more nucleotides in the progesterone receptor (PR) gene of the subject.

### CROSS-REFERENCE TO RELATED PATENT APPLICATIONS

This application claims priority to U.S. Provisional Application No. 61/156,080 filed on Feb. 27, 2009, which is hereby incorporated herein by reference in its entirety.

### STATEMENT REGARDING FEDERALLY SPONSORED RESEARCH

This invention was made with government support under Grants HD27860, HD36801, HD27917, HD21414, HD27861, HD27869, HD27905, HD34208, HD34116, HD21410, HD27915, HD34136, HD34210, HD34122, HD40500, HD40544, HD34116, HD40560, and HD40512 awarded by the Eunice Kennedy Shriver National Institute of Child Health and Human Development (NICHD). The government has certain rights in the invention.

### BACKGROUND

Preterm labor, and subsequent delivery of premature infants, continues to remain a major cause of infant mortality and morbidity, as well as a serious economic burden on society. Premature softening of the uterine cervix during pregnancy results in premature cervical dilatation (opening) and effacement (thinning), and puts pregnant women at high risk of premature delivery. Currently there is no diagnostic method to predict premature dilatation of the cervix, and, therefore, the high-risk of preterm delivery can not be predicted in asymptomatic pregnant women.

While great strides have been made to decrease infant mortality, there has been little advancement in combating premature labor. Premature labor is now the leading factor in determining infant mortality. In North America, approximately one in eight pregnancies is complicated by preterm delivery; this rate is continuing to rise (Hoyert D L, et al. Annual summary of vital statistics: 2004. Pediatrics 2006; 117(1):168-83. While there are many identified causes of preterm labor, such as infection and multiple gestation, for many patients the cause is unknown. There has been little advancement in recent decades to develop a method of accurately predicting and treating preterm labor.

Babies born prematurely consume substantial health care resources. The hospitalization cost of preterm births is estimated to exceed $11 billion annually. The principle short term cost of these births is neonatal intensive care, time spent in the hospital by the parents, social workers and support staff for these parents, loss of earnings and increased travel expense. The long term cost of these premature births relates to the downstream effects of premature birth, such as long term health and developmental problems, increased risk of mental and physical handicap, which can impact both the child's and parent's earning potential.

Diagnosis of preterm labor and delivery has continued to be problematic. Currently there are two categories of preterm labor assessment; traditional diagnosis and biomarkers. While each has its advantages both are still relatively poor at differentiating false preterm labor.

Traditional diagnosis includes evaluation of the frequency of uterine contractions, status of the membranes, dilatation and effacement of the cervix, and gestational age. Clinical preterm labor is defined as progressive cervical dilatation, effacement, or both, with regular contractions leading to birth before 37 weeks gestation.

Cervical changes are also traditional indicators of preterm delivery. Cervical changes include a reduction in effacement, and dilatation. When a cervix is dilated or effaced (i.e. changing from closed and long or thick) in the presence of regular contractions, preterm labor is diagnosed. Cervical length of less than 1.5 cm before 30 weeks gestation was found to be a significant predictor of preterm delivery. However, after 30 weeks this measurement is an unreliable predictor. Cervical effacement of more than 80% was also a strong predictive factor. However, assessment of contraction frequency, regularity, duration, and level of perceived pain does not reliably distinguish preterm labor.

Presently, several biomarkers are used to attempt to predict preterm labor sufficiently in advance to prevent preterm delivery. Those biomarkers include fetal fibronectin, salivary estriol, decidual proteins, and endocrine/paracrine markers. Fetal fibronectin (Terao, et al. U.S. Pat. No. 5,650,394) and salivary estriol have been studied in some detail, and fetal fibronectin is commercially available. The fetal fibronectin test, currently on market as fFN (Adeza Biomedical/RossProducts Division Abbott Laboratories, Inc), is based on the detection of fetal fibronectin (a fetal-specific glycoform of fibronectin) from vaginal secretions. In symptomatic pregnant women, the test has limited effectiveness (58% sensitivity) to predict preterm delivery before the completion of 37 weeks gestation. However, the test has better predictive value for deliveries occurring within 1 week of the test (90% sensitivity). Another value of this test is the ability to exclude the possibility of preterm delivery (98% negative predictive value; Nageotte M P, et al. Fetal Fibronectin in patients at increased risk for premature birth. Am J Obstet Gynecol 1994). Thus, in asymptomatic pregnant women (low-risk of preterm delivery) the fetal fibronectin test has low sensitivity, but high specificity and good negative predictive value for predicting preterm delivery soon after the testing. Needed therefore are improved methods, such as genetic tests, for predicting preterm delivery.

### BRIEF SUMMARY

In accordance with the purpose of this invention, as embodied and broadly described herein, this invention relates to compositions and methods of identifying a subject at risk for preterm birth and selecting effective therapies for preventing preterm birth in the subject. The methods generally involve determining the identity of one or more nucleotides in the progesterone receptor (PR) gene of the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows location and structure of the human progesterone receptor. FIG. 1A shows location and orientation of PGR in the human genome. The gene usually contains eight exons and the full-length receptor spans 92 kb. FIG. 1B shows gene structure of PGR. Translation initiation sites for PR isoforms are indicated with arrows. Exons 1 and 2 are alternatively spliced and shown in gray (not drawn to scale). FIG. 1C shows schematic illustration of preoviously identified functional domains in the human progesterone receptor (hPR). Numbers indicate the amino-acid positions that delineate the beginning and end of each domain while bent arrows point to the translation initiation site of the three isoforms (PR-A, PR-B, and PR-C). AF, activation function region; IF, inhibitory function region; DBD, DNA-binding domain; NLS, nuclear localization signal; H, hinge region; HBD, hormone/ligand binding domain.

FIG. 2 shows a higher reduction in spontaneous preterm birth (SPTB)<37 weeks in African Americans subjects treated with 17-alpha hydroxy-progesterone caproate (17P) that were homozygous for the major allele (A) of rs471767, (dominant model) p=0.0229. Along the Y axis is the percentage of patients delivering preterm less than 37 weeks. Along the x-axis are 2 groups of patients, stratified by their genotype for rs471767. The black bars represent the percentage of patients delivering preterm when receiving the placebo, and the gray bars represent the percentage of patients delivering preterm when receiving 17P. This graph depicts the dominant model of inheritance. “G” is the minor allele, and thus, the AG and GG genotypes are grouped.

FIG. 3 shows a higher reduction in SPTB in African Americans treated with 17P that were carrying at least one major allele (T) of rs578029, (recessive model) p=0.0289. Women homozygous for the minor allele (A) had no reduction in preterm birth rates with 17P. However, women carrying at least one copy of the major allele (T) had a significant reduction in the rate of prematurity, from approximately 55%, seen here in black, to 25%, demonstrated in gray.

FIG. 4 shows a higher reduction in SPTB<32 weeks gestation with 17P in patients carrying at least one minor allele (C) of rs503362 (dominant model). Women with at least one minor allele (C) of rs503362 had a significant reduction in the rate of preterm birth from 17.4% with placebo to 2.6% with 17P. Patients homozygous for the major allele (G) did not have a reduction in the rate of prematurity when receiving 17P.

FIG. 5 shows a higher reduction in SPTB<32 weeks gestation in Caucasian/Hispanic patients treated with 17P when carrying at least one minor allele, “T” of rs666553 (dominant model), p=0.027. Women with at least one minor allele (T) had a reduction in the rate of preterm birth from 26.7% with the placebo to 3.4% with 17P. In contrast, women homozygous for the major allele had a lower rate of early prematurity with the placebo and do not experience any reduction in prematurity with 17P.

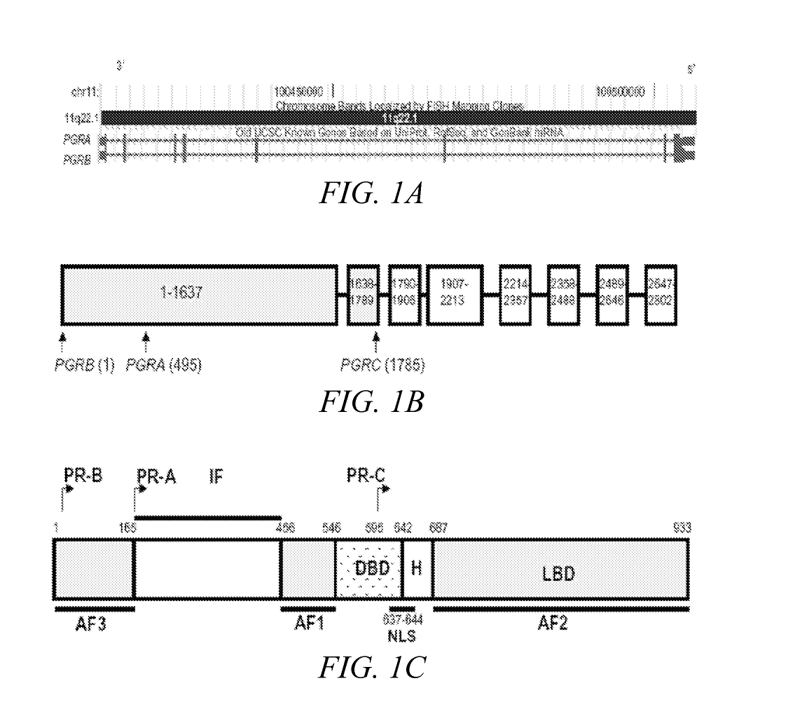

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid is disclosed and discussed and a number of modifications that can be made to a number of molecules including the nucleic acid are discussed, each and every combination and permutation of nucleic acid and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a nucleic acid” includes a plurality of such nucleic acids, reference to “the nucleic acid” is a reference to one or more nucleic acids and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “pharmaceutically effective amount” (or interchangeably referred to herein as “an effective amount”) has its usual meaning in the art, i.e., an amount of a pharmaceutical that is capable of inducing an in vivo and/or clinical response that facilitates management, prophylaxis, or therapy. This term can encompass therapeutic or prophylactic effective amounts, or both. As used herein, the term “suitable” means fit for mammalian, preferably human, use and for the pharmaceutical purposes disclosed herein.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms. In some embodiments, the term “treatment” or “treating” includes ameliorating the symptoms of, curing or healing, and preventing the development of a given disease.

The term “prophylaxis” is intended as an element of “treatment” to encompass both “preventing” and “suppressing,” as defined herein. It will be understood by those skilled in the art that in human medicine it is not always possible to distinguish between “preventing” and “suppressing” since the ultimate inductive event or events may be unknown, latent, or the patient is not ascertained until well after the occurrence of the event or events.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. SPONTANEOUS PRETERM BIRTH

Disclosed herein are compositions and methods relating to the prevention of spontaneous preterm birth. Approximately 20%-30% of births occurring prior to 37 weeks of gestation are the result of a physician's decision to bring about delivery for maternal or fetal indications. The remaining preterm deliveries are spontaneous, usually following the onset of premature labor or rupture of the membranes. The compositions and methods disclosed herein are relevant only to these spontaneous forms of preterm deliver. Thus, “spontaneous preterm birth,” “SPTB,” and “preterm birth” are used herein interchangeably to refer to preterm births that occur without labor being intentionally induced by the physician.

Several risk factors for preterm labor have been identified including: multi-fetal gestation, maternal stress, systemic and intrauterine infection, race, and socioeconomic status. The Preterm Prediction Study found that a history of prior spontaneous preterm delivery was a strong predictor of subsequent preterm delivery with a prior delivery at 23-27 weeks giving rise to an 11-fold increase in the risk. Unfortunately, however, risk assessment methods using only historical risk factors have an unacceptably low sensitivity and poor predictive value. Supplementing historical-based risk assessment with technology, specifically an ultrasonographic assessment of the cervix, adds improved sensitivity and specificity. Ultrasound identification of cervical shortening is correlated with a logarithmic increase in the risk of preterm delivery. See Jams et al, The length of the cervix and the risk of spontaneous premature delivery, N. Engl. J. Med. 1996; 334: 567-572.

Pregnant women who experience spontaneous preterm birth (PTB) begin with preterm labor (also interchangeably referred to herein as “premature labor”) and start having regular contractions that cause their cervix to start to open or thin out (called dilation and effacement) and soften before they reach about 37 weeks of gestation. If a woman delivers a baby before 37 weeks, it is typically and conventionally called a preterm birth and the baby is considered premature.

Preterm birth remains one of the most serious problems in obstetrics, with enormous impact on infants, their families, and our society. According to a recent study published by the Institute of Medicine, the incidence of preterm birth has grown 33% since 1981, and each year approximately 500,000 women deliver prematurely in the U.S. alone resulting in a $26 billion annual cost of premature birth to our nation's healthcare system. It has recently been reported that preterm births occur in 15% of pregnancies in the developed world and 12.7% of all births in the United States in 2006 and 12.4% of all births in the United States in 2004. See, e.g., Use of progesterone to reduce preterm birth, American College of Obstetricians and Gynecologists Committee Opinion No. 291, Vol. 102, No. 5, November 2003, pages 1115-1116; Hamilton, B. E, Annal Summary of Vital Statistics: 2005, Pediatrics, Vol. 119, No. 2, February 2007, pages 345-360.

It is believed that a preterm birth prior to 32 weeks of gestation represents an extremely high risk of morbidity and mortality. Additionally, a preterm birth between 32 and 36 weeks of gestation has been found to be particularly alarming as having a great number of at risk infants. Preterm delivery accounts for 60-70% of infant mortality, and is a leading cause of health care expenditures in both the perinatal period and throughout life for infant survivors. Recent advances in modern obstetric and neonatal care have led to improved infant survival, however, 55% of newborns with an extremely low birth weight (<1000 g) or delivered very premature (<28 weeks) who survive to middle childhood show evidence of clinically significant cognitive, educational, and behavioral impairment.

Of the surviving premature infants, many are afflicted with lifelong difficulties such as cerebral palsy, mental retardation, chronic lung disease, hearing and vision deficits, and learning disabilities. The more mature a child is at birth, the more likely he or she is to survive and the less likely he or she is to have related health problems. Premature babies born between 34 and 37 weeks previously were generally considered relatively healthy. However, an increasing number of studies of these ‘late preterm’ births indicate that these babies have a much higher incidence of respiratory complications, persistent pulmonary hypertension, temperature instability, jaundice, feeding difficulties, and a higher rate of mortality compared to their term counterparts. Evidence is currently emerging that these ‘late preterm’ infants may even have long-term neurodevelopmental consequences as a result of their prematurity. If a woman goes into labor before 34 weeks, however, the risks of adverse health effects and/or medical complications increase.

The length of a woman's cervix is a good indication of whether a pregnant woman will experience preterm labor and preterm birth. Many physicians routinely check the length of a woman's cervix by transvaginal ultrasound at 16-20 weeks gestation if the woman is at high risk for preterm birth, so that they can monitor changes in cervical length as the pregnancy progresses. If a woman's cervix is short or shortening, it means that the cervix is beginning to efface (thin out). The risk of preterm delivery is inversely correlated with cervical length; the shorter the length of the cervix, the higher the risk of preterm birth. Cervical length measurements less than 2.5 cm are considered “short,” and a cervical length less than 1.5 cm is extremely high risk.

As used herein, the term “preterm” generally describes human gestation resulting in birth prior to 37 weeks. Accordingly, “preterm” covers births occurring less than 35 weeks or less than or equal to 32 weeks of gestation. Additionally, another definition of preterm labor includes dilation and/or effacement of the cervix, which is detected by digital examination, associated with persistent uterine contractions before 37 weeks of gestation. In some embodiments, preterm labor comprises 6 or more uterine contractions per hour accompanied by documented cervical change, cervical dilation greater than 2 cm, cervical effacement greater than 80%, or documented change in cervical effacement greater than 50%.

As used herein, short cervix describes a cervical length less than 3.5 cm, including less than 3.0 cm, less than 2.5 cm, and less than 2.0 cm. How to identify and clinically diagnose pregnant women having short cervix would be understood by one skilled in the art, and can include such methods as sonographic examination and clinical examination, for example.

As used herein, “neonatal” encompasses children about 6 months of age or less, including about 3 months of age or less, about 2 months of age or less, and about 1 month of age or less. In certain embodiments, neonatal is used to encompass perinatal.

### C. COMPOSITIONS

Progesterone is critical to pregnancy maintenance; it binds human progesterone receptors (hPR) and modulates gene expression. Patients with a personal or family history of spontaneous preterm birth (SPTB) have elevated risks of SPTB. Disclosed herein are nucleotides in the progesterone receptor (PR) gene that can be used to indicate a subject at risk of preterm birth

1. Progesterone Receptor

The progesterone receptor (PR) also known as NR3C3 (nuclear receptor subfamily 3, group C, member 3), is an intracellular steroid receptor that specifically binds progesterone. PR is encoded by a single gene PGR residing on chromosome 11q22. It has two main isoforms, A and B, transcribed from the same gene, that differ in their molecular weight.

Like all steroid receptors, the progesterone receptor has an amino and a carboxyl terminal, and between them the regulatory domain, a DNA binding domain, the hinge section, and the hormone binding domain. A special transcription activation function (TAF), called TAF-3, is present in the progesterone receptor-B, in a B-upstream segment (BUS) at the amino acid terminal. This segment is not present in the receptor-A.

As demonstrated in PR-deficient mice, the physiological effects of progesterone depend completely on the presence of the human progesterone receptor (hPR), a member of the steroid-receptor superfamily of nuclear receptors. The single-copy human PGR gene uses separate promoters and translational start sites to produce two isoforms, hPR-A and -B, which are identical except for an additional 165 amino acids present only in the N terminus of hPR-B. Although hPR-B shares many important structural domains as hPR-A, they are in fact two functionally distinct transcription factors, mediating their own response genes and physiological effects with little overlap. Selective ablation of PR-A in a mouse model, resulting in exclusive production of PR-B, revealed that PR-B contributes to, rather than inhibits, epithelial cell proliferation both in response to estrogen alone and in the presence of progesterone and estrogen. These results indicate that in the uterus, the PR-A isoform is necessary to oppose estrogen-induced proliferation as well as PR-B-dependent proliferation.

Other studies indicate that the shorter PR-A inhibits the transcription of progesterone receptive genes, including the transcription of PR-B. In contrast, PR-B increases transcription of progesterone-responsive genes and has an overall quiescent effect on the myometrium. Several studies have shown that the transformation of the myometrium from a relaxed to contractile state depends on, or is accompanied by, an abrupt increase in the transcription of PR-A Thus, the responsiveness of target tissues to both endogenous and exogenous progesterone likely depends not only on the level of progesterone but also on the ratio of PR isoforms. A relative increase in the ratio of PR-A to PR-B may contribute to a functional withdrawal of progesterone and subsequent initiation of labor.

Six variable sites, including at least four polymorphisms and five common haplotypes have been identified in the human PR gene. One promoter region polymorphism, +331G/A (rs10895068), creates a unique transcription start site. Biochemical assays showed that the +331G/A polymorphism increases transcription of the PR gene, favoring production of hPR-B in an Ishikawa endometrial cancer cell line.

Estrogen is necessary to induce the progesterone receptors. When no binding hormone is present the carboxyl terminal inhibits transcription. Binding to a hormone induces a structural change that removes the inhibitory action. Progesterone antagonists prevent the structural reconfiguration.

After progesterone binds to the receptor, restructuring with dimerization follows and the complex enters the nucleus and binds to DNA. There transcription takes place, resulting in formation of messenger RNA that is translated by ribosomes to produce specific proteins.

Disclosed herein are single nucleotide polymorphisms (SNPs) of the PR gene that can be used to identify a subject at risk of preterm birth, select therapies for treating a subject at risk of preterm birth, and prolong gestation in subjects at risk of preterm birth. A non-exhaustive list of SNPs for use in the disclosed compositions and methods are provided in Table 1 below. Each SNP has at least two known alleles. Disclosed is a consensus sequence for each SNP wheren the substituted residue is identified with the identifier Y (C or T), R (A or G), W (A or T), S(C or G), or K (G or T). However, also disclosed is a sequence for each SNP where the identified residue is N (A, G, C, or T). Thus, in some aspects, the disclosed methods comprise identifying a residue for each SNP location other than the one present in a control population. In other aspects, the method comprises identifying the residue for each SNP location identified as the 1^{st} or 2^{nd} allele.

TABLE 1
hPR SNPs

| SNP | Chr 11 Location | Base Change | Consensus | 1^{st} Allele | 2^{nd} Allele |
|---|---|---|---|---|---|
| rs1893505 | 100572918 | C/T | SEQ ID NO: 3 | SEQ ID NO: 4 | SEQ ID NO: 5 |
| rs10501973 | 100568786 | A/G | SEQ ID NO: 6 | SEQ ID NO: 7 | SEQ ID NO: 8 |
| rs954723 | 100568141 | C/T | SEQ ID NO: 9 | SEQ ID NO: 10 | SEQ ID NO: 11 |
| rs1942836 | 100554557 | C/T | SEQ ID NO: 12 | SEQ ID NO: 13 | SEQ ID NO: 14 |
| rs471811 | 100549413 | A/G | SEQ ID NO: 15 | SEQ ID NO: 16 | SEQ ID NO: 17 |
| rs568157 | 100529492 | A/G | SEQ ID NO: 18 | SEQ ID NO: 19 | SEQ ID NO: 20 |
| rs474320 | 100549413 | A/T | SEQ ID NO: 21 | SEQ ID NO: 22 | SEQ ID NO: 23 |
| rs4754732 | 100513712 | C/T | SEQ ID NO: 24 | SEQ ID NO: 25 | SEQ ID NO: 26 |
| rs3740753 | 100503981 | C/G | SEQ ID NO: 27 | SEQ ID NO: 28 | SEQ ID NO: 29 |
| rs582691 | 100500076 | A/G | SEQ ID NO: 30 | SEQ ID NO: 31 | SEQ ID NO: 32 |
| rs493957 | 100494658 | A/G | SEQ ID NO: 33 | SEQ ID NO: 34 | SEQ ID NO: 35 |
| rs503362 | 100467037 | C/G | SEQ ID NO: 36 | SEQ ID NO: 37 | SEQ ID NO: 38 |
| rs653752 | 100453320 | C/G | SEQ ID NO: 39 | SEQ ID NO: 40 | SEQ ID NO: 41 |
| rs666553 | 100443878 | C/T | SEQ ID NO: 42 | SEQ ID NO: 43 | SEQ ID NO: 44 |
| rs578029 | 100427614 | A/T | SEQ ID NO: 45 | SEQ ID NO: 46 | SEQ ID NO: 47 |
| rs1042839 | 100427412 | A/G | SEQ ID NO: 48 | SEQ ID NO: 49 | SEQ ID NO: 50 |
| rs1042838 | 100438622 | G/T | SEQ ID NO: 51 | SEQ ID NO: 52 | SEQ ID NO: 53 |
| rs500760 | 100415201 | A/G | SEQ ID NO: 54 | SEQ ID NO: 55 | SEQ ID NO: 56 |
| rs471767 | 100410507 | A/G | SEQ ID NO: 57 | SEQ ID NO: 58 | SEQ ID NO: 59 |
| rs10895068 | 100505424 | G/A | SEQ ID NO: 60 | SEQ ID NO: 61 | SEQ ID NO: 62 |

SNP rs471767 is located just upstream of the progesterone receptor promoter. Given the location of this SNP, polymorphisms may alter the transcription of PR-A and PR-B, or their relative ratios.

SNP rs1042838, which is a G>T substitution in exon 4 of the PGR gene, results in V660L mutation. This SNP is also in complete LD with PROGINS, which has been reported as inversely correlated with risk of breast cancer, ovarian cancer, and endometriosis. However, results in this study did not show a relationship between this SNP and response to 17P.

SNP rs503362 is also in complete LD with V660L and PROGINS. This SNP is associated with prematurity (p=0.008) (Ehn, et al., Pediatric Research 2007).

There are 2 distinct isoforms of hPR, PR-A, and PR-B. PR-B (116 kD) activates progesterone-responsive genes and maintains uterine quiescence & pregnancy. PR-A (94 kD) represses PR-B function and increases prior to labor. Thus, increased PR-A/PR-B ratio can in some aspects lead to functional progesterone withdrawal.

Increased PR-A/PR-B ratio can affect prostaglandins (acting via PKC pathway). For example, overexpression of PRA reverses the inhibitory effect of P on MMP expression. Overexpression of PRB increases progesterone effect and further decreases MMP2 expression and promotor activity.

2. Array

Also disclosed herein is an array of nucleic acid molecules attached to a solid support for use in detecting the PR gene single nucleotide polymorphisms (SNPs) disclosed herein. Thus, disclosed is an array of nucleic acid molecules attached to a solid support, wherein at least one of the nucleic acids comprise a sequence corresponding to SNP rs471767, rs1893505, rs10501973, rs954723, rs1942836, rs471811, rs568157, rs474320, rs4754732, rs3740753, rs582691, rs493957, rs503362, rs653752, rs666553, rs578029, rs1042839, rs1042838, rs500760, or rs10895068, or the complement thereof.

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots.

Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GeneChip® (Affymetrix, Inc which refers to its high density, oligonucleotide-based DNA arrays), and gene array.

A DNA microarray is a collection of microscopic DNA spots attached to a solid surface, such as glass, plastic or silicon chip forming an array for the purpose of expression profiling, monitoring expression levels for thousands of genes simultaneously. DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

The affixed DNA segments are generally known as probes, thousands of which can be placed in known locations on a single DNA microarray. Microarray technology evolved from Southern blotting, whereby fragmented DNA is attached to a substrate and then probed with a known gene or fragment. Measuring gene expression using microarrays is relevant to many areas of biology and medicine, such as studying treatments, disease, and developmental stages. For example, microarrays can be used to identify disease genes by comparing gene expression in diseased and normal cells.

There are two variants of the DNA microarray technology, in terms of the property of arrayed DNA sequence with known identity. Type I microarrays comprise a probe cDNA (500˜5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

Type II microarrays comprise an array of oligonucleotides (20˜80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GeneChip® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

In spotted microarrays (or two-channel or two-colour microarrays), the probes are oligonucleotides, cDNA or small fragments of PCR products corresponding to mRNAs. This type of array is typically hybridized with cDNA from two samples to be compared (e.g., patient and control) that are labeled with two different fluorophores. The samples can be mixed and hybridized to one single microarray that is then scanned, allowing the visualization of up-regulated and down-regulated genes in one go. The downside of this is that the absolute levels of gene expression cannot be observed, but only one chip is needed per experiment. One example of a provider for such microarrays is Eppendorf with their DualChip® platform.

In oligonucleotide microarrays (or single-channel microarrays), the probes are designed to match parts of the sequence of known or predicted mRNAs. There are commercially available designs that cover complete genomes from companies such as GE Healthcare, Affymetrix, Ocimum Biosolutions, or Agilent. These microarrays give estimations of gene expression and therefore the comparison of two conditions requires the use of two separate microarrays.

Long Oligonucleotide Arrays are composed of 60-mers, or 50-mers and are produced by ink jet printing on a silica substrate. Short Oligonucleotide Arrays are composed of 25-mer or 30-mer and are produced by photolithographic synthesis (Affymetrix) on a silica substrate or piezoelectric deposition (GE Healthcare) on an acrylamide matrix. More recently, Maskless Array Synthesis from NimbleGen Systems has combined flexibility with large numbers of probes. Arrays can contain up to 390,000 spots, from a custom array design. New array formats are being developed to study specific pathways or disease states for a systems biology approach.

Oligonucleotide microarrays often contain control probes designed to hybridize with RNA spike-ins. The degree of hybridization between the spike-ins and the control probes is used to normalize the hybridization measurements for the target probes.

SNP microarrays are a particular type of DNA microarrays that are used to identify genetic variation in individuals and across populations. Short oligonucleotide arrays can be used to identify the single nucleotide polymorphisms (SNPs) that are thought to be responsible for genetic variation and the source of susceptibility to genetically caused diseases. Generally termed genotyping applications, DNA microarrays may be used in this fashion for forensic applications, rapidly discovering or measuring genetic predisposition to disease, or identifying DNA-based drug candidates.

These SNP microarrays are also being used to profile somatic mutations in cancer, specifically loss of heterozygosity events and amplifications and deletions of regions of DNA. Amplifications and deletions can also be detected using comparative genomic hybridization in conjunction with microarrays.

Resequencing arrays have also been developed to sequence portions of the genome in individuals. These arrays may be used to evaluate germline mutations in individuals, or somatic mutations in cancers.

Genome tiling arrays include overlapping oligonucleotides designed to blanket an entire genomic region of interest. Many companies have successfully designed tiling arrays that cover whole human chromosomes.

Samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Probes. Part I. Theory and Nucleic Acid Preparation, P. Tijssen, ed. Elsevier (1993). In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one preferred embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York, N.Y.; and Sambrook, J. et al. (1989) Molecular Cloning, A Laboratory Manual, Cold Spring Harbor Press, Plainview, N.Y.).

Methods for detecting complex formation are well known to those skilled in the art. In a preferred embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Microarrays can be fabricated using a variety of technologies, including printing with fine-pointed pins onto glass slides, photolithography using pre-made masks, photolithography using dynamic micromirror devices, ink-jet printing (Lausted C, et al. Genome Biol. 2004; 5(8):R58), or electrochemistry on microelectrode arrays.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734, which are hereby incorporated by reference for the teaching of making and using polynucleotide arrays. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Typical dispensers include a micropipette delivering solution to the substrate with a robotic system to control the position of the micropipette with respect to the substrate. There can be a multiplicity of dispensers so that reagents can be delivered to the reaction regions simultaneously. For example, a microarray can be formed by using ink jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate (Baldeschweiler et al. PCT publication WO95/251116).

Thus, disclosed is an array of nucleic acid molecules attached to a solid support, wherein at least one of the nucleic acids comprise a sequence corresponding to the sequences or complement thereof for SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, or a fragment thereof comprising at least 10 nucleotides and comprising the SNP identified herein.

The disclosed array can comprise an oligonucleotide that hybridizes under stringent conditions to a nucleic acid molecule consisting of SEQ ID NO:4 (SNP rs1893505 C allele) but does not hybridize under stringent conditions to a nucleic acid molecule consisting of SEQ ID NO:5 (SNP rs1893505 T allele). In addition or in the alternative, the disclosed array can comprise an oligonucleotide that hybridizes under stringent conditions to a nucleic acid molecule consisting of SEQ ID NO:5 (SNP rs1893505 T allele) but does not hybridize under stringent conditions to a nucleic acid molecule consisting of SEQ ID NO:4 (SNP rs1893505 C allele).

The disclosed array can comprise an oligonucleotide that hybridizes under stringent conditions to a nucleic acid molecule consisting of SEQ ID NO:7 (SNP rs10501973 A allele) but does not hybridize under stringent conditions to a nucleic acid molecule consisting of SEQ ID NO:8 (SNP rs10501973 G allele). In addition or in the alternative, the disclosed array can comprise an oligonucleotide that hybridizes under stringent conditions to a nucleic acid molecule consisting of SEQ ID NO:8 (SNP rs10501973 G allele) but does not hybridize under stringent conditions to a nucleic acid molecule consisting of SEQ ID NO:7 (SNP rs10501973 A allele).

The disclosed array can comprise an oligonucleotide that hybridizes under stringent conditions to a nucleic acid molecule consisting of SEQ ID NO:10 (SNP rs954723 C allele) but does not hybridize under stringent conditions to a nucleic acid molecule consisting of SEQ ID NO:11 (SNP rs954723 T allele). In addition or in the alternative, the disclosed array can comprise an oligonucleotide that hybridizes under stringent conditions to a nucleic acid molecule consisting of SEQ ID NO:11 (SNP rs954723 T allele) but does not hybridize under stringent conditions to a nucleic acid molecule consisting of SEQ ID NO:10 (SNP rs954723 C allele).

The disclosed array can comprise an oligonucleotide that hybridizes under stringent conditions to a nucleic acid molecule consisting of SEQ ID NO:13 (SNP rs1942836 C allele) but does not hybridize under stringent conditions to a nucleic acid molecule consisting of SEQ ID NO:14 (SNP rs1942836 T allele). In addition or in the alternative, the disclosed array can comprise an oligonucleotide that hybridizes under stringent conditions to a nucleic acid molecule consisting of SEQ ID NO:14 (SNP rs1942836 T allele) but does not hybridize under stringent conditions to a nucleic acid molecule consisting of SEQ ID NO:13 (SNP rs1942836 C allele).

The disclosed array can comprise an oligonucleotide that hybridizes under stringent conditions to a nucleic acid molecule consisting of SEQ ID NO:16 (SNP rs471811 A allele) but does not hybridize under stringent conditions to a nucleic acid molecule consisting of SEQ ID NO:17 (SNP rs471811 G allele). In addition or in the alternative, the disclosed array can comprise an oligonucleotide that hybridizes under stringent conditions to a nucleic acid molecule consisting of SEQ ID NO:17 (SNP rs471811 G allele) but does not hybridize under stringent conditions to a nucleic acid molecule consisting of SEQ ID NO:16 (SNP rs471811 A allele).

The disclosed array can comprise an oligonucleotide that hybridizes under stringent conditions to a nucleic acid molecule consisting of SEQ ID NO:19 (SNP rs568157 A allele) but does not hybridize under stringent conditions to a nucleic acid molecule consisting of SEQ ID NO:20 (SNP rs568157 G allele). In addition or in the alternative, the disclosed array can comprise an oligonucleotide that hybridizes under stringent conditions to a nucleic acid molecule consisting of SEQ ID NO:20 (SNP rs568157 G allele) but does not hybridize under stringent conditions to a nucleic acid molecule consisting of SEQ ID NO:19 (SNP rs568157 A allele).

The disclosed array can comprise an oligonucleotide that hybridizes under stringent conditions to a nucleic acid molecule consisting of SEQ ID NO:22 (SNP rs474320 A allele) but does not hybridize under stringent conditions to a nucleic acid molecule consisting of SEQ ID NO:23 (SNP rs474320 T allele). In addition or in the alternative, the disclosed array can comprise an oligonucleotide that hybridizes under stringent conditions to a nucleic acid molecule consisting of SEQ ID NO:23 (SNP rs474320 T allele) but does not hybridize under stringent conditions to a nucleic acid molecule consisting of SEQ ID NO:22 (SNP rs474320 A allele).

The disclosed array can comprise an oligonucleotide that hybridizes under stringent conditions to a nucleic acid molecule consisting of SEQ ID NO:25 (SNP rs4754732 C allele) but does not hybridize under stringent conditions to a nucleic acid molecule consisting of SEQ ID NO:26 (SNP rs4754732 T allele). In addition or in the alternative, the disclosed array can comprise an oligonucleotide that hybridizes under stringent conditions to a nucleic acid molecule consisting of SEQ ID NO:26 (SNP rs4754732 T allele) but does not hybridize under stringent conditions to a nucleic acid molecule consisting of SEQ ID NO:25 (SNP rs4754732 C allele).

The disclosed array can comprise an oligonucleotide that hybridizes under stringent conditions to a nucleic acid molecule consisting of SEQ ID NO:28 (SNP rs3740753 C allele) but does not hybridize under stringent conditions to a nucleic acid molecule consisting of SEQ ID NO:29 (SNP rs3740753 G allele). In addition or in the alternative, the disclosed array can comprise an oligonucleotide that hybridizes under stringent conditions to a nucleic acid molecule consisting of SEQ ID NO:29 (SNP rs3740753 G allele) but does not hybridize under stringent conditions to a nucleic acid molecule consisting of SEQ ID NO:28 (SNP rs3740753 C allele).

The disclosed array can comprise an oligonucleotide that hybridizes under stringent conditions to a nucleic acid molecule consisting of SEQ ID NO:31 (SNP rs582691 A allele) but does not hybridize under stringent conditions to a nucleic acid molecule consisting of SEQ ID NO:32 (SNP rs582691 G allele). In addition or in the alternative, the disclosed array can comprise an oligonucleotide that hybridizes under stringent conditions to a nucleic acid molecule consisting of SEQ ID NO:32 (SNP rs582691 G allele) but does not hybridize under stringent conditions to a nucleic acid molecule consisting of SEQ ID NO:31 (SNP rs582691 A allele).

The disclosed array can comprise an oligonucleotide that hybridizes under stringent conditions to a nucleic acid molecule consisting of SEQ ID NO:34 (SNP rs493957 A allele) but does not hybridize under stringent conditions to a nucleic acid molecule consisting of SEQ ID NO:35 (SNP rs493957 G allele). In addition or in the alternative, the disclosed array can comprise an oligonucleotide that hybridizes under stringent conditions to a nucleic acid molecule consisting of SEQ ID NO:35 (SNP rs493957 G allele) but does not hybridize under stringent conditions to a nucleic acid molecule consisting of SEQ ID NO:34 (SNP rs493957 A allele).

The disclosed array can comprise an oligonucleotide that hybridizes under stringent conditions to a nucleic acid molecule consisting of SEQ ID NO:37 (SNP rs503362 C allele) but does not hybridize under stringent conditions to a nucleic acid molecule consisting of SEQ ID NO:38 (SNP rs503362 G allele). In addition or in the alternative, the disclosed array can comprise an oligonucleotide that hybridizes under stringent conditions to a nucleic acid molecule consisting of SEQ ID NO:38 (SNP rs503362 G allele) but does not hybridize under stringent conditions to a nucleic acid molecule consisting of SEQ ID NO:37 (SNP rs503362 C allele).

The disclosed array can comprise an oligonucleotide that hybridizes under stringent conditions to a nucleic acid molecule consisting of SEQ ID NO:40 (SNP rs653752 C allele) but does not hybridize under stringent conditions to a nucleic acid molecule consisting of SEQ ID NO:41 (SNP rs653752 G allele). In addition or in the alternative, the disclosed array can comprise an oligonucleotide that hybridizes under stringent conditions to a nucleic acid molecule consisting of SEQ ID NO:41 (SNP rs653752 G allele) but does not hybridize under stringent conditions to a nucleic acid molecule consisting of SEQ ID NO:40 (SNP rs653752 C allele).

The disclosed array can comprise an oligonucleotide that hybridizes under stringent conditions to a nucleic acid molecule consisting of SEQ ID NO:43 (SNP rs666553 C allele) but does not hybridize under stringent conditions to a nucleic acid molecule consisting of SEQ ID NO:44 (SNP rs666553 T allele). In addition or in the alternative, the disclosed array can comprise an oligonucleotide that hybridizes under stringent conditions to a nucleic acid molecule consisting of SEQ ID NO:44 (SNP rs666553 T allele) but does not hybridize under stringent conditions to a nucleic acid molecule consisting of SEQ ID NO:43 (SNP rs666553 C allele).

The disclosed array can comprise an oligonucleotide that hybridizes under stringent conditions to a nucleic acid molecule consisting of SEQ ID NO:46 (SNP rs578029 A allele) but does not hybridize under stringent conditions to a nucleic acid molecule consisting of SEQ ID NO:47 (SNP rs578029 T allele). In addition or in the alternative, the disclosed array can comprise an oligonucleotide that hybridizes under stringent conditions to a nucleic acid molecule consisting of SEQ ID NO:47 (SNP rs578029 T allele) but does not hybridize under stringent conditions to a nucleic acid molecule consisting of SEQ ID NO:46 (SNP rs578029 A allele).

The disclosed array can comprise an oligonucleotide that hybridizes under stringent conditions to a nucleic acid molecule consisting of SEQ ID NO:49 (SNP rs1042839 A allele) but does not hybridize under stringent conditions to a nucleic acid molecule consisting of SEQ ID NO:50 (SNP rs1042839 G allele). In addition or in the alternative, the disclosed array can comprise an oligonucleotide that hybridizes under stringent conditions to a nucleic acid molecule consisting of SEQ ID NO:50 (SNP rs1042839 G allele) but does not hybridize under stringent conditions to a nucleic acid molecule consisting of SEQ ID NO:49 (SNP rs1042839 A allele).

The disclosed array can comprise an oligonucleotide that hybridizes under stringent conditions to a nucleic acid molecule consisting of SEQ ID NO:52 (SNP rs1042838 G allele) but does not hybridize under stringent conditions to a nucleic acid molecule consisting of SEQ ID NO:53 (SNP rs1042838 T allele). In addition or in the alternative, the disclosed array can comprise an oligonucleotide that hybridizes under stringent conditions to a nucleic acid molecule consisting of SEQ ID NO:53 (SNP rs1042838 T allele) but does not hybridize under stringent conditions to a nucleic acid molecule consisting of SEQ ID NO:52 (SNP rs1042838 G allele).

The disclosed array can comprise an oligonucleotide that hybridizes under stringent conditions to a nucleic acid molecule consisting of SEQ ID NO:55 (SNP rs500760 A allele) but does not hybridize under stringent conditions to a nucleic acid molecule consisting of SEQ ID NO:56 (SNP rs500760 G allele). In addition or in the alternative, the disclosed array can comprise an oligonucleotide that hybridizes under stringent conditions to a nucleic acid molecule consisting of SEQ ID NO:56 (SNP rs500760 G allele) but does not hybridize under stringent conditions to a nucleic acid molecule consisting of SEQ ID NO:55 (SNP rs500760 A allele).

The disclosed array can comprise an oligonucleotide that hybridizes under stringent conditions to a nucleic acid molecule consisting of SEQ ID NO:59 (SNP rs471767 G allele) but does not hybridize under stringent conditions to a nucleic acid molecule consisting of SEQ ID NO:58 (SNP rs471767 A allele). In addition or in the alternative, the disclosed array can comprise an oligonucleotide that hybridizes under stringent conditions to a nucleic acid molecule consisting of SEQ ID NO:58 (SNP rs471767 A allele) but does not hybridize under stringent conditions to a nucleic acid molecule consisting of SEQ ID NO:59 (SNP rs471767 G allele).

The disclosed array can comprise an oligonucleotide that hybridizes under stringent conditions to a nucleic acid molecule consisting of SEQ ID NO:61 (SNP rs10895068 G allele) but does not hybridize under stringent conditions to a nucleic acid molecule consisting of SEQ ID NO:62 (SNP rs10895068 A allele). In addition or in the alternative, the disclosed array can comprise an oligonucleotide that hybridizes under stringent conditions to a nucleic acid molecule consisting of SEQ ID NO:62 (SNP rs10895068 A allele) but does not hybridize under stringent conditions to a nucleic acid molecule consisting of SEQ ID NO:61 (SNP rs10895068 G allele).

3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

4. Nucleic Acids

The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86,6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

The sequences for PGR, including human PGR, as well as other analogs, and alleles of these genes, and splice variants and other types of variants, are available in a variety of protein and gene databases, including Genbank. For example, a genomic sequence for human chromosome 11 is disclosed in Accession No. NC_{—}000011. Likewise, a genomic sequence for human PGR is disclosed in Accession No. NT_{—}033899. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

Also disclosed are compositions including primers and probes, which are capable of interacting with the disclosed nucleic acids. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some aspects, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

5. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved. Thus, disclosed are computer readable mediums comprising the sequences and information regarding the sequences set forth herein.

6. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting one or more of the SNPs disclosed herein, the kit comprising, for example, nucleic acid probes that bind to a target nucleic acid having the one or more SNPs but not to a nucleic acid that does not comprise the one or more SNPs. The disclosed kits can also include profiles of SNPs in control populations with instructions for interpreting the results.

7. Uses

The disclosed compositions can be used in a variety of ways as research tools. Other uses are disclosed, apparent from the disclosure, and/or will be understood by those in the art.

### D. METHODS

1. Risk Identification

Disclosed herein is a method of identifying a subject at risk for preterm birth, comprising determining in a sample of nucleic acid from the subject the identity of one or more nucleotides in the progesterone receptor (PR) gene, wherein a substitution of a nucleotide at one or more positions in the PR gene of the subject compared to a control indicates that the subject is at risk of preterm birth.

The term “progesterone receptor gene,” “PR gene,” or “PGR gene” is meant to include genomic DNA encoding progesterone receptor, including introns and exons, as well as 5′ and 3′ untranslated regions (UTR). For example, a sequence for hPR gene is provided in SEQ ID NO:1, a sequence comprising the 5′ UTR is provided in SEQ ID NO:63, and a sequence comprising the 3′ UTR is provided in SEQ ID NO:64, all of which are disclosed herein as nucleotides in the human PR gene.

Thus, the one or more nucleotides in the PR gene can be at least about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, or more nucleotides 5′ to the start codon for PR. Thus, the one or more nucleotides in the PR gene can be at least about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, or more nucleotides 3′ to the stop codon for PR.

The method disclosed herein does not require detection of the substitution directly within the genomic DNA of the subject. The method can comprise detecting nucleotides or amino acid residues in a sample that correspond to nucleotides within the PR gene within the subject. Thus, the method can comprise detecting a nucleotide substitution in mRNA or cDNA that corresponds to a nucleotide within the subject's PR gene. The method can also comprise detecting a mutation within the PR protein (or fragment thereof) of the subject and thereby demonstrate the presence of one or more nucleotide substitutions within the subject's PR gene.

The methods described herein can comprise identifying the residue corresponding to single nucleotide polymorphism (SNP) rs471767, rs1893505, rs10501973, rs954723, rs1942836, rs471811, rs568157, rs474320, rs4754732, rs3740753, rs582691, rs493957, rs503362, rs653752, rs666553, rs578029, rs1042839, rs1042838, rs500760, or rs10895068. For example, disclosed herein are methods of identifying a subject at risk for preterm birth, comprising determining in a sample of nucleic acid from the subject the identity of one or more nucleotides in the progesterone receptor (PR) gene, wherein a substitution of a nucleotide at one or more positions in the PR gene of the subject compared to a control indicates that the subject is at risk of preterm birth, wherein the method comprises identifying the residue corresponding to a single nucleotide polymorphism (SNP) at one or more of the following: rs471767, rs1893505, rs10501973, rs954723, rs1942836, rs471811, rs568157, rs474320, rs4754732, rs3740753, rs582691, rs493957, rs503362, rs653752, rs666553, rs578029, rs1042839, rs1042838, rs500760, rs10895068.

The methods disclosed herein can comprise identifying the residue corresponding to single nucleotide polymorphism (SNP) at a specific location, wherein a specific nucleic acid residue present is indicative that the subject is at risk of preterm birth. For example disclosed herein are methods that comprise identifying the residue corresponding to a single nucleotide polymorphism (SNP) at one or more of the following: rs471767, rs1893505, rs10501973, rs954723, rs1942836, rs471811, rs568157, rs474320, rs4754732, rs3740753, rs582691, rs493957, rs503362, rs653752, rs666553, rs578029, rs1042839, rs1042838, rs500760, rs10895068, wherein: wherein a guanine (G) allele at SNP rs471767, a thymine (T) allele at SNP rs1893505, a guanine (G) allele at SNP rs10501973, a thymine (T) allele at SNP rs954723, a thymine (T) allele at SNP rs1942836, a guanine (G) allele at SNP rs471811, a guanine (G) allele at SNP rs568157, a thymine (T) allele at SNP rs474320, a thymine (T) allele at SNP rs4754732, a guanine (G) allele at SNP rs3740753, a guanine (G) allele at SNP rs582691, a guanine (G) allele at SNP rs493957, a guanine (G) allele at SNP rs503362, a guanine (G) allele at SNP rs653752, a thymine (T) allele at SNP rs666553, a thymine (T) allele at SNP rs578029, a guanine (G) allele at SNP rs1042839, a thymine (T) allele at SNP rs1042838, a guanine (G) allele at SNP rs500760, or an adenine (A) allele at SNP rs10895068 indicates that the subject is at risk of preterm birth

Described herein are methods that comprise identifying the residue corresponding to single nucleotide polymorphism (SNP) rs471767 in the subject, wherein a guanine (G) allele at SNP rs471767 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:59 (G allele) but does not hybridizes under stringent conditions to an oligonucleotide consisting of SEQ ID NO:58 (A allele), wherein hybrization of the probe under stringent conditions to the nucleic acid from the subject indicates that the subject is at risk of preterm birth.

Described herein are methods that comprise identifying the residue corresponding to single nucleotide polymorphism (SNP) rs578029 in the subject, wherein a thymidine (T) allele at SNP rs578029 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:47 (T allele) but does not hybridizes under stringent conditions to an oligonucleotide consisting of SEQ ID NO:46 (A allele), wherein hybrization of the probe under stringent conditions to the nucleic acid from the subject indicates that the subject is at risk of preterm birth.

Described herein are methods that comprise identifying the residue corresponding to single nucleotide polymorphism (SNP) rs10501973 in the subject, wherein a guanine (G) allele at SNP rs10501973 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:8 (G allele) but does not hybridizes under stringent conditions to an oligonucleotide consisting of SEQ ID NO:7 (A allele), wherein hybrization of the probe under stringent conditions to the nucleic acid from the subject indicates that the subject is at risk of preterm birth.

Described herein are methods that comprise identifying the residue corresponding to single nucleotide polymorphism (SNP) rs582691 in the subject, wherein a guanine (G) allele at SNP rs582691 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:32 (G allele) but does not hybridizes under stringent conditions to an oligonucleotide consisting of SEQ ID NO:31 (A allele), wherein hybrization of the probe under stringent conditions to the nucleic acid from the subject indicates that the subject is at risk of preterm birth.

Described herein are methods that comprise identifying the residue corresponding to single nucleotide polymorphism (SNP) rs503362 in the subject, wherein a guanine (G) allele at SNP rs503362 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:38 (G allele) but does not hybridizes under stringent conditions to an oligonucleotide consisting of SEQ ID NO:37 (C allele), wherein hybrization of the probe under stringent conditions to the nucleic acid from the subject indicates that the subject is at risk of preterm birth.

Described herein are methods that comprise identifying the residue corresponding to single nucleotide polymorphism (SNP) rs10895068 in the subject, wherein a guanine (G) allele at SNP rs10895068 indicates that the subject is at risk of preterm birth. For example, the method can comprise hybridizing the sample of nucleic acid from the subject with a probe, wherein the probe hybridizes under stringent conditions to an oligonucleotide consisting of SEQ ID NO:61 (G allele) but does not hybridizes under stringent conditions to an oligonucleotide consisting of SEQ ID NO:62 (A allele), wherein hybrization of the probe under stringent conditions to the nucleic acid from the subject indicates that the subject is at risk of preterm birth.

Described herein are methods that comprise identifying in the subject the residue corresponding to SNP rs578029, rs10501973, rs10501973, rs582691, rs503362, and rs10895068, wherein a guanine (G) allele at SNP rs471767, a thymidine (T) allele at SNP rs578029, a guanine (G) allele at SNP rs10501973, a guanine (G) allele at SNP rs582691, a guanine (G) allele at SNP rs503362, and a guanine (G) allele at SNP rs10895068 indicates that the subject is at risk of preterm birth.

As used herein “allele” such as “G allele” is meant to refer to the SNP residue on either the sense or antisense strand. Thus, reference to “G allele” can refer to either strand and is therefore also a disclosure of “C allele” on the opposite strand. Likewise, reference to a haplotype of SNPs, such as “ATGCCG” for rs471767|rs578029|rs503362|rs582691|rs10895068|rs10501973 includes alleles on either or both sense and antisense strands. For example the “C alleles” in this haplotype are referred to above as “G alleles.”

In some aspects of the methods described herein, the subject has had a prior preterm birth. In some aspects of the method, the subject has a family history of preterm birth.

The identity of one or more nucleotides in the progesterone receptor (PR) gene can be determined by gene sequencing. The identity of one or more nucleotides in the progesterone receptor (PR) gene can be determined by allele specific hybridization.

The disclosed methods can further comprise detecting fetal fibronectin (fNF) in the subject, wherein a positive fNF test in the midtrimester of pregnancy indicates that the subject is at risk of early spontaneous preterm birth. The disclosed methods can further comprise detecting salivary estriol (SalEst) in the subject, wherein a positive SalEst test indicates that the subject is at risk of late preterm birth. In some aspects, the methods further comprise measuring cervical length in the subject, wherein a cervical length less than 25 mm indicates that the subject is at risk of late preterm birth.

Described herein are methods that further comprise measuring cervical length. Cervical length can be measured using standard means, such as by transvaginal ultrasound. In some aspects, a short cervix, which includes cervical lengths less than 35 mm, including less than 25 mm, is associated with increased risk of prematurity. In some aspects, and an ultra-short cervix, which includes cervical lengths less than 15 mm, including less than 10 mm, is associated with an even higher risk.

Described herein are methods that further comprise determining in a sample of nucleic acid from the subject the identity of one or more nucleotides in the tumor necrosis factor-alpha (TNF-α) gene, wherein an adenine (A) at position −308 in the TNF-α gene of the subject indicates that the subject is at risk of preterm birth.

Described herein are methods that further comprise determining in a sample of nucleic acid from the subject the identity of one or more nucleotides in the interleukin-6 (IL-6), wherein a guanine (G) at position −174 in the IL-6 gene of the subject indicates that the subject is at risk of preterm birth, and/or wherein a cytosine (C) at position −174 in the IL-6 gene of the subject indicates that the subject is not at risk of preterm birth.

Described herein are methods that further comprise determining in a sample of nucleic acid from the subject the identity of one or more nucleotides in the interleukin-1β (IL-1β) gene, wherein an thymine (T) at position −511 in the IL-1β gene of the subject and/or an cytosine (C) at position −31 in the IL-1β gene of the subject indicates that the subject is at risk of preterm birth, indicates that the subject is at risk of preterm birth.

2. Therapy Selection

Described herein are methods of selecting a therapy for preventing preterm birth in a subject in need thereof. Also disclosed herein is a method of selecting a therapy for preventing preterm in a subject in need thereof.

By “prevent” or “preventing” is meant reducing the frequency or severity of a condition. The term does not require an absolute preclusion of the condition. Rather, this term includes a decreasing the chance for its occurrence. Thus, also disclosed are methods of selecting a therapy for reducing the occurrence and/or severity of preterm birth in a subject in need thereof. As used herein, “severity” of preterm birth refers to the numbers of days before normal gestation. Thus, birth at 32 days of gestation is more severe than birth at 37 days. Thus, also disclosed are methods of selecting a therapy for prolonging gestation in the subject in need thereof.

Described herein are methods that comprise determining in a sample of nucleic acid from the subject the identity of one or more nucleotides in the progesterone receptor (PR) gene, wherein a substitution of a nucleotide at one or more positions in the PR gene of the subject indicates that a progesterone receptor agonist is an effective therapy.

In some aspects of the disclosed methods, the progesterone receptor agonist is a progestin-agonist. The progestin-agonist can be natural or synthetic. Thus, the progestin-agonist can be progesterone, dydrogesterone, 17α-hydroxyprogesterone (17-OHP), or 17α-hydroxyprogesterone caproate (17-OHPC).

In some aspects, the subject is African-American. In some aspects, the subject is not African-American. For example, in some aspects the subject is Caucasian or Hispanic.

Wherein the subject is African American, in some aspects the methods can comprise identifying the residue corresponding to single nucleotide polymorphism (SNP) rs471767 in the subject, wherein a adenine (A) allele at SNP rs471767 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:58 (A allele) but does not hybridizes under stringent conditions to an oligonucleotide consisting of SEQ ID NO:59 (G allele), wherein hybrization of the probe under stringent conditions to the nucleic acid from the subject indicates that a progesterone receptor agonist is an effective therapy.

Wherein the subject is African American, in some aspects the methods comprise identifying the residue corresponding to single nucleotide polymorphism (SNP) rs578029 in the subject, wherein a thymidine (T) allele at SNP rs578029 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:47 (T allele) but does not hybridizes under stringent conditions to an oligonucleotide consisting of SEQ ID NO:46 (A allele), wherein hybrization of the probe under stringent conditions to the nucleic acid from the subject indicates that a progesterone receptor agonist is an effective therapy.

Wherein the subject is not African American, in some aspects the methods comprise identifying the residue corresponding to single nucleotide polymorphism (SNP) rs471767 in the subject, wherein a guanine (G) allele at SNP rs471767 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:59 (G allele) but does not hybridizes under stringent conditions to an oligonucleotide consisting of SEQ ID NO:58 (A allele), wherein hybrization of the probe under stringent conditions to the nucleic acid from the subject indicates that a progesterone receptor agonist is an effective therapy.

Wherein the subject is not African America, in some aspects the methods comprise identifying the residue corresponding to single nucleotide polymorphism (SNP) rs578029 in the subject, wherein a adenine (A) allele at SNP rs578029 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:46 (A allele) but does not hybridizes under stringent conditions to an oligonucleotide consisting of SEQ ID NO:47 (T allele), wherein hybrization of the probe under stringent conditions to the nucleic acid from the subject indicates that a progesterone receptor agonist is an effective therapy.

Wherein the subject is not African America, in some aspects the methods comprise identifying the residue corresponding to single nucleotide polymorphism (SNP) rs503362 in the subject, wherein a cytosine (C) allele at SNP rs503362 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:37 (C allele) but does not hybridizes under stringent conditions to an oligonucleotide consisting of SEQ ID NO:38 (G allele), wherein hybrization of the probe under stringent conditions to the nucleic acid from the subject indicates that a progesterone receptor agonist is an effective therapy.

Wherein the subject is not African America, in some aspects the methods comprise identifying the residue corresponding to single nucleotide polymorphism (SNP) rs666553 in the subject, wherein a thymidine (T) allele at SNP rs666553 indicates that a progesterone receptor agonist is an effective therapy. For example, the methods can comprise hybridizing the sample of nucleic acid from the subject with a probe, wherein the probe hybridizes under stringent conditions to an oligonucleotide consisting of SEQ ID NO:44 (T allele) but does not hybridizes under stringent conditions to an oligonucleotide consisting of SEQ ID NO:43 (C allele), wherein hybrization of the probe under stringent conditions to the nucleic acid from the subject indicates that a progesterone receptor agonist is an effective therapy.

In some aspects of the methods, the subject has had a prior preterm birth. In some aspects of the method, the subject has a family history of preterm birth.

In some aspects, the methods further comprises detecting fetal fibronectin (fNF) in the subject, wherein a positive fNF test in the midtrimester of pregnancy indicates that a progesterone receptor agonist is an effective therapy. In some aspects, the methods further comprises detecting salivary estriol (SalEst) in the subject, wherein a positive SalEst test indicates that a progesterone receptor agonist is an effective therapy.

In some aspects, the methods further comprises measuring cervical length in the subject, wherein a cervical length less than 25 mm indicates that a progesterone receptor agonist is an effective therapy.

Also disclosed are methods of predicting the effectiveness of a progesterone receptor (PR) antagonist in a cell or in a subject, comprising determining in a sample of nucleic acid from the cell or subject the identity of one or more nucleotides in the PR gene, wherein a substitution of a nucleotide at one or more positions in the PR gene of the cell or subject compared to a control indicates that the progesterone receptor (PR) antagonist is effective in the cell or subject.

3. Prevention

Also disclosed herein are methods of preventing preterm birth in a subject in need thereof. Also disclosed are methods of reducing the occurrence and/or severity of preterm birth in a subject in need thereof. Also disclosed are methods of prolonging gestation in the subject in need thereof.

In some aspects, the disclosed methods comprise determining in a sample of nucleic acid from the subject the identity of one or more nucleotides in the progesterone receptor (PR) gene and administering a therapeutically effective amount of a progesterone receptor agonist to subjects having a substitution of a nucleotide at one or more positions in the PR gene of the subject compared to a control.

In some aspects, the methods comprises detecting an adenine (A) allele at SNP rs471767, a thymidine (T) allele at SNP rs578029, a guanine (G) allele at SNP rs471767, an adenine (A) allele at SNP rs578029, a cytosine (C) allele at SNP rs503362, a thymidine (T) allele at SNP rs666553, or a combination thereof.

In some aspects, the nucleotide at the one or more position in the PR gene of the subject is detected by gene sequencing. In some aspects, the nucleotide at the one or more position in the PR gene of the subject is detected by allele specific hybridization.

For example, the SNP rs471767 G allele can be detected by a process comprising providing a probe that hybridizes under stringent conditions to an oligonucleotide consisting of SEQ ID NO:59 but does not hybridizes under stringent conditions to an oligonucleotide consisting of SEQ ID NO:58, and monitoring hybridization of said probe to the nucleic acid sample under stringent conditions.

The SNP rs578029 T allele can be detected by a process comprising providing a probe hybridizes under stringent conditions to an oligonucleotide consisting of SEQ ID NO:47 (T allele) but does not hybridizes under stringent conditions to an oligonucleotide consisting of SEQ ID NO:46 (A allele), and monitoring hybridization of said probe to the nucleic acid sample under stringent conditions.

The SNP rs471767 G allele can be detected by a process comprising providing a probe hybridizes under stringent conditions to an oligonucleotide consisting of SEQ ID NO:59 (G allele) but does not hybridizes under stringent conditions to an oligonucleotide consisting of SEQ ID NO:58 (A allele), and monitoring hybridization of said probe to the nucleic acid sample under stringent conditions.

The SNP rs578029 A allele can be detected by a process comprising providing a probe hybridizes under stringent conditions to an oligonucleotide consisting of SEQ ID NO:46 (A allele) but does not hybridizes under stringent conditions to an oligonucleotide consisting of SEQ ID NO:47 (T allele), and monitoring hybridization of said probe to the nucleic acid sample under stringent conditions.

The SNP rs503362 C allele can be detected by a process comprising providing a probe hybridizes under stringent conditions to an oligonucleotide consisting of SEQ ID NO:37 (C allele) but does not hybridizes under stringent conditions to an oligonucleotide consisting of SEQ ID NO:38 (G allele), and monitoring hybridization of said probe to the nucleic acid sample under stringent conditions.

The SNP rs666553 T allele can be detected by a process comprising providing a probe hybridizes under stringent conditions to an oligonucleotide consisting of SEQ ID NO:44 (T allele) but does not hybridizes under stringent conditions to an oligonucleotide consisting of SEQ ID NO:43 (C allele), and monitoring hybridization of said probe to the nucleic acid sample under stringent conditions.

Similar means for detecting the other SNPs disclosed herein are appreciable based on the present disclosure.

In some aspects, the progesterone receptor agonist is a progestin-agonist. For example, the progestin-agonist can be progesterone, dydrogesterone, 17α-hydroxyprogesterone (17-OHP), or 17α-hydroxyprogesterone caproate (17-OHPC).

In some aspects of the methods, the subject has had a prior preterm birth. In some aspects of the disclosed methods, the subject has a family history of preterm birth. T

In some aspects of the methods, the subject has a cervical length less than 35 mm. In some aspects of the methods, the subject has a cervical length less than 25 mm. In some aspects of the methods, the subject has less than 15 mm. In some aspects of the methods, the subject has a cervical length less than 10 mm.

i. Progesterone Receptor Agonist

a. Progesterone

The progesterone receptor agonist can be progesterone or derivative thereof. Compositions and methods administer progesterone to a pregnant woman with a short or effaced cervix to prolonging gestation by minimizing the shortening or effacing of the cervix, and possibly the softening and dilation are disclosed in U.S. Pat. Pub. No. 2008/0188829, which is incorporated by referenced herein in its entirety for these teachings.

For example, disclosed are methods of administering an effective amount of progesterone or derivative thereof to pregnant women having short or effaced cervixes. In some aspects, the use of progesterone decreases the morbidity and/or mortality of neonates born to pregnant women symptomatic of a shortened cervical length.

The progesterone of the disclosed compositions and methods can be progesterone molecule (from any source, including natural or synthetic) or progesterone metabolites (from any source, including natural or synthetic), such as 17-alpha-hydroxyprogesterone, for example, or it can be any other progestin. Any combination of these may also be used. In certain embodiments, the term “natural progesterone” includes progesterone and/or a natural progesterone metabolite.

Synthetic progesterone can selected from a group consisting of derivatives of progesterone or of testosterone or derivatives of other molecules and/or compounds with progestogenic activity. The term “derivative” refers to a chemical compound that may be made from or lead to a parent compound resulting from one or more chemical reactions. As used herein, the term “progestin” encompasses natural progesterone, synthetic progesterone, natural or synthetic derivatives of progesterone and/or other progestogenic compounds, or combinations thereof.

Thus progestins include, but are not limited to, 17-alpha-hydroxyprogesterone caproate, medroxyprogesterone acetate, norethindrone, norethindrone acetate, norethindrone enanthate, desogestrel, levonorgestrel, lynestrenol, ethynodiol diacetate, norgestrel, norgestimate, norethynodrel, gestodene, drospirenone, trimegestone, levodesogestrel, gestodyne, nesterone, etonogestrel, and derivatives from 19-nor-testosterone. In some aspects, the progesterone includes either of the natural progestins, progesterone or 17-alpha-hydroxyprogesterone. Some progestins can be delivered vaginally, some by intramuscular injection, some by oral administration, and some by rectal administration, although other routes of administration can be used as known in the art. In some aspects, the progesterone is administered via a drug delivery system that comprises progesterone, a water-soluble, water-swellable cross-linked polycarboxylic acid polymer, and at least one adjuvant.

Progesterone Receptor Agonists disclosed herein can be administered in conjunction with other methods of preventing and/or treating preterm birth and/or short cervix in pregnant women, such as surgical cerclage, administration of complementary/supplementary compositions such as antibiotics, indomethacin, and polymeric compositions, for example. Accordingly, the present invention is suitable for combination therapy.

ii. Carriers

The disclosed progesterone receptor agonist can be combined, conjugated or coupled with or to carriers and other compositions to aid administration, delivery or other aspects of the inhibitors and their use. For convenience, such composition will be referred to herein as carriers. Carriers can, for example, be a small molecule, pharmaceutical drug, fatty acid, detectable marker, conjugating tag, nanoparticle, or enzyme.

The disclosed Progesterone Receptor Agonists can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

In some aspects, the progesterone receptor agonist is provided in solution, such as an oil or otherwise suitable carrier as understood by one skilled in the art. Other methods of delivery, such as oil-based capsules and suppositories, are also available in certain embodiments. For suppositories, any traditional binder and/or carrier may be used, for example, one or more polyalkalene glycols or triglycerides; such suppositories may be formed from mixtures containing the active ingredient in the range of preferably about 0.5% to 10%, more preferably about 1 to 2%. Oral formulations can include such normally employed excipients as, for example, pharmaceutically acceptable mannitol, lactose, starch, magnesium stearate, sodium saccharine, cellulose, magnesium carbonate, and the like, or any combination.

In some aspects, progesterone receptor agonist can be administered together with or in a composition having a pharmaceutically acceptable bioadhesive carrier that comprises a cross-linked carboxylic polymer. Certain variations of these embodiments include a water-swellable polycarboxylic acid polymer, that upon administration provide local directed tissue levels and efficacy without detrimental blood levels of the treating agent.

In some aspects, the progesterone receptor agonist is administered in a bioadhesive formulation of progesterone receptor agonist for vaginal application consisting of a polycarbophil-based gel that contains 8% (wt/wt) progesterone receptor agonist. In some aspects, the progesterone receptor agonist is delivered as 8% progesterone receptor agonist gel and placebo, commonly available as Prochieve™ or Replens™, which are manufactured by Columbia Laboratories, Inc., NJ. In some embodiments, the progesterone receptor agonist is delivered in a prefilled, single-use, disposable plastic applicator that delivers the dose of 1.125 g of gel containing 90 mg of progesterone receptor agonist. In some aspects, progesterone receptor agonist is administered in accordance with U.S. Pat. No. 5,543,150, which is incorporated herein in its entirety.

Additional suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The carrier molecule can be covalently linked to the disclosed inhibitors. The carrier molecule can be linked to the amino terminal end of the disclosed peptides. The carrier molecule can be linked to the carboxy terminal end of the disclosed peptides. The carrier molecule can be linked to an amino acid within the disclosed peptides. The herein provided compositions can further comprise a linker connecting the carrier molecule and disclosed inhibitors. The disclosed inhibitors can also be conjugated to a coating molecule such as bovine serum albumin (BSA) (see Tkachenko et al., (2003) J Am Chem Soc, 125, 4700-4701) that can be used to coat microparticles, nanoparticles of nanoshells with the inhibitors.

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, progesterone receptor agonist can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the disclosed compositions into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

iii. Administration

The disclosed compounds and compositions, such as a progesterone receptor agonist, can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like. Additional formulations that are suitable for other modes of administration include suppositories and, in some cases, through a buccal, sublingual, intraperitoneal, intravaginal, anal or intracranial route. Thus, in some aspects, the method of administration is intravaginal.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, the amount of progesterone receptor agonist administered can be between about 45 mg and 800 mg, including between about 90 mg and 250 mg, based on the progestin effect of natural progesterone receptor agonist administered vaginally, but may be more or less depending on the potency of the progestin, the delivery system, and the route of administration.

The concentration of progesterone receptor agonist can be from about 0.01% to about 50%, including from about 1% to about 40%, including from about 2.5% to about 30%, including from about 5% to about 20%, including from about 6% to about 15%. Thus, in some aspects, the concentration of progesterone receptor agonist is from about 7% to about 9%.

In some aspects, the amount and concentration of the progesterone receptor agonist needs to be sufficient to remain in the subject to provide prophylaxis or treatment, such as for about 1 hour or more, including greater than about 2, 3, 4, 5, 6, 7, 8, 9, 10, 11, 12, 13, 14, 15, 16, 17, 18, 19, 20, 25, 30, 35, 35 hours.

In some aspects, the progesterone receptor agonist is administered daily, via the vaginal route. However, the administration can be as infrequent as weekly or as often as 4 times daily, depending on the characteristics of the progestin and the progestin formulation, including concentration and routes of administration. In some aspects, the progesterone receptor agonist is administered beginning about the 18^{th} to 22^{nd} week of gestation until about the 37^{th} week of gestation, or for approximately 14 to 19 weeks, depending on the gestational age at the beginning of treatment and the date of delivery. In some aspects, the progesterone receptor agonist is administered beginning about the 16^{th} week of gestation until about the 37^{th} week of gestation, or for approximately 21 weeks. In some aspects, the progesterone receptor agonist is administered beginning about the time of a positive pregnancy test until about the 37^{th} week of gestation, or beginning about the 2^{nd} to 4^{th} week of gestation, for approximately 33 to 35 weeks.

The progesterone receptor agonist can be administered to a pregnant woman beginning as early as the onset of gestation and whose cervix has a length greater than about 1.0 cm, including greater than 1.5 cm. In some aspects, the progesterone receptor agonist is administered to a pregnant woman beginning as early as the onset of gestation and whose cervix has a length is at least about 1.0 cm and at most about 8.0 cm. In some aspects, the progesterone receptor agonist is administered to a pregnant woman whose cervix has a length less than or equal to 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0 cm.

Progesterone receptor agonist can be administered as many times per day as needed to be effective to prevent and/or treat preterm birth, i.e., delay birth and increase gestation, including more than or equal to 37 weeks. In certain variations of these embodiments, the dosing of progesterone receptor agonist is 1 to 4 times per day. It should understood to one skilled in the art that the frequency of administration per day varies by the concentration and amount of progesterone receptor agonist delivered. For example, 90 mg of 8% progesterone receptor agonist delivery systems can be administered once per day. In some aspects, 200 mg progesterone receptor agonist an be administered 2 to 4 times per day.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### The Relationship Between Polymorphisms in the Human Progesterone Receptor and Clinical Response to 17 Alpha-Hydroxyprogesterone Caproate for the Prevention of Recurrent Spontaneous Preterm Birth

17 alpha-hydroxyprogesterone caproate (17P) has been shown to reduce the recurrence risk of spontaneous preterm birth (SPTB). The goal of this study was to assess if women with single nucleotide polymorphisms (SNPs) in the human progesterone receptor (hPR) are more or less likely to respond to 17P for the prevention of recurrent SPTB. This study involved secondary analysis of 463 women enrolled in a multicenter, prospective, double-blind study of 17P vs. placebo for the prevention of recurrent SPTB.

Of 463 patients randomized in the original study, 459 were analyzed. Two thirds of women were randomized to 17P and one third were randomized to placebo. Of those patients who received 17P, 111, or 36%, delivered preterm less than 37 weeks, and 195, or 64%, delivered at term. Among those patients who received placebo, the preterm birth rate was higher, as 55% of women delivered preterm. During summer 2008, DNA was extracted from stored saliva samples. A total of 380 patients, or 83% of the original cohort, had DNA available for analysis.

20 SNPs in the hPR gene were selected to encompass the large progesterone receptor haplotype block. The 389 individuals with DNA available were then genotyped using TaqMan chemistry with established primers. Allele and genotype frequencies were calculated and evaluated for evidence of genetic predisposition to 17P response. Women were stratified by self-reported race. Primary outcomes evaluated were preterm birth<37 and <32 weeks

Regression was used to assess for an interaction between genotype, treatment, and clinical outcomes. Models controlled for number of prior spontaneous preterm births, smoking, and pre-pregnancy body mass index.

TABLE 2
Inheritance models studied

| | | |
|---|---|---|
| Dominant | assumes that having at least one copy of | AA vs. Aa & aa |
| model | the minor allele is sufficient for disease | |
| Recessive | assumes that two copies of the minor | AA & Aa vs. aa |
| model | allele are needed for disease | |
| Additive | assumes that having 2 copies of minor | AA = 0, Aa = 1, |
| model | allele has twice the effect of having 1 | aa = 2 |
| | copy of the minor allele | |
| Co- | Assumes that the heterozygote state | Aa vs. AA & aa |
| dominant | confers a variable risk of disease | |
| model | | |

Dominant and recessive models of inheritance were used in this study (see Table 2 above). The dominant model assumes that having at least one copy of the minor allele is sufficient for disease. Patients homozygous for the minor allele and heterozygous patients with one minor allele are grouped together; here “little a/little a” and “Big A/little a” are compared with those homozygous for the major allele, “Big A/Big A.” The recessive model assumes that two copies of the minor allele are needed for disease. Here, those patients who are homozygous for the minor allele “little a/little a” are compared against the others.

Outcomes were available on 459 of the original 463 patients enrolled in the study. The study population of 389 patients was representative of the original cohort (see Table 3 below). The majority of patients in the cohort were African-American. Two-thirds of patients in the cohort received 17P. Furthermore, a similar proportion of patients delivered preterm less than 37 weeks and very preterm, less than 32 weeks, in the cohort when compared to the original study. Additionally, 21.1% of patients smoked during pregnancy. All patients had at least one prior spontaneous preterm birth in order to qualify for the original study. 28% of patients had 2 or more prior spontaneous preterm deliveries. The average pre-pregnancy body mass index was 26.7 kilograms per meter squared. See Table 4 below.

TABLE 3
Demographic Results

| | Original Cohort | This Study |
|---|---|---|
| N | 459 | 380 |
| African American | 59.0% | 59.6% |
| Received 17P | 66.7% | 66.3% |
| Preterm < 37 weeks | 42.5% | 39.6% |
| Preterm < 32 weeks | 14.2% | 12.1% |

On average, more than 96% of samples were successfully genotyped for each SNP. All SNPs were in Hardy-Weinberg equilibrium. SPTB rates in each group were similar to the original cohort. Clinical characteristics, racial distribution, and allele frequencies were not significantly different between cases and controls. The majority of patients in the cohort were African-American.

TABLE 4
Clinical Characteristics

| | Original Cohort | This Study |
|---|---|---|
| Smoked during pregnancy | 21.6% | 21.1% |
| At least 1 prior SPTB | 100% | 100% |
| More than 1 prior SPTB | 32.3% | 28.0% |
| Average pre-pregnancy body | 26.6 | 26.7 |
| mass index (kg/m²) | | |

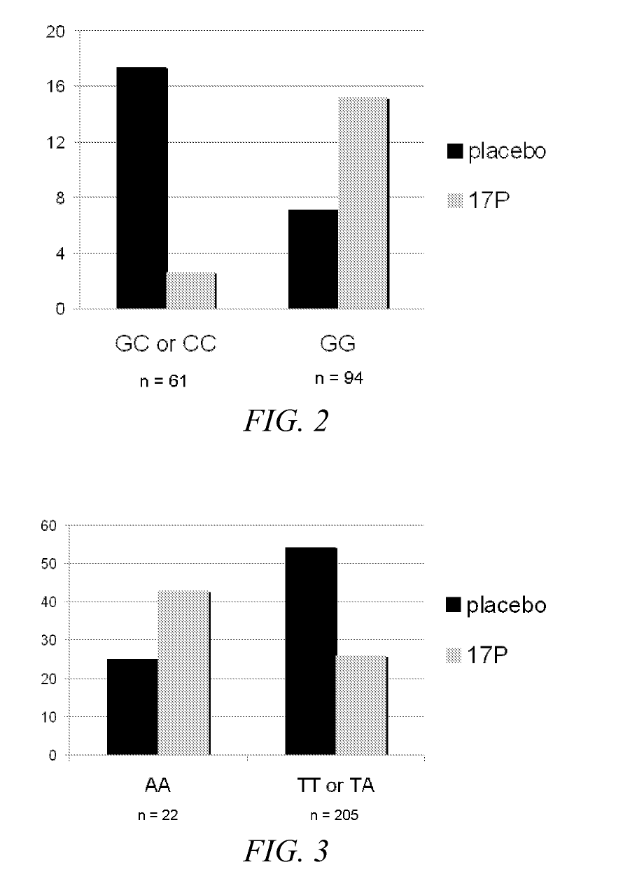

Shown in FIG. 2 are the results for rs471767 in African Americans. There was a higher reduction in SPTB<37 weeks with 17P when homozygous for the major allele (A) (dominant model), p=0.0229. Along the Y axis is the percentage of patients delivering preterm<37 weeks. Along the x-axis are 2 groups of patients, stratified by their genotype for rs471767. The black bars represent the percentage of patients delivering preterm when receiving the placebo, and the gray bars represent the percentage of patients delivering preterm when receiving 17P. This graph depicts the dominant model of inheritance. “G” is the minor allele, and thus, the AG and GG genotypes are grouped. Among women homozygous for the major allele (A) a significant reduction in the rate of preterm birth was found when women received 17P. Nearly 60% of patients with the dominant AA genotype delivered preterm<37 weeks when they received the placebo. When patients with the AA genotype received 17P, they had a significant decrease in the rate of prematurity, from nearly 60% (black bar) to 21% (gray bar). In contrast, those women who carried at least one copy of the minor allele (G) had a lower rate of preterm birth with the placebo, but the rate of prematurity remained similar, just under 40%, when these patients received 17P. Thus, for African Americans, there was a higher reduction in preterm birth<37 weeks with 17P when homozygous for the major allele (A). The rs471767 genotype was significantly correlated with response to 17P.

Shown in FIG. 3 are the results for rs578029 in African Americans. There was a higher reduction in SPTB with 17P when carrying at least one major allele (T) (recessive model), p=0.0289. Here, women homozygous for the minor allele (A) had no reduction in preterm birth rates with 17P. However, women carrying at least one copy of the major allele (T) had a significant reduction in the rate of prematurity, from approximately 55% (black bar) to 25% (gray bar). Thus, for African Americans, there is a higher reduction in the rate of preterm delivery with 17P when given to women who are carrying at least one copy of the major allele (T) for rs578029. Women homozygous for the minor allele did not experience this reduction.

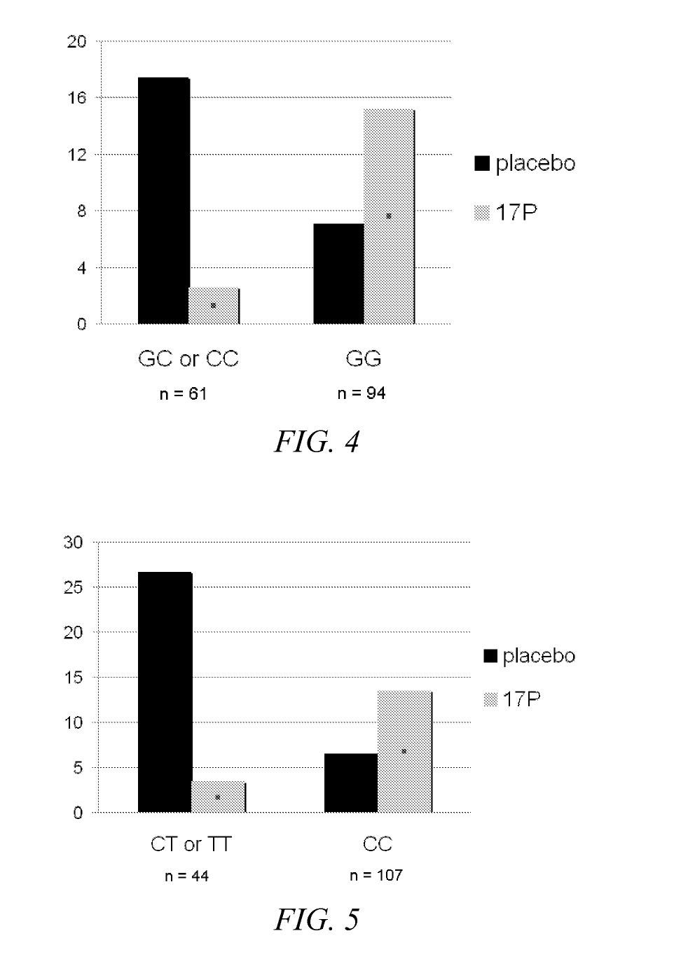

Shown in FIG. 4 are the results for rs503362 in non-African American patients delivering very preterm (<32 weeks gestation) (dominant model). Here, women with at least one minor allele (C) had a significant reduction in the rate of preterm birth from 17.4% with placebo to 2.6% with 17P. Patients homozygous for the major allele (G) did not have a reduction in the rate of prematurity when receiving 17P. Thus, in non-African Americans, there was a higher reduction in preterm birth with 17P in patients carrying at least one minor allele (C) for rs503362.

Shown in FIG. 5 are the results for rs666553 in non-African American patients delivering<32 weeks gestation (dominant model). There was a higher reduction in SPTB with 17P when carrying at least one minor allele (T) (dominant model), p=0.0382. Again, women with at least one minor allele (T) had a reduction in the rate of preterm birth from 26.7% with the placebo to 3.4% with 17P. In contrast, women homozygous for the major allele had a lower rate of early prematurity with the placebo and did not experience any reduction in prematurity with 17P. Thus, non-African American patients experienced a higher reduction in preterm birth with 17P when carrying at least one minor allele (T) for rs666553. See Table 5 below for summary of results.

TABLE 5
Summary of Results

| | | | Unadjusted | Adjusted* |
|---|---|---|---|---|
| Group | SNP | Model | P-value | p-value |
| African-Americans < | rs471767 | Dominant | 0.012 | 0.023 |
| 37 weeks | | Additive | 0.019 | 0.023 |
| | | Co- | 0.026 | 0.058 |
| | | dominant | | |
| | rs578029 | Recessive | 0.047 | 0.028 |
| | | Co- | 0.052 | 0.036 |
| | | dominant | | |
| Caucasians/Hispanics < | Rs500760 | Dominant | 0.036 | 0.043 |
| 37 weeks | | Co- | 0.019 | 0.025 |
| | | dominant | | |
| Caucasians/Hispanics < | Rs503362 | Additive | 0.030 | 0.035 |
| 35 weeks | | Dominant | 0.020 | 0.021 |
| | | Co- | 0.020 | 0.020 |
| | | dominant | | |
| Caucasian/Hispanics < | rs503362 | Dominant | 0.041 | 0.029 |
| 32 weeks | | Additive | 0.064 | 0.045 |
| | | Co- | 0.044 | 0.031 |
| | | dominant | | |
| | Rs578029 | Additive | 0.046 | 0.024 |
| | | Dominant | 0.027 | 0.054 |
| | Rs66553 | Additive | 0.016 | 0.042 |

*p-value adjusted for potential confounders including number of prior preterm births, smoking status, and body mass index

### 2. Example 2

### Variation in the Progesterone Receptor Gene Occurs More Frequently in Women at Very High Risk for Spontaneous Preterm Birth

Progesterone is critical to pregnancy maintenance. Patients with a prior SPTB or family history of SPTB have elevated risks of SPTB. These effects can be additive and women with a high personal/family history SPTB score (explained below) are more likely to have genetic variation in the human progesterone receptor (hPR).

The Utah Population Database was queried for the presence of a personal or family history (1^{st} or 2^{nd} degree relative) of SPTB in a subset of women from a prospectively collected database. A personal/family history SPTB score was calculated; patients received 1 point for a positive family history of SPTB and 1 point for each SPTB. Cases were women with a personal/family history score≧2, and controls were women with only term deliveries and no family history of SPTB (score=0). DNA was extracted from stored buffy coats and genotyped for 6 previously identified hPR SNPs (rs471767, rs578029, rs503362, rs582691, rs10895068, and rs10501973). Allele frequencies were calculated. Linkage disequilibrium (LD) and haplotype frequencies were estimated. PHASE version 2.1 was used to account for haplotype phase uncertainty.

80 patients (41 cases, 39 controls) were genotyped. All women were Caucasian or Hispanic; there were no differences between ethnicities. Of 41 cases, 25 had ≧1 SPTB with a family history of SPTB, and 16 had ≧2 SPTB and no family history of SPTB. Among the 6 hPR SNPs studied, there were no differences in allele frequencies between cases and controls. However, there was a trend towards significance for rs471767; the minor allele frequency (G) was higher in cases vs. controls (0.32 vs 0.18, p=0.051). Haplotype frequencies are summarized in Table 6. For example, in a haplotype analysis across SNPs rs471767 and rs578029, the frequency of the GT haplotype was 0.095 for cases but 0.015 for controls (p=0.039). Likewise, in analysis across a haplotype block encompassing SNPs rs471767|rs578029|rs503362|rs582691|rs10895068|rs10501973, frequency of the ATGCCG haplotype was 0.379 in cases but 0.613 in controls (p=0.004).

TABLE 6
Haplotype Results

| Haplotype | Case | Control | Chisq | P | SNPs |
|---|---|---|---|---|---|
| GA | 0.2297 | 0.1618 | 1.034 | 0.309 | rs471767\|rs578029 |
| GT | 0.09459 | 0.01471 | 4.254 | 0.039 | rs471767\|rs578029 |
| AT | 0.6757 | 0.8235 | 4.093 | 0.043 | rs471767\|rs578029 |
| | | | | | rs471767\|rs578029\|rs503362\| |
| GACTCA | 0.03734 | 0.04491 | 0.055 | 0.816 | rs582691\|rs10895068\|rs10501973 |
| | | | | | rs471767\|rs578029\|rs503362\| |
| ATGCCA | 0.3174 | 0.2002 | 2.651 | 0.104 | rs582691\|rs10895068\|rs10501973 |
| | | | | | rs471767\|rs578029\|rs503362\| |
| GACCTG | 0.01266 | 0.02528 | 0.326 | 0.568 | rs582691\|rs10895068\|rs10501973 |
| | | | | | rs471767\|rs578029\|rs503362\| |
| GACTCG | 0.02434 | 0.0285 | 0.025 | 0.873 | rs582691\|rs10895068\|rs10501973 |
| | | | | | rs471767\|rs578029\|rs503362\| |
| GACCCG | 0.1409 | 0.07083 | 1.904 | 0.168 | rs582691\|rs10895068\|rs10501973 |
| | | | | | rs471767\|rs578029\|rs503362\| |
| GTGCCG | 0.08861 | 0.01697 | 3.702 | 0.054 | rs582691\|rs10895068\|rs10501973 |
| | | | | | rs471767\|rs578029rs\|503362\| |
| ATGCCG | 0.3788 | 0.6133 | 8.214 | 0.004 | rs582691\|rs10895068\|rs10501973 |

Haplotype frequencies across 2 haplotype blocks in the hPR were therefore significantly different among women with multiple prior SPTB or a prior SPTB+family history of SPTB when compared with women with term deliveries and no personal/family history of SPTB. This indicates the hPR gene is associated with familial/recurrent SPTB.

### 3. Example 3

### Family History of Preterm Birth, Progesterone Receptor Polymorphisms, and Subsequent Pregnancy Outcome

Progesterone is critical to pregnancy maintenance; it binds human progesterone receptors (hPR) and modulates gene expression. Patients with a personal or family history of SPTB have elevated risks of SPTB. Women with a documented personal/family history of SPTB can therefore be more likely to have genetic variation in the hPR.

The Utah Population Database was queried for the presence of a personal or family history (1^{st} or 2^{nd} degree relative) of SPTB in a subset of women from a prospectively collected database at the Univ. of Utah. This subset of women had been previously identified by delivery gestational age, and included 62 women delivering 38-41 wks gestation and 92 women with SPTB delivering<37 wks gestation. Cases were defined as women with either a personal or family history of SPTB<37 wks; controls were women without a personal or family history of SPTB; these designations were regardless of delivery gestational age. DNA was extracted from stored buffy coats and genotyped for 6 previously identified hPR SNPs (rs471767, rs578029, rs503362, rs582691, rs10895068, and rs10501973). Allele frequencies were calculated. Linkage disequilibrium and haplotype frequencies were estimated. PHASE was used to account for haplotype phase uncertainty. Chi-square and Fisher's exact were used as appropriate.

154 patients (64 cases, 90 controls) were genotyped. All were Caucasian or Hispanic; there were no differences between ethnicities. Of 64 cases, 20 had a personal history, 34 had a family history, and 10 had both a personal and family history of SPTB. Cases and controls had similar mean delivery gestational ages (35.9 vs. 36.2 wks, p=0.68) and a similar percentage delivered<37 wks gestation (66% vs. 56% preterm, p=0.21). There were no significant differences in minor allele frequencies of individual SNPs between cases and controls. However, in analysis across a haplotype block encompassing SNPs rs471767|rs578029|rs503362|rs582691|rs10895068|rs10501973, frequency of the ATGCCG haplotype was 0.525 in controls but 0.368 in cases (p=0.011).

Although the individual hPR SNPs studied did not occur at a higher rate in women with a personal or family history of SPTB, a haplotype block encompassing 6 hPR SNPs was associated with a personal/family history of SPTB. This indicates the hPR gene is a candidate for association with familial/recurrent SPTB.

### 4. Example 4

### An Uncommon Haplotype in the Progesterone Receptor Gene is Associated with a Significantly Higher Risk of Spontaneous Preterm Birth

Progesterone is critical to pregnancy maintenance; it binds human progesterone receptors (hPR) and modulates gene expression. Genetic variants in the hPR can be more common in women with SPTB.

Patients with SPTB<37 weeks gestation (cases) were identified from a prospectively collected sample database at the University of Utah. Cases were matched with term deliveries at 38-41 weeks gestation (controls). DNA was extracted from stored buffy coats and genotyped for 6 previously identified single nucleotide polymorphisms (SNPs) in the hPR (rs471767, rs578029, rs503362, rs582691, rs10895068, and rs10501973). Genotype and allele frequencies were calculated. Linkage disequilibrium and haplotype frequencies were estimated. PHASE was used to account for haplotype phase uncertainty. Chi-square, Fisher s exact, and logistic regression were used for analysis as appropriate. A p-value<0.05 was considered statistically significant.

154 Caucasian and Hispanic patients (92 cases and 62 controls) were genotyped. There were no differences between Caucasians and Hispanics. Clinical characteristics did not vary between cases and controls. All SNPs were in Hardy-Weinberg equilibrium. Cases delivered earlier than controls (mean 33.8 vs. 39.5 weeks gestation, p<0.001). The minor allele frequency (G) in rs471767 was higher in women delivering preterm vs. term (0.29 vs 0.18, respectively; OR 1.85, 95% CI 1.04-3.26, p=0.035). In a haplotype analysis across SNPs rs471767 and rs578029, the GT haplotype frequency was 0.0096 for term controls but 0.1053 for preterm cases (p=0.002). The GT haplotype was also more frequent in women delivering very preterm (<34 weeks) vs. term controls (0.08 vs. 0.0096, p=0.021). In a regression analysis, a highly significant increased risk of SPTB<37 weeks gestation was found for women with the GT haplotype (OR 16.8, 95% CI 2.1-133.2, p=0.008) and for all women with a history of a prior preterm delivery regardless of haplotype status (OR 9.4, 95% CI 2.6-33.4, p=0.001).

In this cohort of Caucasian and Hispanic women, the uncommon GT haplotype across SNPs rs47176-rs578029 in the hPR was strongly associated with an increased risk of SPTB. Women with a prior preterm delivery and those carrying the minor allele in rs471767 also had an increased risk of SPTB.

### Example 5

### Fetal Progesterone Receptor Genotype

Objective: Various maternal genetic polymorphisms have been associated with an increased risk of preterm birth (PTB), but the contribution of fetal genetics is largely unknown. Progesterone is critical to pregnancy maintenance. We theorize that premature infants are more likely to have genetic variation in the human progesterone receptor (hPR).

Study Design: Mother-infant pairs with PTB<37 weeks gestation (cases) were identified from a prospectively collected sample database at the University of Utah. Cases were matched with term deliveries at 38-41 weeks gestation (controls). DNA was extracted from stored buffy coats and genotyped for 6 single nucleotide polymorphisms (SNPs) in the hPR. Allele and genotype frequencies for each group were compared using chi-squared and Fisher's exact. Haplotype frequencies were estimated using PHASE. A p-value of <0.05 was considered significant.

Results: Fetal genotypes were obtained from 107 Caucasian and Hispanic women (52 cases, 55 controls); there were no significant differences between races. All SNP were in Hardy-Weinberg equilibrium. Cases delivered at a mean gestational age of 39.5 weeks vs. 35.3 weeks for controls (p<0.001). Cases and controls were similar with regard to clinical and demographic characteristics. For rs1050197, fetuses homozygous for the minor allele (A; recessive model) were less likely to have been delivered preterm (OR 0.11, 95% CI 0-0.74, p=0.018). Additionally, in a haplotype analysis across SNPs rs471767 and rs578029, the frequency of the GT haplotype was 0.104 for cases but 0.023 for controls (p=0.024).

Conclusion: In this cohort of Caucasian and Hispanics infants, there were significant differences between preterm and term hPR genotypes.

### 5. Example 6

### Examination of Genetic Variation in the Human Progesterone Receptor Among Women with Preterm Birth and a Family History of Preterm Birth

More than 12% of all pregnancies in the United States are delivered preterm, and rates continue to rise (Ananth et al., 2005). Prematurity is the leading cause of perinatal morbidity and mortality as premature infants have a 40-fold increase in mortality compared with their term counterparts (Goldenberg et al. 1998; Martin et al., 2005).

Progesterone is important to pregnancy maintenance. It binds human progesterone receptors (hPR) and modulates gene expression. The human progesterone receptor (hPR) is a member of the steroid and thyroid receptor superfamily. Studies using supplemental progesterone in varying forms have shown reductions in spontaneous PTB among women at particularly high risk for prematurity (2008 ACOG Committee Opinion No. 419; da Fonseca et al., 2003; Meis et al., 2003).

Single nucleotide polymorphisms (SNPs) within the hPR gene have been identified and have been found to be associated with several reproductive disorders (DeVivo et al., 2002; Risch et al., 2006; Schweikert et al., 2004). Furthermore, genetic variation in the human progesterone receptor gene occurs more frequently among women with preterm birth and a family history of preterm birth. Women with a personal or family history of PTB are more likely to have genetic variation in the hPR.

The strongest predictor of spontaneous PTB is a previous history of spontaneous PTB (Esplin et al., 2008; McManemy et al., 2007). Patients with a family history of spontaneous preterm birth (PTB) also have elevated risks of PTB in future pregnancies. Spontaneous PTB recurs in 35-40% of women, and a tendency for repeat PTB at a similar gestational age has been observed (Esplin et al., 2008; Mercer et al., 1999).

These studies examined whether women with spontaneous PTB are more likely to have genetic variation in the hPR as compared to women with only term deliveries. The studies also examined whether women with both a family and personal history of PTB are more likely to have variation in the hPR compared to women with only term deliveries and no family history of PTB.

Women with a DNA sample available and a singleton spontaneous preterm birth<37 weeks gestation occurring between November 2002 and September 2006 were identified from a prospectively collected obstetric database at the University of Utah (Salt Lake City, Utah). Women delivering a singleton non-anomalous fetus at greater than or equal to 38 weeks gestation at the time of enrollment and with no prior preterm deliveries were also identified from the same prospective database. Women had been recruited at the time of their delivery hospitalization and had previously provided consent for all future biologic tissue analyses, including genetic studies.

These potentially eligible patients were then linked to the Utah Population Database. The Utah Population Database is a unique resource of linked records, including birth certificates, death certificates, pedigrees, and other vital data for over 6 million individuals. This Database has previously been used to assess for familial disposition to pregnancy complications, including preterm delivery, preeclampsia, and operative delivery (Esplin et al., 2008; Porter et al., 1999; Esplin et al., 2001; Varner et al., 1996). Each individual's pedigree was queried for the number of first and second degree relatives with a documented prior delivery less than 37 weeks gestation. Patients with multiple gestations, patients carrying a fetus with suspected major anomalies, and patients that were not linked to the Utah Population Database were excluded. Clinical data were retrieved from the patient's chart at the time of delivery and biologic sample collection. Data were verified by the review of medical records at the time of inclusion in this analysis.

DNA was extracted from stored buffy coats using standard methods. Patients were subsequently genotyped for 6 previously identified single nucleotide polymorphisms (SNPs) in the region of the hPR, including rs471767, rs578029, rs503362, rs582691, rs10985068, and rs10501973, using TaqM an chemistry (Applied Biosystems, Foster City, Calif.).

Clinical characteristics were compared using chi-square and Fisher's exact as appropriate. Minor allele frequencies were calculated for each SNP, and were compared using chi-square and Fisher's exact. Genotypes for each SNP were compared using the “dominant” model of inheritance. Odds ratios of preterm birth were calculated using women homozygous for the major allele as the referent group. Deviation from Hardy-Weinberg equilibrium was assessed. Linkage disequilibrium and haplotype frequencies were estimated, and PHASE was used to account for haplotype phase uncertainty (Stephens et al., 2001). Stepwise multivariable logistic regression was used to correct for possible confounders. Population attributable risk was calculated from the logistic model as described by Greenland (Greenland and Drescher, 1993). Data were analyzed using STATA version 10.0 (College Station, Tex.). Significance was set at p<0.05.

154 patients were genotyped, including 92 preterm delivery cases and 62 term delivery controls. All patients were self-identified as either Caucasian (85.7%) or Hispanic (14.3%); no statistical differences were seen in Caucasian allele frequencies when compared to Hispanic allele frequencies and therefore, the two population were examined together. All SNPs were in Hardy-Weinberg equilibrium. Maternal demographic information and delivery characteristics are displayed in Table 7. Of the 92 preterm delivery cases, 35 (38%) delivered very preterm (i.e., <34 weeks gestation).

TABLE 7
Maternal Demographic and Clinical Characteristics

| | Preterm | Term | |
|---|---|---|---|
| Maternal Demographic or | Delivery | Delivery | |
| Clinical Characteristic | (n = 92) | (n = 62) | p-value |
| Mean maternal age, years | 25.6 +/− 5.7 | 26.6 +/− 5.7 | 0.30 |
| (+/−SD) | | | |
| Married, % | 72.5 | 77.4 | 0.50 |
| Caucasian, % | 85.9 | 85.5 | 0.95 |
| Tobacco use during | 14.1 | 14.5 | 0.95 |
| pregnancy, % | | | |
| Nulliparous, % | 47.8 | 51.6 | 0.65 |
| Cesarean delivery, % | 18.5 | 14.5 | 0.52 |
| Male fetus, % | 54.4 | 48.4 | 0.47 |
| Mean delivery gestational age, | 33.8 +/− 3.4 | 39.5 +/− 0.8 | <0.001 |
| weeks (+/−SD) | | | |
| Mean infant birthweight, | 2357 +/− 690 | 3489 +/− 413 | <0.001 |
| grams (+/−SD) | | | |

Minor allele frequencies were compared between cases and controls for each SNP (Table 8). Genotypes were compared using the dominant model of inheritance (assumes that one copy of the minor allele is sufficient for disease and compares those homozygous for the major allele with the others) (Table 8). Preterm delivery cases were more likely to carry the minor allele, G, for rs471767, when compared with term controls (minor allele frequency 0.29 vs. 0.18, respectively, p=0.035). Carriage of the minor allele, G, for rs471767 conferred an almost twofold increased odds of preterm birth [odds ratio 1.85 (1.04-3.26)]. There were no significant differences in the genotypes or minor allele frequencies between cases and controls for the other 5 SNPs studied. In Table 8, values are given as number of patients (frequency) and odds ratios of preterm delivery were calculated using patients homozygous for the major allele as the referent group.

TABLE 8
Genotype and Allele Frequencies for
Preterm Delivery and Term Delivery

| | | Preterm | | Term | | | |
|---|---|---|---|---|---|---|---|
| | | Delivery | | Delivery | | Odds Ratio | P |
| SNP | | (n = 92) | | (n = 62) | | (95% CI) | value |
| rs471767 | Genotypes | | | | | | |
| | AA | 48 | (0.55) | 38 | (0.67) | | |
| | AG | 27 | (0.31) | 17 | (0.30) | | |
| | GG | 12 | (0.14) | 2 | (0.04) | | |
| | AG + GG | 39 | (0.45) | 19 | (0.34) | 1.63 | 0.17 |
| | | | | | | (0.81-3.24) | |
| | Alleles | | | | | | |
| | A | 125 | (0.71) | 95 | (0.82) | | |
| | G | 51 | (0.29) | 21 | (0.18) | 1.85 | 0.035 |
| | | | | | | (1.04-3.26) | |
| rs503362 | Genotypes | | | | | | |
| | GG | 56 | (0.62) | 41 | (0.70) | | |
| | GC | 29 | (0.32) | 16 | (0.27) | | |
| | CC | 5 | (0.06) | 2 | (0.03) | | |
| | GC + CC | 34 | (0.38) | 18 | (0.30) | 1.38 | 0.36 |
| | | | | | | (0.69-2.77) | |
| | Alleles | | | | | | |
| | G | 143 | (0.79) | 100 | (0.83) | | |
| | C | 39 | (0.21) | 20 | (0.17) | 1.36 | 0.31 |
| | | | | | | (0.75-2.46) | |
| rs578029 | Genotypes | | | | | | |
| | TT | 50 | (0.62) | 39 | (0.70) | | |
| | TA | 25 | (0.31) | 15 | (0.27) | | |
| | AA | 6 | (0.07) | 2 | (0.03) | | |
| | TA + AA | 31 | (0.38) | 17 | (0.30) | 1.42 | 0.34 |
| | | | | | | (0.69-2.91) | |
| | Alleles | | | | | | |
| | T | 127 | (0.77) | 95 | (0.83) | | |
| | A | 37 | (0.23) | 19 | (0.17) | 1.46 | 0.23 |
| | | | | | | (0.79-2.68) | |
| rs582691 | Genotypes | | | | | | |
| | CC | 66 | (0.86) | 51 | (0.88) | | |
| | CT | 11 | (0.14) | 5 | (0.09) | | |
| | TT | 0 | (0.00) | 2 | (0.03) | | |
| | CT + TT | 11 | (0.14) | 7 | (0.12) | 1.21 | 0.71 |
| | | | | | | (0.45-3.25) | |
| | Alleles | | | | | | |
| | C | 145 | (0.93) | 109 | (0.92) | | |
| | T | 11 | (0.07) | 9 | (0.08) | 0.92 | 0.86 |
| | | | | | | (0.38-2.24) | |
| rs10501973 | Genotypes | | | | | | |
| | GG | 30 | (0.47) | 26 | (0.51) | | |
| | GA | 26 | (0.41) | 21 | (0.41) | | |
| | AA | 8 | (0.12) | 4 | (0.08) | | |
| | GA + AA | 34 | (0.53) | 25 | (0.49) | 1.18 | 0.66 |
| | | | | | | (0.57-2.45) | |
| | Alleles | | | | | | |
| | G | 88 | (0.68) | 74 | (0.71) | | |
| | A | 42 | (0.32) | 30 | (0.29) | 1.17 | 0.57 |
| | | | | | | (0.67-2.06) | |
| rs10895068 | Genotypes | | | | | | |
| | CC | 85 | (0.96) | 52 | (0.90) | | |
| | CT | 4 | (0.04) | 6 | (0.10) | | |
| | TT | 0 | (0.00) | 0 | (0.00) | | |
| | CT + TT | 4 | (0.04) | 6 | (0.10) | 0.41 | 0.17 |
| | | | | | | (0.12-1.42) | |
| | Alleles | | | | | | |
| | C | 176 | (0.98) | 112 | (0.95) | | |
| | T | 4 | (0.02) | 6 | (0.05) | 0.42 | 0.18 |
| | | | | | | (0.13-1.43) | |

Haplotype analysis results are shown in Table 9. Cases and controls had significantly different haplotypes across rs471767 and rs578029. The incidence of the uncommon GT haplotype was 0.105 among preterm delivery cases vs. 0.0096 among term delivery controls (p=0.0025).

TABLE 9
Haplotype Frequencies Among Preterm Cases and Term Controls

| | | Preterm | Term | |
|---|---|---|---|---|
| | | Delivery | Delivery | p- |
| Haplotype Block | Haplotype | (n = 92) | (n = 62) | value |
| rs471767\|rs578029 | GA | 0.211 | 0.173 | 0.46 |
| | GT | 0.105 | 0.0096 | 0.0025 |
| | AT | 0.684 | 0.817 | 0.017 |

The role of family history was also examined. A similar percentage of preterm delivery cases and term delivery controls had first- and/or second-degree relatives with documented preterm deliveries (Table 10; data are n(%). There were 25 women who delivered preterm and also had a family history of PTB (preterm and positive family history).

TABLE 10
Documented Family History of Preterm Birth

| | Preterm | | Term | | |
|---|---|---|---|---|---|
| | Delivery | | Delivery | | p- |
| Documented Family History | (n = 92) | | (n = 62) | | value |
| Either first or second | 25 | (27.2) | 18 | (29.0) | 0.80 |
| degree relative | | | | | |
| with documented preterm birth | | | | | |
| First degree relative | 12 | (13.0) | 9 | (14.5) | 0.79 |
| Second degree relative | 21 | (22.8) | 10 | (16.1) | 0.31 |
| First and second degree relative | 8 | (8.7) | 1 | (1.6) | 0.085 |

As shown in Table 11, those women delivering preterm and with a positive family history did not have significantly different minor allele frequencies for the individual SNP studied. However, when those women delivering preterm and with a positive family history were compared with 44 women who delivered at term and had no family history of PTB, there were significantly different haplotype distributions.

TABLE 11
Human Progesterone Receptor Single Nucleotide
Polymorphisms and Haplotype Frequencies

| | | Preterm | Term | |
|---|---|---|---|---|
| | | Delivery | delivery | |
| | Minor | AND positive | and no | |
| | Allele | family | family | |
| | or | history of | history of | |
| SNP or Haplotype | Haplo- | preterm birth | preterm birth | p- |
| Block | type | (n = 25) | (n = 44) | value |
| rs471767 | G | 0.317 | 0.181 | 0.052 |
| rs578029 | A | 0.230 | 0.157 | 0.27 |
| rs503362 | C | 0.220 | 0.162 | 0.36 |
| rs582691 | T | 0.060 | 0.070 | 0.83 |
| rs10895068 | T | 0.012 | 0.042 | 0.25 |
| rs10501973 | A | 0.371 | 0.233 | 0.098 |
| rs471767\|rs578029 | GA | 0.230 | 0.162 | 0.31 |
| | GT | 0.094 | 0.015 | 0.039 |
| | AT | 0.676 | 0.823 | 0.043 |

Variables considered for inclusion in the logistic regression model included maternal age, history of a prior preterm delivery, history of cervical loop electrosurgical excision procedure, family history of PTB, male fetus, marital status, tobacco use during pregnancy, and carriage of the GT haplotype across rs471767 and rs578029. Only carriage of the GT haplotype (OR 14.3, 95% CI 1.81-112.9, p=0.012), history of a prior preterm delivery (OR 7.7, 95% CI 2.5-24.2, p<0.001), and maternal age (OR 0.96, 95% CI 0.90-1.02, p=0.15) remained in the final model. The population attributable risk was calculated from the final regression model, and for the GT haplotype, the risk was found to be 15.2% (95% CI 8.7-21.2%).

In this patient population of Caucasian and Hispanic women, variation in some areas of the hPR occurs more frequently in women with spontaneous PTB less than 37 weeks gestation. Nuclear hPRs primarily exist in 2 different distinct isoforms, PR-A and PR-B, and have been found in gestational tissues including the amion and chorion (Mills et al., 2006). Both are encoded from a single gene by transcription from 2 different promoters and have differing roles. PR-A is smaller and lacks the 164 N-terminal amino acids that form an activation domain on the receptor, and PR-A inhibits the transcription of progesterone receptive genes. PR-B increases transcription of progesterone-responsive genes and has an overall quiescent effect on the myometrium (DeVivo et al., 2002; Kastner et al., 1990).

Given the differing roles of these receptors, studies have proposed that the responsiveness of target tissues to progesterone depends not only on the level of progesterone but also on the ratio of PR isoforms (Conneely et al., 2001; Goldman et al., 2007). Studies have investigated whether a relative increase in the ratio of PR-A to PR-B can contribute to a functional withdrawal of progesterone and lead to the initiation of labor (Merlino et al., 2007; Pieber et al., 2001). Importantly, rs471767 is a SNP located just upstream of the hPR promotor. Thus, genetic variation in this region can alter transcription of PR-A in relation to PR-B.

In these studies, the hPR haplotype block encompassing SNPs rs471767 and rs578029 was associated with preterm birth. In this regression model, carriage of the GT haplotype conferred a 14-fold increased risk of PTB. The population attributable risk of carriage of the GT haplotype was 15.2%. This indicates that some of the markers studied in this region can be in linkage disequilibrium with other functional genes associated with prematurity. Allele and haplotype frequencies in the hPR are significantly different among women with PTB and women with PTB plus a family history of PTB. The haplotype associations were present in all women with preterm delivery as well as those delivering preterm with a positive family history. These data indicate that women at the highest risk for prematurity are also at highest risk for genetic differences in their hPR, and further indicate the hPR gene is a candidate for association with PTB/familial PTB.

What is claimed is:

1. A method of identifying a subject at risk for preterm birth, comprising determining in a sample of nucleic acid from the subject the identity of one or more nucleotides in the progesterone receptor (PR) gene, wherein a substitution of a nucleotide at one or more positions in the PR gene of the subject compared to a control indicates that the subject is at risk of preterm birth.

2. The method of claim 1, wherein the method comprises identifying the residue corresponding to single nucleotide polymorphism (SNP) rs471767 in the subject, wherein a guanine (G) allele at SNP rs471767 indicates that the subject is at risk of preterm birth.

3. The method of claim 2, wherein the method comprises hybridizing the sample of nucleic acid from the subject with a probe, wherein the probe hybridizes under stringent conditions to an oligonucleotide consisting of SEQ ID NO:59 (G allele) but does not hybridizes under stringent conditions to an oligonucleotide consisting of SEQ ID NO:58 (A allele), wherein hybrization of the probe under stringent conditions to the nucleic acid from the subject indicates that the subject is at risk of preterm birth.

4. The method of claim 2, wherein the method further comprises identifying the residue corresponding to single nucleotide polymorphism (SNP) rs578029 in the subject, wherein a thymidine (T) allele at SNP rs578029 indicates that the subject is at risk of preterm birth.

5. The method of claim 4, wherein the method comprises hybridizing the sample of nucleic acid from the subject with a probe, wherein the probe hybridizes under stringent conditions to an oligonucleotide consisting of SEQ ID NO:47 (T allele) but does not hybridizes under stringent conditions to an oligonucleotide consisting of SEQ ID NO:46 (A allele), wherein hybrization of the probe under stringent conditions to the nucleic acid from the subject indicates that the subject is at risk of preterm birth.

6. The method of claim 4, wherein the method further comprises identifying the residue corresponding to single nucleotide polymorphism (SNP) rs10501973 in the subject, wherein a guanine (G) allele at SNP rs10501973 indicates that the subject is at risk of preterm birth.

7. The method of claim 4, wherein the method further comprises identifying the residue corresponding to single nucleotide polymorphism (SNP) rs582691 in the subject, wherein a guanine (G) allele at SNP rs582691 indicates that the subject is at risk of preterm birth.

8. The method of claim 4, wherein the method further comprises identifying the residue corresponding to single nucleotide polymorphism (SNP) rs503362 in the subject, wherein a guanine (G) allele at SNP rs503362 indicates that the subject is at risk of preterm birth.

9. The method of claim 4, wherein the method further comprises identifying the residue corresponding to single nucleotide polymorphism (SNP) rs10895068 in the subject, wherein a guanine (G) allele at SNP rs10895068 indicates that the subject is at risk of preterm birth.

10. The method of claim 4, wherein the method further comprises identifying in the subject the residue corresponding to SNP rs10501973, rs582691, rs503362, and rs10895068, wherein a guanine (G) allele at SNP rs10501973, a guanine (G) allele at SNP rs582691, a guanine (G) allele at SNP rs503362, and a guanine (G) allele at SNP rs10895068 indicates that the subject is at risk of preterm birth.

11. The method of claim 6, wherein the method comprises hybridizing the sample of nucleic acid from the subject with a probe, wherein the probe hybridizes under stringent conditions to an oligonucleotide consisting of SEQ ID NO:8 (G allele) but does not hybridizes under stringent conditions to an oligonucleotide consisting of SEQ ID NO:7 (A allele), wherein hybrization of the probe under stringent conditions to the nucleic acid from the subject indicates that the subject is at risk of preterm birth.

12. The method of claim 7, wherein the method comprises hybridizing the sample of nucleic acid from the subject with a probe, wherein the probe hybridizes under stringent conditions to an oligonucleotide consisting of SEQ ID NO:32 (G allele) but does not hybridizes under stringent conditions to an oligonucleotide consisting of SEQ ID NO:31 (A allele), wherein hybrization of the probe under stringent conditions to the nucleic acid from the subject indicates that the subject is at risk of preterm birth.

13. The method of claim 8, wherein the method comprises hybridizing the sample of nucleic acid from the subject with a probe, wherein the probe hybridizes under stringent conditions to an oligonucleotide consisting of SEQ ID NO:38 (G allele) but does not hybridizes under stringent conditions to an oligonucleotide consisting of SEQ ID NO:37 (C allele), wherein hybrization of the probe under stringent conditions to the nucleic acid from the subject indicates that the subject is at risk of preterm birth.

14. The method of claim 9, wherein the method comprises hybridizing the sample of nucleic acid from the subject with a probe, wherein the probe hybridizes under stringent conditions to an oligonucleotide consisting of SEQ ID NO:61 (G allele) but does not hybridizes under stringent conditions to an oligonucleotide consisting of SEQ ID NO:62 (A allele), wherein hybrization of the probe under stringent conditions to the nucleic acid from the subject indicates that the subject is at risk of preterm birth.

15. The method of claim 1, wherein the subject has had a prior preterm birth.

16. The method of claim 1, wherein the subject has a family history of preterm birth.

17. The method of claim 1, wherein the identity of one or more nucleotides in the progesterone receptor (PR) gene is determined by gene sequencing.

18. The method of claim 1, wherein the identity of one or more nucleotides in the progesterone receptor (PR) gene is determined by allele specific hybridization.

19. The method of claim 1, further comprising detecting fetal fibronectin (fNF) in the subject, wherein a positive fNF test in the midtrimester of pregnancy indicates that the subject is at risk of early spontaneous preterm birth.

20. The method of claim 1, further comprising detecting salivary estriol (SalEst) in the subject, wherein a positive SalEst test indicates that the subject is at risk of late preterm birth.

21. A method of prolonging gestation in a subject in need thereof, comprising determining in a sample of nucleic acid from the subject the identity of one or more nucleotides in the progesterone receptor (PR) gene, wherein a substitution of a nucleotide at one or more positions in the PR gene of the subject indicates that a progesterone receptor agonist is an effective therapy.

22. The method of claim 21, wherein the progesterone receptor agonist is a progestin-agonist.

23. The method of claim 22, wherein the progestin-agonist is progesterone, dydrogesterone, 17α-hydroxyprogesterone (17-OHP), or 17α-hydroxyprogesterone caproate (17-OHPC).

24. The method of claim 21, wherein the subject is African-American, wherein the method comprises identifying the residue corresponding to single nucleotide polymorphism (SNP) rs471767 in the subject, wherein a adenine (A) allele at SNP rs471767 indicates that a progesterone receptor agonist is an effective therapy.

25. The method of claim 24, wherein the method comprises hybridizing the sample of nucleic acid from the subject with a probe, wherein the probe hybridizes under stringent conditions to an oligonucleotide consisting of SEQ ID NO:58 (A allele) but does not hybridizes under stringent conditions to an oligonucleotide consisting of SEQ ID NO:59 (G allele), wherein hybrization of the probe under stringent conditions to the nucleic acid from the subject indicates that a progesterone receptor agonist is an effective therapy.

26. The method of claim 20, wherein the subject is African-American, wherein the method comprises identifying the residue corresponding to single nucleotide polymorphism (SNP) rs578029 in the subject, wherein a thymidine (T) allele at SNP rs578029 indicates that a progesterone receptor agonist is an effective therapy.

27. The method of claim 26, wherein the method comprises hybridizing the sample of nucleic acid from the subject with a probe, wherein the probe hybridizes under stringent conditions to an oligonucleotide consisting of SEQ ID NO:47 (T allele) but does not hybridizes under stringent conditions to an oligonucleotide consisting of SEQ ID NO:46 (A allele), wherein hybrization of the probe under stringent conditions to the nucleic acid from the subject indicates that a progesterone receptor agonist is an effective therapy.

28. The method of claim 20, wherein the subject is not African-American, wherein the method comprises identifying the residue corresponding to single nucleotide polymorphism (SNP) rs471767 in the subject, wherein a guanine (G) allele at SNP rs471767 indicates that a progesterone receptor agonist is an effective therapy.

29. The method of claim 28, wherein the method comprises hybridizing the sample of nucleic acid from the subject with a probe, wherein the probe hybridizes under stringent conditions to an oligonucleotide consisting of SEQ ID NO:59 (G allele) but does not hybridizes under stringent conditions to an oligonucleotide consisting of SEQ ID NO:58 (A allele), wherein hybrization of the probe under stringent conditions to the nucleic acid from the subject indicates that a progesterone receptor agonist is an effective therapy.

30. The method of claim 20, wherein the subject is not African-American, wherein the method comprises identifying the residue corresponding to single nucleotide polymorphism (SNP) rs578029 in the subject, wherein a adenine (A) allele at SNP rs578029 indicates that a progesterone receptor agonist is an effective therapy.

31. The method of claim 30, wherein the method comprises hybridizing the sample of nucleic acid from the subject with a probe, wherein the probe hybridizes under stringent conditions to an oligonucleotide consisting of SEQ ID NO:46 (A allele) but does not hybridizes under stringent conditions to an oligonucleotide consisting of SEQ ID NO:47 (T allele), wherein hybrization of the probe under stringent conditions to the nucleic acid from the subject indicates that a progesterone receptor agonist is an effective therapy.

32. The method of claim 20, wherein the subject is not African-American, wherein the method comprises identifying the residue corresponding to single nucleotide polymorphism (SNP) rs503362 in the subject, wherein a cytosine (C) allele at SNP rs503362 indicates that a progesterone receptor agonist is an effective therapy.

33. The method of claim 30, wherein the method comprises hybridizing the sample of nucleic acid from the subject with a probe, wherein the probe hybridizes under stringent conditions to an oligonucleotide consisting of SEQ ID NO:37 (C allele) but does not hybridizes under stringent conditions to an oligonucleotide consisting of SEQ ID NO:38 (G allele), wherein hybrization of the probe under stringent conditions to the nucleic acid from the subject indicates that a progesterone receptor agonist is an effective therapy.

34. The method of claim 20, wherein the subject is not African-American, wherein the method comprises identifying the residue corresponding to single nucleotide polymorphism (SNP) rs666553 in the subject, wherein a thymidine (T) allele at SNP rs666553 indicates that a progesterone receptor agonist is an effective therapy.

35. The method of claim 30, wherein the method comprises hybridizing the sample of nucleic acid from the subject with a probe, wherein the probe hybridizes under stringent conditions to an oligonucleotide consisting of SEQ ID NO:44 (T allele) but does not hybridizes under stringent conditions to an oligonucleotide consisting of SEQ ID NO:43 (C allele), wherein hybrization of the probe under stringent conditions to the nucleic acid from the subject indicates that a progesterone receptor agonist is an effective therapy.

36. The method of claim 20, wherein the subject has had a prior preterm birth.

37. The method of claim 20, wherein the subject has a family history of preterm birth.

38. The method of claim 20, further comprising detecting fetal fibronectin (fNF) in the subject, wherein a positive fNF test in the midtrimester of pregnancy indicates that a progesterone receptor agonist is an effective therapy.

39. The method of claim 20, further comprising detecting salivary estriol (SalEst) in the subject, wherein a positive SalEst test indicates that a progesterone receptor agonist is an effective therapy.

40. The method of claim 20, further comprising measuring cervical length in the subject, wherein a cervical length less than 25 mm indicates that a progesterone receptor agonist is an effective therapy.

41. A method of prolonging gestation in a subject in need thereof, the method comprising:
a) determining in a sample of nucleic acid from the subject the identity of one or more nucleotides in the progesterone receptor (PR) gene; and
b) administering a therapeutically effective amount of a progesterone receptor agonist to subjects having a substitution of a nucleotide at one or more positions in the PR gene of the subject compared to a control.

42. The method of claim 41, wherein the method comprises detecting an adenine (A) allele at SNP rs471767, a thymidine (T) allele at SNP rs578029, a guanine (G) allele at SNP rs471767, an adenine (A) allele at SNP rs578029, a cytosine (C) allele at SNP rs503362, a thymidine (T) allele at SNP rs666553, or a combination thereof.

43. The method of claim 42, wherein the nucleotide at the one or more position in the PR gene of the subject is detected by allele specific hybridization.

44. The method of claim 43, wherein the adenine (A) allele at SNP rs471767 is detected by a process comprising
a) providing a probe hybridizes under stringent conditions to an oligonucleotide consisting of SEQ ID NO:58 (A allele) but does not hybridizes under stringent conditions to an oligonucleotide consisting of SEQ ID NO:59 (G allele), and
b) monitoring hybridization of said probe to the nucleic acid sample under stringent conditions.

45. The method of claim 43, wherein the thymidine (T) allele at SNP rs578029 is detected by a process comprising
a) providing a probe hybridizes under stringent conditions to an oligonucleotide consisting of SEQ ID NO:47 (T allele) but does not hybridizes under stringent conditions to an oligonucleotide consisting of SEQ ID NO:46 (A allele), and
b) monitoring hybridization of said probe to the nucleic acid sample under stringent conditions.

46. The method of claim 43, wherein the guanine (G) allele at SNP rs471767 is detected by a process comprising
a) providing a probe hybridizes under stringent conditions to an oligonucleotide consisting of SEQ ID NO:59 (G allele) but does not hybridizes under stringent conditions to an oligonucleotide consisting of SEQ ID NO:58 (A allele), and
b) monitoring hybridization of said probe to the nucleic acid sample under stringent conditions.

47. The method of claim 43, wherein the adenine (A) allele at SNP rs578029 is detected by a process comprising
a) providing a probe hybridizes under stringent conditions to an oligonucleotide consisting of SEQ ID NO:46 (A allele) but does not hybridizes under stringent conditions to an oligonucleotide consisting of SEQ ID NO:47 (T allele), and
b) monitoring hybridization of said probe to the nucleic acid sample under stringent conditions.

48. The method of claim 43, wherein the cytosine (C) allele at SNP rs503362 is detected by a process comprising
a) providing a probe hybridizes under stringent conditions to an oligonucleotide consisting of SEQ ID NO:37 (C allele) but does not hybridizes under stringent conditions to an oligonucleotide consisting of SEQ ID NO:38 (G allele), and
b) monitoring hybridization of said probe to the nucleic acid sample under stringent conditions.

49. The method of claim 43, wherein the thymidine (T) allele at SNP rs666553 is detected by a process comprising
a) providing a probe hybridizes under stringent conditions to an oligonucleotide consisting of SEQ ID NO:44 (T allele) but does not hybridizes under stringent conditions to an oligonucleotide consisting of SEQ ID NO:43 (C allele), and
b) monitoring hybridization of said probe to the nucleic acid sample under stringent conditions.

50. The method of claim 42, wherein the nucleotide at the one or more position in the PR gene of the subject is detected by gene sequencing.

51. The method of claim 41, wherein the progesterone receptor agonist is a progestin-agonist.

52. The method of claim 51, wherein the progestin-agonist is progesterone, dydrogesterone, 17α-hydroxyprogesterone (17-OHP), or 17α-hydroxyprogesterone caproate (17-OHPC).

53. The method of claim 41, wherein the subject has had a prior preterm birth.

54. The method of claim 41, wherein the subject has a family history of preterm birth.

55. An array of nucleic acid molecules attached to a solid support, comprising an oligonucleotide that hybridizes under stringent conditions to a nucleic acid molecule consisting of SEQ ID NO:59 (SNP rs471767 G allele) but does not hybridize under stringent conditions to a nucleic acid molecule consisting of SEQ ID NO:58 (SNP rs471767 A allele).

56. A method of predicting the effectiveness of a progesterone receptor (PR) antagonist in a cell or in a subject, comprising determining in a sample of nucleic acid from the cell or subject the identity of one or more nucleotides in the PR gene, wherein a substitution of a nucleotide at one or more positions in the PR gene of the cell or subject compared to a control indicates that the progesterone receptor (PR) antagonist is effective in the cell or subject.
